(19) United States
(12) Patent Application Publication
(10) Pub. No.: US 2009/0197201 A1
(43) Pub. Date: Aug. 6, 2009

(54) FLUOROSURFACTANTS

(76) Inventors: Wolfgang Hierse, Gross-Zimmern (DE); Nikolai (Mykola) Ignatyev, Duisburg (DE); Martin Seidel, Darmstadt (DE); Elvira Montenegro, Weinheim (DE); Peer Kirsch, Yokohama Kanagawa (JP); Andreas Bathe, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/307,290

(22) PCT Filed: Jul. 2, 2007
(86) PCT No.: PCT/EP07/05838

§ 371 Date: Jan. 2, 2009

(30) Foreign Application Priority Data
Jul. 4, 2006 (DE) 10 2006 031 262.7

(51) Int. Cl. C09K 3/00; C07C 381/00; C07C 43/20; C07C 43/02; C07C 307/00; C07C 69/76; C07C 233/07; C07C 211/00; C07D 211/70; C07C 321/10; C07C 49/00; C07F 9/02; C07C 39/24; C09D 7/00; C09D 11/02; G03F 7/00; C11D 1/755; C11D 1/28; C11D 3/32; C11D 3/20; C11D 1/72; C09K 3/18; D06M 15/00; A62D 1/00; C10M 135/22; C10M 135/00; C10M 137/00; C10M 133/00; C10M 131/00; C10M 129/00

(52) U.S. Cl. 430/281.1; 568/74; 568/649; 568/655; 560/14; 560/103; 564/186; 564/442; 546/337; 568/62; 562/431; 568/335; 568/15; 568/775; 106/311; 106/31.13; 430/270.1; 510/492; 510/493; 510/494; 510/501; 510/505; 510/506; 252/70; 252/8.81; 252/182.11; 252/2; 508/244; 508/567; 508/459; 508/551; 508/545; 508/564; 508/580; 508/584; 508/588

(57) ABSTRACT

The present invention relates to the use of end groups Y, where Y stands for (formula I), where Rf stands for CF₃—(CH₂)ᵣ—, CF₃—(CH₂)ᵣ—O—, CF₃—(CH₂)ᵣ—S—, CF₃CF₂—S—, SF₅—(CH₂)ᵣ—, [CF₃—(CH₂)ᵣ]₂N—, [CF₃—(CH₂)ᵣ]NH— or (CF₃)₂N—(CH₂)ᵣ—, B stands for a single bond, O, NH, NR, CH₂, C(O)—O, C(O), S, CH₂—O, O—C(O), N—C(O), C(O)—N, O—C(O)—N, N—C(O)—N, O—SO₂ or SO₂—O, R stands for alkyl having 1 to 4 C atoms, b stands for 0 or 1 and c stands for 0 or 1, q stands for 0 or 1, where at least one radical from b and q stands for 1, and r stands for 0, 1, 2, 3, 4 or 5, as end group in surface-active compounds, to corresponding novel compounds, and to processes for the preparation of these compounds.

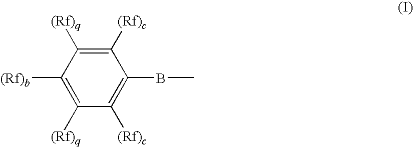

The present invention relates to the use of end groups Y, where Y stands for

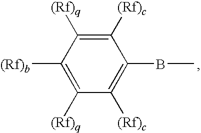

where
Rf stands for CF₃—(CH₂)ᵣ—, CF₃—(CH₂)ᵣ—O—, CF₃—(CH₂)ᵣ—S—, CF₃CF₂—S—, SF₅—(CH₂)ᵣ—, [CF₃—(CH₂)ᵣ]₂N—, [CF₃—(CH₂)ᵣ]NH— or (CF₃)₂N—(CH₂)ᵣ—,
B stands for a single bond, O, NH, NR, CH₂, C(O)—O, C(O), S, CH₂—O, O—C(O), N—C(O), C(O)—N, O—C(O)—N, N—C(O)—N, O—SO₂ or SO₂—O,
R stands for alkyl having 1 to 4 C atoms,
b stands for 0 or 1 and c stands for 0 or 1,
q stands for 0 or 1, where at least one radical from b and q stands for 1, and
r stands for 0, 1, 2, 3, 4 or 5,
as end group in surface-active compounds, to corresponding novel compounds, and to processes for the preparation of these compounds.

Fluorosurfactants have an outstanding ability to reduce surface energy, which is utilised, for example, in the hydrophobicisation of surfaces, such as textile impregnation, hydrophobicisation of glass, or de-icing of aircraft wings.

In general, however, fluorosurfactants contain perfluoroalkyl substituents, which are degraded in the environment by biological and other oxidation processes to give perfluoroalkanecarboxylic acids and -sulfonic acids. These are regarded as persistent and are in some cases suspected of causing health damage (G. L. Kennedy, Jr., J. L. Butenhoff, G. W. Olsen, J. C. O'Connor, A. M. Seacat, R. G. Perkins, L. B. Biegel, S. R. Murphy, D. G. Farrar, Critical Reviews in Toxicology 2004, 34, 351-384). In addition, relatively long-chain perfluoroalkanecarboxylic acids and -sulfonic acids accumulate in the food chain.

There is therefore a demand for novel surface-active substances which have a property profile comparable to the classical fluorosurfactants and which can preferably be degraded oxidatively or reductively. Particularly advantageous compounds here are those which do not leave behind any persistent organofluorine degradation products on degradation.

The Omnova company markets polymers whose side chains contain terminal CF₃ or C₂F₅ groups. International patent application WO 03/010128 describes perfluoroalkyl-substituted amines, acids, amino acids and thio-ether acids which contain a C₃₋₂₀-perfluoroalkyl group.

JP-A-2001/133984 discloses surface-active compounds containing perfluoroalkoxy chains which are suitable for use in antireflection coatings. JP-A-09/111,286 discloses the use of perfluoropolyether surfactants in emulsions.

The earlier German patent application DE 102005000858 A describes compounds which carry at least one terminal pentafluorosulfuranyl group or at least one terminal trifluoromethoxy group and contain a polar end group, are surface-active and are highly suitable as surfactants.

Nevertheless, there continues to be a demand for novel fluorinated end groups and compounds which contain these end groups. It is advantageous if such compounds can be used as surfactants or surfactant precursors.

The present invention therefore relates firstly to the use of end groups Y, where Y stands for

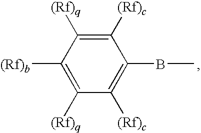

where
Rf stands for CF₃—(CH₂)ᵣ—, CF₃—(CH₂)ᵣ—O—, CF₃—(CH₂)ᵣ—S—, CF₃CF₂—S—, SF₅—(CH₂)ᵣ—, [CF₃—(CH₂)ᵣ]₂N—, [CF₃—(CH₂)ᵣ]NH— or (CF₃)₂N—(CH₂)ᵣ—,
B stands for a single bond, O, NH, NR, CH₂, C(O)—O, C(O), S, CH₂—O, O—C(O), N—C(O), C(O)—N, O—C(O)—N, N—C(O)—N, O—SO₂ or SO₂—O,
R stands for alkyl having 1 to 4 C atoms,
b stands for 0 or 1 and c stands for 0 or 1,
q stands for 0 or 1, where at least one radical from b and q stands for 1, and
r stands for 0, 1, 2, 3, 4 or 5,
as end group in surface-active compounds.

Rf preferably stands for CF₃—(CH₂)ᵣ—, CF₃—(CH₂)ᵣ—O—, CF₃—(CH₂)ᵣ—S or [CF₃—(CH₂)ᵣ]₂N—. A preferred variant of the invention encompasses fluorine groups, also abbreviated to Rf below, in which r stands for 0, 1, 2 or 3, in particular for 0, 1 or 2, where r preferably stands for 0.

In a particularly preferred embodiment of the present invention, Rf stands for CF₃—, CF₃—O—, CF₃—CH₂—CH₂—O—, CF₃—S—, CF₃CF₂—S—, SF₅—, CF₃—CH₂—CH₂—S—, (CF₃)₂—N— and (CF₃—CH₂—CH₂)₂—N—, in particular for CF₃—, CF₃—O—, CF₃—S— and (CF₃)₂—N—.

A further preferred variant of the invention encompasses the groups Rf which are equal to CF₃—, CF₃—S—, CF₃CF₂—S—, SF₅— or (CF₃)₂N—.

Particularly preferred groups B are O, S, CH₂O, CH₂, C(O) and OC(O). In particular, B equal to O and OC(O) are preferred.

A preferred variant of the invention encompasses groups Y which have a combination of the variables in their preferred or particularly preferred ranges.

A particularly preferred variant of the invention encompasses the groups Y which are equal to CF₃—Ar—O, CF₃—O—Ar—O, CF₃—CH₂—CH₂—O—Ar—O, CF₃—S—Ar—O, CF₃CF₂—S—Ar—O, SF₅—Ar—O, CF₃—CH₂—CH₂—S—Ar—O, (CF₃)₂—N—Ar—O, (CF₃—CH₂—CH₂)₂—N—Ar—O, CF₃—Ar—OC(O), CF₃—O—Ar—OC(O), CF₃—CH₂—CH₂—O—Ar—OC(O), CF₃—S—Ar—OC(O), CF₃CF₂—S—Ar—OC(O), SF₅—Ar—OC(O), CF₃—CH₂—CH₂—S—Ar—OC(O), (CF₃)₂—N—Ar—OC(O) and (CF₃—CH₂—CH₂)₂—N—Ar—OC(O), in particular equal to CF₃—Ar—O, CF₃—O—Ar—O, CF₃—S—Ar—O, (CF₃)₂—N—Ar—O, CF₃—Ar—OC(O), CF₃—O—Ar—OC(O), CF₃—S—Ar—OC(O) and (CF₃)₂—N—Ar—OC(O).

A particularly preferred variant of the invention encompasses Y equal to CF₃—Ar—O and CF₃—Ar—OC(O).

R preferably stands for alkyl having 1, 2 or 3 C atoms, in particular having 1 or 2 C atoms.

In a variant of the present invention, it is preferred for q to stand for 0 and for at least one c and/or b each to stand for 1. It is particularly preferred for all c and b to stand for 1, i.e. the aromatic rings are substituted by fluorine groups in the o,p,o-position.

In a further variant of the invention, it is preferred for all q and b each to stand for 0 and for at least one c to stand for 1. It is particularly preferred for both c to stand for 1, i.e. the aromatic rings are substituted by fluorine groups in the o,o-position.

In a further variant of the invention, it is preferred for all c and q each to stand for 0 and for b to stand for 1, i.e. the aromatic rings are substituted by fluorine groups in the p-position.

Particular preference is given to the use of compounds of the formula I which have a combination of the variables in their preferred or particularly preferred ranges.

The end group Y in the surface-active compounds is preferably bonded to a saturated or unsaturated, optionally aromatic, branched or unbranched, optionally substituted, optionally heteroatom-substituted hydrocarbon unit. The hydrocarbon units can be aliphatic or aromatic units, optionally provided with heteroatoms. It is particularly preferred here for the hydrocarbon units or the entire molecule to be free from further fluorine atoms.

Besides the fluorinated end groups mentioned, the compounds to be used in accordance with the invention preferably contain no further fluorinated groups.

In a variant of the invention, the end group Y occurs a number of times in the surface-active compound, and the surface-active compound is preferably an oligomer or polymer.

In another, likewise preferred variant of the invention, the end group Y occurs only once, twice or three times in the surface-active compound, where compounds in which the end group only occurs once are particularly preferred. The compounds to be used in accordance with the invention are preferably low-molecular-weight compounds of the formula I

Y-spacer-X I

where
Y stands for

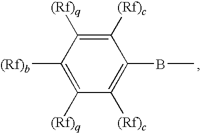

where
Rf stands for CF₃—(CH₂)ᵣ—, CF₃—(CH₂)ᵣ—O—, CF₃—(CH₂)ᵣ—S—, CF₃CF₂—S—, SF₅—(CH₂)ᵣ—, [CF₃—(CH₂)ᵣ]₂N—, [CF₃—(CH₂)ᵣ]NH— or (CF₃)₂N—(CH₂)ᵣ—,
B stands for a single bond, O, NH, NR, CH₂, C(O)—O, C(O), S, CH₂—O, O—C(O), N—C(O), C(O)—N, O—C(O)—N, N—C(O)—N, O—SO₂ or SO₂—O,
R stands for alkyl having 1 to 4 C atoms,
b stands for 0 or 1 and c stands for 0 or 1,
q stands for 0 or 1, where at least one radical from b and q stands for 1,
r stands for 0, 1, 2, 3, 4 or 5,
spacer stands for a saturated or unsaturated, optionally aromatic, branched or unbranched, optionally substituted hydrocarbon unit, and
X stands for a cationic, nonionic, amphoteric or anionic polar group or a polymerisable group.

It is furthermore particularly preferred for the compound of the formula I to be selected from the compounds of the formulae Ia to Ig

Y—(CH₂)ₙ—X Ia

Y—CH₂—CH(Hal)—(CH₂)₍ₙ₋₁₎—X Ib

Y—(CH₂)ₙ₋₁CH═CH—(CH₂)_{(n′-1)}—X Ic

Y—(CH₂)ₙ₋₁—Ar—(CH₂)_{(n′-1)}—X Id

Y—(CH₂)ₙ₋₁—C≡C—(CH₂)ₙ—X Ie

Y—(CH₂)ₙ-Q-(CH₂)_{n′}—X If

Y—[(CH₂)_{z}—O]—[(CH₂)ₓ—O]_{y}—(CH₂)ₜ—X Ig

in which Y stands for

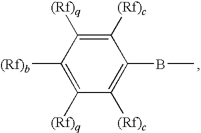

where
Rf stands for CF₃—(CH₂)ᵣ—, CF₃—(CH₂)ᵣ, —, CF₃—(CH₂)ᵣ—S—, CF₃CF₂—S—, SF₅—(CH₂)ᵣ—, [CF₃—(CH₂)ᵣ]₂N—, [CF₃—(CH₂)ᵣ]NH— or (CF₃)₂N—(CH₂)ᵣ—,
B stands for a single bond, O, NH, NR, CH2, C(O)—O, C(O), S, CH₂—O, O—C(O), N—C(O), C(O)—N, O—C(O)—N, N—C(O)—N, O—SO₂ or SO₂—O,
R stands for alkyl having 1 to 4 C atoms,
b stands for 0 or 1 and c stands for 0 or 1,
q stands for 0 or 1, where at least one radical from b and q stands for 1,
r stands for 0, 1, 2, 3, 4 or 5,
x and t, independently of one another, stand for 1-5,
y stands for 0-5,
z stands for 1-12,
n and n′, independently of one another, stand for an integer from the range 1 to 30,
X stands for a cationic, nonionic, amphoteric or anionic polar group or a polymerisable group,
(Hal) stands for F, Cl, Br or I,
Q stands for O, S or N, and
Ar stands for aryl,
   and corresponding salts of the compounds of the formula Ia, Ib or Ic or Id.

Preference is given to compounds of the formulae Ia to Ig in which the group Y is present in the preferred ranges of its variables mentioned above, in particular in the said combinations of the preferred variables.

A particularly preferred variant of the invention encompasses the groups Y equal to CF₃—Ar—O, CF₃—O—Ar—O, CF₃—CH₂—CH₂—O—Ar—O, CF₃—S—Ar—O, CF₃CF₂—S—Ar—O, SF₅—Ar—O, CF₃—CH₂—CH₂—S—Ar—O, (CF₃)₂—N—Ar—O, (CF₃—CH₂—CH₂)₂—N—Ar—O, CF₃—Ar—OC(O), CF₃—O—Ar—OC(O), CF₃—CH₂—CH₂—O—Ar—OC(O), CF₃—S—Ar—OC(O), CF₃CF₂—S—Ar—OC(O), SF₅—Ar—OC(O), CF₃—CH₂—CH₂—S—Ar—OC(O), (CF₃)₂—N—Ar—OC(O) and (CF₃—CH₂—CH₂)₂—N—Ar—OC(O), in particular equal to CF₃—Ar—O, CF₃—O—Ar—O, CF₃—S—Ar—O, (CF₃)₂—N—Ar—O, CF₃—Ar—OC(O), CF₃—O—Ar—OC(O), CF₃—S—Ar—OC(O) and (CF₃)₂—N—Ar—OC(O).

A particularly preferred variant of the invention encompasses Y equal to CF₃—Ar—O and CF₃—Ar—OC(O).

A preferred range for n and/or n′ is 3 to 24, in particular 3 to 18. In particular, compounds where n and/or n′ are in the range from 3 to 16 are preferred, in particular in the range 8-16. In a particularly preferred variant of the invention, n and/or n′ are, independently of one another, even numbers.

Q preferably stands for O or S.

In another, likewise preferred variant of the invention, n in the formula Ia stands for 1 or 2, and X preferably stands for a functional group, preferably selected from —CH═CH₂, —C≡CR², —CHO, —C(═O)CH₃, —COOH, —OH, —SH, —Cl, —Br, —I. These compounds are particularly suitable as intermediates for the construction of further compounds according to the invention.

Particular preference is given to the use of compounds of the formulae Ia to Ig which have a combination of the variables in their preferred or particularly preferred ranges.

Very particular preference is given here to the use of compounds of the formula Ia in which n particularly preferably stands for an integer from the range 3 to 24 and particularly preferably for an integer from the range 3 to 18, in particular 8 to 16. It is in turn preferred in a variant of the invention for n to be an even number.

Particular preference is given in accordance with the invention to the use of the above-mentioned compounds as surfactants.

If the compounds of the formula I are anionic compounds or compounds which can be converted anionically into salts, it is preferred for the counter-ion present to be an alkali metal ion, preferably Li⁺, Na⁺ or K⁺, an alkaline-earth metal ion or NH₄⁺. If the compounds of the formula I are cationic compounds or compounds which can be converted cationically into salts, it is preferred for the counterion present to be a halide, such as Cl⁻, Br⁻, I⁻, or CH₃SO₃⁻, CF₃SO₃⁻, CH₃PhSO₃⁻ or PhSO₃⁻.

Advantages of the compounds according to the invention or of the use according to the invention of the said compounds or of the compositions according to the invention may be, in particular:
a surface activity which may be the same as or superior to that of the conventional hydrocarbon surfactants with respect to efficiency and/or effectiveness, and/or
biological and/or abiotic degradability of the substances without the formation of persistent perfluorinated degradation products, and/or
good processability in formulations, and/or
storage stability.

In a preferred group of compounds of the formula I to be employed in accordance with the invention, X stands for an anionic polar group selected from —COOM, —SO₃M, —OSO₃M, —PO₃M₂, —OPO₃M₂, —(OCH₂CHR)ₘ—O—(CH₂)ₒ—COOM, —(OCH₂CHR)ₘ—O—(CH₂)ₒ—SO₃M, —(OCH₂CHR)ₘ—O—(CH₂)ₒ—OSO₃M, —(OCH₂CHR)ₘ—O—(CH₂)ₒ—PO₃M₂, —(OCH₂CHR)ₘ—O—(CH₂)ₒ—OPO₃M₂, where M stands for H or an alkali metal ion, preferably Li⁺, Na⁺ or K⁺, or NH₄⁺ or tetra-C₁₋₆-alkylammonium or tetra-C₁₋₆-alkylphosphonium, R stands for H or C₁₋₄-alkyl, m stands for an integer from the range from 1 to 1000, and o stands for an integer selected from 1, 2, 3 or 4.

The preferred anionic groups here include, in particular, —COOM, —SO₃M, —OSO₃M, and —(OCH₂CHR)ₘ—O—(CH₂)ₒ—COOM, —(OCH₂CHR)ₘ—O—(CH₂)ₒ—SO₃M and —(OCH₂CHR)ₘ—O—(CH₂)ₒ—OSO₃M, where each individual one of these groups per se may be preferred.

In another, likewise preferred group of compounds of the formula I to be employed in accordance with the invention, X stands for a cationic polar group selected from —NR¹R²R³⁺Z⁻, —PR¹R²R³⁺Z⁻,

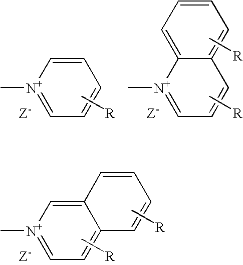

where R stands for H or C₁₋₄-alkyl in any desired position,
Z⁻ stands for Cl⁻, Br⁻, I⁻, CH₃SO₃⁻, CF₃SO₃⁻, CH₃PhSO₃⁻, PhSO₃⁻,
R¹, R² and R³ each, independently of one another, stand for H, C₁₋₃₀-alkyl, Ar or —CH₂Ar, and
Ar stands for an unsubstituted or mono- or polysubstituted aromatic ring or condensed ring system having 6 to 18 C atoms, in which, in addition, one or two CH groups may be replaced by N.

The preferred cationic groups here include, in particular, —NR¹R²R³⁺Z⁻ and

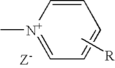

where each individual one of these groups per se may be preferred.

In a further preferred group of compounds of the formula I or formulae Ia to Ig to be employed in accordance with the invention, X stands for a nonionic polar group selected from —Cl, —Br, —I, —(OCH₂CHR)ₘ—OH, —(OCH₂CHR)ₘ—SH, —O-(glycoside)ₒ, —(OCH₂CHR)ₘ—OCH₂—CHOH—CH₂—OH, —(OCH₂CHR)ₘ—OCH₂Ar(—NCO)ₚ, —(OCH₂CHR)ₘ—OAr(—NCO)ₚ, —SiR¹R²Z, —SiR¹Z₂, —SiZ₃, —COZ, —(OCH₂CHR)ₘ—SO₂CH═CH₂, —SO₂Z,

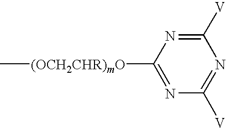

m stands for an integer from the range from 0 to 1000,
n stands for 0 or 1,
o stands for an integer from the range from 1 to 10,
p stands for 1 or 2,
R stands for H or C₁₋₄-alkyl,
R¹ and R² each, independently of one another, stand for C₁₋₃₀-alkyl, Ar or —CH₂Ar,
Ar stands for an unsubstituted, mono- or polysubstituted aromatic ring or condensed ring system having 6 to 18 C atoms, in which, in addition, one or two CH groups may be replaced by C═O, and
glycoside stands for an etherified carbohydrate, preferably for a mono-, di-, tri- or oligoglucoside,
all Z each, independently of one another, stand for —H, —Cl, —F, —NR¹R², —OR¹ or —N-imidazolyl, and
V stands for Cl or F.

The preferred nonionic polar groups here include, in particular, —(OCH₂CHR)ₘ—OH and —O-(glycoside)ₒ, where each individual one of these groups per se may be preferred.

In addition, compounds of the formula I in which X stands for a polymerisable group selected from —(OCH₂CHR)ₘOCOCR═CH₂, —(OCH₂CHR)ₘ—, OCR═CH₂,

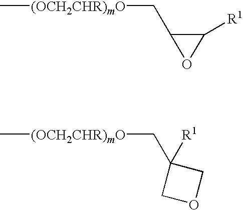

where m stands for an integer from the range from 0 to 1000, and R and R¹ stand for H or C₁₋₄-alkyl or Y-spacer-(OCH₂CHR)ₘ—OCH₂—, may be preferred or preferably used in accordance with the invention.

These compounds are preferably converted into polymers containing corresponding side chains, which can themselves again be employed in the sense according to the invention. The present invention also relates to the use of these polymers.

In addition, compounds of the formula I in which X stands for a functional group selected from —CR²═CR³R⁴, —C═CH, —CHO, —C(═O)CH₃, —COOH, —OH, —SH, —Cl, —Br, —I, where R², R³ and R⁴ each, independently of one another, stand for H or Y-spacer- or C₁₋₄-alkyl, may be preferred or preferably used in accordance with the invention.

In addition, compounds in which X stands for an amphoteric group selected from the functional groups of acetyldiamines, N-alkylamino acids, betaines, amine oxides or corresponding derivatives may be preferred or preferably used in accordance with the invention. In preferred compounds from this class of substances, X is a group selected from

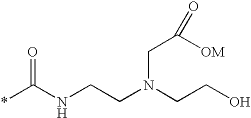

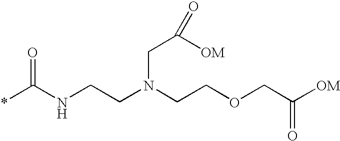

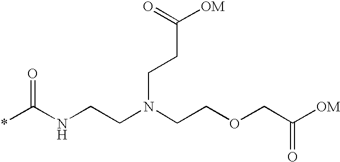

—[C(═O)—NH—(CH₂)₍₁₋₈₎]_{(0 or 1)}—N⁺R¹R²—O⁻,
where R¹ and R² each,
independently of one another, stand for a C1-8-alkyl radical, preferably
methyl or ethyl
—NH—CH₂—COOM; —NH—CH₂—CH₂—COOM

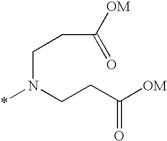

—[C(═O)—NH—(CH₂)₍₁₋₈₎]_{(0 or 1)}—N⁺R¹R²—CH₂—COO⁻,
where R¹ and R² each,
independently of one another, stand for a C1-8-alkyl radical, preferably
methyl or ethyl
—[C(═O)—NH—(CH₂)₁₋₃—N⁺R¹R²—CH₂—CH(OH)—CH₂—(O)_{(0 or 1)}—(S or P)O₃⁻,
where R¹ and R² each, independently of one another, stand for a C1-8-
alkyl radical, preferably methyl or ethyl

The particularly preferred compounds according to the invention include the compounds shown in the following table. These compounds may themselves be surfactants or they are the corresponding acids of surfactants or the precursors of surfactants. A particularly preferred variant of the invention encompasses compounds containing CF₃—Ar—O— and CF₃—Ar—OC(O)— groups. In a variant of the invention, it is in turn preferred for n and/or n′ to be even numbers.

TABLE
CF₃—Ar—O—(CH₂)₃—COOH; CF₃—Ar—O—(CH₂)₃—SO₃H; CF₃—Ar—O—(CH₂)₃—O—SO₃H; CF₃—Ar—O—(CH₂)₃—SH;
CF₃—Ar—O—(CH₂)₃—OH; CF₃—Ar—O—(CH₂)₃—N⁺R¹R²R³Z⁻; CF₃—Ar—O—(CH₂)₃—P⁺R¹R²R³Z⁻;
CF₃—Ar—O—(CH₂)₃—O-glucoside; CF₃—Ar—O—(CH₂)₃—O—PO₃H;
CF₃—Ar—O—(CH₂)₄—COOH; CF₃—Ar—O—(CH₂)₄—SO₃H; CF₃—Ar—O—(CH₂)₄—O—SO₃H; CF₃—Ar—O—(CH₂)₄—SH;
CF₃—Ar—O—(CH₂)₄—OH; CF₃—Ar—O—(CH₂)₄—N⁺R¹R²R³Z⁻; CF₃—Ar—O—(CH₂)₄—P⁺R¹R²R³Z⁻;
CF₃—Ar—O—(CH₂)₄—O-glucoside; CF₃—Ar—O—(CH₂)₄—O—PO₃H;
CF₃—Ar—O—(CH₂)₅—COOH; CF₃—Ar—O—(CH₂)₅—SO₃H; CF₃—Ar—O—(CH₂)₅—O—SO₃H; CF₃—Ar—O—(CH₂)₅—SH;
CF₃—Ar—O—(CH₂)₅—OH; CF₃—Ar—O—(CH₂)₅—N⁺R¹R²R³Z⁻; CF₃—Ar—O—(CH₂)₅—P⁺R¹R²R³Z⁻;
CF₃—Ar—O—(CH₂)₅—O-glucoside, CF₃—Ar—O—(CH₂)₅—O—PO₃H
CF₃—Ar—O—(CH₂)₆—COOH; CF₃—Ar—O—(CH₂)₆—SO₃H; CF₃—Ar—O—(CH₂)₆—O—SO₃H; CF₃—Ar—O—(CH₂)₆—SH;
CF₃—Ar—O—(CH₂)₆—OH; CF₃—Ar—O—(CH₂)₆—N⁺R¹R²R³Z⁻; CF₃—Ar—O—(CH₂)₆—P⁺R¹R²R³Z⁻;
CF₃—Ar—O—(CH₂)₆—O-glucoside; CF₃—Ar—O—(CH₂)₆—OPO₃H
CF₃—Ar—O—(CH₂)₇—COOH; CF₃—Ar—O—(CH₂)₇—SO₃H; CF₃—Ar—O—(CH₂)₇—O—SO₃H; CF₃—Ar—O—(CH₂)₇—SH;
CF₃—Ar—O—(CH₂)₇—OH; CF₃—Ar—O—(CH₂)₇—N⁺R¹R²R³Z⁻; CF₃—Ar—O—(CH₂)₇—P⁺R¹R²R³Z⁻;
CF₃—Ar—O—(CH₂)₇—O-glucoside; CF₃—Ar—O—(CH₂)₇—OPO₃H
CF₃—Ar—O—(CH₂)₈—COOH; CF₃—Ar—O—(CH₂)₈—SO₃H; CF₃—Ar—O—(CH₂)₈—O—SO₃H; CF₃—Ar—O—(CH₂)₈—SH;
CF₃—Ar—O—(CH₂)₈—OH; CF₃—Ar—O—(CH₂)₈—N⁺R¹R²R³Z⁻; CF₃—Ar—O—(CH₂)₈—P⁺R¹R²R³Z⁻;
CF₃—Ar—O—(CH₂)₈—O-glucoside; CF₃—Ar—O—(CH₂)₈—OPO₃H
CF₃—Ar—O—(CH₂)₉—COOH; CF₃—Ar—O—(CH₂)₉—SO₃H; CF₃—Ar—O—(CH₂)₉—O—SO₃H;; CF₃—Ar—O—(CH₂)₉—SH;
CF₃—Ar—O—(CH₂)₉—OH; CF₃—Ar—O—(CH₂)₉—N⁺R¹R²R³Z⁻; CF₃—Ar—O—(CH₂)₉—P⁺R¹R²R³Z⁻;
CF₃—Ar—O—(CH₂)₉—O-glucoside; CF₃—Ar—O—(CH₂)₉—OPO₃H
CF₃—Ar—O—(CH₂)₁₀—COOH; CF₃—Ar—O—(CH₂)₁₀—SO₃H; CF₃—Ar—O—(CH₂)₁₀—O—SO₃H;
CF₃—Ar—O—(CH₂)₁₀—SH; CF₃—Ar—O—(CH₂)₁₀—OH; CF₃—Ar—O—(CH₂)₁₀—N⁺R¹R²R³Z⁻; CF₃—Ar—O—(CH₂)₁₀—P⁺R¹R²R³Z⁻;
CF₃—Ar—O—(CH₂)₁₀—O-glucoside; CF₃—Ar—O—(CH₂)₁₀—OPO₃H
CF₃—Ar—O—(CH₂)₁₁—COOH; CF₃—Ar—O—(CH₂)₁₁—SO₃H; CF₃—Ar—O—(CH₂)₁₁—O—SO₃H;
CF₃—Ar—O—(CH₂)₁₁—SH; CF₃—Ar—O—(CH₂)₁₁—OH; CF₃—Ar—O—(CH₂)₁₁—N⁺R¹R²R³Z⁻; CF₃—Ar—O—(CH₂)₁₁—P⁺R¹R²R³Z⁻;
CF₃—Ar—O—(CH₂)₁₁—O-glucoside; CF₃—Ar—O—(CH₂)₁₁—OPO₃H
CF₃—Ar—O—(CH₂)₁₂—COOH; CF₃—Ar—O—(CH₂)₁₂—SO₃H; CF₃—Ar—O—(CH₂)₁₂—O—SO₃H;
CF₃—Ar—O—(CH₂)₁₂—SH; CF₃—Ar—O—(CH₂)₁₂—OH; CF₃—Ar—O—(CH₂)₁₂—N⁺R¹R²R³Z⁻; CF₃—Ar—O—(CH₂)₁₂—P⁺R¹R²R³Z⁻;
CF₃—Ar—O—(CH₂)₁₂—O-glucoside; CF₃—Ar—O—(CH₂)₁₂—OPO₃H
CF₃—Ar—O—(CH₂)₁₃—COOH; CF₃—Ar—O—(CH₂)₁₃—SO₃H; CF₃—Ar—O—(CH₂)₁₃—O—SO₃H;
CF₃—Ar—O—(CH₂)₁₃—SH; CF₃—Ar—O—(CH₂)₁₃—OH; CF₃—Ar—O—(CH₂)₁₃—N⁺R¹R²R³Z⁻; CF₃—Ar—O—(CH₂)₁₃—P⁺R¹R²R³Z⁻;
CF₃—Ar—O—(CH₂)₁₃—O-glucoside; CF₃—Ar—O—(CH₂)₁₃—OPO₃H
CF₃—Ar—O—(CH₂)₁₄COOH; CF₃—Ar—O—(CH₂)₁₄—SO₃H; CF₃—Ar—O—(CH₂)₁₄—O—SO₃H;
CF₃—Ar—O—(CH₂)₁₄—SH; CF₃—Ar—O—(CH₂)₁₄—OH; CF₃—Ar—O—(CH₂)₁₄—N⁺R¹R²R³Z⁻; CF₃—Ar—O—(CH₂)₁₄—P⁺R¹R²R³Z⁻;
CF₃—Ar—O—(CH₂)₁₄—O-glucoside; CF₃—Ar—O—(CH₂)₁₄—OPO₃H
CF₃—Ar—O—(CH₂)₁₅—COOH; CF₃—Ar—O—(CH₂)₁₅—SO₃H; CF₃—Ar—O—(CH₂)₁₅—O—SO₃H;;
CF₃—Ar—O—(CH₂)₁₅—SH; CF₃—Ar—O—(CH₂)₁₅—OH; CF₃—Ar—O—(CH₂)₁₅—N⁺R¹R²R³Z⁻; CF₃—Ar—O—(CH₂)₁₅—P⁺R¹R²R³Z⁻;
CF₃—Ar—O—(CH₂)₁₅—O-glucoside; CF₃—Ar—O—(CH₂)₁₅—OPO₃H
CF₃—Ar—O—(CH₂)₁₆—COOH; CF₃—Ar—O—(CH₂)₁₆—SO₃H; CF₃—Ar—O—(CH₂)₁₆—O—SO₃H;
CF₃—Ar—O—(CH₂)₁₆—SH; CF₃—Ar—O—(CH₂)₁₆—OH; CF₃—Ar—O—(CH₂)₁₆—N⁺R¹R²R³Z⁻; CF₃—Ar—O—(CH₂)₁₆—P⁺R¹R²R³Z⁻;
CF₃—Ar—O—(CH₂)₁₆—O-glucoside; CF₃—Ar—O—(CH₂)₁₆—OPO₃H
CF₃—O—Ar—O—(CH₂)₃—COOH; CF₃—O—Ar—O—(CH₂)₃—SO₃H; CF₃—O—Ar—O—(CH₂)₃—O—SO₃H;
CF₃—O—Ar—O—(CH₂)₃—SH, CF₃—O—Ar—O—(CH₂)₃—OH; CF₃—O—Ar—O—(CH₂)₃—N⁺R¹R²R³Z⁻;
CF₃—O—Ar—O—(CH₂)₃—P⁺R¹R²R³Z⁻; CF₃—O—Ar—O—(CH₂)₃—O-glucoside;
CF₃—O—Ar—O—(CH₂)₃—OPO₃H
CF₃—O—Ar—O—(CH₂)₄—COOH; CF₃—O—Ar—O—(CH₂)₄—SO₃H; CF₃—O—Ar—O—(CH₂)₄—O—SO₃H;
CF₃—O—Ar—O—(CH₂)₄—SH, CF₃—O—Ar—O—(CH₂)₄—OH; CF₃—O—Ar—O—(CH₂)₄—N⁺R¹R²R³Z⁻;
CF₃—O—Ar—O—(CH₂)₄—P⁺R¹R²R³Z⁻; CF₃—O—Ar—O—(CH₂)₄—O-glucoside;
CF₃—O—Ar—O—(CH₂)₄—OPO₃H
CF₃—O—Ar—O—(CH₂)₅—COOH; CF₃—O—Ar—O—(CH₂)₅—SO₃H; CF₃—O—Ar—O—(CH₂)₅—O—SO₃H;
CF₃—O—Ar—O—(CH₂)₅—SH; CF₃—O—Ar—O—(CH₂)₅—OH; CF₃—O—Ar—O—(CH₂)₅—N⁺R¹R²R³Z⁻;
CF₃—O—Ar—O—(CH₂)₅—P⁺R¹R²R³Z⁻; CF₃—O—Ar—O—(CH₂)₅—O-glucoside,
CF₃—O—Ar—O—(CH₂)₅—O—PO₃H
CF₃—O—Ar—O—(CH₂)₆—COOH; CF₃—O—Ar—O—(CH₂)₆—SO₃H; CF₃—O—Ar—O—(CH₂)₆—O—SO₃H;
CF₃—O—Ar—O—(CH₂)₆—SH; CF₃—O—Ar—O—(CH₂)₆—OH; CF₃—O—Ar—O—(CH₂)₆—N⁺R¹R²R³Z⁻;
CF₃—O—Ar—O—(CH₂)₆—P⁺R¹R²R³Z⁻; CF₃—O—Ar—O—(CH₂)₆—O-glucoside;
CF₃—O—Ar—O—(CH₂)₆—OPO₃H
CF₃—O—Ar—O—(CH₂)₇—COOH; CF₃—O—Ar—O—(CH₂)₇—SO₃H; CF₃—O—Ar—O—(CH₂)₇—O—SO₃H;
CF₃—O—Ar—O—(CH₂)₇—SH; CF₃—O—Ar—O—(CH₂)₇—OH; CF₃—O—Ar—O—(CH₂)₇—N⁺R¹R²R³Z⁻;
CF₃—O—Ar—O—(CH₂)₇—P⁺R¹R²R³Z⁻; CF₃—O—Ar—O—(CH₂)₇—O-glucoside;
CF₃—O—Ar—O—(CH₂)₇—OPO₃H
CF₃—O—Ar—O—(CH₂)₈—COOH; CF₃—O—Ar—O—(CH₂)₈—SO₃H; CF₃—O—Ar—O—(CH₂)₈—O—SO₃H;
CF₃—O—Ar—O—(CH₂)₈—SH; CF₃—O—Ar—O—(CH₂)₈—OH; CF₃—O—Ar—O—(CH₂)₈—N⁺R¹R²R³Z⁻;
CF₃—O—Ar—O—(CH₂)₈—P⁺R¹R²R³Z⁻; CF₃—O—Ar—O—(CH₂)₈—O-glucoside;
CF₃—O—Ar—O—(CH₂)₈—OPO₃H
CF₃—O—Ar—O—(CH₂)₉—COOH; CF₃—O—Ar—O—(CH₂)₉—SO₃H; CF₃—O—Ar—O—(CH₂)₉—O—SO₃H;
CF₃—O—Ar—O—(CH₂)₉—SH; CF₃—O—Ar—O—(CH₂)₉—OH; CF₃—O—Ar—O—(CH₂)₉—N⁺R¹R²R³Z⁻;
CF₃—O—Ar—O—(CH₂)₉—P⁺R¹R²R³Z⁻; CF₃—O—Ar—O—(CH₂)₉—O-glucoside;
CF₃—O—Ar—O—(CH₂)₉—OPO₃H
CF₃—O—Ar—O—(CH₂)₁₀—COOH; CF₃—O—Ar—O—(CH₂)₁₀—SO₃H; CF₃—O—Ar—O—(CH₂)₁₀—O—SO₃H;
CF₃—O—Ar—O—(CH₂)₁₀—SH; CF₃—O—Ar—O—(CH₂)₁₀—OH; CF₃—O—Ar—O—(CH₂)₁₀—N⁺R¹R²R³Z⁻;
CF₃—O—Ar—O—(CH₂)₁₀—P⁺R¹R²R³Z⁻; CF₃—O—Ar—O—(CH₂)₁₀—O-glucoside;
CF₃—O—Ar—O—(CH₂)₁₀—OPO₃H
CF₃—O—Ar—O—(CH₂)₁₁—COOH; CF₃—O—Ar—O—(CH₂)₁₁—SO₃H; CF₃—O—Ar—O—(CH₂)₁₁—O—SO₃H;
CF₃—O—Ar—O—(CH₂)₁₁—SH; CF₃—O—Ar—O—(CH₂)₁₁—OH; CF₃—O—Ar—O—(CH₂)₁₁—N⁺R¹R²R³Z⁻;
CF₃—O—Ar—O—(CH₂)₁₁—P⁺R¹R²R³Z⁻; CF₃—O—Ar—O—(CH₂)₁₁—O-glucoside;
CF₃—O—Ar—O—(CH₂)₁₁—OPO₃H
CF₃—O—Ar—O—(CH₂)₁₂—COOH; CF₃—O—Ar—O—(CH₂)₁₂—SO₃H; CF₃—O—Ar—O—(CH₂)₁₂—O—SO₃H;
CF₃—O—Ar—O—(CH₂)₁₂—SH; CF₃—O—Ar—O—(CH₂)₁₂—OH; CF₃—O—Ar—O—(CH₂)₁₂—N⁺R¹R²R³Z⁻;
CF₃—O—Ar—O—(CH₂)₁₂—P⁺R¹R²R³Z⁻; CF₃—O—Ar—O—(CH₂)₁₂—O-glucoside;
CF₃—O—Ar—O—(CH₂)₁₂—OPO₃H
CF₃—O—Ar—O—(CH₂)₁₃—COOH; CF₃—O—Ar—O—(CH₂)₁₃—SO₃H; CF₃—O—Ar—O—(CH₂)₁₃—O—SO₃H;
CF₃—O—Ar—O—(CH₂)₁₃—SH; CF₃—O—Ar—O—(CH₂)₁₃—OH; CF₃—O—Ar—O—(CH₂)₁₃—N⁺R¹R²R³Z⁻;
CF₃—O—Ar—O—(CH₂)₁₃—P⁺R¹R²R³Z⁻; CF₃—O—Ar—O—(CH₂)₁₃—O-glucoside;
CF₃—O—Ar—O—(CH₂)₁₃—OPO₃H
CF₃—O—Ar—O—(CH₂)₁₄—COOH; CF₃—O—Ar—O—(CH₂)₁₄—SO₃H; CF₃—O—Ar—O—(CH₂)₁₄—O—SO₃H;
CF₃—O—Ar—O—(CH₂)₁₄—SH; CF₃—O—Ar—O—(CH₂)₁₄—OH; CF₃—O—Ar—O—(CH₂)₁₄—N⁺R¹R²R³Z⁻;
CF₃—O—Ar—O—(CH₂)₁₄—P⁺R¹R²R³Z⁻; CF₃—O—Ar—O—(CH₂)₁₄—O-glucoside;
CF₃—O—Ar—O—(CH₂)₁₄—OPO₃H
CF₃—O—Ar—O—(CH₂)₁₅—COOH; CF₃—O—Ar—O—(CH₂)₁₅—SO₃H; CF₃—O—Ar—O—(CH₂)₁₅—O—SO₃H;
CF₃—O—Ar—O—(CH₂)₁₅—SH; CF₃—O—Ar—O—(CH₂)₁₅—OH; CF₃—O—Ar—O—(CH₂)₁₅—N⁺R¹R²R³Z⁻;
CF₃—O—Ar—O—(CH₂)₁₅—P⁺R¹R²R³Z⁻; CF₃—O—Ar—O—(CH₂)₁₅—O-glucoside;
CF₃—O—Ar—O—(CH₂)₁₅—OPO₃H
CF₃—O—Ar—O—(CH₂)₁₆—COOH; CF₃—O—Ar—O—(CH₂)₁₆—SO₃H; CF₃—O—Ar—O—(CH₂)₁₆—O—SO₃H;
CF₃—O—Ar—O—(CH₂)₁₆—SH; CF₃—O—Ar—O—(CH₂)₁₆—OH; CF₃—O—Ar—O—(CH₂)₁₆—N⁺R¹R²R³Z⁻;
CF₃—O—Ar—O—(CH₂)₁₆—P⁺R¹R²R³Z⁻; CF₃—O—Ar—O—(CH₂)₁₆—O-glucoside;
CF₃—O—Ar—O—(CH₂)₁₆—OPO₃H
CF₃—Ar—O—CH₂—CH═CH—(CH₂)₃—COOH; CF₃—Ar—O—CH₂—CH═CH—(CH₂)₃—SO₃H;
CF₃—Ar—O—CH₂—CH═CH—(CH₂)₃—O—SO₃H;
CF₃—Ar—O—CH₂—CH═CH—(CH₂)₃—SH; CF₃—Ar—O—CH₂—CH═CH—(CH₂)₃—OH;
CF₃—Ar—O—CH₂—CH═CH—(CH₂)₃—N⁺R¹R²R³Z⁻; CF₃—Ar—O—CH₂—CH═CH—(CH₂)₃—P⁺R¹R²R³Z⁻;
CF₃—Ar—O—CH₂—CH═CH—(CH₂)₃—O-glucoside; CF₃—Ar—O—CH₂—CH═CH—(CH₂)₃—O—PO₃H;
CF₃—Ar—O—CH₂—CH═CH—(CH₂)₄—COOH; CF₃—Ar—O—CH₂—CH═CH—(CH₂)₄—SO₃H;
CF₃—Ar—O—CH₂—CH═CH—(CH₂)₄—O—SO₃H;
CF₃—Ar—O—CH₂—CH═CH—(CH₂)₄—SH; CF₃—Ar—O—CH₂—CH═CH—(CH₂)₄—OH;
CF₃—Ar—O—CH₂—CH═CH—(CH₂)₄—N⁺R¹R²R³Z⁻; CF₃—Ar—O—CH₂—CH═CH—(CH₂)₄—P⁺R¹R²R³Z⁻;
CF₃—Ar—O—CH₂—CH═CH—(CH₂)₄—O-glucoside; CF₃—Ar—O—CH₂—CH═CH—(CH₂)₄—O—PO₃H;
CF₃—Ar—O—CH₂—CH═CH—(CH₂)₅—COOH; CF₃—Ar—O—CH₂—CH═CH—(CH₂)₅—SO₃H;
CF₃—Ar—O—CH₂—CH═CH—(CH₂)₅—O—SO₃H;
CF₃—Ar—O—CH₂—CH═CH—(CH₂)₅—SH; CF₃—Ar—O—CH₂—CH═CH—(CH₂)₅—OH;
CF₃—Ar—O—CH₂—CH═CH—(CH₂)₅—N⁺R¹R²R³Z⁻; CF₃—Ar—O—CH₂—CH═CH—(CH₂)₅—P⁺R¹R²R³Z⁻;
CF₃—Ar—O—CH₂—CH═CH—(CH₂)₅—O-glucoside; CF₃—Ar—O—CH₂—CH═CH—(CH₂)₅—O—PO₃H;
CF₃—Ar—O—CH₂—CH═CH—(CH₂)₆—COOH; CF₃—Ar—O—CH₂—CH═CH—(CH₂)₆—SO₃H;
CF₃—Ar—O—CH₂—CH═CH—(CH₂)₆—O—SO₃H;
CF₃—Ar—O—CH₂—CH═CH—(CH₂)₆—SH; CF₃—Ar—O—CH₂—CH═CH—(CH₂)₆—OH;
CF₃—Ar—O—CH₂—CH═CH—(CH₂)₆—N⁺R¹R²R³Z⁻; CF₃—Ar—O—CH₂—CH═CH—(CH₂)₆—P⁺R¹R²R³Z⁻;
CF₃—Ar—O—CH₂—CH═CH—(CH₂)₆—O-glucoside; CF₃—Ar—O—CH₂—CH═CH—(CH₂)₆—OPO₃H
CF₃—Ar—O—CH₂—CH═CH—(CH₂)₇—COOH; CF₃—Ar—O—CH₂—CH═CH—(CH₂)₇—SO₃H;
CF₃—Ar—O—CH₂—CH═CH—(CH₂)₇—O—SO₃H;
CF₃—Ar—O—CH₂—CH═CH—(CH₂)₇—SH; CF₃—Ar—O—CH₂—CH═CH—(CH₂)₇—OH;
CF₃—Ar—O—CH₂—CH═CH—(CH₂)₇—N⁺R¹R²R³Z⁻; CF₃—Ar—O—CH₂—CH═CH—(CH₂)₇—P⁺R¹R²R³Z⁻;
CF₃—Ar—O—CH₂—CH═CH—(CH₂)₇—O-glucoside; CF₃—Ar—O—CH₂—CH═CH—(CH₂)₇—OPO₃H
CF₃—Ar—O—CH₂—CH═CH—(CH₂)₈—COOH; CF₃—Ar—O—CH₂—CH═CH—(CH₂)₈—SO₃H;
CF₃—Ar—O—CH₂—CH═CH—(CH₂)₈—O—SO₃H;
CF₃—Ar—O—CH₂—CH═CH—(CH₂)₈—SH; CF₃—Ar—O—CH₂—CH═CH—(CH₂)₈—OH;
CF₃—Ar—O—CH₂—CH═CH—(CH₂)₈—N⁺R¹R²R³Z⁻; CF₃—Ar—O—CH₂—CH═CH—(CH₂)₈—P⁺R¹R²R³Z⁻;
CF₃—Ar—O—CH₂—CH═CH—(CH₂)₈—O-glucoside; CF₃—Ar—O—CH₂—CH═CH—(CH₂)₈—OPO₃H
CF₃—Ar—O—CH₂—CH═CH—(CH₂)₉—COOH; CF₃—Ar—O—CH₂—CH═CH—(CH₂)₉—SO₃H;
CF₃—Ar—O—CH₂—CH═CH—(CH₂)₉—O—SO₃H;;
CF₃—Ar—O—CH₂—CH═CH—(CH₂)₉—SH; CF₃—Ar—O—CH₂—CH═CH—(CH₂)₉—OH;
CF₃—Ar—O—CH₂—CH═CH—(CH₂)₉—N⁺R¹R²R³Z⁻; CF₃—Ar—O—CH₂—CH═CH—(CH₂)₉—P⁺R¹R²R³Z⁻;
CF₃—Ar—O—CH₂—CH═CH—(CH₂)₉—O-glucoside; CF₃—Ar—O—CH₂—CH═CH—(CH₂)₉—OPO₃H
CF₃—Ar—O—CH₂—CH═CH—(CH₂)₁₀—COOH; CF₃—Ar—O—CH₂—CH═CH—(CH₂)₁₀—SO₃H;
CF₃—Ar—O—CH₂—CH═CH—(CH₂)₁₀—O—SO₃H;
CF₃—Ar—O—CH₂—CH═CH—(CH₂)₁₀—SH; CF₃—Ar—O—CH₂—CH═CH—(CH₂)₁₀—OH;
CF₃—Ar—O—CH₂—CH═CH—(CH₂)₁₀—N⁺R¹R²R³Z⁻; CF₃—Ar—O—CH₂—CH═CH—(CH₂)₁₀—P⁺R¹R²R³Z⁻;
CF₃—Ar—O—CH₂—CH═CH—(CH₂)₁₀—O-glucoside;
CF₃—Ar—O—CH₂—CH═CH—(CH₂)₁₀—OPO₃H
CF₃—Ar—O—CH₂—CH═CH—(CH₂)₁₁—COOH; CF₃—Ar—O—CH₂—CH═CH—(CH₂)₁₁—SO₃H;
CF₃—Ar—O—CH₂—CH═CH—(CH₂)₁₁—O—SO₃H;
CF₃—Ar—O—CH₂—CH═CH—(CH₂)₁₁—SH; CF₃—Ar—O—CH₂—CH═CH—(CH₂)₁₁—OH;
CF₃—Ar—O—CH₂—CH═CH—(CH₂)₁₁—N⁺R¹R²R³Z⁻; CF₃—Ar—O—CH₂—CH═CH—(CH₂)₁₁—P⁺R¹R²R³Z⁻;
CF₃—Ar—O—CH₂—CH═CH—(CH₂)₁₁—O-glucoside;
CF₃—Ar—O—CH₂—CH═CH—(CH₂)₁₁—OPO₃H
CF₃—Ar—O—CH₂—CH═CH—(CH₂)₁₂—COOH; CF₃—Ar—O—CH₂—CH═CH—(CH₂)₁₂—SO₃H;
CF₃—Ar—O—CH₂—CH═CH—(CH₂)₁₂—O—SO₃H;
CF₃—Ar—O—CH₂—CH═CH—(CH₂)₁₂—SH; CF₃—Ar—O—CH₂—CH═CH—(CH₂)₁₂—OH;
CF₃—Ar—O—CH₂—CH═CH—(CH₂)₁₂—N⁺R¹R²R³Z⁻; CF₃—Ar—O—CH₂—CH═CH—(CH₂)₁₂—P⁺R¹R²R³Z⁻;
CF₃—Ar—O—CH₂—CH═CH—(CH₂)₁₂—O-glucoside;
CF₃—Ar—O—CH₂—CH═CH—(CH₂)₁₂—OPO₃H
CF₃—Ar—O—CH₂—CH═CH—(CH₂)₁₃—COOH; CF₃—Ar—O—CH₂—CH═CH—(CH₂)₁₃—SO₃H;
CF₃—Ar—O—CH₂—CH═CH—(CH₂)₁₃—O—SO₃H;
CF₃—Ar—O—CH₂—CH═CH—(CH₂)₁₃—SH; CF₃—Ar—O—CH₂—CH═CH—(CH₂)₁₃—OH;
CF₃—Ar—O—CH₂—CH═CH—(CH₂)₁₃—N⁺R¹R²R³Z⁻; CF₃—Ar—O—CH₂—CH═CH—(CH₂)₁₃—P⁺R¹R²R³Z⁻;
CF₃—Ar—O—CH₂—CH═CH—(CH₂)₁₃—O-glucoside;
CF₃—Ar—O—CH₂—CH═CH—(CH₂)₁₃—OPO₃H
CF₃—Ar—O—CH₂—CH═CH—(CH₂)₁₄—COOH; CF₃—Ar—O—CH₂—CH═CH—(CH₂)₁₄—SO₃H;
CF₃—Ar—O—CH₂—CH═CH—(CH₂)₁₄—O—SO₃H;
CF₃—Ar—O—CH₂—CH═CH—(CH₂)₁₄—SH; CF₃—Ar—O—CH₂—CH═CH—(CH₂)₁₄—OH;
CF₃—Ar—O—CH₂—CH═CH—(CH₂)₁₄—N⁺R¹R²R³Z⁻; CF₃—Ar—O—CH₂—CH═CH—(CH₂)₁₄—P⁺R¹R²R³Z⁻;
CF₃—Ar—O—CH₂—CH═CH—(CH₂)₁₄—O-glucoside;
CF₃—Ar—O—CH₂—CH═CH—(CH₂)₁₄—OPO₃H
CF₃—Ar—O—CH₂—CH═CH—(CH₂)₁₅—COOH; CF₃—Ar—O—CH₂—CH═CH—(CH₂)₁₅—SO₃H;
CF₃—Ar—O—CH₂—CH═CH—(CH₂)₁₅—O—SO₃H;
CF₃—Ar—O—CH₂—CH═CH—(CH₂)₁₅—SH; CF₃—Ar—O—CH₂—CH═CH—(CH₂)₁₅—OH;
CF₃—Ar—O—CH₂—CH═CH—(CH₂)₁₅—N⁺R¹R²R³Z⁻; CF₃—Ar—OCH₂—CH═CH—(CH₂)₁₅—P⁺R¹R²R³Z⁻;
CF₃—Ar—O—CH₂—CH═CH—(CH₂)₁₅—O-glucoside;
CF₃—Ar—O—CH₂—CH═CH—(CH₂)₁₅—OPO₃H
CF₃—Ar—O—CH₂—CH═CH—(CH₂)₁₆—COOH; CF₃—Ar—O—CH₂—CH═CH—(CH₂)₁₆—SO₃H;
CF₃—Ar—O—CH₂—CH═CH—(CH₂)₁₆—O—SO₃H;
CF₃—Ar—O—CH₂—CH═CH—(CH₂)₁₆—SH; CF₃—Ar—O—CH₂—CH═CH—(CH₂)₁₆—OH;
CF₃—Ar—O—CH₂—CH═CH—(CH₂)₁₆—N⁺R¹R²R³Z⁻; CF₃—Ar—O—CH₂—CH═CH—(CH₂)₁₆—P⁺R¹R²R³Z⁻;
CF₃—Ar—O—CH₂—CH═CH—(CH₂)₁₆—O-glucoside;
CF₃—Ar—O—CH₂—CH═CH—(CH₂)₁₆—OPO₃H
CF₃—O—Ar—O—CH₂—CH═CH—(CH₂)₃—COOH; CF₃—O—Ar—O—CH₂—CH═CH—(CH₂)₃—SO₃H;
CF₃—O—Ar—O—CH₂—CH═CH—(CH₂)₃—O—SO₃H; CF₃—O—Ar—O—CH₂—CH═CH—(CH₂)₃—SH;
CF₃—O—Ar—O—CH₂—CH═CH—(CH₂)₃—OH; CF₃—O—Ar—O—CH₂—CH═CH—(CH₂)₃—N⁺R¹R²R³Z⁻;
CF₃—O—Ar—O—CH₂—CH═CH—(CH₂)₃—P⁺R¹R²R³Z⁻; CF₃—O—Ar—O—CH₂—CH═CH—(CH₂)₃—O-
glucoside; CF₃—O—Ar—O—CH₂—CH═CH—(CH₂)₃—O—PO₃H;
CF₃—O—Ar—O—CH₂—CH═CH—(CH₂)₄—COOH; CF₃—O—Ar—O—CH₂—CH═CH—(CH₂)₄—SO₃H;
CF₃—O—Ar—O—CH₂—CH═CH—(CH₂)₄—O—SO₃H; CF₃—O—Ar—O—CH₂—CH═CH—(CH₂)₄—SH;
CF₃—O—Ar—O—CH₂—CH═CH—(CH₂)₄—OH; CF₃—O—Ar—O—CH₂—CH═CH—(CH₂)₄—N⁺R¹R²R³Z⁻;
CF₃—O—Ar—O—CH₂—CH═CH—(CH₂)₄—P⁺R¹R²R³Z⁻; CF₃—O—Ar—O—CH₂—CH═CH—(CH₂)₄—O-
glucoside; CF₃—O—Ar—O—CH₂—CH═CH—(CH₂)₄—O—PO₃H;
CF₃—O—Ar—O—CH₂—CH═CH—(CH₂)₅—COOH; CF₃—O—Ar—O—CH₂—CH═CH—(CH₂)₅—SO₃H;
CF₃—O—Ar—O—CH₂—CH═CH—(CH₂)₅—O—SO₃H; CF₃—O—Ar—O—CH₂—CH═CH—(CH₂)₅—SH;
CF₃—O—Ar—O—CH₂—CH═CH—(CH₂)₅—OH; CF₃—O—Ar—O—CH₂—CH═CH—(CH₂)₅—N⁺R¹R²R³Z⁻;
CF₃—O—Ar—O—CH₂—CH═CH—(CH₂)₅—P⁺R¹R²R³Z⁻; CF₃—O—Ar—O—CH₂—CH═CH—(CH₂)₅—O-
glucoside; CF₃—O—Ar—O—CH₂—CH═CH—(CH₂)₅—O—PO₃H
CF₃—O—Ar—O—CH₂—CH═CH—(CH₂)₆—COOH; CF₃—O—Ar—O—CH₂—CH═CH—(CH₂)₆—SO₃H;
CF₃—O—Ar—O—CH₂—CH═CH—(CH₂)₆—O—SO₃H; CF₃—O—Ar—O—CH₂—CH═CH—(CH₂)₆—SH;
CF₃—O—Ar—O—CH₂—CH═CH—(CH₂)₆—OH; CF₃—O—Ar—O—CH₂—CH═CH—(CH₂)₆—N⁺R¹R²R³Z⁻;
CF₃—O—Ar—O—CH₂—CH═CH—(CH₂)₆—P⁺R¹R²R³Z⁻; CF₃—O—Ar—O—CH₂—CH═CH—(CH₂)₆—O-
glucoside; CF₃—O—Ar—O—CH₂—CH═CH—(CH₂)₆—OPO₃H
CF₃—O—Ar—O—CH₂—CH═CH—(CH₂)₇—COOH; CF₃—O—Ar—O—CH₂—CH═CH—(CH₂)₇—SO₃H;
CF₃—O—Ar—O—CH₂—CH═CH—(CH₂)₇—O—SO₃H; CF₃—O—Ar—O—CH₂—CH═CH—(CH₂)₇—SH;
CF₃—O—Ar—O—CH₂—CH═CH—(CH₂)₇—OH; CF₃—O—Ar—O—CH₂—CH═CH—(CH₂)₇—N⁺R¹R²R³Z⁻;
CF₃—O—Ar—O—CH₂—CH═CH—(CH₂)₇—P⁺R¹R²R³Z⁻; CF₃—O—Ar—O—CH₂—CH═CH—(CH₂)₇—O-
glucoside; CF₃—O—Ar—O—CH₂—CH═CH—(CH₂)₇—OPO₃H
CF₃—O—Ar—O—CH₂—CH═CH—(CH₂)₈—COOH; CF₃—O—Ar—O—CH₂—CH═CH—(CH₂)₈—SO₃H;
CF₃—O—Ar—O—CH₂—CH═CH—(CH₂)₈—O—SO₃H; CF₃—O—Ar—O—CH₂—CH═CH—(CH₂)₈—SH;
CF₃—O—Ar—O—CH₂—CH═CH—(CH₂)₈—OH; CF₃—O—Ar—O—CH₂—CH═CH—(CH₂)₈—N⁺R¹R²R³Z⁻;
CF₃—O—Ar—O—CH₂—CH═CH—(CH₂)₈—P⁺R¹R²R³Z⁻; CF₃—O—Ar—O—CH₂—CH═CH—(CH₂)₈—O-
glucoside; CF₃—O—Ar—O—CH₂—CH═CH—(CH₂)₈—OPO₃H
CF₃—O—Ar—O—CH₂—CH═CH—(CH₂)₉—COOH; CF₃—O—Ar—O—CH₂—CH═CH—(CH₂)₉—SO₃H;
CF₃—O—Ar—O—CH₂—CH═CH—(CH₂)₉—O—SO₃H;; CF₃—O—Ar—O—CH₂—CH═CH—(CH₂)₉—SH;
CF₃—O—Ar—O—CH₂—CH═CH—(CH₂)₉—OH; CF₃—O—Ar—O—CH₂—CH═CH—(CH₂)₉—N⁺R¹R²R³Z⁻;
CF₃—O—Ar—O—CH₂—CH═CH—(CH₂)₉—P⁺R¹R²R³Z⁻; CF₃—O—Ar—O—CH₂—CH═CH—(CH₂)₉—O-
glucoside; CF₃—O—Ar—O—CH₂—CH═CH—(CH₂)₉—OPO₃H
CF₃—O—Ar—O—CH₂—CH═CH—(CH₂)₁₀—COOH; CF₃—O—Ar—O—CH₂—CH═CH—(CH₂)₁₀—SO₃H;
CF₃—O—Ar—O—CH₂—CH═CH—(CH₂)₁₀—O—SO₃H; CF₃—O—Ar—O—CH₂—CH═CH—(CH₂)₁₀—SH;
CF₃—O—Ar—O—CH₂—CH═CH—(CH₂)₁₀—OH; CF₃—O—Ar—O—CH₂—CH═CH—(CH₂)₁₀—N⁺R¹R²R³Z⁻;
CF₃—O—Ar—O—CH₂—CH═CH—(CH₂)₁₀—P⁺R¹R²R³Z⁻; CF₃—O—Ar—O—CH₂—CH═CH—(CH₂)₁₀—O-
glucoside; CF₃—O—Ar—O—CH₂—CH═CH—(CH₂)₁₀—OPO₃H
CF₃—O—Ar—O—CH₂—CH═CH—(CH₂)₁₁—COOH; CF₃—O—Ar—O—CH₂—CH═CH—(CH₂)₁₁—SO₃H;
CF₃—O—Ar—O—CH₂—CH═CH—(CH₂)₁₁—O—SO₃H; CF₃—O—Ar—O—CH₂—CH═CH—(CH₂)₁₁—SH;
CF₃—O—Ar—O—CH₂—CH═CH—(CH₂)₁₁—OH; CF₃—O—Ar—O—CH₂—CH═CH—(CH₂)₁₁—N⁺R¹R²R³Z⁻;
CF₃—O—Ar—O—CH₂—CH═CH—(CH₂)₁₁—P⁺R¹R²R³Z⁻; CF₃—O—Ar—O—CH₂—CH═CH—(CH₂)₁₁—O-
glucoside; CF₃—O—Ar—O—CH₂—CH═CH—(CH₂)₁₁—OPO₃H
CF₃—O—Ar—O—CH₂—CH═CH—(CH₂)₁₂—COOH; CF₃—O—Ar—O—CH₂—CH═CH—(CH₂)₁₂—SO₃H;
CF₃—O—Ar—O—CH₂—CH═CH—(CH₂)₁₂—O—SO₃H; CF₃—O—Ar—O—CH₂—CH═CH—(CH₂)₁₂—SH;
CF₃—O—Ar—O—CH₂—CH═CH—(CH₂)₁₂—OH; CF₃—O—Ar—O—CH₂—CH═CH—(CH₂)₁₂—N⁺R¹R²R³Z⁻;
CF₃—O—Ar—O—CH₂—CH═CH—(CH₂)₁₂—P⁺R¹R²R³Z⁻; CF₃—O—Ar—O—CH₂—CH═CH—(CH₂)₁₂—O-
glucoside; CF₃—O—Ar—O—CH₂—CH═CH—(CH₂)₁₂—OPO₃H
CF₃—O—Ar—O—CH₂—CH═CH—(CH₂)₁₃—COOH; CF₃—O—Ar—O—CH₂—CH═CH—(CH₂)₁₃—SO₃H;
CF₃—O—Ar—O—CH₂—CH═CH—(CH₂)₁₃—O—SO₃H; CF₃—O—Ar—O—CH₂—CH═CH—(CH₂)₁₃—SH;
CF₃—O—Ar—O—CH₂—CH═CH—(CH₂)₁₃—OH; CF₃—O—Ar—O—CH₂—CH═CH—(CH₂)₁₃—N⁺R¹R²R³Z⁻;
CF₃—O—Ar—O—CH₂—CH═CH—(CH₂)₁₃—P⁺R¹R²R³Z⁻; CF₃—O—Ar—O—CH₂—CH═CH—(CH₂)₁₃—O-
glucoside; CF₃—O—Ar—O—CH₂—CH═CH—(CH₂)₁₃—OPO₃H
CF₃—O—Ar—O—CH₂—CH═CH—(CH₂)₁₄—COOH; CF₃—O—Ar—O—CH₂—CH═CH—(CH₂)₁₄—SO₃H;
CF₃—O—Ar—O—CH₂—CH═CH—(CH₂)₁₄—O—SO₃H; CF₃—O—Ar—O—CH₂—CH═CH—(CH₂)₁₄—SH;
CF₃—O—Ar—O—CH₂—CH═CH—(CH₂)₁₄—OH; CF₃—O—Ar—O—CH₂—CH═CH—(CH₂)₁₄—N⁺R¹R²R³Z⁻;
CF₃—O—Ar—O—CH₂—CH═CH—(CH₂)₁₄—OH; CF₃—O—Ar—O—CH₂—CH═CH—(CH₂)₁₄—N⁺R¹R²R³Z⁻;
CF₃—O—Ar—O—CH₂—CH═CH—(CH₂)₁₄—P⁺R¹R²R³Z⁻; CF₃—O—Ar—O—CH₂—CH═CH—(CH₂)₁₄—O-
glucoside; CF₃—O—Ar—O—CH₂—CH═CH—(CH₂)₁₄—OPO₃H
CF₃—O—Ar—O—CH₂—CH═CH—(CH₂)₁₅—COOH; CF₃—O—Ar—O—CH₂—CH═CH—(CH₂)₁₅—SO₃H;
CF₃—O—Ar—O—CH₂—CH═CH—(CH₂)₁₅—O—SO₃H; CF₃—O—Ar—O—CH₂—CH═CH—(CH₂)₁₅—SH;
CF₃—O—Ar—O—CH₂—CH═CH—(CH₂)₁₅—OH; CF₃—O—Ar—O—CH₂—CH═CH—(CH₂)₁₅—N⁺R¹R²R³Z⁻;
CF₃—O—Ar—O—CH₂—CH═CH—(CH₂)₁₅—P⁺R¹R²R³Z⁻; CF₃—O—Ar—O—CH₂—CH═CH—(CH₂)₁₅—O-
glucoside; CF₃—O—Ar—O—CH₂—CH═CH—(CH₂)₁₅—OPO₃H
CF₃—O—Ar—O—CH₂—CH═CH—(CH₂)₁₆—COOH; CF₃—O—Ar—O—CH₂—CH═CH—(CH₂)₁₆—SO₃H;
CF₃—O—Ar—O—CH₂—CH═CH—(CH₂)₁₆—O—SO₃H; CF₃—O—Ar—O—CH₂—CH═CH—(CH₂)₁₆—SH;
CF₃—O—Ar—O—CH₂—CH═CH—(CH₂)₁₆—OH; CF₃—O—Ar—O—CH₂—CH═CH—(CH₂)₁₆—N⁺R¹R²R³Z⁻;
CF₃—O—Ar—O—CH₂—CH═CH—(CH₂)₁₆—P⁺R¹R²R³Z⁻; CF₃—O—Ar—O—CH₂—CH═CH—(CH₂)₁₆—O-
glucoside; CF₃—O—Ar—O—CH₂—CH═CH—(CH₂)₁₆—OPO₃H
CF₃—CH₂—CH₂—O—Ar—O—(CH₂)₃—COOH; CF₃—CH₂—CH₂—O—Ar—O—(CH₂)₃—SO₃H;
CF₃—CH₂—CH₂—O—Ar—O—(CH₂)₃—O—SO₃H;
CF₃—CH₂—CH₂—O—Ar—O—(CH₂)₃—SH, CF₃—CH₂—CH₂—O—Ar—O—(CH₂)₃—OH;
CF₃—CH₂—CH₂—O—Ar—O—(CH₂)₃—N⁺R¹R²R³Z⁻; CF₃—CH₂—CH₂—O—Ar—O—(CH₂)₃—P⁺R¹R²R³Z⁻;
CF₃—CH₂—CH₂—O—Ar—O—(CH₂)₃—O-glucoside; CF₃—CH₂—CH₂—O—Ar—O—(CH₂)₃—OPO₃H
CF₃—CH₂—CH₂—O—Ar—O—(CH₂)₄—COOH; CF₃—CH₂—CH₂—O—Ar—O—(CH₂)₄—SO₃H;
CF₃—CH₂—CH₂—O—Ar—O—(CH₂)₄—O—SO₃H;
CF₃—CH₂—CH₂—O—Ar—O—(CH₂)₄—SH, CF₃—CH₂—CH₂—O—Ar—O—(CH₂)₄—OH;
CF₃—CH₂—CH₂—O—Ar—O—(CH₂)₄—N⁺R¹R²R³Z⁻; CF₃—CH₂—CH₂—O—Ar—O—(CH₂)₄—P⁺R¹R²R³Z⁻;
CF₃—CH₂—CH₂—O—Ar—O—(CH₂)₄—O-glucoside; CF₃—CH₂—CH₂—O—Ar—O—(CH₂)₄—OPO₃H
CF₃—CH₂—CH₂—O—Ar—O—(CH₂)₅—COOH; CF₃—CH₂—CH₂—O—Ar—O—(CH₂)₅—SO₃H;
CF₃—CH₂—CH₂—O—Ar—O—(CH₂)₅—O—SO₃H;
CF₃—CH₂—CH₂—O—Ar—O—(CH₂)₅—SH, CF₃—CH₂—CH₂—O—Ar—O—(CH₂)₅—OH;
CF₃—CH₂—CH₂—O—Ar—O—(CH₂)₅—N⁺R¹R²R³Z⁻; CF₃—CH₂—CH₂—O—Ar—O—(CH₂)₅—P⁺R¹R²R³Z⁻;
CF₃—CH₂—CH₂—O—Ar—O—(CH₂)₅—O-glucoside; CF₃—CH₂—CH₂—O—Ar—O—(CH₂)₅—OPO₃H
CF₃—CH₂—CH₂—O—Ar—O—(CH₂)₆—COOH; CF₃—CH₂—CH₂—O—Ar—O—(CH₂)₆—SO₃H;
CF₃—CH₂—CH₂—O—Ar—O—(CH₂)₆—O—SO₃H;
CF₃—CH₂—CH₂—O—Ar—O—(CH₂)₆—SH, CF₃—CH₂—CH₂—O—Ar—O—(CH₂)₆—OH;
CF₃—CH₂—CH₂—O—Ar—O—(CH₂)₆—N⁺R¹R²R³Z⁻; CF₃—CH₂—CH₂—O—Ar—O—(CH₂)₆—P⁺R¹R²R³Z⁻;
CF₃—CH₂—CH₂—O—Ar—O—(CH₂)₆—O-glucoside; CF₃—CH₂—CH₂—O—Ar—O—(CH₂)₆—OPO₃H
CF₃—CH₂—CH₂—O—Ar—O—(CH₂)₇—COOH; CF₃—CH₂—CH₂—O—Ar—O—(CH₂)₇—SO₃H;
CF₃—CH₂—CH₂—O—Ar—O—(CH₂)₇—O—SO₃H;
CF₃—CH₂—CH₂—O—Ar—O—(CH₂)₇—SH, CF₃—CH₂—CH₂—O—Ar—O—(CH₂)₇—OH;
CF₃—CH₂—CH₂—O—Ar—O—(CH₂)₇—N⁺R¹R²R³Z⁻; CF₃—CH₂—CH₂—O—Ar—O—(CH₂)₇—P⁺R¹R²R³Z⁻;
CF₃—CH₂—CH₂—O—Ar—O—(CH₂)₇—O-glucoside; CF₃—CH₂—CH₂—O—Ar—O—(CH₂)₇—OPO₃H
CF₃—CH₂—CH₂—O—Ar—O—(CH₂)₈—COOH; CF₃—CH₂—CH₂—O—Ar—O—(CH₂)₈—SO₃H;
CF₃—CH₂—CH₂—O—Ar—O—(CH₂)₈—O—SO₃H;
CF₃—CH₂—CH₂—O—Ar—O—(CH₂)₈—SH, CF₃—CH₂—CH₂—O—Ar—O—(CH₂)₈—OH;
CF₃—CH₂—CH₂—O—Ar—O—(CH₂)₈—N⁺R¹R²R³Z⁻; CF₃—CH₂—CH₂—O—Ar—O—(CH₂)₈—P⁺R¹R²R³Z⁻;
CF₃—CH₂—CH₂—O—Ar—O—(CH₂)₈—O-glucoside; CF₃—CH₂—CH₂—O—Ar—O—(CH₂)₈—OPO₃H
CF₃—CH₂—CH₂—O—Ar—O—(CH₂)₉—COOH; CF₃—CH₂—CH₂—O—Ar—O—(CH₂)₉—SO₃H;
CF₃—CH₂—CH₂—O—Ar—O—(CH₂)₉—O—SO₃H;
CF₃—CH₂—CH₂—O—Ar—O—(CH₂)₉—SH, CF₃—CH₂—CH₂—O—Ar—O—(CH₂)₉—OH;
CF₃—CH₂—CH₂—O—Ar—O—(CH₂)₉—N⁺R¹R²R³Z⁻; CF₃—CH₂—CH₂—O—Ar—O—(CH₂)₉—P⁺R¹R²R³Z⁻;
CF₃—CH₂—CH₂—O—Ar—O—(CH₂)₉—O-glucoside; CF₃—CH₂—CH₂—O—Ar—O—(CH₂)₉—OPO₃H
CF₃—CH₂—CH₂—O—Ar—O—(CH₂)₁₀—COOH; CF₃—CH₂—CH₂—O—Ar—O—(CH₂)₁₀—SO₃H;
CF₃—CH₂—CH₂—O—Ar—O—(CH₂)₁₀—O—SO₃H;
CF₃—CH₂—CH₂—O—Ar—O—(CH₂)₁₀—SH, CF₃—CH₂—CH₂—O—Ar—O—(CH₂)₁₀—OH;
CF₃—CH₂—CH₂—O—Ar—O—(CH₂)₁₀—N⁺R¹R²R³Z⁻; CF₃—CH₂—CH₂—O—Ar—O—(CH₂)₁₀—P⁺R¹R²R³Z⁻;
CF₃—CH₂—CH₂—O—Ar—O—(CH₂)₁₀—O-glucoside; CF₃—CH₂—CH₂—O—Ar—O—(CH₂)₁₀—OPO₃H
CF₃—CH₂—CH₂—O—Ar—O—(CH₂)₁₁—COOH; CF₃—CH₂—CH₂—O—Ar—O—(CH₂)₁₁—SO₃H;
CF₃—CH₂—CH₂—O—Ar—O—(CH₂)₁₁—O—SO₃H;
CF₃—CH₂—CH₂—O—Ar—O—(CH₂)₁₁—SH, CF₃—CH₂—CH₂—O—Ar—O—(CH₂)₁₁—OH;
CF₃—CH₂—CH₂—O—Ar—O—(CH₂)₁₁—N⁺R¹R²R³Z⁻; CF₃—CH₂—CH₂—O—Ar—O—(CH₂)₁₁—P⁺R¹R²R³Z⁻;
CF₃—CH₂—CH₂—O—Ar—O—(CH₂)₁₁—O-glucoside; CF₃—CH₂—CH₂—O—Ar—O—(CH₂)₁₁—OPO₃H
CF₃—CH₂—CH₂—O—Ar—O—(CH₂)₁₂—COOH; CF₃—CH₂—CH₂—O—Ar—O—(CH₂)₁₂—SO₃H;
CF₃—CH₂—CH₂—O—Ar—O—(CH₂)₁₂—O—SO₃H;
CF₃—CH₂—CH₂—O—Ar—O—(CH₂)₁₂—SH, CF₃—CH₂—CH₂—O—Ar—O—(CH₂)₁₂—OH;
CF₃—CH₂—CH₂—O—Ar—O—(CH₂)₁₂—N⁺R¹R²R³Z⁻; CF₃—CH₂—CH₂—O—Ar—O—(CH₂)₁₂—P⁺R¹R²R³Z⁻;
CF₃—CH₂—CH₂—O—Ar—O—(CH₂)₁₂—O-glucoside; CF₃—CH₂—CH₂—O—Ar—O—(CH₂)₁₂—OPO₃H
CF₃—CH₂—CH₂—O—Ar—O—(CH₂)₁₃—COOH; CF₃—CH₂—CH₂—O—Ar—O—(CH₂)₁₃—SO₃H;
CF₃—CH₂—CH₂—O—Ar—O—(CH₂)₁₃—O—SO₃H;
CF₃—CH₂—CH₂—O—Ar—O—(CH₂)₁₃—SH, CF₃—CH₂—CH₂—O—Ar—O—(CH₂)₁₃—OH;
CF₃—CH₂—CH₂—O—Ar—O—(CH₂)₁₃—N⁺R¹R²R³Z⁻; CF₃—CH₂—CH₂—O—Ar—O—(CH₂)₁₃—P⁺R¹R²R³Z⁻;
CF₃—CH₂—CH₂—O—Ar—O—(CH₂)₁₃—O-glucoside; CF₃—CH₂—CH₂—O—Ar—O—(CH₂)₁₃—OPO₃H
CF₃—CH₂—CH₂—O—Ar—O—(CH₂)₁₄—COOH; CF₃—CH₂—CH₂—O—Ar—O—(CH₂)₁₄—SO₃H;
CF₃—CH₂—CH₂—O—Ar—O—(CH₂)₁₄—O—SO₃H;
CF₃—CH₂—CH₂—O—Ar—O—(CH₂)₁₄—SH, CF₃—CH₂—CH₂—O—Ar—O—(CH₂)₁₄—OH;
CF₃—CH₂—CH₂—O—Ar—O—(CH₂)₁₄—N⁺R¹R²R³Z⁻; CF₃—CH₂—CH₂—O—Ar—O—(CH₂)₁₄—P⁺R¹R²R³Z⁻;
CF₃—CH₂—CH₂—O—Ar—O—(CH₂)₁₄—O-glucoside; CF₃—CH₂—CH₂—O—Ar—O—(CH₂)₁₄—OPO₃H
CF₃—CH₂—CH₂—O—Ar—O—(CH₂)₁₅—COOH; CF₃—CH₂—CH₂—O—Ar—O—(CH₂)₁₅—SO₃H;
CF₃—CH₂—CH₂—O—Ar—O—(CH₂)₁₅—O—SO₃H;
CF₃—CH₂—CH₂—O—Ar—O—(CH₂)₁₅—SH, CF₃—CH₂—CH₂—O—Ar—O—(CH₂)₁₅—OH;
CF₃—CH₂—CH₂—O—Ar—O—(CH₂)₁₅—N⁺R¹R²R³Z⁻; CF₃—CH₂—CH₂—O—Ar—O—(CH₂)₁₅—P⁺R¹R²R³Z⁻;
CF₃—CH₂—CH₂—O—Ar—O—(CH₂)₁₅—O-glucoside; CF₃—CH₂—CH₂—O—Ar—O—(CH₂)₁₅—OPO₃H
CF₃—CH₂—CH₂—O—Ar—O—(CH₂)₁₆—COOH; CF₃—CH₂—CH₂—O—Ar—O—(CH₂)₁₆—SO₃H;
CF₃—CH₂—CH₂—O—Ar—O—(CH₂)₁₆—O—SO₃H;
CF₃—CH₂—CH₂—O—Ar—O—(CH₂)₁₆—SH, CF₃—CH₂—CH₂—O—Ar—O—(CH₂)₁₆—OH;
CF₃—CH₂—CH₂—O—Ar—O—(CH₂)₁₆—N⁺R¹R²R³Z⁻; CF₃—CH₂—CH₂—O—Ar—O—(CH₂)₁₆—P⁺R¹R²R³Z⁻;
CF₃—CH₂—CH₂—O—Ar—O—(CH₂)₁₆—O-glucoside; CF₃—CH₂—CH₂—O—Ar—O—(CH₂)₁₆—OPO₃H
CF₃—CH₂—CH₂—O—Ar—O—CH₂—CH═CH—(CH₂)₃—COOH; CF₃—CH₂—CH₂—O—Ar—O—CH₂—CH═CH—(CH₂)₃—SO₃H;
CF₃—CH₂—CH₂—O—Ar—O—CH₂—CH═CH—(CH₂)₃—O—SO₃H; CF₃—CH₂—CH₂—O—Ar—O—CH₂—CH═CH—(CH₂)₃—SH;
CF₃—CH₂—CH₂—O—Ar—O—CH₂—CH═CH—(CH₂)₃—OH;
CF₃—CH₂—CH₂—O—Ar—O—CH₂—CH═CH—(CH₂)₃—N⁺R¹R²R³Z⁻; CF₃—CH₂—CH₂—O—Ar—O—CH₂—CH═CH—(CH₂)₃—P⁺R¹R²R³Z⁻;
CF₃—CH₂—CH₂—O—Ar—O—CH₂—CH═CH—(CH₂)₃—O-
glucoside; CF₃—CH₂—CH₂—O—Ar—O—CH₂—CH═CH—(CH₂)₃—O—PO₃H;
CF₃—CH₂—CH₂—O—Ar—O—CH₂—CH═CH—(CH₂)₄—COOH; CF₃—CH₂—CH₂—O—Ar—O—CH₂—CH═CH—(CH₂)₄—SO₃H;
CF₃—CH₂—CH₂—O—Ar—O—CH₂—CH═CH—(CH₂)₄—O—SO₃H; CF₃—CH₂—CH₂—O—Ar—O—CH₂—CH═CH—(CH₂)₄—SH;
CF₃—CH₂—CH₂—O—Ar—O—CH₂—CH═CH—(CH₂)₄—OH;
CF₃—CH₂—CH₂—O—Ar—O—CH₂—CH═CH—(CH₂)₄—N⁺R¹R²R³Z⁻; CF₃—CH₂—CH₂—O—Ar—O—CH₂—CH═CH—(CH₂)₄—P⁺R¹R²R³Z⁻;
CF₃—CH₂—CH₂—O—Ar—O—CH₂—CH═CH—(CH₂)₄—O-
glucoside; CF₃—CH₂—CH₂—O—Ar—O—CH₂—CH═CH—(CH₂)₄—O—PO₃H;
CF₃—CH₂—CH₂—O—A—O—CH₂—CH═CH—(CH₂)₅—COOH; CF₃—CH₂—CH₂—O—Ar—O—CH₂—CH═CH—(CH₂)₅—SO₃H;
CF₃—CH₂—CH₂—O—Ar—O—CH₂—CH═CH—(CH₂)₅—O—SO₃H; CF₃—CH₂—CH₂—O—Ar—O—CH₂—CH═CH—(CH₂)₅—SH;
CF₃—CH₂—CH₂—O—Ar—O—CH₂—CH═CH—(CH₂)₅—OH;
CF₃—CH₂—CH₂—O—Ar—O—CH₂—CH═CH—(CH₂)₅—N⁺R¹R²R³Z⁻; CF₃—CH₂—CH₂—O—Ar—O—CH₂—CH═CH—(CH₂)₅—P⁺R¹R²R³Z⁻;
CF₃—CH₂—CH₂—O—Ar—O—CH₂—CH═CH—(CH₂)₅—O-
glucoside, CF₃—CH₂—CH₂—O—Ar—O—CH₂—CH═CH—(CH₂)₅—O—PO₃H
CF₃—CH₂—CH₂—O—Ar—O—CH₂—CH═CH—(CH₂)₆—COOH; CF₃—CH₂—CH₂—O—Ar—O—CH₂—CH═CH—(CH₂)₆—SO₃H;
CF₃—CH₂—CH₂—O—Ar—O—CH₂—CH═CH—(CH₂)₆—O—SO₃H; CF₃—CH₂—CH₂—O—Ar—O—CH₂—CH═CH—(CH₂)₆—SH;
CF₃—CH₂—CH₂—O—Ar—O—CH₂—CH═CH—(CH₂)₆—OH;
CF₃—CH₂—CH₂—O—Ar—O—CH₂—CH═CH—(CH₂)₆—N⁺R¹R²R³Z⁻; CF₃—CH₂—CH₂—O—Ar—O—CH₂—CH═CH—(CH₂)₆—P⁺R¹R²R³Z⁻;
CF₃—CH₂—CH₂—O—Ar—O—CH₂—CH═CH—(CH₂)₆—O-
glucoside; CF₃—CH₂—CH₂—O—Ar—O—CH₂—CH═CH—(CH₂)₆—OPO₃H
CF₃—CH₂—CH₂—O—Ar—O—CH₂—CH═CH—(CH₂)₇—COOH; CF₃—CH₂—CH₂—O—Ar—O—CH₂—CH═CH—(CH₂)₇—SO₃H;
CF₃—CH₂—CH₂—O—Ar—O—CH₂—CH═CH—(CH₂)₇—O—SO₃H; CF₃—CH₂—CH₂—O—Ar—O—CH₂—CH═CH—(CH₂)₇—SH;
CF₃—CH₂—CH₂—O—Ar—O—CH₂—CH═CH—(CH₂)₇—OH;
CF₃—CH₂—CH₂—O—Ar—O—CH₂—CH═CH—(CH₂)₇—N⁺R¹R²R³Z⁻; CF₃—CH₂—CH₂—O—Ar—O—CH₂—CH═CH—(CH₂)₇—P⁺R¹R²R³Z⁻;
CF₃—CH₂—CH₂—O—Ar—O—CH₂—CH═CH—(CH₂)₇—O-
glucoside; CF₃—CH₂—CH₂—O—Ar—O—CH₂—CH═CH—(CH₂)₇—OPO₃H
CF₃—CH₂—CH₂—O—Ar—O—CH₂—CH═CH—(CH₂)₈—COOH; CF₃—CH₂—CH₂—O—Ar—O—CH₂—CH═CH—(CH₂)₈—SO₃H;
CF₃—CH₂—CH₂—O—Ar—O—CH₂—CH═CH—(CH₂)₈—O—SO₃H; CF₃—CH₂—CH₂—O—Ar—O—CH₂—CH═CH—(CH₂)₈—SH;
CF₃—CH₂—CH₂—O—Ar—O—CH₂—OH═CH—(CH₂)₈—OH;
CF₃—CH₂—CH₂—O—Ar—O—CH₂—CH═CH—(CH₂)₈—N⁺R¹R²R³Z⁻; CF₃—CH₂—CH₂—O—Ar—O—CH₂—CH═CH—(CH₂)₈—P⁺R¹R²R³Z⁻;
CF₃—CH₂—CH₂—O—Ar—O—CH₂—CH═CH—(CH₂)₈—O-
glucoside; CF₃—CH₂—CH₂—O—Ar—O—CH₂—CH═CH—(CH₂)₈—OPO₃H
CF₃—CH₂—CH₂—O—Ar—O—CH₂—CH═CH—(CH₂)₉—COOH; CF₃—CH₂—CH₂—O—Ar—O—CH₂—CH═CH—(CH₂)₉—SO₃H;;
CF₃—CH₂—CH₂—O—Ar—O—CH₂—CH═CH—(CH₂)₉—O—SO₃H; CF₃—CH₂—CH₂—O—Ar—O—CH₂—CH═CH—(CH₂)₉—SH;
CF₃—CH₂—CH₂—O—Ar—O—CH₂—CH═CH—(CH₂)₉—OH;
CF₃—CH₂—CH₂—O—Ar—O—CH₂—CH═CH—(CH₂)₉—N⁺R¹R²R³Z⁻; CF₃—CH₂—CH₂—O—Ar—O—CH₂—CH═CH—(CH₂)₉—P⁺R¹R²R³Z⁻;
CF₃—CH₂—CH₂—O—Ar—O—CH₂—CH═CH—(CH₂)₉—O-
glucoside; CF₃—CH₂—CH₂—O—Ar—O—CH₂—CH═CH—(CH₂)₉—OPO₃H
CF₃—CH₂—CH₂—O—Ar—O—CH₂—CH═CH—(CH₂)₁₀—COOH; CF₃—CH₂—CH₂—O—Ar—O—CH₂—CH═CH—(CH₂)₁₀—SO₃H;
CF₃—CH₂—CH₂—O—Ar—O—CH₂—CH═CH—(CH₂)₁₀—O—SO₃H; CF₃—CH₂—CH₂—O—Ar—O—CH₂—CH═CH—(CH₂)₁₀—SH;
CF₃—CH₂—CH₂—O—Ar—O—CH₂—CH═CH—(CH₂)₁₀—OH;
CF₃—CH₂—CH₂—O—Ar—O—CH₂—CH═CH—(CH₂)₁₀—N⁺R¹R²R³Z⁻; CF₃—CH₂—CH₂—O—Ar—O—CH₂—CH═CH—(CH₂)₁₀—P⁺R¹R²R³Z⁻;
CF₃—CH₂—CH₂—O—Ar—O—CH₂—CH═CH—(CH₂)₁₀—O-
glucoside; CF₃—CH₂—CH₂—O—Ar—O—CH₂—CH═CH—(CH₂)₁₀—OPO₃H
CF₃—CH₂—CH₂—O—Ar—O—CH₂—CH═CH—(CH₂)₁₁—COOH; CF₃—CH₂—CH₂—O—Ar—O—CH₂—CH═CH—(CH₂)₁₁—SO₃H;
CF₃—CH₂—CH₂—O—Ar—O—CH₂—CH═CH—(CH₂)₁₁—O—SO₃H; CF₃—CH₂—CH₂—O—Ar—O—CH₂—CH═CH—(CH₂)₁₁—SH;
CF₃—CH₂—CH₂—O—Ar—O—CH₂—CH═CH—(CH₂)₁₁—OH;
CF₃—CH₂—CH₂—O—Ar—O—CH₂—CH═CH—(CH₂)₁₁—N⁺R¹R²R³Z⁻; CF₃—CH₂—CH₂—O—Ar—O—CH₂—CH═CH—(CH₂)₁₁—P⁺R¹R²R³Z⁻;
CF₃—CH₂—CH₂—O—Ar—O—CH₂—CH═CH—(CH₂)₁₁—O-
glucoside; CF₃—CH₂—CH₂—O—Ar—O—CH₂—CH═CH—(CH₂)₁₁—OPO₃H
CF₃—CH₂—CH₂—O—Ar—O—CH₂—CH═CH—(CH₂)₁₂—COOH; CF₃—CH₂—CH₂—O—Ar—O—CH₂—CH═CH—(CH₂)₁₂—SO₃H;
CF₃—CH₂—CH₂—O—Ar—O—CH₂—CH═CH—(CH₂)₁₂—O—SO₃H; CF₃—CH₂—CH₂—O—Ar—O—CH₂—CH═CH—(CH₂)₁₂—SH;
CF₃—CH₂—CH₂—O—Ar—O—CH₂—CH═CH—(CH₂)₁₂—OH;
CF₃—CH₂—CH₂—O—Ar—O—CH₂—CH═CH—(CH₂)₁₂—N⁺R¹R²R³Z⁻; CF₃—CH₂—CH₂—O—Ar—O—CH₂—CH═CH—(CH₂)₁₂—P⁺R¹R²R³Z⁻;
CF₃—CH₂—CH₂—O—Ar—O—CH₂—CH═CH—(CH₂)₁₂—O-
glucoside; CF₃—CH₂—CH₂—O—Ar—O—CH₂—CH═CH—(CH₂)₁₂—OPO₃H
CF₃—CH₂—CH₂—O—Ar—O—CH₂—CH═CH—(CH₂)₁₃—COOH; CF₃—CH₂—CH₂—O—Ar—O—CH₂—CH═CH—(CH₂)₁₃—SO₃H;
CF₃—CH₂—CH₂—O—Ar—O—CH₂—CH═CH—(CH₂)₁₃—O—SO₃H; CF₃—CH₂—CH₂—O—Ar—O—CH₂—CH═CH—(CH₂)₁₃—SH;
CF₃—CH₂—CH₂—O—Ar—O—CH₂—CH═CH—(CH₂)₁₃—OH;
CF₃—CH₂—CH₂—O—Ar—O—CH₂—CH═CH—(CH₂)₁₃—N⁺R¹R²R³Z⁻; CF₃—CH₂—CH₂—O—Ar—O—CH₂—CH═CH—(CH₂)₁₃—P⁺R¹R²R³Z⁻;
CF₃—CH₂—CH₂—O—Ar—O—CH₂—CH═CH—(CH₂)₁₃—O-
glucoside; CF₃—CH₂—CH₂—O—Ar—O—CH₂—CH═CH—(CH₂)₁₃—OPO₃H
CF₃—CH₂—CH₂—O—Ar—O—CH₂—CH═CH—(CH₂)₁₄—COOH; CF₃—CH₂—CH₂—O—Ar—O—CH₂—CH═CH—(CH₂)₁₄—SO₃H;
CF₃—CH₂—CH₂—O—Ar—O—CH₂—CH═CH—(CH₂)₁₄—O—SO₃H; CF₃—CH₂—CH₂—O—Ar—O—CH₂—CH═CH—(CH₂)₁₄—SH;
CF₃—CH₂—CH₂—O—Ar—O—CH₂—CH═CH—(CH₂)₁₄—OH;
CF₃—CH₂—CH₂—O—Ar—O—CH₂—CH═CH—(CH₂)₁₄—N⁺R¹R²R³Z⁻; CF₃—CH₂—CH₂—O—Ar—O—CH₂—CH═CH—(CH₂)₁₄—P⁺R¹R²R³Z⁻;
CF₃—CH₂—CH₂—O—Ar—O—CH₂—CH═CH—(CH₂)₁₄—O-
glucoside; CF₃—CH₂—CH₂—O—Ar—O—CH₂—CH═CH—(CH₂)₁₄—OPO₃H
CF₃—CH₂—CH₂—O—Ar—O—CH₂—CH═CH—(CH₂)₁₅—COOH; CF₃—CH₂—CH₂—O—Ar—O—CH₂—CH═CH—(CH₂)₁₅—SO₃H;
CF₃—CH₂—CH₂—O—Ar—O—CH₂—CH═CH—(CH₂)₁₅—O—SO₃H; CF₃—CH₂—CH₂—O—Ar—O—CH₂—CH═CH—(CH₂)₁₅—SH;
CF₃—CH₂—CH₂—O—Ar—O—CH₂—CH═CH—(CH₂)₁₅—OH;
CF₃—CH₂—CH₂—O—Ar—O—CH₂—CH═CH—(CH₂)₁₅—N⁺R¹R²R³Z⁻; CF₃—CH₂—CH₂—O—Ar—O—CH₂—CH═CH—(CH₂)₁₅—P⁺R¹R²R³Z⁻;
CF₃—CH₂—CH₂—O—Ar—O—CH₂—CH═CH—(CH₂)₁₅—O-
glucoside; CF₃—CH₂—CH₂—O—Ar—O—CH₂—CH═CH—(CH₂)₁₅—OPO₃H
CF₃—CH₂—CH₂—O—Ar—O—CH₂—CH═CH—(CH₂)₁₆—COOH; CF₃—CH₂—CH₂—O—Ar—O—CH₂—CH═CH—(CH₂)₁₆—SO₃H;
CF₃—CH₂—CH₂—O—Ar—O—CH₂—CH═CH—(CH₂)₁₆—O—SO₃H; CF₃—CH₂—CH₂—O—Ar—O—CH₂—CH═CH—(CH₂)₁₆—SH;
CF₃—CH₂—CH₂—O—Ar—O—CH₂—CH═CH—(CH₂)₁₆—OH;
CF₃—CH₂—CH₂—O—Ar—O—CH₂—CH═CH—(CH₂)₁₆—N⁺R¹R²R³Z⁻; CF₃—CH₂—CH₂—O—Ar—O—CH₂—CH═CH—(CH₂)₁₆—P⁺R¹R²R³Z⁻;
CF₃—CH₂—CH₂—O—Ar—O—CH₂—CH═CH—(CH₂)₁₆—O-
glucoside; CF₃—CH₂—CH₂—O—Ar—O—CH₂—CH═CH—(CH₂)₁₆—OPO₃H
CF₃—Ar—O—C(O)—(CH₂)₃—COOH; CF₃—Ar—O—C(O)—(CH₂)₃—SO₃H; CF₃—Ar—O—C(O)—(CH₂)₃—O—SO₃H;
CF₃—Ar—O—C(O)—(CH₂)₃—SH; CF₃—Ar—O—C(O)—(CH₂)₃—OH; CF₃—Ar—O—C(O)—(CH₂)₃—N⁺R¹R²R³Z⁻;
CF₃—Ar—O—C(O)—(CH₂)₃—P⁺R¹R²R³Z⁻; CF₃—Ar—O—C(O)—(CH₂)₃—O-
glucoside; CF₃—Ar—O—C(O)—(CH₂)₃—O—PO₃H;
CF₃—Ar—O—C(O)—(CH₂)₄—COOH; CF₃—Ar—O—C(O)—(CH₂)₄—SO₃H; CF₃—Ar—O—C(O)—(CH₂)₄—O—SO₃H;
CF₃—Ar—O—C(O)—(CH₂)₄—SH; CF₃—Ar—O—C(O)—(CH₂)₄—OH; CF₃—Ar—O—C(O)—(CH₂)₄—N⁺R¹R²R³Z⁻;
CF₃—Ar—O—C(O)—(CH₂)₄—P⁺R¹R²R³Z⁻; CF₃—Ar—O—C(O)—(CH₂)₄—O-
glucoside; CF₃—Ar—O—C(O)—(CH₂)₄—O—PO₃H;
CF₃—Ar—O—C(O)—(CH₂)₅—COOH; CF₃—Ar—O—C(O)—(CH₂)₅—SO₃H; CF₃—Ar—O—C(O)—(CH₂)₅—O—SO₃H;
CF₃—Ar—O—C(O)—(CH₂)₅—SH; CF₃—Ar—O—C(O)—(CH₂)₅—OH; CF₃—Ar—O—C(O)—(CH₂)₅—N⁺R¹R²R³Z⁻;
CF₃—Ar—O—C(O)—(CH₂)₅—P⁺R¹R²R³Z⁻; CF₃—Ar—O—C(O)—(CH₂)₅—O-
glucoside; CF₃—Ar—O—C(O)—(CH₂)₅—O—PO₃H
CF₃—Ar—O—C(O)—(CH₂)₆—COOH; CF₃—Ar—O—C(O)—(CH₂)₆—SO₃H; CF₃—Ar—O—C(O)—(CH₂)₆—O—SO₃H;
CF₃—Ar—O—C(O)—(CH₂)₆—SH; CF₃—Ar—O—C(O)—(CH₂)₆—OH; CF₃—Ar—O—C(O)—(CH₂)₆—N⁺R¹R²R³Z⁻;
CF₃—Ar—O—C(O)—(CH₂)₆—P⁺R¹R²R³Z⁻; CF₃—Ar—O—C(O)—(CH₂)₆—O-
glucoside; CF₃—Ar—O—C(O)—(CH₂)₆—OPO₃H
CF₃—Ar—O—C(O)—(CH₂)₇—COOH; CF₃—Ar—O—C(O)—(CH₂)₇—SO₃H; CF₃—Ar—O—C(O)—(CH₂)₇—O—SO₃H;
CF₃—Ar—O—C(O)—(CH₂)₇—SH; CF₃—Ar—O—C(O)—(CH₂)₇—OH; CF₃—Ar—O—C(O)—(CH₂)₇—N⁺R¹R²R³Z⁻;
CF₃—Ar—O—C(O)—(CH₂)₇—P⁺R¹R²R³Z⁻; CF₃—Ar—O—C(O)—(CH₂)₇—O-
glucoside; CF₃—Ar—O—C(O)—(CH₂)₇—OPO₃H
CF₃—Ar—O—C(O)—(CH₂)₈—COOH; CF₃—Ar—O—C(O)—(CH₂)₈—SO₃H; CF₃—Ar—O—C(O)—(CH₂)₈—O—SO₃H;
CF₃—Ar—O—C(O)—(CH₂)₈—SH; CF₃—Ar—O—C(O)—(CH₂)₈—OH; CF₃—Ar—O—C(O)—(CH₂)₈—N⁺R¹R²R³Z⁻;
CF₃—Ar—O—C(O)—(CH₂)₈—P⁺R¹R²R³Z⁻; CF₃—Ar—O—C(O)—(CH₂)₈—O-
glucoside; CF₃—Ar—O—C(O)—(CH₂)₈—OPO₃H
CF₃—Ar—O—C(O)—(CH₂)₉—COOH; CF₃—Ar—O—C(O)—(CH₂)₉—SO₃H; CF₃—Ar—O—C(O)—(CH₂)₉—O—SO₃H;;
CF₃—Ar—O—C(O)—(CH₂)₉—SH; CF₃—Ar—O—C(O)—(CH₂)₉—OH; CF₃—Ar—O—C(O)—(CH₂)₉—N⁺R¹R²R³Z⁻;
CF₃—Ar—O—C(O)—(CH₂)₉—P⁺R¹R²R³Z⁻; CF₃—Ar—O—C(O)—(CH₂)₉—O-
glucoside; CF₃—Ar—O—C(O)—(CH₂)₉—OPO₃H
CF₃—Ar—O—C(O)—(CH₂)₁₀—COOH; CF₃—Ar—O—C(O)—(CH₂)₁₀—SO₃H; CF₃—Ar—O—C(O)—(CH₂)₁₀—O—SO₃H;
CF₃—Ar—O—C(O)—(CH₂)₁₀—SH; CF₃—Ar—O—C(O)—(CH₂)₁₀—OH; CF₃—Ar—O—C(O)—(CH₂)₁₀—N⁺R¹R²R³Z⁻;
CF₃—Ar—O—C(O)—(CH₂)₁₀—P⁺R¹R²R³Z⁻; CF₃—Ar—O—C(O)—(CH₂)₁₀—O-
glucoside; CF₃—Ar—O—C(O)—(CH₂)₁₀—OPO₃H
CF₃—Ar—O—C(O)—(CH₂)₁₁—COOH; CF₃—Ar—O—C(O)—(CH₂)₁₁—SO₃H; CF₃—Ar—O—C(O)—(CH₂)₁₁—O—SO₃H;
CF₃—Ar—O—C(O)—(CH₂)₁₁—SH; CF₃—Ar—O—C(O)—(CH₂)₁₁—OH; CF₃—Ar—O—C(O)—(CH₂)₁₁—N⁺R¹R²R³Z⁻;
CF₃—Ar—O—C(O)—(CH₂)₁₁—P⁺R¹R²R³Z⁻; CF₃—Ar—O—C(O)—(CH₂)₁₁—O-
glucoside; CF₃—Ar—O—C(O)—(CH₂)₁₁—OPO₃H
CF₃—Ar—O—C(O)—(CH₂)₁₂—COOH; CF₃—Ar—O—C(O)—(CH₂)₁₂—SO₃H; CF₃—Ar—O—C(O)—(CH₂)₁₂—O—SO₃H;
CF₃—Ar—O—C(O)—(CH₂)₁₂—SH; CF₃—Ar—O—C(O)—(CH₂)₁₂—OH; CF₃—Ar—O—C(O)—(CH₂)₁₂—N⁺R¹R²R³Z⁻;
CF₃—Ar—O—C(O)—(CH₂)₁₂—P⁺R¹R²R³Z⁻; CF₃—Ar—O—C(O)—(CH₂)₁₂—O-
glucoside; CF₃—Ar—O—C(O)—(CH₂)₁₂—OPO₃H
CF₃—Ar—O—C(O)—(CH₂)₁₃—COOH; CF₃—Ar—O—C(O)—(CH₂)₁₃—SO₃H; CF₃—Ar—O—C(O)—(CH₂)₁₃—O—SO₃H;
CF₃—Ar—O—C(O)—(CH₂)₁₃—SH; CF₃—Ar—O—C(O)—(CH₂)₁₃—OH; CF₃—Ar—O—C(O)—(CH₂)₁₃—N⁺R¹R²R³Z⁻;
CF₃—Ar—O—C(O)—(CH₂)₁₃—P⁺R¹R²R³Z⁻; CF₃—Ar—O—C(O)—(CH₂)₁₃—O-
glucoside; CF₃—Ar—O—C(O)—(CH₂)₁₃—OPO₃H
CF₃—Ar—O—C(O)—(CH₂)₁₄—COOH; CF₃—Ar—O—C(O)—(CH₂)₁₄—SO₃H; CF₃—Ar—O—C(O)—(CH₂)₁₄—O—SO₃H;
CF₃—Ar—O—C(O)—(CH₂)₁₄—SH; CF₃—Ar—O—C(O)—(CH₂)₁₄—OH; CF₃—Ar—O—C(O)—(CH₂)₁₄—N⁺R¹R²R³Z⁻;
CF₃—Ar—O—C(O)—(CH₂)₁₄—P⁺R¹R²R³Z⁻; CF₃—Ar—O—C(O)—(CH₂)₁₄—O-
glucoside; CF₃—Ar—O—C(O)—(CH₂)₁₄—OPO₃H
CF₃—Ar—O—C(O)—(CH₂)₁₅—COOH; CF₃—Ar—O—C(O)—(CH₂)₁₅—SO₃H; CF₃—Ar—O—C(O)—(CH₂)₁₅—O—SO₃H;;
CF₃—Ar—O—C(O)—(CH₂)₁₅—SH; CF₃—Ar—O—C(O)—(CH₂)₁₅—OH; CF₃—Ar—O—C(O)—(CH₂)₁₅—N⁺R¹R²R³Z⁻;
CF₃—Ar—O—C(O)—(CH₂)₁₅—P⁺R¹R²R³Z⁻; CF₃—Ar—O—C(O)—(CH₂)₁₅—O-
glucoside; CF₃—Ar—O—C(O)—(CH₂)₁₅—OPO₃H
CF₃—Ar—O—C(O)—(CH₂)₁₆—COOH; CF₃—Ar—O—C(O)—(CH₂)₁₆—SO₃H; CF₃—Ar—O—C(O)—(CH₂)₁₆—O—SO₃H;
CF₃—Ar—O—C(O)—(CH₂)₁₆—SH; CF₃—Ar—O—C(O)—(CH₂)₁₆—OH; CF₃—Ar—O—C(O)—(CH₂)₁₆—N⁺R¹R²R³Z⁻;
CF₃—Ar—O—C(O)—(CH₂)₁₆—P⁺R¹R²R³Z⁻; CF₃—Ar—O—C(O)—(CH₂)₁₆—O-
glucoside; CF₃—Ar—O—C(O)—(CH₂)₁₆—OPO₃H
CF₃—Ar—O—C(O)—CH₂—CH═CH—(CH₂)₃—COOH; CF₃—Ar—O—C(O)—CH₂—CH═CH—(CH₂)₃—SO₃H;
CF₃—Ar—O—C(O)—CH₂—CH═CH—(CH₂)₃—O—SO₃H; CF₃—Ar—O—C(O)—CH₂—CH═CH—(CH₂)₃—SH;
CF₃—Ar—O—C(O)—CH₂—CH═CH—(CH₂)₃—OH; CF₃—Ar—O—C(O)—CH₂—CH═CH—(CH₂)₃—N⁺R¹R²R³Z⁻;
CF₃—Ar—O—C(O)—CH₂—CH═CH—(CH₂)₃—P⁺R¹R²R³Z⁻; CF₃—Ar—O—C(O)—CH₂—CH═CH—(CH₂)₃—O-
glucoside; CF₃—Ar—O—C(O)—CH₂—CH═CH—(CH₂)₃—O—PO₃H;
CF₃—Ar—O—C(O)—CH₂—CH═CH—(CH₂)₄—COOH; CF₃—Ar—O—C(O)—CH₂—CH═CH—(CH₂)₄—SO₃H;
CF₃—Ar—O—C(O)—CH₂—CH═CH—(CH₂)₄—O—SO₃H; CF₃—Ar—O—C(O)—CH₂—CH═CH—(CH₂)₄—SH;
CF₃—Ar—O—C(O)—CH₂—CH═CH—(CH₂)₄—OH; CF₃—Ar—O—C(O)—CH₂—CH═CH—(CH₂)₄—N⁺R¹R²R³Z⁻;
CF₃—Ar—O—C(O)—O—CH₂—CH═CH—(CH₂)₄—P⁺R¹R²R³Z⁻; CF₃—Ar—O—C(O)—CH₂—CH═CH—(CH₂)₄—O-
glucoside; CF₃—Ar—O—C(O)—CH₂—CH═CH—(CH₂)₄—O—PO₃H;
CF₃—Ar—O—C(O)—CH₂—CH═CH—(CH₂)₅—COOH; CF₃—Ar—O—C(O)—CH₂—CH═CH—(CH₂)₅—SO₃H;
CF₃—Ar—O—C(O)—CH₂—CH═CH—(CH₂)₅—O—SO₃H; CF₃—Ar—O—C(O)—CH₂—CH═CH—(CH₂)₅—SH;
CF₃—Ar—O—C(O)—CH₂—CH═CH—(CH₂)₅—OH; CF₃—Ar—O—C(O)—CH₂—CH═CH—(CH₂)₅—N⁺R¹R²R³Z⁻;
CF₃—Ar—O—C(O)—CH₂—CH═CH—(CH₂)₅—P⁺R¹R²R³Z⁻; CF₃—Ar—O—C(O)—CH₂—CH═CH—(CH₂)₅—O-
glucoside, CF₃—Ar—O—C(O)—CH₂—CH═CH—(CH₂)₅—O—PO₃H
CF₃—Ar—O—C(O)—CH₂—CH═CH—(CH₂)₆—COOH; CF₃—Ar—O—C(O)—CH₂—CH═CH—(CH₂)₆—SO₃H;
CF₃—Ar—O—C(O)—CH₂—CH═CH—(CH₂)₆—O—SO₃H; CF₃—Ar—O—C(O)—CH₂—CH═CH—(CH₂)₆—SH;
CF₃—Ar—O—C(O)—CH₂—CH═CH—(CH₂)₆—OH; CF₃—Ar—O—C(O)—O—CH₂—CH═CH—(CH₂)₆—N⁺R¹R²R³Z⁻;
CF₃—Ar—O—C(O)—CH₂—CH═CH—(CH₂)₆—P⁺R¹R²R³Z⁻; CF₃—Ar—O—C(O)—CH═CH—(CH₂)₆—O-
glucoside; CF₃—Ar—O—C(O)—CH₂—CH═CH—(CH₂)₆—OPO₃H
CF₃—Ar—O—C(O)—CH₂—CH═CH—(CH₂)₇—COOH; CF₃—Ar—O—C(O)—CH₂—CH═CH—(CH₂)₇—SO₃H;
CF₃—Ar—O—C(O)—CH₂—CH═CH—(CH₂)₇—O—SO₃H; CF₃—Ar—O—C(O)—CH₂—CH═CH—(CH₂)₇—SH;
CF₃—Ar—O—C(O)—CH₂—CH═CH—(CH₂)₇—OH; CF₃—Ar—O—C(O)—CH₂—CH═CH—(CH₂)₇—N⁺R¹R²R³Z⁻;
CF₃—Ar—O—C(O)—CH₂—CH═CH—(CH₂)₇—P⁺R¹R²R³Z⁻; CF₃—Ar—O—C(O)—CH₂—CH═CH—(CH₂)₇—O-
glucoside; CF₃—Ar—O—C(O)—CH₂—CH═CH—(CH₂)₇—OPO₃H
CF₃—Ar—O—C(O)—CH₂—CH═CH—(CH₂)₈—COOH; CF₃—Ar—O—C(O)—CH₂—CH═CH—(CH₂)₈—SO₃H;
CF₃—Ar—O—C(O)—CH₂—CH═CH—(CH₂)₈—O—SO₃H; CF₃—Ar—O—C(O)—CH₂—CH═CH—(CH₂)₈—SH;
CF₃—Ar—O—C(O)—CH₂—CH═CH—(CH₂)₈—OH; CF₃—Ar—O—C(O)—CH₂—CH═CH—(CH₂)₈—N⁺R¹R²R³Z⁻;
CF₃—Ar—O—C(O)—CH₂—CH═CH—(CH₂)₈—P⁺R¹R²R³Z⁻; CF₃—Ar—O—C(O)—CH₂—CH═CH—(CH₂)₈—O-
glucoside; CF₃—Ar—O—C(O)—CH₂—CH═CH—(CH₂)₈—OPO₃H
CF₃—Ar—O—C(O)—CH₂—CH═CH—(CH₂)₉—COOH; CF₃—Ar—O—C(O)—CH₂—CH═CH—(CH₂)₉—SO₃H;
CF₃—Ar—O—C(O)—CH₂—CH═CH—(CH₂)₉—O—SO₃H;; CF₃—Ar—O—C(O)—CH═CH—(CH₂)₉—SH;
CF₃—Ar—O—C(O)—CH₂—CH═CH—(CH₂)₉—OH; CF₃—Ar—O—C(O)—CH₂—CH═CH—(CH₂)₉—N⁺R¹R²R³Z⁻;
CF₃—Ar—O—C(O)—CH₂—CH═CH—(CH₂)₉—P⁺R¹R²R³Z⁻; CF₃—Ar—O—C(O)—CH₂—CH═CH—(CH₂)₉—O-
glucoside; CF₃—Ar—O—C(O)—CH₂—CH═CH—(CH₂)₉—OPO₃H
CF₃—Ar—O—C(O)—CH₂—CH═CH—(CH₂)₁₀—COOH; CF₃—Ar—O—C(O)—CH₂—CH═CH—(CH₂)₁₀—SO₃H;
CF₃—Ar—O—C(O)—CH₂—CH═CH—(CH₂)₁₀—O—SO₃H; CF₃—Ar—O—C(O)—CH₂—CH═CH—(CH₂)₁₀—SH;
CF₃—Ar—O—C(O)—CH₂—CH═CH—(CH₂)₁₀—OH; CF₃—Ar—O—C(O)—CH₂—CH═CH—(CH₂)₁₀—N⁺R¹R²R³Z⁻;
CF₃—Ar—O—C(O)—CH₂—CH═CH—(CH₂)₁₀—P⁺R¹R²R³Z⁻;
CF₃—Ar—O—C(O)—CH₂—CH═CH—(CH₂)₁₀—O-glucoside; CF₃—Ar—O—C(O)—CH₂—CH═CH—(CH₂)₁₀—OPO₃H
CF₃—Ar—O—C(O)—CH₂—CH═CH—(CH₂)₁₁—COOH; CF₃—Ar—O—C(O)—CH₂—CH═CH—(CH₂)₁₁—SO₃H;
CF₃—Ar—O—C(O)—CH₂—CH═CH—(CH₂)₁₁—O—SO₃H; CF₃—Ar—O—C(O)—CH₂—CH═CH—(CH₂)₁₁—SH;
CF₃—Ar—O—C(O)—CH₂—CH═CH—(CH₂)₁₁—OH; CF₃—Ar—O—C(O)—CH₂—CH═CH—(CH₂)₁₁—N⁺R¹R²R³Z⁻;
CF₃—Ar—O—C(O)—CH₂—CH═CH—(CH₂)₁₁—P⁺R¹R²R³Z⁻;
CF₃—Ar—O—C(O)—CH₂—CH═CH—(CH₂)₁₁—O-glucoside; CF₃—Ar—O—C(O)—CH₂—CH═CH—(CH₂)₁₁—OPO₃H
CF₃—Ar—O—C(O)—CH₂—CH═CH—(CH₂)₁₂—COOH; CF₃—Ar—O—C(O)—CH₂—CH═CH—(CH₂)₁₂—SO₃H;
CF₃—Ar—O—C(O)—CH₂—CH═CH—(CH₂)₁₂—O—SO₃H; CF₃—Ar—O—C(O)—CH₂—CH═CH—(CH₂)₁₂—SH;
CF₃—Ar—O—C(O)—CH₂—CH═CH—(CH₂)₁₂—OH; CF₃—Ar—O—C(O)—CH₂—CH═CH—(CH₂)₁₂—N⁺R¹R²R³Z⁻;
CF₃—Ar—O—C(O)—CH₂—CH═CH—(CH₂)₁₂—P⁺R¹R²R³Z⁻;
CF₃—Ar—O—C(O)—CH₂—CH═CH—(CH₂)₁₂—O-glucoside; CF₃—Ar—O—C(O)—CH₂—CH═CH—(CH₂)₁₂—OPO₃H
CF₃—Ar—O—C(O)—CH₂—CH═CH—(CH₂)₁₃—COOH; CF₃—Ar—O—C(O)—CH₂—CH═CH—(CH₂)₁₃—SO₃H;
CF₃—Ar—O—C(O)—CH₂—CH═CH—(CH₂)₁₃—O—SO₃H; CF₃—Ar—O—C(O)—CH₂—CH═CH—(CH₂)₁₃—SH;
CF₃—Ar—O—C(O)—CH₂—CH═CH—(CH₂)₁₃—OH; CF₃—Ar—O—C(O)—CH₂—CH═CH—(CH₂)₁₃—N⁺R¹R²R³Z⁻;
CF₃—Ar—O—C(O)—CH₂—CH═CH—(CH₂)₁₃—P⁺R¹R²R³Z⁻;
CF₃—Ar—O—C(O)—CH₂—CH═CH—(CH₂)₁₃—O-glucoside; CF₃—Ar—O—C(O)—CH₂—CH═CH—(CH₂)₁₃—OPO₃H
CF₃—Ar—O—C(O)—CH₂—CH═CH—(CH₂)₁₄—COOH; CF₃—Ar—O—C(O)—CH₂—CH═CH—(CH₂)₁₄—SO₃H;
CF₃—Ar—O—C(O)—CH₂—CH═CH—(CH₂)₁₄—O—SO₃H; CF₃—Ar—O—C(O)—CH₂—CH═CH—(CH₂)₁₄—SH;
CF₃—Ar—O—C(O)—CH₂—CH═CH—(CH₂)₁₄—OH; CF₃—Ar—O—C(O)—CH₂—CH═CH—(CH₂)₁₄—N⁺R¹R²R³Z⁻;
CF₃—Ar—O—C(O)—CH₂—CH═CH—(CH₂)₁₄—P⁺R¹R²R³Z⁻;
CF₃—Ar—O—C(O)—CH₂—CH═CH—(CH₂)₁₄—O-glucoside; CF₃—Ar—O—C(O)—CH₂—CH═CH—(CH₂)₁₄—OPO₃H
CF₃—Ar—O—C(O)—CH₂—CH═CH—(CH₂)₁₅—COOH; CF₃—Ar—O—C(O)—CH₂—CH═CH—(CH₂)₁₅—SO₃H;
CF₃—Ar—O—C(O)—CH₂—CH═CH—(CH₂)₁₅—O—SO₃H; CF₃—Ar—O—C(O)—CH₂—CH═CH—(CH₂)₁₅—SH;
CF₃—Ar—O—C(O)—CH₂—CH═CH—(CH₂)₁₅—OH; CF₃—Ar—O—C(O)—CH₂—CH═CH—(CH₂)₁₅—N⁺R¹R²R³Z⁻;
CF₃—Ar—O—C(O)—CH₂—CH═CH—(CH₂)₁₅—P⁺R¹R²R³Z⁻;
CF₃—Ar—O—C(O)—CH₂—CH═CH—(CH₂)₁₅—O-glucoside; CF₃—Ar—O—C(O)—CH₂—CH═CH—(CH₂)₁₅—OPO₃H
CF₃—Ar—O—C(O)—CH₂—CH═CH—(CH₂)₁₆—COOH; CF₃—Ar—O—C(O)—CH₂—CH═CH—(CH₂)₁₆—SO₃H;
CF₃—Ar—O—C(O)—CH₂—CH═CH—(CH₂)₁₆—O—SO₃H; CF₃—Ar—O—C(O)—CH₂—CH═CH—(CH₂)₁₆—SH;
CF₃—Ar—O—C(O)—CH₂—CH═CH—(CH₂)₁₆—OH; CF₃—Ar—O—C(O)—CH₂—CH═CH—(CH₂)₁₆—N⁺R¹R²R³Z⁻;
CF₃—Ar—O—C(O)—CH₂—CH═CH—(CH₂)₁₆—P⁺R¹R²R³Z⁻;
CF₃—Ar—O—C(O)—CH₂—CH═CH—(CH₂)₁₆—O-glucoside; CF₃—Ar—O—C(O)—CH₂—CH═CH—(CH₂)₁₆—OPO₃H
CF₃—O—Ar—O—C(O)—(CH₂)₃—COOH; CF₃—O—Ar—O—C(O)—(CH₂)₃—SO₃H; CF₃—O—Ar—O—C(O)—(CH₂)₃—O—SO₃H;
CF₃—O—Ar—O—C(O)—(CH₂)₃—COOH; CF₃—O—Ar—O—C(O)—(CH₂)₃—SO₃H; CF₃—O—Ar—O—C(O)—(CH₂)₃—O—SO₃H;
CF₃—O—Ar—O—C(O)—(CH₂)₃—SH; CF₃—O—Ar—O—C(O)—(CH₂)₃—OH;
CF₃—O—Ar—O—C(O)—(CH₂)₃—N⁺R¹R²R³Z⁻; CF₃—O—Ar—O—C(O)—(CH₂)₃—P⁺R¹R²R³Z⁻; CF₃—O—Ar—O—C(O)—(CH₂)₃—O-
glucoside; CF₃—O—Ar—O—C(O)—(CH₂)₃—O—PO₃H;
CF₃—O—Ar—O—C(O)—(CH₂)₄—COOH; CF₃—O—Ar—O—C(O)—(CH₂)₄—SO₃H; CF₃—O—Ar—O—C(O)—(CH₂)₄—O—SO₃H;
CF₃—O—Ar—O—C(O)—(CH₂)₄—SH; CF₃—O—Ar—O—C(O)—(CH₂)₄—OH;
CF₃—O—Ar—O—C(O)—(CH₂)₄—N⁺R¹R²R³Z⁻; CF₃—O—Ar—O—C(O)—(CH₂)₄—P⁺R¹R²R³Z⁻; CF₃—O—Ar—O—C(O)—(CH₂)₄—O-
glucoside; CF₃—O—Ar—O—C(O)—(CH₂)₄—O—PO₃H;
CF₃—O—Ar—O—C(O)—(CH₂)₅—COOH; CF₃—O—Ar—O—C(O)—(CH₂)₅—SO₃H; CF₃—O—Ar—O—C(O)—(CH₂)₅—O—SO₃H;
CF₃—O—Ar—O—C(O)—(CH₂)₅—SH; CF₃—O—Ar—O—C(O)—(CH₂)₅—OH;
CF₃—O—Ar—O—C(O)—(CH₂)₅—N⁺R¹R²R³Z⁻; CF₃—O—Ar—O—C(O)—(CH₂)₅—P⁺R¹R²R³Z⁻; CF₃—O—Ar—O—C(O)—(CH₂)₅—O-
glucoside; CF₃—O—Ar—O—C(O)—(CH₂)₅—O—PO₃H
CF₃—O—Ar—O—C(O)—(CH₂)₆—COOH; CF₃—O—Ar—O—C(O)—(CH₂)₆—SO₃H; CF₃—O—Ar—O—C(O)—(CH₂)₆—O—SO₃H;
CF₃—O—Ar—O—C(O)—(CH₂)₆—SH; CF₃—O—Ar—O—C(O)—(CH₂)₆—OH;
CF₃—O—Ar—O—C(O)—(CH₂)₆—N⁺R¹R²R³Z⁻; CF₃—O—Ar—O—C(O)—(CH₂)₆—P⁺R¹R²R³Z⁻; CF₃—O—Ar—O—C(O)—(CH₂)₆—O-
glucoside; CF₃—O—Ar—O—C(O)—(CH₂)₆—OPO₃H
CF₃—O—Ar—O—C(O)—(CH₂)₇—COOH; CF₃—O—Ar—O—C(O)—(CH₂)₇—SO₃H; CF₃—O—Ar—O—C(O)—(CH₂)₇—O—SO₃H;
CF₃—O—Ar—O—C(O)—(CH₂)₇—SH; CF₃—O—Ar—O—C(O)—(CH₂)₇—OH;
CF₃—O—Ar—O—C(O)—(CH₂)₇—N⁺R¹R²R³Z⁻; CF₃—O—Ar—O—C(O)—(CH₂)₇—P⁺R¹R²R³Z⁻; CF₃—O—Ar—O—C(O)—(CH₂)₇—O-
glucoside; CF₃—O—Ar—O—C(O)—(CH₂)₇—OPO₃H
CF₃—O—Ar—O—C(O)—(CH₂)₈—COOH; CF₃—O—Ar—O—C(O)—(CH₂)₈—SO₃H; CF₃—O—Ar—O—C(O)—(CH₂)₈—O—SO₃H;
CF₃—O—Ar—O—C(O)—(CH₂)₈—SH; CF₃—O—Ar—O—C(O)—(CH₂)₈—OH;
CF₃—O—Ar—O—C(O)—(CH₂)₈—N⁺R¹R²R³Z⁻; CF₃—O—Ar—O—C(O)—(CH₂)₈—P⁺R¹R²R³Z⁻; CF₃—O—Ar—O—C(O)—(CH₂)₈—O-
glucoside; CF₃—O—Ar—O—C(O)—(CH₂)₈—OPO₃H
CF₃—O—Ar—O—C(O)—(CH₂)₉—COOH; CF₃—O—Ar—O—C(O)—(CH₂)₉—SO₃H; CF₃—O—Ar—O—C(O)—(CH₂)₉—O—SO₃H;;
CF₃—O—Ar—O—C(O)—(CH₂)₉—SH; CF₃—O—Ar—O—C(O)—(CH₂)₉—OH;
CF₃—O—Ar—O—C(O)—(CH₂)₉—N⁺R¹R²R³Z⁻; CF₃—O—Ar—O—C(O)—(CH₂)₉—P⁺R¹R²R³Z⁻; CF₃—O—Ar—O—C(O)—(CH₂)₉—O-
glucoside; CF₃—O—Ar—O—C(O)—(CH₂)₉—OPO₃H
CF₃—O—Ar—O—C(O)—(CH₂)₁₀—COOH; CF₃—O—Ar—O—C(O)—(CH₂)₁₀—SO₃H; CF₃—O—Ar—O—C(O)—(CH₂)₁₀—O—SO₃H;
CF₃—O—Ar—O—C(O)—(CH₂)₁₀—SH; CF₃—O—Ar—O—C(O)—(CH₂)₁₀—OH;
CF₃—O—Ar—O—C(O)—(CH₂)₁₀—N⁺R¹R²R³Z⁻; CF₃—O—Ar—O—C(O)—(CH₂)₁₀—P⁺R¹R²R³Z⁻;
CF₃—O—Ar—O—C(O)—(CH₂)₁₀—O-glucoside; CF₃—O—Ar—O—C(O)—(CH₂)₁₀—OPO₃H
CF₃—O—Ar—O—C(O)—(CH₂)₁₁—COOH; CF₃—O—Ar—O—C(O)—(CH₂)₁₁—SO₃H; CF₃—O—Ar—O—C(O)—(CH₂)₁₁—O—SO₃H;
CF₃—O—Ar—O—C(O)—(CH₂)₁₁—SH; CF₃—O—Ar—O—C(O)—(CH₂)₁₁—OH;
CF₃—O—Ar—O—C(O)—(CH₂)₁₁—N⁺R¹R²R³Z⁻; CF₃—O—Ar—O—C(O)—(CH₂)₁₁—P⁺R¹R²R³Z⁻;
CF₃—O—Ar—O—C(O)—(CH₂)₁₁—O-glucoside; CF₃—O—Ar—O—C(O)—(CH₂)₁₁—OPO₃H
CF₃—O—Ar—O—C(O)—(CH₂)₁₂—COOH; CF₃—O—Ar—O—C(O)—(CH₂)₁₂—SO₃H; CF₃—O—Ar—O—C(O)—(CH₂)₁₂—O—SO₃H;
CF₃—O—Ar—O—C(O)—(CH₂)₁₂—SH; CF₃—O—Ar—O—C(O)—(CH₂)₁₂—OH;
CF₃—O—Ar—O—C(O)—(CH₂)₁₂—N⁺R¹R²R³Z⁻; CF₃—O—Ar—O—C(O)—(CH₂)₁₂—P⁺R¹R²R³Z⁻;
CF₃—O—Ar—O—C(O)—(CH₂)₁₂—O-glucoside; CF₃—O—Ar—O—C(O)—(CH₂)₁₂—OPO₃H
CF₃—O—Ar—O—C(O)—(CH₂)₁₃—COOH; CF₃—O—Ar—O—C(O)—(CH₂)₁₃—SO₃H; CF₃—O—Ar—O—C(O)—(CH₂)₁₃—O—SO₃H;
CF₃—O—Ar—O—C(O)—(CH₂)₁₃—SH; CF₃—O—Ar—O—C(O)—(CH₂)₁₃—OH;
CF₃—O—Ar—O—C(O)—(CH₂)₁₃—N⁺R¹R²R³Z⁻; CF₃—O—Ar—O—C(O)—(CH₂)₁₃—P⁺R¹R²R³Z⁻;
CF₃—O—Ar—O—C(O)—(CH₂)₁₃—O-glucoside; CF₃—O—Ar—O—C(O)—(CH₂)₁₃—OPO₃H
CF₃—O—Ar—O—C(O)—(CH₂)₁₄—COOH; CF₃—O—Ar—O—C(O)—(CH₂)₁₄—SO₃H; CF₃—O—Ar—O—C(O)—(CH₂)₁₄—O—SO₃H;
CF₃—O—Ar—O—C(O)—(CH₂)₁₄—SH; CF₃—O—Ar—O—C(O)—(CH₂)₁₄—OH;
CF₃—O—Ar—O—C(O)—(CH₂)₁₄—N⁺R¹R²R³Z⁻; CF₃—O—Ar—O—C(O)—(CH₂)₁₄—P⁺R¹R²R³Z⁻;
CF₃—O—Ar—O—C(O)—(CH₂)₁₄—O-glucoside; CF₃—O—Ar—O—C(O)—(CH₂)₁₄—OPO₃H
CF₃—O—Ar—O—C(O)—(CH₂)₁₅—COOH; CF₃—O—Ar—O—C(O)—(CH₂)₁₅—SO₃H; CF₃—O—Ar—O—C(O)—(CH₂)₁₅—O—SO₃H;;
CF₃—O—Ar—O—C(O)—(CH₂)₁₅—SH; CF₃—O—Ar—O—C(O)—(CH₂)₁₅—OH;
CF₃—O—Ar—O—C(O)—(CH₂)₁₅—N⁺R¹R²R³Z⁻; CF₃—O—Ar—O—C(O)—(CH₂)₁₅—P⁺R¹R²R³Z⁻;
CF₃—O—Ar—O—C(O)—(CH₂)₁₅—O-glucoside; CF₃—O—Ar—O—C(O)—(CH₂)₁₅—OPO₃H
CF₃—O—Ar—O—C(O)—(CH₂)₁₆—COOH; CF₃—O—Ar—O—C(O)—(CH₂)₁₆—SO₃H; CF₃—O—Ar—O—C(O)—(CH₂)₁₆—O—SO₃H;
CF₃—O—Ar—O—C(O)—(CH₂)₁₆—SH; CF₃—O—Ar—O—C(O)—(CH₂)₁₆—OH;
CF₃—O—Ar—O—C(O)—(CH₂)₁₆—N⁺R¹R²R³Z⁻; CF₃—O—Ar—O—C(O)—(CH₂)₁₆—P⁺R¹R²R³Z⁻;
CF₃—O—Ar—O—C(O)—(CH₂)₁₆—O-glucoside; CF₃—O—Ar—O—C(O)—(CH₂)₁₆—OPO₃H
CF₃—O—Ar—O—C(O)—CH₂—CH═CH—(CH₂)₃—COOH; CF₃—O—Ar—O—C(O)—CH₂—CH═CH—(CH₂)₃—SO₃H;
CF₃—O—Ar—O—C(O)—CH₂—CH═CH—(CH₂)₃—O—SO₃H; CF₃—O—Ar—O—C(O)—CH₂—CH═CH—(CH₂)₃—SH;
CF₃—O—Ar—O—C(O)—CH₂—CH═CH—(CH₂)₃—OH; CF₃—O—Ar—O—C(O)—CH₂—CH═CH—(CH₂)₃—N⁺R¹R²R³Z⁻;
CF₃—O—Ar—O—C(O)—CH₂—CH═CH—(CH₂)₃—P⁺R¹R²R³Z⁻;
CF₃—O—Ar—O—C(O)—CH₂—CH═CH—(CH₂)₃—O-glucoside; CF₃—O—Ar—O—C(O)—CH₂—CH═CH—(CH₂)₃—O—PO₃H;
CF₃—O—Ar—O—C(O)—CH₂—CH═CH—(CH₂)₄—COOH; CF₃—O—Ar—O—C(O)—CH₂—CH═CH—(CH₂)₄—SO₃H;
CF₃—O—Ar—O—C(O)—CH₂—CH═CH—(CH₂)₄—O—SO₃H; CF₃—O—Ar—O—C(O)—CH₂—CH═CH—(CH₂)₄—SH;
CF₃—O—Ar—O—C(O)—CH₂—CH═CH—(CH₂)₄—OH; CF₃—O—Ar—O—C(O)—CH₂—CH═CH—(CH₂)₄—N⁺R¹R²R³Z⁻;
CF₃—O—Ar—O—C(O)—CH₂—CH═CH—(CH₂)₄—P⁺R¹R²R³Z⁻;
CF₃—O—Ar—O—C(O)—CH₂—CH═CH—(CH₂)₄—O-glucoside; CF₃—O—Ar—O—C(O)—CH₂—CH═CH—(CH₂)₄—O—PO₃H;
CF₃—O—Ar—O—C(O)—CH₂—CH═CH—(CH₂)₅—COOH; CF₃—O—Ar—O—C(O)—CH₂—CH═CH—(CH₂)₅—SO₃H;
CF₃—O—Ar—O—C(O)—CH₂—CH═CH—(CH₂)₅—O—SO₃H; CF₃—O—Ar—O—C(O)—CH₂—CH═CH—(CH₂)₅—SH;
CF₃—O—Ar—O—C(O)—CH₂—CH═CH—(CH₂)₅—OH; CF₃—O—Ar—O—C(O)—CH₂—CH═CH—(CH₂)₅—N⁺R¹R²R³Z⁻;
CF₃—O—Ar—O—C(O)—CH₂—CH═CH—(CH₂)₅—P⁺R¹R²R³Z⁻;
CF₃—O—Ar—O—C(O)—CH₂—CH═CH—(CH₂)₅—O-glucoside; CF₃—O—Ar—O—C(O)—CH₂—CH═CH—(CH₂)₅—O—PO₃H
CF₃—O—Ar—O—C(O)—CH₂—CH═CH—(CH₂)₆—COOH; CF₃—O—Ar—O—C(O)—CH₂—CH═CH—(CH₂)₆—SO₃H;
CF₃—O—Ar—O—C(O)—CH₂—CH═CH—(CH₂)₆—O—SO₃H; CF₃—O—Ar—O—C(O)—CH₂—CH═CH—(CH₂)₆—SH;
CF₃—O—Ar—O—C(O)—CH₂—CH═CH—(CH₂)₆—OH; CF₃—O—Ar—O—C(O)—CH₂—CH═CH—(CH₂)₆—N⁺R¹R²R³Z⁻;
CF₃—O—Ar—O—C(O)—CH₂—CH═CH—(CH₂)₆—P⁺R¹R²R³Z⁻;
CF₃—O—Ar—O—C(O)—CH₂—CH═CH—(CH₂)₆—O-glucoside; CF₃—O—Ar—O—C(O)—CH₂—CH═CH—(CH₂)₆—OPO₃H
CF₃—O—Ar—O—C(O)—CH₂—CH═CH—(CH₂)₇—COOH; CF₃—O—Ar—O—C(O)—CH₂—CH═CH—(CH₂)₇—SO₃H;
CF₃—O—Ar—O—C(O)—CH₂—CH═CH—(CH₂)₇—O—SO₃H; CF₃O——Ar—O—C(O)—CH₂—CH═CH—(CH₂)₇—SH;
CF₃—O—Ar—O—C(O)—CH₂—CH═CH—(CH₂)₇—OH; CF₃—O—Ar—O—C(O)—CH₂—CH═CH—(CH₂)₇—N⁺R¹R²R³Z⁻;
CF₃—O—Ar—O—C(O)—CH₂—CH═CH—(CH₂)₇—P⁺R¹R²R³Z⁻;
CF₃—O—Ar—O—C(O)—CH₂—CH═CH—(CH₂)₇—O-glucoside; CF₃—O—Ar—O—C(O)—CH₂—CH═CH—(CH₂)₇—OPO₃H
CF₃—O—Ar—O—C(O)—CH₂—CH═CH—(CH₂)₈—COOH; CF₃—O—Ar—O—C(O)—CH₂—CH═CH—(CH₂)₈—SO₃H;
CF₃—O—Ar—O—C(O)—CH₂—CH═CH—(CH₂)₈—O—SO₃H; CF₃—O—Ar—O—C(O)—CH₂—CH═CH—(CH₂)₈—SH;
CF₃—O—Ar—O—C(O)—CH₂—CH═CH—(CH₂)₈—OH; CF₃—O—Ar—O—C(O)—CH₂—CH═CH—(CH₂)₈—N⁺R¹R²R³Z⁻;
CF₃—O—Ar—O—C(O)—CH₂—CH═CH—(CH₂)₈—P⁺R¹R²R³Z⁻;
CF₃—O—Ar—O—C(O)—CH₂—CH═CH—(CH₂)₈—O-glucoside; CF₃—O—Ar—O—C(O)—CH₂—CH═CH—(CH₂)₈—OPO₃H
CF₃—O—Ar—O—C(O)—CH₂—CH═CH—(CH₂)₉—COOH; CF₃—O—Ar—O—C(O)—CH₂—CH═CH—(CH₂)₉—SO₃H;
CF₃—O—Ar—O—C(O)—CH₂—CH═CH—(CH₂)₉—O—SO₃H;; CF₃—O—Ar—O—C(O)—CH₂—CH═CH—(CH₂)₉—SH;
CF₃—O—Ar—O—C(O)—CH₂—CH═CH—(CH₂)₉—OH; CF₃—O—Ar—O—C(O)—CH₂—CH═CH—(CH₂)₉—N⁺R¹R²R³Z⁻;
CF₃—O—Ar—O—C(O)—CH₂—CH═CH—(CH₂)₉—P⁺R¹R²R³Z⁻;
CF₃—O—Ar—O—C(O)—CH₂—CH═CH—(CH₂)₉—O-glucoside; CF₃—O—Ar—O—C(O)—CH₂—CH═CH—(CH₂)₉—OPO₃H
CF₃—O—Ar—O—C(O)—CH₂—CH═CH—(CH₂)₁₀—COOH; CF₃—O—Ar—O—C(O)—CH₂—CH═CH—(CH₂)₁₀—SO₃H;
CF₃—O—Ar—O—C(O)—CH₂—CH═CH—(CH₂)₁₀—O—SO₃H; CF₃—O—Ar—O—C(O)—CH₂—CH═CH—(CH₂)₁₀—SH;
CF₃—O—Ar—O—C(O)—CH₂—CH═CH—(CH₂)₁₀—OH; CF₃—O—Ar—O—C(O)—CH₂—CH═CH—(CH₂)₁₀—N⁺R¹R²R³Z⁻;
CF₃—O—Ar—O—C(O)—CH₂—CH═CH—(CH₂)₁₀—P⁺R¹R²R³Z⁻;
CF₃—O—Ar—O—C(O)—CH₂—CH═CH—(CH₂)₁₀—O-glucoside; CF₃—O—Ar—O—C(O)—CH₂—CH═CH—(CH₂)₁₀—OPO₃H
CF₃—O—Ar—O—C(O)—CH₂—CH═CH—(CH₂)₁₁—COOH; CF₃—O—Ar—O—C(O)—CH₂—CH═CH—(CH₂)₁₁—SO₃H;
CF₃—O—Ar—O—C(O)—CH₂—CH═CH—(CH₂)₁₁—O—SO₃H; CF₃—O—Ar—O—C(O)—CH₂—CH═CH—(CH₂)₁₁—SH;
CF₃—O—Ar—O—C(O)—CH₂—CH═CH—(CH₂)₁₁—OH; CF₃—O—Ar—O—C(O)—CH₂—CH═CH—(CH₂)₁₁—N⁺R¹R²R³Z⁻;
CF₃—O—Ar—O—C(O)—CH₂—CH═CH—(CH₂)₁₁—P⁺R¹R²R³Z⁻;
CF₃—O—Ar—O—C(O)—CH₂—CH═CH—(CH₂)₁₁—O-glucoside; CF₃—O—Ar—O—C(O)—CH₂—CH═CH—(CH₂)₁₁—OPO₃H
CF₃—O—Ar—O—C(O)—CH₂—CH═CH—(CH₂)₁₂—COOH; CF₃—O—Ar—O—C(O)—CH₂—CH═CH—(CH₂)₁₂—SO₃H;
CF₃—O—Ar—O—C(O)—CH₂—CH═CH—(CH₂)₁₂—O—SO₃H; CF₃—O—Ar—O—C(O)—CH₂—CH═CH—(CH₂)₁₂—SH;
CF₃—O—Ar—O—C(O)—CH₂—CH═CH—(CH₂)₁₂—OH; CF₃—O—Ar—O—C(O)—CH₂—CH═CH—(CH₂)₁₂—N⁺R¹R²R³Z⁻;
CF₃—O—Ar—O—C(O)—CH₂—CH═CH—(CH₂)₁₂—P⁺R¹R²R³Z⁻;
CF₃—O—Ar—O—C(O)—CH₂—CH═CH—(CH₂)₁₂—O-glucoside; CF₃—O—Ar—O—C(O)—CH₂—CH═CH—(CH₂)₁₂—OPO₃H
CF₃—O—Ar—O—C(O)—CH₂—CH═CH—(CH₂)₁₃—COOH; CF₃—O—Ar—O—C(O)—CH₂—CH═CH—(CH₂)₁₃—SO₃H;
CF₃—O—Ar—O—C(O)—CH₂—CH═CH—(CH₂)₁₃—O—SO₃H; CF₃—O—Ar—O—C(O)—CH₂—CH═CH—(CH₂)₁₃—SH;
CF₃—O—Ar—O—C(O)—CH₂—CH═CH—(CH₂)₁₃—OH; CF₃—O—Ar—O—C(O)—CH₂—CH═CH—(CH₂)₁₃—N⁺R¹R²R³Z⁻;
CF₃—O—Ar—O—C(O)—CH₂—CH═CH—(CH₂)₁₃—P⁺R¹R²R³Z⁻;
CF₃—O—Ar—O—C(O)—CH₂—CH═CH—(CH₂)₁₃—O-glucoside; CF₃—O—Ar—O—C(O)—CH₂—CH═CH—(CH₂)₁₃—OPO₃H
CF₃—O—Ar—O—C(O)—CH₂—CH═CH—(CH₂)₁₄—COOH; CF₃—O—Ar—O—C(O)—CH₂—CH═CH—(CH₂)₁₄—SO₃H;
CF₃—O—Ar—O—C(O)—CH₂—CH═CH—(CH₂)₁₄—O—SO₃H; CF₃—O—Ar—O—C(O)—CH₂—CH═CH—(CH₂)₁₄—SH;
CF₃—O—Ar—O—C(O)—CH₂—CH═CH—(CH₂)₁₄—OH; CF₃—O—Ar—O—C(O)—CH₂—CH═CH—(CH₂)₁₄—N⁺R¹R²R³Z⁻;
CF₃—O—Ar—O—C(O)—CH₂—CH═CH—(CH₂)₁₄—P⁺R¹R²R³Z⁻;
CF₃—O—Ar—O—C(O)—CH₂—CH═CH—(CH₂)₁₄—O-glucoside; CF₃—O—Ar—O—C(O)—CH₂—CH═CH—(CH₂)₁₄—OPO₃H
CF₃—O—Ar—O—C(O)—CH₂—CH═CH—(CH₂)₁₅—COOH; CF₃—O—Ar—O—C(O)—CH₂—CH═CH—(CH₂)₁₅—SO₃H;
CF₃—O—Ar—O—C(O)—CH₂—CH═CH—(CH₂)₁₅—O—SO₃H; CF₃—O—Ar—O—C(O)—CH₂—CH═CH—(CH₂)₁₅—SH;
CF₃—O—Ar—O—C(O)—CH₂—CH═CH—(CH₂)₁₅—OH; CF₃—O—Ar—O—C(O)—CH₂—CH═CH—(CH₂)₁₅—N⁺R¹R²R³Z⁻;
CF₃—O—Ar—O—C(O)—CH₂—CH═CH—(CH₂)₁₅—P⁺R¹R²R³Z⁻;
CF₃—O—Ar—O—C(O)—CH₂—CH═CH—(CH₂)₁₅—O-glucoside; CF₃—O—Ar—O—C(O)—CH₂—CH═CH—(CH₂)₁₅—OPO₃H
CF₃—O—Ar—O—C(O)—CH₂—CH═CH—(CH₂)₁₆—COOH; CF₃—O—Ar—O—C(O)—CH₂—CH═CH—(CH₂)₁₆—SO₃H;
CF₃—O—Ar—O—C(O)—CH₂—CH═CH—(CH₂)₁₆—O—SO₃H; CF₃—O—Ar—O—C(O)—CH₂—CH═CH—(CH₂)₁₆—SH;
CF₃—O—Ar—O—C(O)—CH₂—CH═CH—(CH₂)₁₆—OH; CF₃—O—Ar—O—C(O)—CH₂—CH═CH—(CH₂)₁₆—N⁺R¹R²R³Z⁻;
CF₃—O—Ar—O—C(O)—CH₂—CH═CH—(CH₂)₁₆—P⁺R¹R²R³Z⁻;
CF₃—O—Ar—O—C(O)—CH₂—CH═CH—(CH₂)₁₆—O-glucoside; CF₃—O—Ar—O—C(O)—CH₂—CH═CH—(CH₂)₁₆—OPO₃H
CF₃—CH₂—CH₂—O—Ar—O—C(O)—(CH₂)₃—COOH; CF₃—CH₂—CH₂—O—Ar—O—C(O)—(CH₂)₃—SO₃H;
CF₃—CH₂—CH₂—O—Ar—O—C(O)—(CH₂)₃—O—SO₃H; CF₃—CH₂—CH₂—O—Ar—O—C(O)—(CH₂)₃—SH;
CF₃—CH₂—CH₂—O—Ar—O—C(O)—(CH₂)₃—OH; CF₃—CH₂—CH₂—O—Ar—O—C(O)—(CH₂)₃—N⁺R¹R²R³Z⁻;
CF₃—CH₂—CH₂—O—Ar—O—C(O)—(CH₂)₃—P⁺R¹R²R³Z⁻; CF₃—CH₂—CH₂—O—Ar—O—C(O)—(CH₂)₃—O-
glucoside; CF₃—CH₂—CH₂—O—Ar—O—C(O)—(CH₂)₃—O—PO₃H;
CF₃—CH₂—CH₂—O—Ar—O—C(O)—(CH₂)₄—COOH; CF₃—CH₂—CH₂—O—Ar—O—C(O)—(CH₂)₄—SO₃H;
CF₃—CH₂—CH₂—O—Ar—O—C(O)—(CH₂)₄—O—SO₃H; CF₃—CH₂—CH₂—O—Ar—O—C(O)—(CH₂)₄—SH;
CF₃—CH₂—CH₂—O—Ar—O—C(O)—(CH₂)₄—OH; CF₃—CH₂—CH₂—O—Ar—O—C(O)—(CH₂)₄—N⁺R¹R²R³Z⁻;
CF₃—CH₂—CH₂—O—Ar—O—C(O)—(CH₂)₄—P⁺R¹R²R³Z⁻; CF₃—CH₂—CH₂—O—Ar—O—C(O)—(CH₂)₄—O-
glucoside; CF₃—CH₂—CH₂—O—Ar—O—C(O)—(CH₂)₄—O—PO₃H;
CF₃—CH₂—CH₂—O—Ar—O—C(O)—(CH₂)₅—COOH; CF₃—CH₂—CH₂—O—Ar—O—C(O)—(CH₂)₅—SO₃H;
CF₃—CH₂—CH₂—O—Ar—O—C(O)—(CH₂)₅—O—SO₃H; CF₃—CH₂—CH₂—O—Ar—O—C(O)—(CH₂)₅—SH;
CF₃—CH₂—CH₂—O—Ar—O—C(O)—(CH₂)₅—OH; CF₃—CH₂—CH₂—O—Ar—O—C(O)—(CH₂)₅—N⁺R¹R²R³Z⁻;
CF₃—CH₂—CH₂—O—Ar—O—C(O)—(CH₂)₅—P⁺R¹R²R³Z⁻; CF₃—CH₂—CH₂—O—Ar—O—C(O)—(CH₂)₅—O-
glucoside; CF₃—CH₂—CH₂—O—Ar—O—C(O)—(CH₂)₅—O—PO₃H
CF₃—CH₂—CH₂—O—Ar—O—C(O)—(CH₂)₆—COOH; CF₃—CH₂—CH₂—O—Ar—O—C(O)—(CH₂)₆—SO₃H;
CF₃—CH₂—CH₂—O—Ar—O—C(O)—(CH₂)₆—O—SO₃H; CF₃—CH₂—CH₂—O—Ar—O—C(O)—(CH₂)₆—SH;
CF₃—CH₂—CH₂—O—Ar—O—C(O)—(CH₂)₆—OH; CF₃—CH₂—CH₂—O—Ar—O—C(O)—(CH₂)₆—N⁺R¹R²R³Z⁻;
CF₃—CH₂—CH₂—O—Ar—O—C(O)—(CH₂)₆—P⁺R¹R²R³Z⁻; CF₃—CH₂—CH₂—O—Ar—O—C(O)—(CH₂)₆—O-
glucoside; CF₃—CH₂—CH₂—O—Ar—O—C(O)—(CH₂)₆—OPO₃H
CF₃—CH₂—CH₂—O—Ar—O—C(O)—(CH₂)₇—COOH; CF₃—CH₂—CH₂—O—Ar—O—C(O)—(CH₂)₇—SO₃H;
CF₃—CH₂—CH₂—O—Ar—O—C(O)—(CH₂)₇—O—SO₃H; CF₃—CH₂—CH₂—O—Ar—O—C(O)—(CH₂)₇—SH;
CF₃—CH₂—CH₂—O—Ar—O—C(O)—(CH₂)₇—OH; CF₃—CH₂—CH₂—O—Ar—O—C(O)—(CH₂)₇—N⁺R¹R²R³Z⁻;
CF₃—CH₂—CH₂—O—Ar—O—C(O)—(CH₂)₇—P⁺R¹R²R³Z⁻; CF₃—CH₂—CH₂—O—Ar—O—C(O)—(CH₂)₇—O-
glucoside; CF₃—CH₂—CH₂—O—Ar—O—C(O)—(CH₂)₇O—OPO₃H
CF₃—CH₂—CH₂—O—Ar—O—C(O)—(CH₂)₈—COOH; CF₃—CH₂—CH₂—O—Ar—O—C(O)—(CH₂)₈—SO₃H;
CF₃—CH₂—CH₂—O—Ar—O—C(O)—(CH₂)₈—O—SO₃H; CF₃—CH₂—CH₂—O—Ar—O—C(O)—(CH₂)₈—SH;
CF₃—CH₂—CH₂—O—Ar—O—C(O)—(CH₂)₈—OH; CF₃—CH₂—CH₂—O—Ar—O—C(O)—(CH₂)₈—N⁺R¹R²R³Z⁻;
CF₃—CH₂—CH₂—O—Ar—O—C(O)—(CH₂)₈—P⁺R¹R²R³Z⁻; CF₃—CH₂—CH₂—O—Ar—O—C(O)—(CH₂)₈—O-
glucoside; CF₃—CH₂—CH₂—O—Ar—O—C(O)—(CH₂)₈—OPO₃H
CF₃—CH₂—CH₂—O—Ar—O—C(O)—(CH₂)₉—COOH; CF₃—CH₂—CH₂—O—Ar—O—C(O)—(CH₂)₉—SO₃H;
CF₃—CH₂—CH₂—O—Ar—O—C(O)—(CH₂)₉—O—SO₃H;; CF₃—CH₂—CH₂—O—Ar—O—C(O)—(CH₂)₉—SH;
CF₃—CH₂—CH₂—O—Ar—O—C(O)—(CH₂)₉—OH; CF₃—CH₂—CH₂—O—Ar—O—C(O)—(CH₂)₉—N⁺R¹R²R³Z⁻;
CF₃—CH₂—CH₂—O—Ar—O—C(O)—(CH₂)₉—P⁺R¹R²R³Z⁻; CF₃—CH₂—CH₂—O—Ar—O—C(O)—(CH₂)₉—O-
glucoside; CF₃—CH₂—CH₂—O—Ar—O—C(O)—(CH₂)₉—OPO₃H
CF₃—CH₂—CH₂—O—Ar—O—C(O)—(CH₂)₁₀—COOH; CF₃—CH₂—CH₂—O—Ar—O—C(O)—(CH₂)₁₀—SO₃H;
CF₃—CH₂—CH₂—O—Ar—O—C(O)—(CH₂)₁₀—O—SO₃H; CF₃—CH₂—CH₂—O—Ar—O—C(O)—(CH₂)₁₀—SH;
CF₃—CH₂—CH₂—O—Ar—O—C(O)—(CH₂)₁₀—OH; CF₃—CH₂—CH₂—O—Ar—O—C(O)—(CH₂)₁₀—N⁺R¹R²R³Z⁻;
CF₃—CH₂—CH₂—O—Ar—O—C(O)—(CH₂)₁₀—P⁺R¹R²R³Z⁻; CF₃—CH₂—CH₂—O—Ar—O—C(O)—(CH₂)₁₀—O-
glucoside; CF₃—CH₂—CH₂—O—Ar—O—C(O)—(CH₂)₁₀—OPO₃H
CF₃—CH₂—CH₂—O—Ar—O—C(O)—(CH₂)₁₁—COOH; CF₃—CH₂—CH₂—O—Ar—O—C(O)—(CH₂)₁₁—SO₃H;
CF₃—CH₂—CH₂—O—Ar—O—C(O)—(CH₂)₁₁—O—SO₃H; CF₃—CH₂—CH₂—O—Ar—O—C(O)—(CH₂)₁₁—SH;
CF₃—CH₂—CH₂—O—Ar—O—C(O)—(CH₂)₁₁—OH; CF₃—CH₂—CH₂—O—Ar—O—C(O)—(CH₂)₁₁—N⁺R¹R²R³Z⁻;
CF₃—CH₂—CH₂—O—Ar—O—C(O)—(CH₂)₁₁—P⁺R¹R²R³Z⁻; CF₃—CH₂—CH₂—O—Ar—O—C(O)—(CH₂)₁₁—O-
glucoside; CF₃—CH₂—CH₂—O—Ar—O—C(O)—(CH₂)₁₁—OPO₃H
CF₃—CH₂—CH₂—O—Ar—O—C(O)—(CH₂)₁₂—COOH; CF₃—CH₂—CH₂—O—Ar—O—C(O)—(CH₂)₁₂—SO₃H;
CF₃—CH₂—CH₂—O—Ar—O—C(O)—(CH₂)₁₂—O—SO₃H; CF₃—CH₂—CH₂—O—Ar—O—C(O)—(CH₂)₁₂—SH;
CF₃—CH₂—CH₂—O—Ar—O—C(O)—(CH₂)₁₂—OH; CF₃—CH₂—CH₂—O—Ar—O—C(O)—(CH₂)₁₂—N⁺R¹R²R³Z⁻;
CF₃—CH₂—CH₂—O—Ar—O—C(O)—(CH₂)₁₂—P⁺R¹R²R³Z⁻; CF₃—CH₂—CH₂—O—Ar—O—C(O)—(CH₂)₁₂—O-
glucoside; CF₃—CH₂—CH₂—O—Ar—O—C(O)—(CH₂)₁₂—OPO₃H
CF₃—CH₂—CH₂—O—Ar—O—C(O)—(CH₂)₁₃—COOH; CF₃—CH₂—CH₂—O—Ar—O—C(O)—(CH₂)₁₃—SO₃H;
CF₃—CH₂—CH₂—O—Ar—O—C(O)—(CH₂)₁₃—O—SO₃H; CF₃—CH₂—CH₂—O—Ar—O—C(O)—(CH₂)₁₃—SH;
CF₃—CH₂—CH₂—O—Ar—O—C(O)—(CH₂)₁₃—OH; CF₃—CH₂—CH₂—O—Ar—O—C(O)—(CH₂)₁₃—N⁺R¹R²R³Z⁻;
CF₃—CH₂—CH₂—O—Ar—O—C(O)—(CH₂)₁₃—P⁺R¹R²R³Z⁻; CF₃—CH₂—CH₂—O—Ar—O—C(O)—(CH₂)₁₃—O-
glucoside; CF₃—CH₂—CH₂—O—Ar—O—C(O)—(CH₂)₁₃—OPO₃H
CF₃—CH₂—CH₂—O—Ar—O—C(O)—(CH₂)₁₄—COOH; CF₃—CH₂—CH₂—O—Ar—O—C(O)—(CH₂)₁₄—SO₃H;
CF₃—CH₂—CH₂—O—Ar—O—C(O)—(CH₂)₁₄—O—SO₃H; CF₃—CH₂—CH₂—O—Ar—O—C(O)—(CH₂)₁₄—SH;
CF₃—CH₂—CH₂—O—Ar—O—C(O)—(CH₂)₁₄—OH; CF₃—CH₂—CH₂—O—Ar—O—C(O)—(CH₂)₁₄—N⁺R¹R²R³Z⁻;
CF₃—CH₂—CH₂—O—Ar—O—C(O)—(CH₂)₁₄—P⁺R¹R²R³Z⁻; CF₃—CH₂—CH₂—O—Ar—O—C(O)—(CH₂)₁₄—O-
glucoside; CF₃—CH₂—CH₂—O—Ar—O—C(O)—(CH₂)₁₄—OPO₃H
CF₃—CH₂—CH₂—O—Ar—O—C(O)—(CH₂)₁₅—COOH; CF₃—CH₂—CH₂—O—Ar—O—C(O)—(CH₂)₁₅—SO₃H;
CF₃—CH₂—CH₂—O—Ar—O—C(O)—(CH₂)₁₅—O—SO₃H;; CF₃—CH₂—CH₂—O—Ar—O—C(O)—(CH₂)₁₅—SH;
CF₃—CH₂—CH₂—O—Ar—O—C(O)—(CH₂)₁₅—OH; CF₃—CH₂—CH₂—O—Ar—O—C(O)—(CH₂)₁₅—N⁺R¹R²R³Z⁻;
CF₃—CH₂—CH₂—O—Ar—O—C(O)—(CH₂)₁₅—P⁺R¹R²R³Z⁻; CF₃—CH₂—CH₂—O—Ar—O—C(O)—(CH₂)₁₅—O-
glucoside; CF₃—CH₂—CH₂—O—Ar—O—C(O)—(CH₂)₁₅—OPO₃H
CF₃—CH₂—CH₂—O—Ar—O—C(O)—(CH₂)₁₆—COOH; CF₃—CH₂—CH₂—O—Ar—C(O)—(CH₂)₁₆—SO₃H;
CF₃—CH₂—CH₂—O—Ar—O—C(O)—(CH₂)₁₆—O—SO₃H; CF₃—CH₂—CH₂—O—Ar—O—C(O)—(CH₂)₁₆—SH;
CF₃—CH₂—CH₂—O—Ar—O—C(O)—(CH₂)₁₆—OH; CF₃—CH₂—CH₂—O—Ar—O—C(O)—(CH₂)₁₆—N⁺R¹R²R³Z⁻;
CF₃—CH₂—CH₂—O—Ar—O—C(O)—(CH₂)₁₆—P⁺R¹R²R³Z⁻; CF₃—CH₂—CH₂—O—Ar—O—C(O)—(CH₂)₁₆—O-
glucoside; CF₃—CH₂—CH₂—O—Ar—O—C(O)—(CH₂)₁₆—OPO₃H
CF₃—CH₂—CH₂—O—Ar—O—C(O)—CH₂—CH═CH—(CH₂)₃—COOH; CF₃—CH₂—CH₂—O—Ar—O—C(O)—CH₂—CH═CH—(CH₂)₃—SO₃H;
CF₃—CH₂—CH₂—O—Ar—O—C(O)—CH₂—CH═CH—(CH₂)₃—O—SO₃H;
CF₃—CH₂—CH₂—O—Ar—O—C(O)—CH₂—CH═CH—(CH₂)₃—SH; CF₃—CH₂—CH₂—O—Ar—O—C(O)—CH₂—CH═CH—(CH₂)₃—OH;
CF₃—CH₂—CH₂—O—Ar—O—C(O)—CH₂—CH═CH—(CH₂)₃—N⁺R¹R²R³Z⁻;
CF₃—CH₂—CH₂—O—Ar—O—C(O)—CH₂—CH═CH—(CH₂)₃—P⁺R¹R²R³Z⁻;
CF₃—CH₂—CH₂—O—Ar—O—C(O)—CH₂—CH═CH—(CH₂)₃—O-glucoside;
CF₃—CH₂—CH₂—O—Ar—O—C(O)—CH₂—CH═CH—(CH₂)₃—O—PO₃H;
CF₃—CH₂—CH₂—O—Ar—O—C(O)—CH₂—CH═CH—(CH₂)₄—COOH; CF₃—CH₂—CH₂—O—Ar—O—C(O)—CH₂—CH═CH—(CH₂)₄—SO₃H;
CF₃—CH₂—CH₂—O—Ar—O—C(O)—CH₂—CH═CH—(CH₂)₄—O—SO₃H;
CF₃—CH₂—CH₂—O—Ar—O—C(O)—CH₂—CH═CH—(CH₂)₄—SH; CF₃—CH₂—CH₂—O—Ar—O—C(O)—CH₂—CH═CH—(CH₂)₄—OH;
CF₃—CH₂—CH₂—O—Ar—O—C(O)—CH₂—CH═CH—(CH₂)₄—N⁺R¹R²R³Z⁻;
CF₃—CH₂—CH₂—O—Ar—O—C(O)—O—CH₂—CH═CH—(CH₂)₄—P⁺R¹R²R³Z⁻;
CF₃—CH₂—CH₂—O—Ar—O—C(O)—CH₂—CH═CH—(CH₂)₄—O-glucoside;
CF₃—CH₂—CH₂—O—Ar—O—C(O)—CH₂—CH═CH—(CH₂)₄—O—PO₃H;
CF₃—CH₂—CH₂—O—Ar—O—C(O)—CH₂—CH═CH—(CH₂)₅—COOH; CF₃—CH₂—CH₂—O—Ar—O—C(O)—CH₂—CH═CH—(CH₂)₅—SO₃H;
CF₃—CH₂—CH₂—O—Ar—O—C(O)—CH₂—CH═CH—(CH₂)₅—O—SO₃H;
CF₃—CH₂—CH₂—O—Ar—O—C(O)—CH₂—CH═CH—(CH₂)₅—SH; CF₃—CH₂—CH₂—O—Ar—O—C(O)—CH₂—CH═CH—(CH₂)₅—OH;
CF₃—CH₂—CH₂—O—Ar—O—C(O)—CH₂—CH═CH—(CH₂)₅—N⁺R¹R²R³Z⁻;
CF₃—CH₂—CH₂—O—Ar—O—C(O)—CH₂—CH═CH—(CH₂)₅—P⁺R¹R²R³Z⁻;
CF₃—CH₂—CH₂—O—Ar—O—C(O)—CH₂—CH═CH—(CH₂)₅—O-glucoside,
CF₃—CH₂—CH₂—O—Ar—O—C(O)—CH₂—CH═CH—(CH₂)₅—O—PO₃H
CF₃—CH₂—CH₂—O—Ar—O—C(O)—CH₂—CH═CH—(CH₂)₆—COOH; CF₃—CH₂—CH₂—O—Ar—O—C(O)—CH₂—CH═CH—(CH₂)₆—SO₃H;
CF₃—CH₂—CH₂—O—Ar—O—C(O)—CH₂—CH═CH—(CH₂)₆—O—SO₃H;
CF₃—CH₂—CH₂—O—Ar—O—C(O)—CH₂—CH═CH—(CH₂)₆—SH; CF₃—CH₂—CH₂—O—Ar—O—C(O)—CH₂—CH═CH—(CH₂)₆—OH;
CF₃—CH₂—CH₂—O—Ar—C(O)—O—CH₂—CH═CH—(CH₂)₆—N⁺R¹R²R³Z
CF₃—CH₂—CH₂—O—Ar—O—C(O)—CH₂—CH═CH—(CH₂)₆—P⁺R¹R²R³Z⁻;
CF₃—CH₂—CH₂—O—Ar—O—C(O)—CH₂—CH═CH—(CH₂)₆—O-
glucoside; CF₃—CH₂—CH₂—O—Ar—O—C(O)—CH₂—CH═CH—(CH₂)₆—OPO₃H
CF₃—CH₂—CH₂—O—Ar—O—C(O)—CH₂—CH═CH—(CH₂)₇—COOH; CF₃—CH₂—CH₂—O—Ar—O—C(O)—CH₂—CH═CH—(CH₂)₇—SO₃H;
CF₃—CH₂—CH₂—O—Ar—O—C(O)—CH₂—CH═CH—(CH₂)₇—O—SO₃H;
CF₃—CH₂—CH₂—O—Ar—O—C(O)—CH₂—CH═CH—(CH₂)₇—SH; CF₃—CH₂—CH₂—O—Ar—O—C(O)—CH₂—CH═CH—(CH₂)₇—OH;
CF₃—CH₂—CH₂—O—Ar—O—C(O)—CH₂—CH═CH—(CH₂)₇—N⁺R¹R²R³Z⁻;
CF₃—CH₂—CH₂—O—Ar—O—C(O)—CH₂—CH═CH—(CH₂)₇—P⁺R¹R²R³Z⁻;
CF₃—CH₂—CH₂—O—Ar—O—C(O)—CH₂—CH═CH—(CH₂)₇—O-glucoside;
CF₃—CH₂—CH₂—O—Ar—O—C(O)—CH₂—CH═CH—(CH₂)₇—OPO₃H
CF₃—CH₂—CH₂—O—Ar—O—C(O)—CH₂—CH═CH—(CH₂)₈—COOH; CF₃—CH₂—CH₂—O—Ar—O—C(O)—CH₂—CH═CH—(CH₂)₈—SO₃H;
CF₃—CH₂—CH₂—O—Ar—O—C(O)—CH₂—CH═CH—(CH₂)₈—O—SO₃H;
CF₃—CH₂—CH₂—O—Ar—O—C(O)—CH₂—CH═CH—(CH₂)₈—SH; CF₃—CH₂—CH₂—O—Ar—O—C(O)—CH₂—CH═CH—(CH₂)₈—OH;
CF₃—CH₂—CH₂—O—Ar—O—C(O)—CH₂—CH═CH—(CH₂)₈—N⁺R¹R²R³Z⁻;
CF₃—CH₂—CH₂—O—Ar—O—C(O)—CH₂—CH═CH—(CH₂)₈—P⁺R¹R²R³Z⁻;
CF₃—CH₂—CH₂—O—Ar—O—C(O)—CH₂—CH═CH—(CH₂)₈—O-glucoside;
CF₃—CH₂—CH₂—O—Ar—O—C(O)—CH₂—CH═CH—(CH₂)₈—OPO₃H
CF₃—CH₂—CH₂—O—Ar—O—C(O)—CH₂—CH═CH—(CH₂)₉—COOH; CF₃—CH₂—CH₂—O—Ar—O—C(O)—CH₂—CH═CH—(CH₂)₉—SO₃H;
CF₃—CH₂—CH₂—O—Ar—O—C(O)—CH₂—CH═CH—(CH₂)₉—O—SO₃H;;
CF₃—CH₂—CH₂—O—Ar—O—C(O)—CH═CH—(CH₂)₉—SH; CF₃—CH₂—CH₂—O—Ar—O—C(O)—CH₂—CH═CH—(CH₂)₉—OH;
CF₃—CH₂—CH₂—O—Ar—O—C(O)—CH₂—CH═CH—(CH₂)₉—N⁺R¹R²R³Z⁻;
CF₃—CH₂—CH₂—O—Ar—O—C(O)—CH₂—CH═CH—(CH₂)₉—P⁺R¹R²R³Z⁻;
CF₃—CH₂—CH₂—O—Ar—O—C(O)—CH₂—CH═CH—(CH₂)₉—O-glucoside;
CF₃—CH₂—CH₂—O—Ar—O—C(O)—CH₂—CH═CH—(CH₂)₉—OPO₃H
CF₃—CH₂—CH₂—O—Ar—O—C(O)—CH₂—CH═CH—(CH₂)₁₀—COOH;
CF₃—CH₂—CH₂—O—Ar—O—C(O)—CH₂—CH═CH—(CH₂)₁₀—SO₃H;
CF₃—CH₂—CH₂—O—Ar—O—C(O)—CH₂—CH═CH—(CH₂)₁₀—O—SO₃H;
CF₃—CH₂—CH₂—O—Ar—O—C(O)—CH₂—CH═CH—(CH₂)₁₀—SH; CF₃—CH₂—CH₂—O—Ar—O—C(O)—CH₂—CH═CH—(CH₂)₁₀—OH;
CF₃—CH₂—CH₂—O—Ar—O—C(O)—CH₂—CH═CH—(CH₂)₁₀—N⁺R¹R²R³Z⁻;
CF₃—CH₂—CH₂—O—Ar—O—C(O)—CH₂—CH═CH—(CH₂)₁₀—P⁺R¹R²R³Z⁻;
CF₃—CH₂—CH₂—O—Ar—O—C(O)—CH₂—CH═CH—(CH₂)₁₀—O-glucoside;
CF₃—CH₂—CH₂—O—Ar—O—C(O)—CH₂—CH═CH—(CH₂)₁₀—OPO₃H
CF₃—CH₂—CH₂—O—Ar—O—C(O)—CH₂—CH═CH—(CH₂)₁₁—COOH;
CF₃—CH₂—CH₂—O—Ar—O—C(O)—CH₂—CH═CH—(CH₂)₁₁—SO₃H;
CF₃—CH₂—CH₂—O—Ar—O—C(O)—CH₂—CH═CH—(CH₂)₁₁—O—SO₃H;
CF₃—CH₂—CH₂—O—Ar—O—C(O)—CH₂—CH═CH—(CH₂)₁₁—SH; CF₃—CH₂—CH₂—O—Ar—O—C(O)—CH₂—CH═CH—(CH₂)₁₁—OH;
CF₃—CH₂—CH₂—O—Ar—O—C(O)—CH₂—CH═CH—(CH₂)₁₁—N⁺R¹R²R³Z⁻;
CF₃—CH₂—CH₂—O—Ar—O—C(O)—CH₂—CH═CH—(CH₂)₁₁—P⁺R¹R²R³Z⁻;
CF₃—CH₂—CH₂—O—Ar—O—C(O)—CH₂—CH═CH—(CH₂)₁₁—O-glucoside;
CF₃—CH₂—CH₂—O—Ar—O—C(O)—CH₂—CH═CH—(CH₂)₁₁—OPO₃H
CF₃—CH₂—CH₂—O—Ar—O—C(O)—CH₂—CH═CH—(CH₂)₁₂—COOH;
CF₃—CH₂—CH₂—O—Ar—O—C(O)—CH₂—CH═CH—(CH₂)₁₂—SO₃H;
CF₃—CH₂—CH₂—O—Ar—O—C(O)—CH₂—CH═CH—(CH₂)₁₂—O—SO₃H;
CF₃—CH₂—CH₂—O—Ar—O—C(O)—CH₂—CH═CH—(CH₂)₁₂—SH; CF₃—CH₂—CH₂—O—Ar—O—C(O)—CH₂—CH═CH—(CH₂)₁₂—OH;
CF₃—CH₂—CH₂—O—Ar—O—C(O)—CH₂—CH═CH—(CH₂)₁₂—N⁺R¹R²R³Z⁻;
CF₃—CH₂—CH₂—O—Ar—O—C(O)—CH₂—CH═CH—(CH₂)₁₂—P⁺R¹R²R³Z⁻;
CF₃—CH₂—CH₂—O—Ar—O—C(O)—CH₂—CH═CH—(CH₂)₁₂—O-glucoside;
CF₃—CH₂—CH₂—O—Ar—O—C(O)—CH₂—CH═CH—(CH₂)₁₂—OPO₃H
CF₃—CH₂—CH₂—O—Ar—O—C(O)—CH₂—CH═CH—(CH₂)₁₃—COOH;
CF₃—CH₂—CH₂—O—Ar—O—C(O)—CH₂—CH═CH—(CH₂)₁₃—SO₃H;
CF₃—CH₂—CH₂—O—Ar—O—C(O)—CH₂—CH═CH—(CH₂)₁₃—O—SO₃H;
CF₃—CH₂—CH₂—O—Ar—O—C(O)—CH₂—CH═CH—(CH₂)₁₃—SH; CF₃—CH₂—CH₂—O—Ar—O—C(O)—CH₂—CH═CH—(CH₂)₁₃—OH;
CF₃—CH₂—CH₂—O—Ar—O—C(O)—CH₂—CH═CH—(CH₂)₁₃—N⁺R¹R²R³Z⁻;
CF₃—CH₂—CH₂—O—Ar—O—C(O)—CH₂—CH═CH—(CH₂)₁₃—P⁺R¹R²R³Z⁻;
CF₃—CH₂—CH₂—O—Ar—O—C(O)—CH₂—CH═CH—(CH₂)₁₃—O-glucoside;
CF₃—CH₂—CH₂—O—Ar—O—C(O)—CH₂—CH═CH—(CH₂)₁₃—OPO₃H
CF₃—CH₂—CH₂—O—Ar—O—C(O)—CH₂—CH═CH—(CH₂)₁₄—COOH;
CF₃—CH₂—CH₂—O—Ar—O—C(O)—CH₂—CH═CH—(CH₂)₁₄—SO₃H;
CF₃—CH₂—CH₂—O—Ar—O—C(O)—CH₂—CH═CH—(CH₂)₁₄—O—SO₃H;
CF₃—CH₂—CH₂—O—Ar—O—C(O)—CH₂—CH═CH—(CH₂)₁₄—SH; CF₃—CH₂—CH₂—O—Ar—O—C(O)—CH₂—CH═CH—(CH₂)₁₄—OH;
CF₃—CH₂—CH₂—O—Ar—O—C(O)—CH₂—CH═CH—(CH₂)₁₄—N⁺R¹R²R³Z⁻;
CF₃—CH₂—CH₂—O—Ar—O—C(O)—CH₂—CH═CH—(CH₂)₁₄—P⁺R¹R²R³Z⁻;
CF₃—CH₂—CH₂—O—Ar—O—C(O)—CH₂—CH═CH—(CH₂)₁₄—O-glucoside;
CF₃—CH₂—CH₂—O—Ar—O—C(O)—CH₂—CH═CH—(CH₂)₁₄—OPO₃H
CF₃—CH₂—CH₂—O—Ar—O—C(O)—CH₂—CH═CH—(CH₂)₁₅—COOH;
CF₃—CH₂—CH₂—O—Ar—O—C(O)—CH₂—CH═CH—(CH₂)₁₅—SO₃H;
CF₃—CH₂—CH₂—O—Ar—O—C(O)—CH₂—CH═CH—(CH₂)₁₅—O—SO₃H;
CF₃—CH₂—CH₂—O—Ar—O—C(O)—CH₂—CH═CH—(CH₂)₁₅—SH; CF₃—CH₂—CH₂—O—Ar—O—C(O)—CH₂—CH═CH—(CH₂)₁₅—OH;
CF₃—CH₂—CH₂—O—Ar—O—C(O)—CH₂—CH═CH—(CH₂)₁₅—N⁺R¹R²R³Z⁻;
CF₃—CH₂—CH₂—O—Ar—O—C(O)—CH₂—CH═CH—(CH₂)₁₅—P⁺R¹R²R³Z⁻;
CF₃—CH₂—CH₂—O—Ar—O—C(O)—CH₂—CH═CH—(CH₂)₁₅—O-glucoside;
CF₃—CH₂—CH₂—O—Ar—O—C(O)—CH₂—CH═CH—(CH₂)₁₅—OPO₃H
CF₃—CH₂—CH₂—O—Ar—O—C(O)—CH₂—CH═CH—(CH₂)₁₆—COOH;
CF₃—CH₂—CH₂—O—Ar—O—C(O)—CH₂—CH═CH—(CH₂)₁₆—SO₃H;
CF₃—CH₂—CH₂—O—Ar—O—C(O)—CH₂—CH═CH—(CH₂)₁₆—O—SO₃H;
CF₃—CH₂—CH₂—O—Ar—O—C(O)—CH₂—CH═CH—(CH₂)₁₆—SH; CF₃—CH₂—CH₂—O—Ar—O—C(O)—CH₂—CH═CH—(CH₂)₁₆—OH;
CF₃—CH₂—CH₂—O—Ar—O—C(O)—CH₂—CH═CH—(CH₂)₁₆—N⁺R¹R²R³Z⁻;
CF₃—CH₂—CH₂—O—Ar—O—C(O)—CH₂—CH═CH—(CH₂)₁₆—P⁺R¹R²R³Z⁻;
CF₃—CH₂—CH₂—O—Ar—O—C(O)—CH₂—CH═CH—(CH₂)₁₆—O-glucoside;
CF₃—CH₂—CH₂—O—Ar—O—C(O)—CH₂—CH═CH—(CH₂)₁₆—OPO₃H
CF₃—Ar—O—(CH₂)ₙ—O—(CH₂)_{n′}—N⁺R¹R²R³Z⁻; CF₃—Ar—O—(CH₂)ₙ—O—(CH₂)_{n′}—P⁺R¹R²R³Z⁻;
CF₃—Ar—O—(CH₂)ₙ—O—(CH₂)_{n′}—O-glucoside; CF₃—Ar—O—(CH₂)ₙ—O—(CH₂)_{n′}—COOH; CF₃—Ar—O—(CH₂)ₙ—O—(CH₂)_{n′}SO₃H;
CF₃—Ar—O—(CH₂)ₙ—O—(CH₂)_{n′}—O—SO₃H; CF₃—Ar—O—((CH₂)ₙ—O—(CH₂)_{n′}—OH;
CF₃—Ar—O—(CH₂)ₙ—O—(CH₂)_{n′}—SH; CF₃—Ar—O—(CH₂)ₙ—O—(CH₂)_{n′}—O—PO₃H;
CF₃—Ar—O—C(O)—(CH₂)ₙ—O—(CH₂)_{n′}—N⁺R¹R²R³Z⁻; CF₃—Ar—O—C(O)—(CH₂)ₙ—O—(CH₂)_{n′}—P⁺R¹R²R³Z⁻;
CF₃—Ar—O—C(O)—(CH₂)ₙ—O—(CH₂)_{n′}—O-glucoside; CF₃—Ar—O—C(O)—(CH₂)ₙ—O—(CH₂)_{n′}—COOH;
CF₃—Ar—O—C(O)—(CH₂)ₙ—O—(CH₂)_{n′}SO₃H; CF₃—Ar—O—C(O)—(CH₂)ₙ—O—(CH₂)_{n′}—O—SO₃H;
CF₃—Ar—O—C(O)—(CH₂)ₙ—O—(CH₂)_{n′}—OH; CF₃—Ar—O—C(O)—(CH₂)ₙ—O—(CH₂)_{n′}—SH;
CF₃—Ar—O—C(O)—(CH₂)ₙ—O—(CH₂)_{n′}—O—PO₃H;
CF₃—O—Ar—O—(CH₂)ₙ—O—(CH₂)_{n′}—N⁺R¹R²R³Z⁻; CF₃—O—Ar—O—(CH₂)ₙ—O—(CH₂)_{n′}—P⁺R¹R²R³Z⁻;
CF₃—O—Ar—O—(CH₂)ₙ—S—(CH₂)_{n′}—O-glucoside; CF₃—O—Ar—O—(CH₂)ₙ—O—(CH₂)_{n′}—COOH;
CF₃—O—Ar—O—(CH₂)ₙ—O—(CH₂)_{n′}—SO₃H; CF₃—O—Ar—O—(CH₂)ₙ—O—(CH₂)_{n′}—O—SO₃H;
CF₃—O—Ar—O—(CH₂)ₙ—O—(CH₂)_{n′}—OH; CF₃—O—Ar—O—(CH₂)ₙ—O—(CH₂)_{n′}—SH; CF₃—O—Ar—O—(CH₂)ₙ—O—(CH₂)_{n′}—O—PO₃H;
CF₃—O—Ar—O—C(O)—(CH₂)ₙ—O—(CH₂)_{n′}—N⁺R¹R²R³Z⁻; CF₃—O—Ar—O—C(O)—(CH₂)ₙ—O—(CH₂)_{n′}—P⁺R¹R²R³Z⁻;
CF₃—O—Ar—O—C(O)—(CH₂)ₙ—O—(CH₂)_{n′}—O-glucoside; CF₃—O—Ar—O—C(O)—(CH₂)ₙ—O—(CH₂)_{n′}—COOH;
CF₃—O—Ar—O—C(O)—(CH₂)ₙ—O—(CH₂)_{n′}—SO₃H; CF₃—O—Ar—O—C(O)—(CH₂)ₙ—O—(CH₂)_{n′}—S—SO₃H;
CF₃—O—Ar—O—C(O)—(CH₂)ₙ—O—(CH₂)_{n′}—OH; CF₃—O—Ar—O—C(O)—(CH₂)ₙ—O—(CH₂)_{n′}—SH;
CF₃—O—Ar—O—C(O)—(CH₂)ₙ—O—(CH₂)_{n′}—O—PO₃H;
CF₃—CH₂—CH₂—O—Ar—O—(CH₂)ₙ—O—(CH₂)_{n′}—N⁺R¹R²R³Z⁻; CF₃—CH₂—CH₂—O—Ar—O—(CH₂)ₙ—O—(CH₂)_{n′}—P⁺R¹R²R³Z⁻;
CF₃—CH₂—CH₂—O—Ar—O—(CH₂)ₙ—O—(CH₂)_{n′}—O-glucoside; CF₃—CH₂—CH₂—O—Ar—O—(CH₂)ₙ—O—(CH₂)_{n′}COOH;
CF₃—CH₂—CH₂—O—Ar—O—(CH₂)ₙ—O—(CH₂)_{n′}—SO₃H;
CF₃—CH₂—CH₂—O—Ar—O—(CH₂)ₙ—O—(CH₂)_{n′}—O—SO₃H; CF₃—CH₂—CH₂—O—Ar—O—(CH₂)ₙ—O—(CH₂)_{n′}—OH;
CF₃—CH₂—CH₂—O—Ar—O—(CH₂)ₙ—O—(CH₂)_{n′}—SH; CF₃—CH₂—CH₂—O—Ar—O—(CH₂)ₙ—O—(CH₂)_{n′}—O—PO₃H;
CF₃—CH₂—CH₂—O—Ar—O—C(O)—(CH₂)ₙ—O—(CH₂)_{n′}—N⁺R¹R²R³Z⁻;
CF₃—CH₂—CH₂—O—Ar—O—C(O)—(CH₂)ₙ—O—(CH₂)_{n′}—P⁺R¹R²R³Z⁻;
CF₃—CH₂—CH₂—O—Ar—O—C(O)—(CH₂)ₙ—S—(CH₂)_{n′}—O-glucoside;
CF₃—CH₂—CH₂—O—Ar—O—C(O)—(CH₂)ₙ—O—(CH₂)_{n′}—COOH; CF₃—CH₂—CH₂—O—Ar—O—C(O)—(CH₂)ₙ—O—(CH₂)_{n′}—SO₃H;
CF₃—CH₂—CH₂—O—Ar—O—C(O)—(CH₂)ₙ—O—(CH₂)_{n′}—O—SO₃H;
CF₃—CH₂—CH₂—O—Ar—O—C(O)—(CH₂)ₙ—O—(CH₂)_{n′}—OH; CF₃—CH₂—CH₂—O—Ar—O—C(O)—(CH₂)ₙ—O—(CH₂)_{n′}—SH;
CF₃—CH₂—CH₂—O—Ar—O—C(O)—(CH₂)ₙ—O—(CH₂)_{n′}—O—PO₃H;
CF₃—S—Ar—O—(CH₂)₃—COOH; CF₃—S—Ar—O—(CH₂)₃—SO₃H; CF₃—S—Ar—O—(CH₂)₃—O—SO₃H;
CF₃—S—Ar—O—(CH₂)₃—SH; CF₃—S—Ar—O—(CH₂)₃—OH; CF₃—S—Ar—O—(CH₂)₃—N⁺R¹R²R³Z⁻;
CF₃—S—Ar—O—(CH₂)₃—P⁺R¹R²R³Z⁻; CF₃—S—Ar—O—(CH₂)₃—O-glucoside; CF₃—S—Ar—O—(CH₂)₃—O—PO₃H
CF₃—S—Ar—O—(CH₂)₄—COOH; CF₃—S—Ar—O—(CH₂)₄—SO₃H; CF₃—S—Ar—O—(CH₂)₄—O—SO₃H;
CF₃—S—Ar—O—(CH₂)₄—SH; CF₃—S—Ar—O—(CH₂)₄—OH; CF₃—S—Ar—O—(CH₂)₄—N⁺R¹R²R³Z⁻;
CF₃—S—Ar—O—(CH₂)₄—P⁺R¹R²R³Z⁻; CF₃—S—Ar—O—(CH₂)₄—O-glucoside; CF₃—S—Ar—O—(CH₂)₄—O—PO₃H
CF₃—S—Ar—O—(CH₂)₅—COOH; CF₃—S—Ar—O—(CH₂)₅—SO₃H; CF₃—S—Ar—O—(CH₂)₅—O—SO₃H;
CF₃—S—Ar—O—(CH₂)₅—SH; CF₃—S—Ar—O—(CH₂)₅—OH; CF₃—S—Ar—O—(CH₂)₅—N⁺R¹R²R³Z⁻;
CF₃—S—Ar—O—(CH₂)₅—P⁺R¹R²R³Z⁻; CF₃—S—Ar—O—(CH₂)₅—O-glucoside; CF₃—S—Ar—O—(CH₂)₅—O—PO₃H
CF₃—S—Ar—O—(CH₂)₆—COOH; CF₃—S—Ar—O—(CH₂)₆—SO₃H; CF₃—S—Ar—O—(CH₂)₆—O—SO₃H;
CF₃—S—Ar—O—(CH₂)₆—SH; CF₃—S—Ar—O—(CH₂)₆—OH; CF₃—S—Ar—O—(CH₂)₆—N⁺R¹R²R³Z⁻;
CF₃—S—Ar—O—(CH₂)₆—P⁺R¹R²R³Z⁻; CF₃—S—Ar—O—(CH₂)₆—O-glucoside; CF₃—S—Ar—O—(CH₂)₆—O—PO₃H
CF₃—S—Ar—O—(CH₂)₇—COOH; CF₃—S—Ar—O—(CH₂)₇—SO₃H; CF₃—S—Ar—O—(CH₂)₇—O—SO₃H;
CF₃—S—Ar—O—(CH₂)₇—SH; CF₃—S—Ar—O—(CH₂)₇—OH; CF₃—S—Ar—O—(CH₂)₇—N⁺R¹R²R³Z⁻;
CF₃—S—Ar—O—(CH₂)₇—P⁺R¹R²R³Z⁻; CF₃—S—Ar—O—(CH₂)₇—O-glucoside; CF₃—S—Ar—O—(CH₂)₇—O—PO₃H
CF₃—S—Ar—O—(CH₂)₈—COOH; CF₃—S—Ar—O—(CH₂)₈—SO₃H; CF₃—S—Ar—O—(CH₂)₈—O—SO₃H;
CF₃—S—Ar—O—(CH₂)₈—SH; CF₃—S—Ar—O—(CH₂)₈—OH; CF₃—S—Ar—O—(CH₂)₈—N⁺R¹R²R³Z⁻;
CF₃—S—Ar—O—(CH₂)₈—P⁺R¹R²R³Z⁻; CF₃—S—Ar—O—(CH₂)₈—O-glucoside; CF₃—S—Ar—O—(CH₂)₈—O—PO₃H
CF₃—S—Ar—O—(CH₂)₉—COOH; CF₃—S—Ar—O—(CH₂)₉—SO₃H; CF₃—S—Ar—O—(CH₂)₉—O—SO₃H;
CF₃—S—Ar—O—(CH₂)₉—SH; CF₃—S—Ar—O—(CH₂)₉—OH; CF₃—S—Ar—O—(CH₂)₉—N⁺R¹R²R³Z⁻;
CF₃—S—Ar—O—(CH₂)₉—P⁺R¹R²R³Z⁻; CF₃—S—Ar—O—(CH₂)₉—O-glucoside; CF₃—S—Ar—O—(CH₂)₉—O—PO₃H
CF₃—S—Ar—O—(CH₂)₁₀—COOH; CF₃—S—Ar—O—(CH₂)₁₀—SO₃H; CF₃—S—Ar—O—(CH₂)₁₀—O—SO₃H;
CF₃—S—Ar—O—(CH₂)₁₀—SH; CF₃—S—Ar—O—(CH₂)₁₀—OH; CF₃—S—Ar—O—(CH₂)₁₀—N⁺R¹R²R³Z⁻;
CF₃—S—Ar—O—(CH₂)₁₀—P⁺R¹R²R³Z⁻; CF₃—S—Ar—O—(CH₂)₁₀—O-glucoside;
CF₃—S—Ar—O—(CH₂)₁₀—O—PO₃H
CF₃—S—Ar—O—(CH₂)₁₁—COOH; CF₃—S—Ar—O—(CH₂)₁₁—SO₃H; CF₃—S—Ar—O—(CH₂)₁₁—O—SO₃H;
CF₃—S—Ar—O—(CH₂)₁₁—SH; CF₃—S—Ar—O—(CH₂)₁₁—OH; CF₃—S—Ar—O—(CH₂)₁₁—N⁺R¹R²R³Z⁻;
CF₃—S—Ar—O—(CH₂)₁₁—P⁺R¹R²R³Z⁻; CF₃—S—Ar—O—(CH₂)₁₁—O-glucoside;
CF₃—S—Ar—O—(CH₂)₁₁—O—PO₃H
CF₃—S—Ar—O—(CH₂)₁₂—COOH; CF₃—S—Ar—O—(CH₂)₁₂—SO₃H; CF₃—S—Ar—O—(CH₂)₁₂—O—SO₃H;
CF₃—S—Ar—O—(CH₂)₁₂—SH; CF₃—S—Ar—O—(CH₂)₁₂—OH; CF₃—S—Ar—O—(CH₂)₁₂—N⁺R¹R²R³Z⁻;
CF₃—S—Ar—O—(CH₂)₁₂—P⁺R¹R²R³Z⁻; CF₃—S—Ar—O—(CH₂)₁₂—O-glucoside;
CF₃—S—Ar—O—(CH₂)₁₂—O—PO₃H
CF₃—S—Ar—O—(CH₂)₁₃—COOH; CF₃—S—Ar—O—(CH₂)₁₃—SO₃H; CF₃—S—Ar—O—(CH₂)₁₃—O—SO₃H;
CF₃—S—Ar—O—(CH₂)₁₃—SH; CF₃—S—Ar—O—(CH₂)₁₃—OH; CF₃—S—Ar—O—(CH₂)₁₃—N⁺R¹R²R³Z⁻;
CF₃—S—Ar—O—(CH₂)₁₃—P⁺R¹R²R³Z⁻; CF₃—S—Ar—O—(CH₂)₁₃—O-glucoside;
CF₃—S—Ar—O—(CH₂)₁₃—O—PO₃H
CF₃—S—Ar—O—(CH₂)₁₄—COOH; CF₃—S—Ar—O—(CH₂)₁₄—SO₃H; CF₃—S—Ar—O—(CH₂)₁₄—O—SO₃H;
CF₃—S—Ar—O—(CH₂)₁₄—SH; CF₃—S—Ar—O—(CH₂)₁₄—OH; CF₃—S—Ar—O—(CH₂)₁₄—N⁺R¹R²R³Z⁻;
CF₃—S—Ar—O—(CH₂)₁₄—P⁺R¹R²R³Z⁻; CF₃—S—Ar—O—(CH₂)₁₄—O-glucoside;
CF₃—S—Ar—O—(CH₂)₁₄—O—PO₃H
CF₃—S—Ar—O—(CH₂)₁₅—COOH; CF₃—S—Ar—O—(CH₂)₁₅—SO₃H; CF₃—S—Ar—O—(CH₂)₁₅—O—SO₃H;
CF₃—S—Ar—O—(CH₂)₁₅—SH; CF₃—S—Ar—O—(CH₂)₁₅—OH; CF₃—S—Ar—O—(CH₂)₁₅—N⁺R¹R²R³Z⁻;
CF₃—S—Ar—O—(CH₂)₁₅—P⁺R¹R²R³Z⁻; CF₃—S—Ar—O—(CH₂)₁₅—O-glucoside;
CF₃—S—Ar—O—(CH₂)₁₅—O—PO₃H
CF₃—S—Ar—O—(CH₂)₁₆—COOH; CF₃—S—Ar—O—(CH₂)₁₆—SO₃H; CF₃—S—Ar—O—(CH₂)₁₆—O—SO₃H;
CF₃—S—Ar—O—(CH₂)₁₆—SH; CF₃—S—Ar—O—(CH₂)₁₆—OH; CF₃—S—Ar—O—(CH₂)₁₆—N⁺R¹R²R³Z⁻;
CF₃—S—Ar—O—(CH₂)₁₆—P⁺R¹R²R³Z⁻; CF₃—S—Ar—O—(CH₂)₁₆—O-glucoside;
CF₃—S—Ar—O—(CH₂)₁₆—O—PO₃H
CF₃—S—Ar—O—(CH₂)ₙ—O—(CH₂)_{n′}—COOH;
CF₃—S—Ar—O—(CH₂)ₙ—O—(CH₂)_{n′}—SO₃H; CF₃—S—Ar—O—(CH₂)ₙ—O—(CH₂)_{n′}—O—SO₃H;
CF₃—S—Ar—O—(CH₂)ₙ—O—(CH₂)_{n′}—SH; CF₃—S—Ar—O—(CH₂)ₙ—O—(CH₂)_{n′}—OH;
CF₃—S—Ar—O—(CH₂)ₙ—O—(CH₂)_{n′}—N⁺R¹R²R³Z⁻; CF₃—S—Ar—O—(CH₂)ₙ—O—(CH₂)_{n′}—P⁺R¹R²R³Z⁻;
CF₃—S—Ar—O—(CH₂)ₙ—O—(CH₂)_{n′}—O-glucoside; CF₃—S—Ar—O—(CH₂)ₙ—O—(CH₂)_{n′}—O—PO₃H
CF₃—S—Ar—O—CH₂—CH═CH—(CH₂)₃—COOH; CF₃—S—Ar—O—CH₂—CH═CH—(CH₂)₃—SO₃H;
CF₃—S—Ar—O—CH₂—CH═CH—(CH₂)₃—O—SO₃H; CF₃—S—Ar—O—CH₂—CH═CH—(CH₂)₃—SH;
CF₃—S—Ar—O—CH₂—CH═CH—(CH₂)₃—OH; CF₃—S—Ar—O—CH₂—CH═CH—(CH₂)₃—N⁺R¹R²R³Z⁻;
CF₃—S—Ar—O—CH₂—CH═CH—(CH₂)₃—P⁺R¹R²R³Z⁻; CF₃—S—Ar—O—CH₂—CH═CH—(CH₂)₃—O-
glucoside; CF₃—S—Ar—O—CH₂—CH═CH—(CH₂)₃—O—PO₃H
CF₃—S—Ar—O—CH₂—CH═CH—(CH₂)₄—COOH; CF₃—S—Ar—O—CH₂—CH═CH—(CH₂)₄—SO₃H;
CF₃—S—Ar—O—CH₂—CH═CH—(CH₂)₄—O—SO₃H; CF₃—S—Ar—O—CH₂—CH═CH—(CH₂)₄—SH;
CF₃—S—Ar—O—CH₂—CH═CH—(CH₂)₄—OH; CF₃—S—Ar—O—CH₂—CH═CH—(CH₂)₄—N⁺R¹R²R³Z⁻;
CF₃—S—Ar—O—CH₂—CH═CH—(CH₂)₄—P⁺R¹R²R³Z⁻; CF₃—S—Ar—O—CH₂—CH═CH—(CH₂)₄—O-
glucoside; CF₃—S—Ar—O—CH₂—CH═CH—(CH₂)₄—O—PO₃H
CF₃—S—Ar—O—CH₂—CH═CH—(CH₂)₅—COOH; CF₃—S—Ar—O—CH₂—CH═CH—(CH₂)₅—SO₃H;
CF₃—S—Ar—O—CH₂—CH═CH—(CH₂)₅—O—SO₃H; CF₃—S—Ar—O—CH₂—CH═CH—(CH₂)₅—SH;
CF₃—S—Ar—O—CH₂—CH═CH—(CH₂)₅—OH; CF₃—S—Ar—O—CH₂—CH═CH—(CH₂)₅—N⁺R¹R²R³Z⁻;
CF₃—S—Ar—O—CH₂—CH═CH—(CH₂)₅—P⁺R¹R²R³Z⁻; CF₃—S—Ar—O—CH₂—CH═CH—(CH₂)₅—O-
glucoside; CF₃—S—Ar—O—CH₂—CH═CH—(CH₂)₅—O—PO₃H
CF₃—S—Ar—O—CH₂—CH═CH—(CH₂)₆—COOH; CF₃—S—Ar—O—CH₂—CH═CH—(CH₂)₆—SO₃H;
CF₃—S—Ar—O—CH₂—CH═CH—(CH₂)₆—O—SO₃H; CF₃—S—Ar—O—CH₂—CH═CH—(CH₂)₆—SH;
CF₃—S—Ar—O—CH₂—CH═CH—(CH₂)₆—OH; CF₃—S—Ar—O—CH₂—CH═CH—(CH₂)₆—N⁺R¹R²R³Z⁻;
CF₃—S—Ar—O—CH₂—CH═CH—(CH₂)₆—P⁺R¹R²R³Z⁻; CF₃—S—Ar—O—CH₂—CH═CH—(CH₂)₆—O-
glucoside; CF₃—S—Ar—O—CH₂—CH═CH—(CH₂)₆—O—PO₃H
CF₃—S—Ar—O—CH₂—CH═CH—(CH₂)₇—COOH; CF₃—S—Ar—O—CH₂—CH═CH—(CH₂)₇—SO₃H;
CF₃—S—Ar—O—CH₂—CH═CH—(CH₂)₇—O—SO₃H; CF₃—S—Ar—O—CH₂—CH═CH—(CH₂)₇—SH;
CF₃—S—Ar—O—CH₂—CH═CH—(CH₂)₇—OH; CF₃—S—Ar—O—CH₂—CH═CH—(CH₂)₇—N⁺R¹R²R³Z⁻;
CF₃—S—Ar—O—CH₂—CH═CH—(CH₂)₇—P⁺R¹R²R³Z⁻; CF₃—S—Ar—O—CH₂—CH═CH—(CH₂)₇—O-
glucoside; CF₃—S—Ar—O—CH₂—CH═CH—(CH₂)₇—O—PO₃H
CF₃—S—Ar—O—CH₂—CH═CH—(CH₂)₈—COOH; CF₃—S—Ar—O—CH₂—CH═CH—(CH₂)₈—SO₃H;
CF₃—S—Ar—O—CH₂—CH═CH—(CH₂)₈—O—SO₃H; CF₃—S—Ar—O—CH₂—CH═CH—(CH₂)₈—SH;
CF₃—S—Ar—O—CH₂—CH═CH—(CH₂)₈—OH; CF₃—S—Ar—O—CH₂—CH═CH—(CH₂)₈—N⁺R¹R²R³Z⁻;
CF₃—S—Ar—O—CH₂—CH═CH—(CH₂)₈—P⁺R¹R²R³Z⁻; CF₃—S—Ar—O—CH₂—CH═CH—(CH₂)₈—O-
glucoside; CF₃—S—Ar—O—CH₂—CH═CH—(CH₂)₈—O—PO₃H
CF₃—S—Ar—O—CH₂—CH═CH—(CH₂)₉—COOH; CF₃—S—Ar—O—CH₂—CH═CH—(CH₂)₉—SO₃H;
CF₃—S—Ar—O—CH₂—CH═CH—(CH₂)₉—O—SO₃H; CF₃—S—Ar—O—CH₂—CH═CH—(CH₂)₉—SH;
CF₃—S—Ar—O—CH₂—CH═CH—(CH₂)₉—OH; CF₃—S—Ar—O—CH₂—CH═CH—(CH₂)₉—N⁺R¹R²R³Z⁻;
CF₃—S—Ar—O—CH₂—CH═CH—(CH₂)₉—P⁺R¹R²R³Z⁻; CF₃—S—Ar—O—CH₂—CH═CH—(CH₂)₉—O-
glucoside; CF₃—S—Ar—O—CH₂—CH═CH—(CH₂)₉—O—PO₃H
CF₃—S—Ar—O—CH₂—CH═CH—(CH₂)₁₀—COOH; CF₃—S—Ar—O—CH₂—CH═CH—(CH₂)₁₀—SO₃H;
CF₃—S—Ar—O—CH₂—CH═CH—(CH₂)₁₀—O—SO₃H; CF₃—S—Ar—O—CH₂—CH═CH—(CH₂)₁₀—SH;
CF₃—S—Ar—O—CH₂—CH═CH—(CH₂)₁₀—OH; CF₃—S—Ar—O—CH₂—CH═CH—(CH₂)₁₀—N⁺R¹R²R³Z⁻;
CF₃—S—Ar—O—CH₂—CH═CH—(CH₂)₁₀—P⁺R¹R²R³Z⁻; CF₃—S—Ar—O—CH₂—CH═CH—(CH₂)₁₀—O-
glucoside; CF₃—S—Ar—O—CH₂—CH═CH—(CH₂)₁₀—O—PO₃H
CF₃—S—Ar—O—CH₂—CH═CH—(CH₂)₁₁—COOH; CF₃—S—Ar—O—CH₂—CH═CH—(CH₂)₁₁—SO₃H;
CF₃—S—Ar—O—CH₂—CH═CH—(CH₂)₁₁—O—SO₃H; CF₃—S—Ar—O—CH₂—CH═CH—(CH₂)₁₁—SH;
CF₃—S—Ar—O—CH₂—CH═CH—(CH₂)₁₁—OH; CF₃—S—Ar—O—CH₂—CH═CH—(CH₂)₁₁—N⁺R¹R²R³Z⁻;
CF₃—S—Ar—O—CH₂—CH═CH—(CH₂)₁₁—P⁺R¹R²R³Z⁻; CF₃—S—Ar—O—CH₂—CH═CH—(CH₂)₁₁—O-
glucoside; CF₃—S—Ar—O—CH₂—CH═CH—(CH₂)₁₁—O—PO₃H
CF₃—S—Ar—O—CH₂—CH═CH—(CH₂)₁₂—COOH; CF₃—S—Ar—O—CH₂—CH═CH—(CH₂)₁₂—SO₃H;
CF₃—S—Ar—O—CH₂—CH═CH—(CH₂)₁₂—O—SO₃H; CF₃—S—Ar—O—CH₂—CH═CH—(CH₂)₁₂—SH;
CF₃—S—Ar—O—CH₂—CH═CH—(CH₂)₁₂—OH; CF₃—S—Ar—O—CH₂—CH═CH—(CH₂)₁₂—N⁺R¹R²R³Z⁻;
CF₃—S—Ar—O—CH₂—CH═CH—(CH₂)₁₂—P⁺R¹R²R³Z⁻; CF₃—S—Ar—O—CH₂—CH═CH—(CH₂)₁₂—O-
glucoside; CF₃—S—Ar—O—CH₂—CH═CH—(CH₂)₁₂—O—PO₃H
CF₃—S—Ar—O—CH₂—CH═CH—(CH₂)₁₃—COOH; CF₃—S—Ar—O—CH₂—CH═CH—(CH₂)₁₃—SO₃H;
CF₃—S—Ar—O—CH₂—CH═CH—(CH₂)₁₃—O—SO₃H; CF₃—S—Ar—O—CH₂—CH═CH—(CH₂)₁₃—SH;
CF₃—S—Ar—O—CH₂—CH═CH—(CH₂)₁₃—OH; CF₃—S—Ar—O—CH₂—CH═CH—(CH₂)₁₃—N⁺R¹R²R³Z⁻;
CF₃—S—Ar—O—CH₂—CH═CH—(CH₂)₁₃—P⁺R¹R²R³Z⁻; CF₃—S—Ar—O—CH₂—CH═CH—(CH₂)₁₃—O-
glucoside; CF₃—S—Ar—O—CH₂—CH═CH—(CH₂)₁₃—O—PO₃H
CF₃—S—Ar—O—CH₂—CH═CH—(CH₂)₁₄—COOH; CF₃—S—Ar—O—CH₂—CH═CH—(CH₂)₁₄—SO₃H;
CF₃—S—Ar—O—CH₂—CH═CH—(CH₂)₁₄—O—SO₃H; CF₃—S—Ar—O—CH₂—CH═CH—(CH₂)₁₄—SH;
CF₃—S—Ar—O—CH₂—CH═CH—(CH₂)₁₄OH; CF₃—S—Ar—O—CH₂—CH═CH—(CH₂)₁₄—N⁺R¹R²R³Z⁻;
CF₃—S—Ar—O—CH₂—CH═CH—(CH₂)₁₄—P⁺R¹R²R³Z⁻; CF₃—S—Ar—O—CH₂—CH═CH—(CH₂)₁₄—O-
glucoside; CF₃—S—Ar—O—CH₂—CH═CH—(CH₂)₁₄—O—PO₃H
CF₃—S—Ar—O—CH₂—CH═CH—(CH₂)₁₅—COOH; CF₃—S—Ar—O—CH₂—CH═CH—(CH₂)₁₅SO₃H;
CF₃—S—Ar—O—CH₂—CH═CH—(CH₂)₁₅—O—SO₃H; CF₃—S—Ar—O—CH₂—CH═CH—(CH₂)₁₅SH;
CF₃—S—Ar—O—CH₂—CH═CH—(CH₂)₁₅OH; CF₃—S—Ar—O—CH₂—CH═CH—(CH₂)₁₅N⁺R¹R²R³Z⁻;
CF₃—S—Ar—O—CH₂—CH═CH—(CH₂)₁₅P⁺R¹R²R³Z⁻; CF₃—S—Ar—O—CH₂—CH═CH—(CH₂)₁₅—O-
glucoside; CF₃—S—Ar—O—CH₂—CH═CH—(CH₂)₁₅—O—PO₃H
CF₃—S—Ar—O—CH₂—CH═CH—(CH₂)₁₆COOH; CF₃—S—Ar—O—CH₂—CH═CH—(CH₂)₁₆SO₃H;
CF₃—S—Ar—O—CH₂—CH═CH—(CH₂)₁₆—O—SO₃H; CF₃—S—Ar—O—CH₂—CH═CH—(CH₂)₁₆—SH;
CF₃—S—Ar—O—CH₂—CH═CH—(CH₂)₁₆—OH; CF₃—S—Ar—O—CH₂—CH═CH—(CH₂)₁₆—N⁺R¹R²R³Z⁻;
CF₃—S—Ar—O—CH₂—CH═CH—(CH₂)₁₆—P⁺R¹R²R³Z⁻; CF₃—S—Ar—O—CH₂—CH═CH—(CH₂)₁₆—O-
glucoside; CF₃—S—Ar—O—CH₂—CH═CH—(CH₂)₁₆—O—PO₃H
CF₃—S—Ar—O—C(═O)—(CH₂)₃—COOH; CF₃—S—Ar—O—C(═O)—(CH₂)₃—SO₃H; CF₃—S—Ar—O—C(═O)—(CH₂)₃—O—SO₃H;
CF₃—S—Ar—O—C(═O)—(CH₂)₃—SH; CF₃—S—Ar—O—C(═O)—(CH₂)₃—OH;
CF₃—S—Ar—O—C(═O)—(CH₂)₃—N⁺R¹R²R³Z⁻; CF₃—S—Ar—O—C(═O)—(CH₂)₃—P⁺R¹R²R³Z⁻;
CF₃—S—Ar—O—C(═O)—(CH₂)₃—O-glucoside; CF₃—S—Ar—O—C(═O)—(CH₂)₃—O—PO₃H
CF₃—S—Ar—O—C(═O)—(CH₂)₄—COOH; CF₃—S—Ar—O—C(═O)—(CH₂)₄—SO₃H; CF₃—S—Ar—O—C(═O)—(CH₂)₄—O—SO₃H;
CF₃—S—Ar—O—C(═O)—(CH₂)₄—SH; CF₃—S—Ar—O—C(═O)—(CH₂)₄—OH;
CF₃—S—Ar—O—C(═O)—(CH₂)₄—N⁺R¹R²R³Z⁻; CF₃—S—Ar—O—C(═O)—(CH₂)₄—P⁺R¹R²R³Z⁻;
CF₃—S—Ar—O—C(═O)—(CH₂)₄—O-glucoside; CF₃—S—Ar—O—C(═O)—(CH₂)₄—O—PO₃H
CF₃—S—Ar—O—C(═O)—(CH₂)₅—COOH; CF₃—S—Ar—O—C(═O)—(CH₂)₅—SO₃H; CF₃—S—Ar—O—C(═O)—(CH₂)₅—O—SO₃H;
CF₃—S—Ar—O—C(═O)—(CH₂)₅—SH; CF₃—S—Ar—O—C(═O)—(CH₂)₅—OH;
CF₃—S—Ar—O—C(═O)—(CH₂)₅—N⁺R¹R²R³Z⁻; CF₃—S—Ar—O—C(═O)—(CH₂)₅—P⁺R¹R²R³Z⁻;
CF₃—S—Ar—O—C(═O)—(CH₂)₅—O-glucoside; CF₃—S—Ar—O—C(═O)—(CH₂)₅—O—PO₃H
CF₃—S—Ar—O—C(═O)—(CH₂)₆—COOH; CF₃—S—Ar—O—C(═O)—(CH₂)₆—SO₃H; CF₃—S—Ar—O—C(═O)—(CH₂)₆—O—SO₃H;
CF₃—S—Ar—O—C(═O)—(CH₂)₆—SH; CF₃—S—Ar—O—C(═O)—(CH₂)₆—OH;
CF₃—S—Ar—O—C(═O)—(CH₂)₆—N⁺R¹R²R³Z⁻; CF₃—S—Ar—O—C(═O)—(CH₂)₆—P⁺R¹R²R³Z⁻;
CF₃—S—Ar—O—C(═O)—(CH₂)₆—O-glucoside; CF₃—S—Ar—O—C(═O)—(CH₂)₆—O—PO₃H
CF₃—S—Ar—O—C(═O)—(CH₂)₇—COOH; CF₃—S—Ar—O—C(═O)—(CH₂)₇—SO₃H; CF₃—S—Ar—O—C(═O)—(CH₂)₇—O—SO₃H;
CF₃—S—Ar—O—C(═O)—(CH₂)₇—SH; CF₃—S—Ar—O—C(═O)—(CH₂)₇—OH;
CF₃—S—Ar—O—C(═O)—(CH₂)₇—N⁺R¹R²R³Z⁻; CF₃—S—Ar—O—C(═O)—(CH₂)₇—P⁺R¹R²R³Z⁻;
CF₃—S—Ar—O—C(═O)—(CH₂)₇—O-glucoside; CF₃—S—Ar—O—C(═O)—(CH₂)₇—O—PO₃H
CF₃—S—Ar—O—C(═O)—(CH₂)₈—COOH; CF₃—S—Ar—O—C(═O)—(CH₂)₈—SO₃H; CF₃—S—Ar—O—C(═O)—(CH₂)₈—O—SO₃H;
CF₃—S—Ar—O—C(═O)—(CH₂)₈—SH; CF₃—S—Ar—O—C(═O)—(CH₂)₈—OH;
CF₃—S—Ar—O—C(═O)—(CH₂)₈—N⁺R¹R²R³Z⁻; CF₃—S—Ar—O—C(═O)—(CH₂)₈—P⁺R¹R²R³Z⁻;
CF₃—S—Ar—O—C(═O)—(CH₂)₈—O-glucoside; CF₃—S—Ar—O—C(═O)—(CH₂)₈—O—PO₃H
CF₃—S—Ar—O—C(═O)—(CH₂)₉—COOH; CF₃—S—Ar—O—C(═O)—(CH₂)₉—SO₃H; CF₃—S—Ar—O—C(═O)—(CH₂)₉—O—SO₃H;
CF₃—S—Ar—O—C(═O)—(CH₂)₉—SH; CF₃—S—Ar—O—C(═O)—(CH₂)₉—OH;
CF₃—S—Ar—O—C(═O)—(CH₂)₉—N⁺R¹R²R³Z⁻; CF₃—S—Ar—O—C(═O)—(CH₂)₉—P⁺R¹R²R³Z⁻;
CF₃—S—Ar—O—C(═O)—(CH₂)₉—O-glucoside; CF₃—S—Ar—O—C(═O)—(CH₂)₉—O—PO₃H
CF₃—S—Ar—O—C(═O)—(CH₂)₁₀—COOH; CF₃—S—Ar—O—C(═O)—(CH₂)₁₀—SO₃H; CF₃—S—Ar—O—C(═O)—(CH₂)₁₀—O—SO₃H;
CF₃—S—Ar—O—C(═O)—(CH₂)₁₀—SH; CF₃—S—Ar—O—C(═O)—(CH₂)₁₀—OH;
CF₃—S—Ar—O—C(═O)—(CH₂)₁₀—N⁺R¹R²R³Z⁻; CF₃—S—Ar—O—C(═O)—(CH₂)₁₀—P⁺R¹R²R³Z⁻;
CF₃—S—Ar—O—C(═O)—(CH₂)₁₀—O-glucoside; CF₃—S—Ar—O—C(═O)—(CH₂)₁₀—O—PO₃H
CF₃—S—Ar—O—C(═O)—(CH₂)₁₁—COOH; CF₃—S—Ar—O—C(═O)—(CH₂)₁₁—SO₃H; CF₃—S—Ar—O—C(═O)—(CH₂)₁₁—O—SO₃H;
CF₃—S—Ar—O—C(═O)—(CH₂)₁₁—SH; CF₃—S—Ar—O—C(═O)—(CH₂)₁₁—OH;
CF₃—S—Ar—O—C(═O)—(CH₂)₁₁—N⁺R¹R²R³Z⁻; CF₃—S—Ar—O—C(═O)—(CH₂)₁₁—P⁺R¹R²R³Z⁻;
CF₃—S—Ar—O—C(═O)—(CH₂)₁₁—O-glucoside; CF₃—S—Ar—O—C(═O)—(CH₂)₁₁—O—PO₃H
CF₃—S—Ar—O—C(═O)—(CH₂)₁₂—COOH; CF₃—S—Ar—O—C(═O)—(CH₂)₁₂—SO₃H; CF₃—S—Ar—O—C(═O)—(CH₂)₁₂—O—SO₃H;
CF₃—S—Ar—O—C(═O)—(CH₂)₁₂—SH; CF₃—S—Ar—O—C(═O)—(CH₂)₁₂—OH;
CF₃—S—Ar—O—C(═O)—(CH₂)₁₂—N⁺R¹R²R³Z⁻; CF₃—S—Ar—O—C(═O)—(CH₂)₁₂—P⁺R¹R²R³Z⁻;
CF₃—S—Ar—O—C(═O)—(CH₂)₁₂—O-glucoside; CF₃—S—Ar—O—C(═O)—(CH₂)₁₂—O—PO₃H
CF₃—S—Ar—O—C(═O)—(CH₂)₁₃—COOH; CF₃—S—Ar—O—C(═O)—(CH₂)₁₃—SO₃H; CF₃—S—Ar—O—C(═O)—(CH₂)₁₃—O—SO₃H;
CF₃—S—Ar—O—C(═O)—(CH₂)₁₃—SH; CF₃—S—Ar—O—C(═O)—(CH₂)₁₃—OH;
CF₃—S—Ar—O—C(═O)—(CH₂)₁₃—N⁺R¹R²R³Z⁻; CF₃—S—Ar—O—C(═O)—(CH₂)₁₃—P⁺R¹R²R³Z⁻;
CF₃—S—Ar—O—C(═O)—(CH₂)₁₃—O-glucoside; CF₃—S—Ar—O—C(═O)—(CH₂)₁₃—O—PO₃H
CF₃—S—Ar—O—C(═O)—(CH₂)₁₄—COOH; CF₃—S—Ar—O—C(═O)—(CH₂)₁₄—SO₃H; CF₃—S—Ar—O—C(═O)—(CH₂)₁₄—O—SO₃H;
CF₃—S—Ar—O—C(═O)—(CH₂)₁₄—SH; CF₃—S—Ar—O—C(═O)—(CH₂)₁₄—OH;
CF₃—S—Ar—O—C(═O)—(CH₂)₁₄—N⁺R¹R²R³Z⁻; CF₃—S—Ar—O—C(═O)—(CH₂)₁₄—P⁺R¹R²R³Z⁻;
CF₃—S—Ar—O—C(═O)—(CH₂)₁₄—O-glucoside; CF₃—S—Ar—O—C(═O)—(CH₂)₁₄—O—PO₃H
CF₃—S—Ar—O—C(═O)—(CH₂)₁₅—COOH; CF₃—S—Ar—O—C(═O)—(CH₂)₁₅—SO₃H; CF₃—S—Ar—O—C(═O)—(CH₂)₁₅—O—SO₃H;
CF₃—S—Ar—O—C(═O)—(CH₂)₁₅—SH; CF₃—S—Ar—O—C(═O)—(CH₂)₁₅—OH;
CF₃—S—Ar—O—C(═O)—(CH₂)₁₅—N⁺R¹R²R³Z⁻; CF₃—S—Ar—O—C(═O)—(CH₂)₁₅—P⁺R¹R²R³Z⁻;
CF₃—S—Ar—O—C(═O)—(CH₂)₁₅—O-glucoside; CF₃—S—Ar—O—C(═O)—(CH₂)₁₅—O—PO₃H
CF₃—S—Ar—O—C(═O)—(CH₂)₁₆—COOH; CF₃—S—Ar—O—C(═O)—(CH₂)₁₆—SO₃H; CF₃—S—Ar—O—C(═O)—(CH₂)₁₆—O—SO₃H;
CF₃—S—Ar—O—C(═O)—(CH₂)₁₆—SH; CF₃—S—Ar—O—C(═O)—(CH₂)₁₆—OH;
CF₃—S—Ar—O—C(═O)—(CH₂)₁₆—N⁺R¹R²R³Z⁻; CF₃—S—Ar—O—C(═O)—(CH₂)₁₆—P⁺R¹R²R³Z⁻;
CF₃—S—Ar—O—C(═O)—(CH₂)₁₆—O-glucoside; CF₃—S—Ar—O—C(═O)—(CH₂)₁₆—O—PO₃H
CF₃—S—Ar—O—C(═O)—(CH₂)ₙ—O—(CH₂)_{n′}—COOH; CF₃—S—Ar—O—C(═O)—(CH₂)ₙ—O—(CH₂)_{n′}—SO₃H;
CF₃—S—Ar—O—C(═O)—(CH₂)ₙ—O—(CH₂)_{n′}—O—SO₃H; CF₃—S—Ar—O—C(═O)—(CH₂)ₙ—O—(CH₂)_{n′}—SH;
CF₃—S—Ar—O—C(═O)—(CH₂)ₙ—O—(CH₂)_{n′}—OH; CF₃—S—Ar—O—C(═O)—(CH₂)ₙ—O—(CH₂)_{n′}—N⁺R¹R²R³Z⁻;
CF₃—S—Ar—O—C(═O)—(CH₂)ₙ—O—(CH₂)_{n′}—P⁺R¹R²R³Z⁻; CF₃—S—Ar—O—C(═O)—(CH₂)ₙ—O—(CH₂)_{n′}—O-
glucoside; CF₃—S—Ar—O—C(═O)—(CH₂)ₙ—O—(CH₂)_{n′}—O—PO₃H
CF₃—S—Ar—O—C(═O)—CH₂—CH═CH—(CH₂)₃—COOH; CF₃—S—Ar—O—C(═O)—CH₂—CH═CH—(CH₂)₃—SO₃H;
CF₃—S—Ar—O—C(═O)—CH₂—CH═CH—(CH₂)₃—O—SO₃H; CF₃—S—Ar—O—C(═O)—CH₂—CH═CH—(CH₂)₃—SH;
CF₃—S—Ar—O—C(═O)—CH₂—CH═CH—(CH₂)₃—OH; CF₃—S—Ar—O—C(═O)—CH₂—CH═CH—(CH₂)₃—N⁺R¹R²R³Z⁻;
CF₃—S—Ar—O—C(═O)—CH₂—CH═CH—(CH₂)₃—P⁺R¹R²R³Z⁻;
CF₃—S—Ar—O—C(═O)—CH₂—CH═CH—(CH₂)₃—O-glucoside; CF₃—S—Ar—O—C(═O)—CH₂—CH═CH—(CH₂)₃—O—PO₃H
CF₃—S—Ar—O—C(═O)—CH₂—CH═CH—(CH₂)₄—COOH; CF₃—S—Ar—O—C(═O)—CH₂—CH═CH—(CH₂)₄—SO₃H;
CF₃—S—Ar—O—C(═O)—CH₂—CH═CH—(CH₂)₄—O—SO₃H; CF₃—S—Ar—O—C(═O)—CH₂—CH═CH—(CH₂)₄—SH;
CF₃—S—Ar—O—C(═O)—CH₂—CH═CH—(CH₂)₄—O—SO₃H; CF₃—S—Ar—O—C(═O)—CH₂—CH═CH—(CH₂)₄—SH;
CF₃—S—Ar—O—C(═O)—CH₂—CH═CH—(CH₂)₄—OH; CF₃—S—Ar—O—C(═O)—CH₂—CH═CH—(CH₂)₄—N⁺R¹R²R³Z⁻;
CF₃—S—Ar—O—C(═O)—CH₂—CH═CH—(CH₂)₄—P⁺R¹R²R³Z⁻;
CF₃—S—Ar—O—C(═O)—CH₂—CH═CH—(CH₂)₄—O-glucoside; CF₃—S—Ar—O—C(═O)—CH₂—CH═CH—(CH₂)₄—O—PO₃H
CF₃—S—Ar—O—C(═O)—CH₂—CH═CH—(CH₂)₅—COOH; CF₃—S—Ar—O—C(═O)—CH₂—CH═CH—(CH₂)₅—SO₃H;
CF₃—S—Ar—O—C(═O)—CH₂—CH═CH—(CH₂)₅—O—SO₃H; CF₃—S—Ar—O—C(═O)—CH₂—CH═CH—(CH₂)₅—SH;
CF₃—S—Ar—O—C(═O)—CH₂—CH═CH—(CH₂)₅—OH; CF₃—S—Ar—O—C(═O)—CH₂—CH═CH—(CH₂)₅—N⁺R¹R²R³Z⁻;
CF₃—S—Ar—O—C(═O)—CH₂—CH═CH—(CH₂)₅—P⁺R¹R²R³Z⁻;
CF₃—S—Ar—O—C(═O)—CH₂—CH═CH—(CH₂)₅—O-glucoside; CF₃—S—Ar—O—C(═O)—CH₂—CH═CH—(CH₂)₅—O—PO₃H
CF₃—S—Ar—O—C(═O)—CH₂—CH═CH—(CH₂)₆—COOH; CF₃—S—Ar—O—C(═O)—CH₂—CH═CH—(CH₂)₆—SO₃H;
CF₃—S—Ar—O—C(═O)—CH₂—CH═CH—(CH₂)₆—O—SO₃H; CF₃—S—Ar—O—C(═O)—CH₂—CH═CH—(CH₂)₆—SH;
CF₃—S—Ar—O—C(═O)—CH₂—CH═CH—(CH₂)₆—OH; CF₃—S—Ar—O—C(═O)—CH₂—CH═CH—(CH₂)₆—N⁺R¹R²R³Z⁻;
CF₃—S—Ar—O—C(═O)—CH₂—CH═CH—(CH₂)₆—P⁺R¹R²R³Z⁻;
CF₃—S—Ar—O—C(═O)—CH₂—CH═CH—(CH₂)₆—O-glucoside; CF₃—S—Ar—O—C(═O)—CH₂—CH═CH—(CH₂)₆—O—PO₃H
CF₃—S—Ar—O—C(═O)—CH₂—CH═CH—(CH₂)₇—COOH; CF₃—S—Ar—O—C(═O)—CH₂—CH═CH—(CH₂)₇—SO₃H;
CF₃—S—Ar—O—C(═O)—CH₂—CH═CH—(CH₂)₇—O—SO₃H; CF₃—S—Ar—O—C(═O)—CH₂—CH═CH—(CH₂)₇—SH;
CF₃—S—Ar—O—C(═O)—CH₂—CH═CH—(CH₂)₇—OH; CF₃—S—Ar—O—C(═O)—CH₂—CH═CH—(CH₂)₇—N⁺R¹R²R³Z⁻;
CF₃—S—Ar—O—C(═O)—CH₂—CH═CH—(CH₂)₇—P⁺R¹R²R³Z⁻;
CF₃—S—Ar—O—C(═O)—CH₂—CH═CH—(CH₂)₇—O-glucoside; CF₃—S—Ar—O—C(═O)—CH₂—CH═CH—(CH₂)₇—O—PO₃H
CF₃—S—Ar—O—C(═O)—CH₂—CH═CH—(CH₂)₈—COOH; CF₃—S—Ar—O—C(═O)—CH₂—CH═CH—(CH₂)₈—SO₃H;
CF₃—S—Ar—O—C(═O)—CH₂—CH═CH—(CH₂)₈—O—SO₃H; CF₃—S—Ar—O—C(═O)—CH₂—CH═CH—(CH₂)₈—SH;
CF₃—S—Ar—O—C(═O)—CH₂—CH═CH—(CH₂)₈—OH; CF₃—S—Ar—O—C(═O)—CH₂—CH═CH—(CH₂)₈—N⁺R¹R²R³Z⁻;
CF₃—S—Ar—O—C(═O)—CH₂—CH═CH—(CH₂)₈—P⁺R¹R²R³Z⁻;
CF₃—S—Ar—O—C(═O)—CH₂—CH═CH—(CH₂)₈—O-glucoside; CF₃—S—Ar—O—C(═O)—CH₂—CH═CH—(CH₂)₈—O—PO₃H
CF₃—S—Ar—O—C(═O)—CH₂—CH═CH—(CH₂)₉—COOH; CF₃—S—Ar—O—C(═O)—CH₂—CH═CH—(CH₂)₉—SO₃H;
CF₃—S—Ar—O—C(═O)—CH₂—CH═CH—(CH₂)₉—O—SO₃H; CF₃—S—Ar—O—C(═O)—CH₂—CH═CH—(CH₂)₉—SH;
CF₃—S—Ar—O—C(═O)—CH₂—CH═CH—(CH₂)₉—OH; CF₃—S—Ar—O—C(═O)—CH₂—CH═CH—(CH₂)₉—N⁺R¹R²R³Z⁻;
CF₃—S—Ar—O—C(═O)—CH₂—CH═CH—(CH₂)₉—P⁺R¹R²R³Z⁻;
CF₃—S—Ar—O—C(═O)—CH₂—CH═CH—(CH₂)₉—O-glucoside; CF₃—S—Ar—O—C(═O)—CH₂—CH═CH—(CH₂)₉—O—PO₃H
CF₃—S—Ar—O—C(═O)—CH₂—CH═CH—(CH₂)₁₀—COOH; CF₃—S—Ar—O—C(═O)—CH₂—CH═CH—(CH₂)₁₀—SO₃H;
CF₃—S—Ar—O—C(═O)—CH₂—CH═CH—(CH₂)₁₀—O—SO₃H; CF₃—S—Ar—O—C(═O)—CH₂—CH═CH—(CH₂)₁₀—SH;
CF₃—S—Ar—O—C(═O)—CH₂—CH═CH—(CH₂)₁₀—OH; CF₃—S—Ar—O—C(═O)—CH₂—CH═CH—(CH₂)₁₀—N⁺R¹R²R³Z⁻;
CF₃—S—Ar—O—C(═O)—CH₂—CH═CH—(CH₂)₁₀—P⁺R¹R²R³Z⁻;
CF₃—S—Ar—O—C(═O)—CH₂—CH═CH—(CH₂)₁₀—O-glucoside; CF₃—S—Ar—O—C(═O)—CH₂—CH═CH—(CH₂)₁₀—O—PO₃H
CF₃—S—Ar—O—C(═O)—CH₂—CH═CH—(CH₂)₁₁—COOH; CF₃—S—Ar—O—C(═O)—CH₂—CH═CH—(CH₂)₁₁—SO₃H;
CF₃—S—Ar—O—C(═O)—CH₂—CH═CH—(CH₂)₁₁—O—SO₃H; CF₃—S—Ar—O—C(═O)—CH₂—CH═CH—(CH₂)₁₁—SH;
CF₃—S—Ar—O—C(═O)—CH₂—CH═CH—(CH₂)₁₁—OH; CF₃—S—Ar—O—C(═O)—CH₂—CH═CH—(CH₂)₁₁—N⁺R¹R²R³Z⁻;
CF₃—S—Ar—O—C(═O)—CH₂—CH═CH—(CH₂)₁₁—P⁺R¹R²R³Z⁻;
CF₃—S—Ar—O—C(═O)—CH₂—CH═CH—(CH₂)₁₁—O-glucoside; CF₃—S—Ar—O—C(═O)—CH₂—CH═CH—(CH₂)₁₁—O—PO₃H
CF₃—S—Ar—O—C(═O)—CH₂—CH═CH—(CH₂)₁₂—COOH; CF₃—S—Ar—O—C(═O)—CH₂—CH═CH—(CH₂)₁₂—SO₃H;
CF₃—S—Ar—O—C(═O)—CH₂—CH═CH—(CH₂)₁₂—O—SO₃H; CF₃—S—Ar—O—C(═O)—CH₂—CH═CH—(CH₂)₁₂—SH;
CF₃—S—Ar—O—C(═O)—CH₂—CH═CH—(CH₂)₁₂—OH; CF₃—S—Ar—O—C(═O)—CH₂—CH═CH—(CH₂)₁₂—N⁺R¹R²R³Z⁻;
CF₃—S—Ar—O—C(═O)—CH₂—CH═CH—(CH₂)₁₂—P⁺R¹R²R³Z⁻;
CF₃—S—Ar—O—C(═O)—CH₂—CH═CH—(CH₂)₁₂—O-glucoside; CF₃—S—Ar—O—C(═O)—CH₂—CH═CH—(CH₂)₁₂—O—PO₃H
CF₃—S—Ar—O—C(═O)—CH₂—CH═CH—(CH₂)₁₃—COOH; CF₃—S—Ar—O—C(═O)—CH₂—CH═CH—(CH₂)₁₃—SO₃H;
CF₃—S—Ar—O—C(═O)—CH₂—CH═CH—(CH₂)₁₃—O—SO₃H; CF₃—S—Ar—O—C(═O)—CH₂—CH═CH—(CH₂)₁₃—SH;
CF₃—S—Ar—O—C(═O)—CH₂—CH═CH—(CH₂)₁₃—OH; CF₃—S—Ar—O—C(═O)—CH₂—CH═CH—(CH₂)₁₃—N⁺R¹R²R³Z⁻;
CF₃—S—Ar—O—C(═O)—CH₂—CH═CH—(CH₂)₁₃—P⁺R¹R²R³Z⁻;
CF₃—S—Ar—O—C(═O)—CH₂—CH═CH—(CH₂)₁₃—O-glucoside; CF₃—S—Ar—O—C(═O)—CH₂—CH═CH—(CH₂)₁₃—O—PO₃H
CF₃—S—Ar—O—C(═O)—CH₂—CH═CH—(CH₂)₁₄—COOH; CF₃—S—Ar—O—C(═O)—CH₂—CH═CH—(CH₂)₁₄—SO₃H;
CF₃—S—Ar—O—C(═O)—CH₂—CH═CH—(CH₂)₁₄—O—SO₃H; CF₃—S—Ar—O—C(═O)—CH₂—CH═CH—(CH₂)₁₄—SH;
CF₃—S—Ar—O—C(═O)—CH₂—CH═CH—(CH₂)₁₄—OH; CF₃—S—Ar—O—C(═O)—CH₂—CH═CH—(CH₂)₁₄—N⁺R¹R²R³Z⁻;
CF₃—S—Ar—O—C(═O)—CH₂—CH═CH—(CH₂)₁₄—P⁺R¹R²R³Z⁻;
CF₃—S—Ar—O—C(═O)—CH₂—CH═CH—(CH₂)₁₄—O-glucoside; CF₃—S—Ar—O—C(═O)—CH₂—CH═CH—(CH₂)₁₄—O—PO₃H
CF₃—S—Ar—O—C(═O)—CH₂—CH═CH—(CH₂)₁₅—COOH; CF₃—S—Ar—O—C(═O)—CH₂—CH═CH—(CH₂)₁₅—SO₃H;
CF₃—S—Ar—O—C(═O)—CH₂—CH═CH—(CH₂)₁₅—O—SO₃H; CF₃—S—Ar—O—C(═O)—CH₂—CH═CH—(CH₂)₁₅—SH;
CF₃—S—Ar—O—C(═O)—CH₂—CH═CH—(CH₂)₁₅—OH; CF₃—S—Ar—O—C(═O)—CH₂—CH═CH—(CH₂)₁₅—N⁺R¹R²R³Z⁻;
CF₃—S—Ar—O—C(═O)—CH₂—CH═CH—(CH₂)₁₅—P⁺R¹R²R³Z⁻;
CF₃—S—Ar—O—C(═O)—CH₂—CH═CH—(CH₂)₁₅—O-glucoside; CF₃—S—Ar—O—C(═O)—CH₂—CH═CH—(CH₂)₁₅—O—PO₃H
CF₃—S—Ar—O—C(═O)—CH₂—CH═CH—(CH₂)₁₆—COOH; CF₃—S—Ar—O—C(═O)—CH₂—CH═CH—(CH₂)₁₆—SO₃H;
CF₃—S—Ar—O—C(═O)—CH₂—CH═CH—(CH₂)₁₆—O—SO₃H; CF₃—S—Ar—O—C(═O)—CH₂—CH═CH—(CH₂)₁₆—SH;
CF₃—S—Ar—O—C(═O)—CH₂—CH═CH—(CH₂)₁₆—OH; CF₃—S—Ar—O—C(═O)—CH₂—CH═CH—(CH₂)₁₆—N⁺R¹R²R³Z⁻;
CF₃—S—Ar—O—C(═O)—CH₂—CH═CH—(CH₂)₁₆—P⁺R¹R²R³Z⁻;
CF₃—S—Ar—O—C(═O)—CH₂—CH═CH—(CH₂)₁₆—O-glucoside; CF₃—S—Ar—O—C(═O)—CH₂—CH═CH—(CH₂)₁₆—O—PO₃H
CF₃—CH₂—CH₂—S—Ar—O—(CH₂)₃—COOH;
CF₃—CH₂—CH₂—S—Ar—O—(CH₂)₃—SO₃H; CF₃—CH₂—CH₂—S—Ar—O—(CH₂)₃—O—SO₃H;
CF₃—CH₂—CH₂—S—Ar—O—(CH₂)₃—SH; CF₃—CH₂—CH₂—S—Ar—O—(CH₂)₃—OH;
CF₃—CH₂—CH₂—S—Ar—O—(CH₂)₃—N⁺R¹R²R³Z⁻; CF₃—CH₂—CH₂—S—Ar—O—(CH₂)₃—P⁺R¹R²R³Z⁻;
CF₃—CH₂—CH₂—S—Ar—O—(CH₂)₃—O-glucoside; CF₃—CH₂—CH₂—S—Ar—O—(CH₂)₃—O—PO₃H
CF₃—CH₂—CH₂—S—Ar—O—(CH₂)₄—COOH;
CF₃—CH₂—CH₂—S—Ar—O—(CH₂)₄—SO₃H; CF₃—CH₂—CH₂—S—Ar—O—(CH₂)₄—O—SO₃H;
CF₃—CH₂—CH₂—S—Ar—O—(CH₂)₄—SH; CF₃—CH₂—CH₂—S—Ar—O—(CH₂)₄—OH;
CF₃—CH₂—CH₂—S—Ar—O—(CH₂)₄—N⁺R¹R²R³Z⁻; CF₃—CH₂—CH₂—S—Ar—O—(CH₂)₄—P⁺R¹R²R³Z⁻;
CF₃—CH₂—CH₂—S—Ar—O—(CH₂)₄—O-glucoside; CF₃—CH₂—CH₂—S—Ar—O—(CH₂)₄—O—PO₃H
CF₃—CH₂—CH₂—S—Ar—O—(CH₂)₅—COOH;
CF₃—CH₂—CH₂—S—Ar—O—(CH₂)₅—SO₃H; CF₃—CH₂—CH₂—S—Ar—O—(CH₂)₅—O—SO₃H;
CF₃—CH₂—CH₂—S—Ar—O—(CH₂)₅—SH; CF₃—CH₂—CH₂—S—Ar—O—(CH₂)₅—OH;
CF₃—CH₂—CH₂—S—Ar—O—(CH₂)₅—N⁺R¹R²R³Z⁻; CF₃—CH₂—CH₂—S—Ar—O—(CH₂)₅—P⁺R¹R²R³Z⁻;
CF₃—CH₂—CH₂—S—Ar—O—(CH₂)₅—O-glucoside; CF₃—CH₂—CH₂—S—Ar—O—(CH₂)₅—O—PO₃H
CF₃—CH₂—CH₂—S—Ar—O—(CH₂)₆—COOH;
CF₃—CH₂—CH₂—S—Ar—O—(CH₂)₆—SO₃H; CF₃—CH₂—CH₂—S—Ar—O—(CH₂)₆—O—SO₃H;
CF₃—CH₂—CH₂—S—Ar—O—(CH₂)₆—SH; CF₃—CH₂—CH₂—S—Ar—O—(CH₂)₆—OH;
CF₃—CH₂—CH₂—S—Ar—O—(CH₂)₆—N⁺R¹R²R³Z⁻; CF₃—CH₂—CH₂—S—Ar—O—(CH₂)₆—P⁺R¹R²R³Z⁻;
CF₃—CH₂—CH₂—S—Ar—O—(CH₂)₆—O-glucoside;
CF₃—CH₂—CH₂—S—Ar—O—(CH₂)₆—O—PO₃H
CF₃—CH₂—CH₂—S—Ar—O—(CH₂)₇—COOH;
CF₃—CH₂—CH₂—S—Ar—O—(CH₂)₇—SO₃H; CF₃—CH₂—CH₂—S—Ar—O—(CH₂)₇—O—SO₃H;
CF₃—CH₂—CH₂—S—Ar—O—(CH₂)₇—SH; CF₃—CH₂—CH₂—S—Ar—O—(CH₂)₇—OH;
CF₃—CH₂—CH₂—S—Ar—O—(CH₂)₇—N⁺R¹R²R³Z⁻; CF₃—CH₂—CH₂—S—Ar—O—(CH₂)₇—P⁺R¹R²R³Z⁻;
CF₃—CH₂—CH₂—S—Ar—O—(CH₂)₇—O-glucoside; CF₃—CH₂—CH₂—S—Ar—O—(CH₂)₇—O—PO₃H
CF₃—CH₂—CH₂—S—Ar—O—(CH₂)₈—COOH;
CF₃—CH₂—CH₂—S—Ar—O—(CH₂)₈—SO₃H; CF₃—CH₂—CH₂—S—Ar—O—(CH₂)₈—O—SO₃H;
CF₃—CH₂—CH₂—S—Ar—O—(CH₂)₈—SH; CF₃—CH₂—CH₂—S—Ar—O—(CH₂)₈—OH;
CF₃—CH₂—CH₂—S—Ar—O—(CH₂)₈—N⁺R¹R²R³Z⁻; CF₃—CH₂—CH₂—S—Ar—O—(CH₂)₈—P⁺R¹R²R³Z⁻;
CF₃—CH₂—CH₂—S—Ar—O—(CH₂)₈—O-glucoside; CF₃—CH₂—CH₂—S—Ar—O—(CH₂)₈—O—PO₃H
CF₃—CH₂—CH₂—S—Ar—O—(CH₂)₉—COOH;
CF₃—CH₂—CH₂—S—Ar—O—(CH₂)₉—SO₃H; CF₃—CH₂—CH₂—S—Ar—O—(CH₂)₉—O—SO₃H;
CF₃—CH₂—CH₂—S—Ar—O—(CH₂)₉—SH; CF₃—CH₂—CH₂—S—Ar—O—(CH₂)₉—OH;
CF₃—CH₂—CH₂—S—Ar—O—(CH₂)₉—N⁺R¹R²R³Z⁻; CF₃—CH₂—CH₂—S—Ar—O—(CH₂)₉—P⁺R¹R²R³Z⁻;
CF₃—CH₂—CH₂—S—Ar—O—(CH₂)₉—O-glucoside; CF₃—CH₂—CH₂—S—Ar—O—(CH₂)₉—O—PO₃H
CF₃—CH₂—CH₂—S—Ar—O—(CH₂)₁₀—COOH;
CF₃—CH₂—CH₂—S—Ar—O—(CH₂)₁₀—SO₃H; CF₃—CH₂—CH₂—S—Ar—O—(CH₂)₁₀—O—SO₃H;
CF₃—CH₂—CH₂—S—Ar—O—(CH₂)₁₀—SH; CF₃—CH₂—CH₂—S—Ar—O—(CH₂)₁₀—OH;
CF₃—CH₂—CH₂—S—Ar—O—(CH₂)₁₀—N⁺R¹R²R³Z⁻; CF₃—CH₂—CH₂—S—Ar—O—(CH₂)₁₀—P⁺R¹R²R³Z⁻;
CF₃—CH₂—CH₂—S—Ar—O—(CH₂)₁₀—O-glucoside; CF₃—CH₂—CH₂—S—Ar—O—(CH₂)₁₀—O—PO₃H
CF₃—CH₂—CH₂—S—Ar—O—(CH₂)₁₁—COOH;
CF₃—CH₂—CH₂—S—Ar—O—(CH₂)₁₁—SO₃H; CF₃—CH₂—CH₂—S—Ar—O—(CH₂)₁₁—O—SO₃H;
CF₃—CH₂—CH₂—S—Ar—O—(CH₂)₁₁—SH; CF₃—CH₂—CH₂—S—Ar—O—(CH₂)₁₁—OH;
CF₃—CH₂—CH₂—S—Ar—O—(CH₂)₁₁—N⁺R¹R²R³Z⁻; CF₃—CH₂—CH₂—S—Ar—O—(CH₂)₁₁—P⁺R¹R²R³Z⁻;
CF₃—CH₂—CH₂—S—Ar—O—(CH₂)₁₁—O-glucoside; CF₃—CH₂—CH₂—S—Ar—O—(CH₂)₁₁—O—PO₃H
CF₃—CH₂—CH₂—S—Ar—O—(CH₂)₁₂—COOH;
CF₃—CH₂—CH₂—S—Ar—O—(CH₂)₁₂—SO₃H; CF₃—CH₂—CH₂—S—Ar—O—(CH₂)₁₂—O—SO₃H;
CF₃—CH₂—CH₂—S—Ar—O—(CH₂)₁₂—SH; CF₃—CH₂—CH₂—S—Ar—O—(CH₂)₁₂—OH;
CF₃—CH₂—CH₂—S—Ar—O—(CH₂)₁₂—N⁺R¹R²R³Z⁻; CF₃—CH₂—CH₂—S—Ar—O—(CH₂)₁₂—P⁺R¹R²R³Z⁻;
CF₃—CH₂—CH₂—S—Ar—O—(CH₂)₁₂—O-glucoside; CF₃—CH₂—CH₂—S—Ar—O—(CH₂)₁₂—O—PO₃H
CF₃—CH₂—CH₂—S—Ar—O—(CH₂)₁₃—COOH;
CF₃—CH₂—CH₂—S—Ar—O—(CH₂)₁₃—SO₃H; CF₃—CH₂—CH₂—S—Ar—O—(CH₂)₁₃—O—SO₃H;
CF₃—CH₂—CH₂—S—Ar—O—(CH₂)₁₃—SH; CF₃—CH₂—CH₂—S—Ar—O—(CH₂)₁₃—OH;
CF₃—CH₂—CH₂—S—Ar—O—(CH₂)₁₃—N⁺R¹R²R³Z⁻; CF₃—CH₂—CH₂—S—Ar—O—(CH₂)₁₃—P⁺R¹R²R³Z⁻;
CF₃—CH₂—CH₂—S—Ar—O—(CH₂)₁₃—O-glucoside; CF₃—CH₂—CH₂—S—Ar—O—(CH₂)₁₃—O—PO₃H
CF₃—CH₂—CH₂—S—Ar—O—(CH₂)₁₄—COOH;
CF₃—CH₂—CH₂—S—Ar—O—(CH₂)₁₄—SO₃H; CF₃—CH₂—CH₂—S—Ar—O—(CH₂)₁₄—O—SO₃H;
CF₃—CH₂—CH₂—S—Ar—O—(CH₂)₁₄—SH; CF₃—CH₂—CH₂—S—Ar—O—(CH₂)₁₄—OH;
CF₃—CH₂—CH₂—S—Ar—O—(CH₂)₁₄—N⁺R¹R²R³Z⁻; CF₃—CH₂—CH₂—S—Ar—O—(CH₂)₁₄—P⁺R¹R²R³Z⁻;
CF₃—CH₂—CH₂—S—Ar—O—(CH₂)₁₄—O-glucoside; CF₃—CH₂—CH₂—S—Ar—O—(CH₂)₁₄—O—PO₃H
CF₃—CH₂—CH₂—S—Ar—O—(CH₂)₁₅—COOH;
CF₃—CH₂—CH₂—S—Ar—O—(CH₂)₁₅—SO₃H; CF₃—CH₂—CH₂—S—Ar—O—(CH₂)₁₅—O—SO₃H;
CF₃—CH₂—CH₂—S—Ar—O—(CH₂)₁₅—SH; CF₃—CH₂—CH₂—S—Ar—O—(CH₂)₁₅—OH;
CF₃—CH₂—CH₂—S—Ar—O—(CH₂)₁₅—N⁺R¹R²R³Z⁻; CF₃—CH₂—CH₂—S—Ar—O—(CH₂)₁₅—P⁺R¹R²R³Z⁻;
CF₃—CH₂—CH₂—S—Ar—O—(CH₂)₁₅—O-glucoside; CF₃—CH₂—CH₂—S—Ar—O—(CH₂)₁₅—O—PO₃H
CF₃—CH₂—CH₂—S—Ar—O—(CH₂)₁₆—COOH;
CF₃—CH₂—CH₂—S—Ar—O—(CH₂)₁₆—SO₃H; CF₃—CH₂—CH₂—S—Ar—O—(CH₂)₁₆—O—SO₃H;
CF₃—CH₂—CH₂—S—Ar—O—(CH₂)₁₆—SH; CF₃—CH₂—CH₂—S—Ar—O—(CH₂)₁₆—OH;
CF₃—CH₂—CH₂—S—Ar—O—(CH₂)₁₆—N⁺R¹R²R³Z⁻; CF₃—CH₂—CH₂—S—Ar—O—(CH₂)₁₆—P⁺R¹R²R³Z⁻;
CF₃—CH₂—CH₂—S—Ar—O—(CH₂)₁₆—O-glucoside; CF₃—CH₂—CH₂—S—Ar—O—(CH₂)₁₆—O—PO₃H
CF₃—CH₂—CH₂—S—Ar—O—(CH₂)ₙ—O—(CH₂)_{n′}—COOH; CF₃—CH₂—CH₂—S—Ar—O—(CH₂)ₙ—O—(CH₂)_{n′}—SO₃H;
CF₃—CH₂—CH₂—S—Ar—O—(CH₂)ₙ—O—(CH₂)_{n′}—O—SO₃H; CF₃—CH₂—CH₂—S—Ar—O—(CH₂)ₙ—O—(CH₂)_{n′}—SH;
CF₃—CH₂—CH₂—S—Ar—O—(CH₂)ₙ—O—(CH₂)_{n′}—OH; CF₃—CH₂—CH₂—S—Ar—O—(CH₂)ₙ—O—(CH₂)_{n′}—N⁺R¹R²R³Z⁻;
CF₃—CH₂—CH₂—S—Ar—O—(CH₂)ₙ—O—(CH₂)_{n′}—P⁺R¹R²R³Z⁻;
CF₃—CH₂—CH₂—S—Ar—O—(CH₂)ₙ—O—(CH₂)_{n′}—O-glucoside; CF₃—CH₂—CH₂—S—Ar—O—(CH₂)ₙ—O—(CH₂)_{n′}—O—PO₃H
CF₃—CH₂—CH₂—S—Ar—O—CH₂—CH═CH—(CH₂)₃—COOH; CF₃—CH₂—CH₂—S—Ar—O—CH₂—CH═CH—(CH₂)₃—SO₃H;
CF₃—CH₂—CH₂—S—Ar—O—CH₂—CH═CH—(CH₂)₃—O—SO₃H; CF₃—CH₂—CH₂—S—Ar—O—CH₂—CH═CH—(CH₂)₃—SH;
CF₃—CH₂—CH₂—S—Ar—O—CH₂—CH═CH—(CH₂)₃—OH;
CF₃—CH₂—CH₂—S—Ar—O—CH₂—CH═CH—(CH₂)₃—N⁺R¹R²R³Z⁻; CF₃—CH₂—CH₂—S—Ar—O—CH₂—CH═CH—(CH₂)₃—P⁺R¹R²R³Z⁻;
CF₃—CH₂—CH₂—S—Ar—O—CH₂—CH═CH—(CH₂)₃—O-
glucoside; CF₃—CH₂—CH₂—S—Ar—O—CH₂—CH═CH—(CH₂)₃—O—PO₃H
CF₃—CH₂—CH₂—S—Ar—O—CH₂—CH═CH—(CH₂)₄—COOH; CF₃—CH₂—CH₂—S—Ar—O—CH₂—CH═CH—(CH₂)₄—SO₃H;
CF₃—CH₂—CH₂—S—Ar—O—CH₂—CH═CH—(CH₂)₄—O—SO₃H; CF₃—CH₂—CH₂—S—Ar—O—CH₂—CH═CH—(CH₂)₄—SH;
CF₃—CH₂—CH₂—S—Ar—O—CH₂—CH═CH—(CH₂)₄—OH;
CF₃—CH₂—CH₂—S—Ar—O—CH₂—CH═CH—(CH₂)₄—N⁺R¹R²R³Z⁻; CF₃—CH₂—CH₂—S—Ar—O—CH₂—CH═CH—(CH₂)₄—P⁺R¹R²R³Z⁻;
CF₃—CH₂—CH₂—S—Ar—O—CH₂—CH═CH—(CH₂)₄—O-
glucoside; CF₃—CH₂—CH₂—S—Ar—O—CH₂—CH═CH—(CH₂)₄—O—PO₃H
CF₃—CH₂—CH₂—S—Ar—O—CH₂—CH═CH—(CH₂)₅—COOH; CF₃—CH₂—CH₂—S—Ar—O—CH₂—CH═CH—(CH₂)₅—SO₃H;
CF₃—CH₂—CH₂—S—Ar—O—CH₂—CH═CH—(CH₂)₅—O—SO₃H; CF₃—CH₂—CH₂—S—Ar—O—CH₂—CH═CH—(CH₂)₅—SH;
CF₃—CH₂—CH₂—S—Ar—O—CH₂—CH═CH—(CH₂)₅—OH;
CF₃—CH₂—CH₂—S—Ar—O—CH₂—CH═CH—(CH₂)₅—N⁺R¹R²R³Z⁻; CF₃—CH₂—CH₂—S—Ar—O—CH₂—CH═CH—(CH₂)₅—P⁺R¹R²R³Z⁻;
CF₃—CH₂—CH₂—S—Ar—O—CH₂—CH═CH—(CH₂)₅—O-
glucoside; CF₃—CH₂—CH₂—S—Ar—O—CH₂—CH═CH—(CH₂)₅—O—PO₃H
CF₃—CH₂—CH₂—S—Ar—O—CH₂—CH═CH—(CH₂)₆—COOH; CF₃—CH₂—CH₂—S—Ar—O—CH₂—CH═CH—(CH₂)₆—SO₃H;
CF₃—CH₂—CH₂—S—Ar—O—CH₂—CH═CH—(CH₂)₆—O—SO₃H; CF₃—CH₂—CH₂—S—Ar—O—CH₂—CH═CH—(CH₂)₆—SH;
CF₃—CH₂—CH₂—S—Ar—O—CH₂—CH═CH—(CH₂)₆—OH;
CF₃—CH₂—CH₂—S—Ar—O—CH₂—CH═CH—(CH₂)₆—N⁺R¹R²R³Z⁻; CF₃—CH₂—CH₂—S—Ar—O—CH₂—CH═CH—(CH₂)₆—P⁺R¹R²R³Z⁻;
CF₃—CH₂—CH₂—S—Ar—O—CH₂—CH═CH—(CH₂)₆—O-
glucoside; CF₃—CH₂—CH₂—S—Ar—O—CH₂—CH═CH—(CH₂)₆—O—PO₃H
CF₃—CH₂—CH₂—S—Ar—O—CH₂—CH═CH—(CH₂)₇—COOH; CF₃—CH₂—CH₂—S—Ar—O—CH₂—CH═CH—(CH₂)₇—SO₃H;
CF₃—CH₂—CH₂—S—Ar—O—CH₂—CH═CH—(CH₂)₇—O—SO₃H; CF₃—CH₂—CH₂—S—Ar—O—CH₂—CH═CH—(CH₂)₇—SH;
CF₃—CH₂—CH₂—S—Ar—O—CH₂—CH═CH—(CH₂)₇—OH;
CF₃—CH₂—CH₂—S—Ar—O—CH₂—CH═CH—(CH₂)₇—N⁺R¹R²R³Z⁻; CF₃—CH₂—CH₂—S—Ar—O—CH₂—CH═CH—(CH₂)₇—P⁺R¹R²R³Z⁻;
CF₃—CH₂—CH₂—S—Ar—O—CH₂—CH═CH—(CH₂)₇—O-
glucoside; CF₃—CH₂—CH₂—S—Ar—O—CH₂—CH═CH—(CH₂)₇—O—PO₃H
CF₃—CH₂—CH₂—S—Ar—O—CH₂—CH═CH—(CH₂)₈—COOH; CF₃—CH₂—CH₂—S—Ar—O—CH₂—CH═CH—(CH₂)₈—SO₃H;
CF₃—CH₂—CH₂—S—Ar—O—CH₂—CH═CH—(CH₂)₈—O—SO₃H; CF₃—CH₂—CH₂—S—Ar—O—CH₂—CH═CH—(CH₂)₈—SH;
CF₃—CH₂—CH₂—S—Ar—O—CH₂—CH═CH—(CH₂)₈—OH;
CF₃—CH₂—CH₂—S—Ar—O—CH₂—CH═CH—(CH₂)₈—N⁺R¹R²R³Z⁻; CF₃—CH₂—CH₂—S—Ar—O—CH₂—CH═CH—(CH₂)₈—P⁺R¹R²R³Z⁻;
CF₃—CH₂—CH₂—S—Ar—O—CH₂—CH═CH—(CH₂)₈—O-
glucoside; CF₃—CH₂—CH₂—S—Ar—O—CH₂—CH═CH—(CH₂)₈—O—PO₃H
CF₃—CH₂—CH₂—S—Ar—O—CH₂—CH═CH—(CH₂)₉—COOH; CF₃—CH₂—CH₂—S—Ar—O—CH₂—CH═CH—(CH₂)₉—SO₃H;
CF₃—CH₂—CH₂—S—Ar—O—CH₂—CH═CH—(CH₂)₉—O—SO₃H; CF₃—CH₂—CH₂—S—Ar—O—CH₂—CH═CH—(CH₂)₉—SH;
CF₃—CH₂—CH₂—S—Ar—O—CH₂—CH═CH—(CH₂)₉—OH;
CF₃—CH₂—CH₂—S—Ar—O—CH₂—CH═CH—(CH₂)₉—N⁺R¹R²R³Z⁻; CF₃—CH₂—CH₂—S—Ar—O—CH₂—CH═CH—(CH₂)₉—P⁺R¹R²R³Z⁻;
CF₃—CH₂—CH₂—S—Ar—O—CH₂—CH═CH—(CH₂)₉—O-
glucoside; CF₃—CH₂—CH₂—S—Ar—O—CH₂—CH═CH—(CH₂)₉—O—PO₃H
CF₃—CH₂—CH₂—S—Ar—O—CH₂—CH═CH—(CH₂)₁₀—COOH; CF₃—CH₂—CH₂—S—Ar—O—CH₂—CH═CH—(CH₂)₁₀—SO₃H;
CF₃—CH₂—CH₂—S—Ar—O—CH₂—CH═CH—(CH₂)₁₀—O—SO₃H; CF₃—CH₂—CH₂—S—Ar—O—CH₂—CH═CH—(CH₂)₁₀—SH;
CF₃—CH₂—CH₂—S—Ar—O—CH₂—CH═CH—(CH₂)₁₀—OH;
CF₃—CH₂—CH₂—S—Ar—O—CH₂—CH═CH—(CH₂)₁₀—N⁺R¹R²R³Z⁻; CF₃—CH₂—CH₂—S—Ar—O—CH₂—CH═CH—(CH₂)₁₀—P⁺R¹R²R³Z⁻;
CF₃—CH₂—CH₂—S—Ar—O—CH₂—CH═CH—(CH₂)₁₀—O-
glucoside; CF₃—CH₂—CH₂—S—Ar—O—CH₂—CH═CH—(CH₂)₁₀—O—PO₃H
CF₃—CH₂—CH₂—S—Ar—O—CH₂—CH═CH—(CH₂)₁₁—COOH; CF₃—CH₂—CH₂—S—Ar—O—CH₂—CH═CH—(CH₂)₁₁—SO₃H;
CF₃—CH₂—CH₂—S—Ar—O—CH₂—CH═CH—(CH₂)₁₁—O—SO₃H; CF₃—CH₂—CH₂—S—Ar—O—CH₂—CH═CH—(CH₂)₁₁—SH;
CF₃—CH₂—CH₂—S—Ar—O—CH₂—CH═CH—(CH₂)₁₁—OH;
CF₃—CH₂—CH₂—S—Ar—O—CH₂—CH═CH—(CH₂)₁₁—N⁺R¹R²R³Z⁻; CF₃—CH₂—CH₂—S—Ar—O—CH₂—CH═CH—(CH₂)₁₁—P⁺R¹R²R³Z⁻;
CF₃—CH₂—CH₂—S—Ar—O—CH₂—CH═CH—(CH₂)₁₁—O-
glucoside; CF₃—CH₂—CH₂—S—Ar—O—CH₂—CH═CH—(CH₂)₁₁—O—PO₃H
CF₃—CH₂—CH₂—S—Ar—O—CH₂—CH═CH—(CH₂)₁₂—COOH; CF₃—CH₂—CH₂—S—Ar—O—CH₂—CH═CH—(CH₂)₁₂—SO₃H;
CF₃—CH₂—CH₂—S—Ar—O—CH₂—CH═CH—(CH₂)₁₂—O—SO₃H; CF₃—CH₂—CH₂—S—Ar—O—CH₂—CH═CH—(CH₂)₁₂—SH;
CF₃—CH₂—CH₂—S—Ar—O—CH₂—CH═CH—(CH₂)₁₂—OH;
CF₃—CH₂—CH₂—S—Ar—O—CH₂—CH═CH—(CH₂)₁₂—N⁺R¹R²R³Z⁻; CF₃—CH₂—CH₂—S—Ar—O—CH₂—CH═CH—(CH₂)₁₂—P⁺R¹R²R³Z⁻;
CF₃—CH₂—CH₂—S—Ar—O—CH₂—CH═CH—(CH₂)₁₂—O-
glucoside; CF₃—CH₂—CH₂—S—Ar—O—CH₂—CH═CH—(CH₂)₁₂—O—PO₃H
CF₃—CH₂—CH₂—S—Ar—O—CH₂—CH═CH—(CH₂)₁₃—COOH; CF₃—CH₂—CH₂—S—Ar—O—CH₂—CH═CH—(CH₂)₁₃—SO₃H;
CF₃—CH₂—CH₂—S—Ar—O—CH₂—CH═CH—(CH₂)₁₃—O—SO₃H; CF₃—CH₂—CH₂—S—Ar—O—CH₂—CH═CH—(CH₂)₁₃—SH;
CF₃—CH₂—CH₂—S—Ar—O—CH₂—CH═CH—(CH₂)₁₃—OH;
CF₃—CH₂—CH₂—S—Ar—O—CH₂—CH═CH—(CH₂)₁₃—N⁺R¹R²R³Z⁻; CF₃—CH₂—CH₂—S—Ar—O—CH₂—CH═CH—(CH₂)₁₃—P⁺R¹R²R³Z⁻;
CF₃—CH₂—CH₂—S—Ar—O—CH₂—CH═CH—(CH₂)₁₃—O-
glucoside; CF₃—CH₂—CH₂—S—Ar—O—CH₂—CH═CH—(CH₂)₁₃—O—PO₃H
CF₃—CH₂—CH₂—S—Ar—O—CH₂—CH═CH—(CH₂)₁₄—COOH; CF₃—CH₂—CH₂—S—Ar—O—CH₂—CH═CH—(CH₂)₁₄—SO₃H;
CF₃—CH₂—CH₂—S—Ar—O—CH₂—CH═CH—(CH₂)₁₄—O—SO₃H; CF₃—CH₂—CH₂—S—Ar—O—CH₂—CH═CH—(CH₂)₁₄—SH;
CF₃—CH₂—CH₂—S—Ar—O—CH₂—CH═CH—(CH₂)₁₄—OH;
CF₃—CH₂—CH₂—S—Ar—O—CH₂—CH═CH—(CH₂)₁₄—N⁺R¹R²R³Z⁻; CF₃—CH₂—CH₂—S—Ar—O—CH₂—CH═CH—(CH₂)₁₄—P⁺R¹R²R³Z⁻;
CF₃—CH₂—CH₂—S—Ar—O—CH₂—CH═CH—(CH₂)₁₄—O-
glucoside; CF₃—CH₂—CH₂—S—Ar—O—CH₂—CH═CH—(CH₂)₁₄—O—PO₃H
CF₃—CH₂—CH₂—S—Ar—O—CH₂—CH═CH—(CH₂)₁₅—COOH; CF₃—CH₂—CH₂—S—Ar—O—CH₂—CH═CH—(CH₂)₁₅—SO₃H;
CF₃—CH₂—CH₂—S—Ar—O—CH₂—CH═CH—(CH₂)₁₅—O—SO₃H; CF₃—CH₂—CH₂—S—Ar—O—CH₂—CH═CH—(CH₂)₁₅—SH;
CF₃—CH₂—CH₂—S—Ar—O—CH₂—CH═CH—(CH₂)₁₅—OH;
CF₃—CH₂—CH₂—S—Ar—O—CH₂—CH═CH—(CH₂)₁₅—N⁺R¹R²R³Z⁻; CF₃—CH₂—CH₂—S—Ar—O—CH₂—CH═CH—(CH₂)₁₅—P⁺R¹R²R³Z⁻;
CF₃—CH₂—CH₂—S—Ar—O—CH₂—CH═CH—(CH₂)₁₅—O-
glucoside; CF₃—CH₂—CH₂—S—Ar—O—CH₂—CH═CH—(CH₂)₁₅—O—PO₃H
CF₃—CH₂—CH₂—S—Ar—O—CH₂—CH═CH—(CH₂)₁₆—COOH; CF₃—CH₂—CH₂—S—Ar—O—CH₂—CH═CH—(CH₂)₁₆—SO₃H;
CF₃—CH₂—CH₂—S—Ar—O—CH₂—CH═CH—(CH₂)₁₆—O—SO₃H; CF₃—CH₂—CH₂—S—Ar—O—CH₂—CH═CH—(CH₂)₁₆—SH;
CF₃—CH₂—CH₂—S—Ar—O—CH₂—CH═CH—(CH₂)₁₆—OH;
CF₃—CH₂—CH₂—S—Ar—O—CH₂—CH═CH—(CH₂)₁₆—N⁺R¹R²R³Z⁻; CF₃—CH₂—CH₂—S—Ar—O—CH₂—CH═CH—(CH₂)₁₆—P⁺R¹R²R³Z⁻;
CF₃—CH₂—CH₂—S—Ar—O—CH₂—CH═CH—(CH₂)₁₆—O-
glucoside; CF₃—CH₂—CH₂—S—Ar—O—CH₂—CH═CH—(CH₂)₁₆—O—PO₃H
CF₃—CH₂—CH₂—S—Ar—O—C(═O)—(CH₂)₃—COOH; CF₃—CH₂—CH₂—S—Ar—O—C(═O)—(CH₂)₃—SO₃H;
CF₃—CH₂—CH₂—S—Ar—O—C(═O)—(CH₂)₃—O—SO₃H; CF₃—CH₂—CH₂—S—Ar—O—C(═O)—(CH₂)₃—SH;
CF₃—CH₂—CH₂—S—Ar—O—C(═O)—(CH₂)₃—OH; CF₃—CH₂—CH₂—S—Ar—O—C(═O)—(CH₂)₃—N⁺R¹R²R³Z⁻;
CF₃—CH₂—CH₂—S—Ar—O—C(═O)—(CH₂)₃—P⁺R¹R²R³Z⁻; CF₃—CH₂—CH₂—S—Ar—O—C(═O)—(CH₂)₃—O-
glucoside; CF₃—CH₂—CH₂—S—Ar—O—C(═O)—(CH₂)₃—O—PO₃H
CF₃—CH₂—CH₂—S—Ar—O—C(═O)—(CH₂)₄—COOH; CF₃—CH₂—CH₂—S—Ar—O—C(═O)—(CH₂)₄—SO₃H;
CF₃—CH₂—CH₂—S—Ar—O—C(═O)—(CH₂)₄—O—SO₃H; CF₃—CH₂—CH₂—S—Ar—O—C(═O)—(CH₂)₄—SH;
CF₃—CH₂—CH₂—S—Ar—O—C(═O)—(CH₂)₄—OH; CF₃—CH₂—CH₂—S—Ar—O—C(═O)—(CH₂)₄—N⁺R¹R²R³Z⁻;
CF₃—CH₂—CH₂—S—Ar—O—C(═O)—(CH₂)₄—P⁺R¹R²R³Z⁻; CF₃—CH₂—CH₂—S—Ar—O—C(═O)—(CH₂)₄—O-
glucoside; CF₃—CH₂—CH₂—S—Ar—O—C(═O)—(CH₂)₄—O—PO₃H
CF₃—CH₂—CH₂—S—Ar—O—C(═O)—(CH₂)₅—COOH; CF₃—CH₂—CH₂—S—Ar—O—C(═O)—(CH₂)₅—SO₃H;
CF₃—CH₂—CH₂—S—Ar—O—C(═O)—(CH₂)₅—O—SO₃H; CF₃—CH₂—CH₂—S—Ar—O—C(═O)—(CH₂)₅—SH;
CF₃—CH₂—CH₂—S—Ar—O—C(═O)—(CH₂)₅—OH; CF₃—CH₂—CH₂—S—Ar—O—C(═O)—(CH₂)₅—N⁺R¹R²R³Z⁻;
CF₃—CH₂—CH₂—S—Ar—O—C(═O)—(CH₂)₅—P⁺R¹R²R³Z⁻; CF₃—CH₂—CH₂—S—Ar—O—C(═O)—(CH₂)₅—O-
glucoside; CF₃—CH₂—CH₂—S—Ar—O—C(═O)—(CH₂)₅—O—PO₃H
CF₃—CH₂—CH₂—S—Ar—O—C(═O)—(CH₂)₆—COOH; CF₃—CH₂—CH₂—S—Ar—O—C(═O)—(CH₂)₆—SO₃H;
CF₃—CH₂—CH₂—S—Ar—O—C(═O)—(CH₂)₆—O—SO₃H; CF₃—CH₂—CH₂—S—Ar—O—C(═O)—(CH₂)₆—SH;
CF₃—CH₂—CH₂—S—Ar—O—C(═O)—(CH₂)₆—OH; CF₃—CH₂—CH₂—S—Ar—O—C(═O)—(CH₂)₆—N⁺R¹R²R³Z⁻;
CF₃—CH₂—CH₂—S—Ar—O—C(═O)—(CH₂)₆—P⁺R¹R²R³Z⁻; CF₃—CH₂—CH₂—S—Ar—O—C(═O)—(CH₂)₆—O-
glucoside; CF₃—CH₂—CH₂—S—Ar—O—C(═O)—(CH₂)₆—O—PO₃H
CF₃—CH₂—CH₂—S—Ar—O—C(═O)—(CH₂)₇—COOH; CF₃—CH₂—CH₂—S—Ar—O—C(═O)—(CH₂)₇—SO₃H;
CF₃—CH₂—CH₂—S—Ar—O—C(═O)—(CH₂)₇—O—SO₃H; CF₃—CH₂—CH₂—S—Ar—O—C(═O)—(CH₂)₇—SH;
CF₃—CH₂—CH₂—S—Ar—O—C(═O)—(CH₂)₇—OH; CF₃—CH₂—CH₂—S—Ar—O—C(═O)—(CH₂)₇—N⁺R¹R²R³Z⁻;
CF₃—CH₂—CH₂—S—Ar—O—C(═O)—(CH₂)₇—P⁺R¹R²R³Z⁻; CF₃—CH₂—CH₂—S—Ar—O—C(═O)—(CH₂)₇—O-
glucoside; CF₃—CH₂—CH₂—S—Ar—O—C(═O)—(CH₂)₇—O—PO₃H
CF₃—CH₂—CH₂—S—Ar—O—C(═O)—(CH₂)₈—COOH; CF₃—CH₂—CH₂—S—Ar—O—C(═O)—(CH₂)₈—SO₃H;
CF₃—CH₂—CH₂—S—Ar—O—C(═O)—(CH₂)₈—O—SO₃H; CF₃—CH₂—CH₂—S—Ar—O—C(═O)—(CH₂)₈—SH;
CF₃—CH₂—CH₂—S—Ar—O—C(═O)—(CH₂)₈—OH; CF₃—CH₂—CH₂—S—Ar—O—C(═O)—(CH₂)₈—N⁺R¹R²R³Z⁻;
CF₃—CH₂—CH₂—S—Ar—O—C(═O)—(CH₂)₈—P⁺R¹R²R³Z⁻; CF₃—CH₂—CH₂—S—Ar—O—C(═O)—(CH₂)₈—O-
glucoside; CF₃—CH₂—CH₂—S—Ar—O—C(═O)—(CH₂)₈—O—PO₃H
CF₃—CH₂—CH₂—S—Ar—O—C(═O)—(CH₂)₉—COOH; CF₃—CH₂—CH₂—S—Ar—O—C(═O)—(CH₂)₉—SO₃H;
CF₃—CH₂—CH₂—S—Ar—O—C(═O)—(CH₂)₉—O—SO₃H; CF₃—CH₂—CH₂—S—Ar—O—C(═O)—(CH₂)₉—SH;
CF₃—CH₂—CH₂—S—Ar—O—C(═O)—(CH₂)₉—OH; CF₃—CH₂—CH₂—S—Ar—O—C(═O)—(CH₂)₉—N⁺R¹R²R³Z⁻;
CF₃—CH₂—CH₂—S—Ar—O—C(═O)—(CH₂)₉—P⁺R¹R²R³Z⁻; CF₃—CH₂—CH₂—S—Ar—O—C(═O)—(CH₂)₉—O-
glucoside; CF₃—CH₂—CH₂—S—Ar—O—C(═O)—(CH₂)₉—O—PO₃H
CF₃—CH₂—CH₂—S—Ar—O—C(═O)—(CH₂)₁₀—COOH; CF₃—CH₂—CH₂—S—Ar—O—C(═O)—(CH₂)₁₀—SO₃H;
CF₃—CH₂—CH₂—S—Ar—O—C(═O)—(CH₂)₁₀—O—SO₃H; CF₃—CH₂—CH₂—S—Ar—O—C(═O)—(CH₂)₁₀—SH;
CF₃—CH₂—CH₂—S—Ar—O—C(═O)—(CH₂)₁₀—OH; CF₃—CH₂—CH₂—S—Ar—O—C(═O)—(CH₂)₁₀—N⁺R¹R²R³Z⁻;
CF₃—CH₂—CH₂—S—Ar—O—C(═O)—(CH₂)₁₀—P⁺R¹R²R³Z⁻; CF₃—CH₂—CH₂—S—Ar—O—C(═O)—(CH₂)₁₀—O-
glucoside; CF₃—CH₂—CH₂—S—Ar—O—C(═O)—(CH₂)₁₀—O—PO₃H
CF₃—CH₂—CH₂—S—Ar—O—C(═O)—(CH₂)₁₁—COOH; CF₃—CH₂—CH₂—S—Ar—O—C(═O)—(CH₂)₁₁—SO₃H;
CF₃—CH₂—CH₂—S—Ar—O—C(═O)—(CH₂)₁₁—O—SO₃H; CF₃—CH₂—CH₂—S—Ar—O—C(═O)—(CH₂)₁₁—SH;
CF₃—CH₂—CH₂—S—Ar—O—C(═O)—(CH₂)₁₁—OH; CF₃—CH₂—CH₂—S—Ar—O—C(═O)—(CH₂)₁₁—N⁺R¹R²R³Z⁻;
CF₃—CH₂—CH₂—S—Ar—O—C(═O)—(CH₂)₁₁—P⁺R¹R²R³Z⁻; CF₃—CH₂—CH₂—S—Ar—O—C(═O)—(CH₂)₁₁—O-
glucoside; CF₃—CH₂—CH₂—S—Ar—O—C(═O)—(CH₂)₁₁—O—PO₃H
CF₃—CH₂—CH₂—S—Ar—O—C(═O)—(CH₂)₁₂—COOH; CF₃—CH₂—CH₂—S—Ar—O—C(═O)—(CH₂)₁₂—SO₃H;
CF₃—CH₂—CH₂—S—Ar—O—C(═O)—(CH₂)₁₂—O—SO₃H; CF₃—CH₂—CH₂—S—Ar—O—C(═O)—(CH₂)₁₂—SH;
CF₃—CH₂—CH₂—S—Ar—O—C(═O)—(CH₂)₁₂—OH; CF₃—CH₂—CH₂—S—Ar—O—C(═O)—(CH₂)₁₂—N⁺R¹R²R³Z⁻;
CF₃—CH₂—CH₂—S—Ar—O—C(═O)—(CH₂)₁₂—P⁺R¹R²R³Z⁻; CF₃—CH₂—CH₂—S—Ar—O—C(═O)—(CH₂)₁₂—O-
glucoside; CF₃—CH₂—CH₂—S—Ar—O—C(═O)—(CH₂)₁₂—O—PO₃H
CF₃—CH₂—CH₂—S—Ar—O—C(═O)—(CH₂)₁₃—COOH; CF₃—CH₂—CH₂—S—Ar—O—C(═O)—(CH₂)₁₃—SO₃H;
CF₃—CH₂—CH₂—S—Ar—O—C(═O)—(CH₂)₁₃—O—SO₃H; CF₃—CH₂—CH₂—S—Ar—O—C(═O)—(CH₂)₁₃—SH;
CF₃—CH₂—CH₂—S—Ar—O—C(═O)—(CH₂)₁₃—OH; CF₃—CH₂—CH₂—S—Ar—O—C(═O)—(CH₂)₁₃—N⁺R¹R²R³Z⁻;
CF₃—CH₂—CH₂—S—Ar—O—C(═O)—(CH₂)₁₃—P⁺R¹R²R³Z⁻; CF₃—CH₂—CH₂—S—Ar—O—C(═O)—(CH₂)₁₃—O-
glucoside; CF₃—CH₂—CH₂—S—Ar—O—C(═O)—(CH₂)₁₃—O—PO₃H
CF₃—CH₂—CH₂—S—Ar—O—C(═O)—(CH₂)₁₄—COOH; CF₃—CH₂—CH₂—S—Ar—O—C(═O)—(CH₂)₁₄—SO₃H;
CF₃—CH₂—CH₂—S—Ar—O—C(═O)—(CH₂)₁₄—O—SO₃H; CF₃—CH₂—CH₂—S—Ar—O—C(═O)—(CH₂)₁₄—SH;
CF₃—CH₂—CH₂—S—Ar—O—C(═O)—(CH₂)₁₄—OH; CF₃—CH₂—CH₂—S—Ar—O—C(═O)—(CH₂)₁₄—N⁺R¹R²R³Z⁻;
CF₃—CH₂—CH₂—S—Ar—O—C(═O)—(CH₂)₁₄—P⁺R¹R²R³Z⁻; CF₃—CH₂—CH₂—S—Ar—O—C(═O)—(CH₂)₁₄—O-
glucoside; CF₃—CH₂—CH₂—S—Ar—O—C(═O)—(CH₂)₁₄—O—PO₃H
CF₃—CH₂—CH₂—S—Ar—O—C(═O)—(CH₂)₁₅—COOH; CF₃—CH₂—CH₂—S—Ar—O—C(═O)—(CH₂)₁₅—SO₃H;
CF₃—CH₂—CH₂—S—Ar—O—C(═O)—(CH₂)₁₅—O—SO₃H; CF₃—CH₂—CH₂—S—Ar—O—C(═O)—(CH₂)₁₅—SH;
CF₃—CH₂—CH₂—S—Ar—O—C(═O)—(CH₂)₁₅—OH; CF₃—CH₂—CH₂—S—Ar—O—C(═O)—(CH₂)₁₅—N⁺R¹R²R³Z⁻;
CF₃—CH₂—CH₂—S—Ar—O—C(═O)—(CH₂)₁₅—P⁺R¹R²R³Z⁻; CF₃—CH₂—CH₂—S—Ar—O—C(═O)—(CH₂)₁₅—O-
glucoside; CF₃—CH₂—CH₂—S—Ar—O—C(═O)—(CH₂)₁₅—O—PO₃H
CF₃—CH₂—CH₂—S—Ar—O—C(═O)—(CH₂)₁₆—COOH; CF₃—CH₂—CH₂—S—Ar—O—C(═O)—(CH₂)₁₆—SO₃H;
CF₃—CH₂—CH₂—S—Ar—O—C(═O)—(CH₂)₁₆—O—SO₃H; CF₃—CH₂—CH₂—S—Ar—O—C(═O)—(CH₂)₁₆—SH;
CF₃—CH₂—CH₂—S—Ar—O—C(═O)—(CH₂)₁₆—OH; CF₃—CH₂—CH₂—S—Ar—O—C(═O)—(CH₂)₁₆—N⁺R¹R²R³Z⁻;
CF₃—CH₂—CH₂—S—Ar—O—C(═O)—(CH₂)₁₆—P⁺R¹R²R³Z⁻; CF₃—CH₂—CH₂—S—Ar—O—C(═O)—(CH₂)₁₆—O-
glucoside; CF₃—CH₂—CH₂—S—Ar—O—C(═O)—(CH₂)₁₆—O—PO₃H
CF₃—CH₂—CH₂—S—Ar—O—C(═O)—(CH₂)ₙ—O—(CH₂)_{n′}—COOH; CF₃—CH₂—CH₂—S—Ar—O—C(═O)—(CH₂)ₙ—O—(CH₂)_{n′}—SO₃H;
CF₃—CH₂—CH₂—S—Ar—O—C(═O)—(CH₂)ₙ—O—(CH₂)_{n′}—O—SO₃H;
CF₃—CH₂—CH₂—S—Ar—O—C(═O)—(CH₂)ₙ—O—(CH₂)_{n′}—SH; CF₃—CH₂—CH₂—S—Ar—O—C(═O)—(CH₂)ₙ—O—(CH₂)_{n′}—OH;
CF₃—CH₂—CH₂—S—Ar—O—C(═O)—(CH₂)ₙ—O—(CH₂)_{n′}—N⁺R¹R²R³Z⁻;
CF₃—CH₂—CH₂—S—Ar—O—C(═O)—(CH₂)ₙ—O—(CH₂)_{n′}—P⁺R¹R²R³Z⁻; CF₃—CH₂—CH₂—S—Ar—O—C(═O)—(CH₂)ₙ—O—(CH₂)_{n′}—O-
glucoside; CF₃—CH₂—CH₂—S—Ar—O—C(═O)—(CH₂)ₙ—O—(CH₂)_{n′}—O—PO₃H
CF₃—CH₂—CH₂—S—Ar—O—C(═O)—CH₂—CH═CH—(CH₂)₃—COOH;
CF₃—CH₂—CH₂—S—Ar—O—C(═O)—CH₂—CH═CH—(CH₂)₃—SO₃H;
CF₃—CH₂—CH₂—S—Ar—O—C(═O)—CH₂—CH═CH—(CH₂)₃—O—SO₃H;
CF₃—CH₂—CH₂—S—Ar—O—C(═O)—CH₂—CH═CH—(CH₂)₃—SH; CF₃—CH₂—CH₂—S—Ar—O—C(═O)—CH₂—CH═CH—(CH₂)₃—OH;
CF₃—CH₂—CH₂—S—Ar—O—C(═O)—CH₂—CH═CH—(CH₂)₃—N⁺R¹R²R³Z⁻;
CF₃—CH₂—CH₂—S—Ar—O—C(═O)—CH₂—CH═CH—(CH₂)₃—P⁺R¹R²R³Z⁻;
CF₃—CH₂—CH₂—S—Ar—O—C(═O)—CH₂—CH═CH—(CH₂)₃—O-glucoside;
CF₃—CH₂—CH₂—S—Ar—O—C(═O)—CH₂—CH═CH—(CH₂)₃—O—PO₃H
CF₃—CH₂—CH₂—S—Ar—O—C(═O)—CH₂—CH═CH—(CH₂)₄—COOH;
CF₃—CH₂—CH₂—S—Ar—O—C(═O)—CH₂—CH═CH—(CH₂)₄—SO₃H;
CF₃—CH₂—CH₂—S—Ar—O—C(═O)—CH₂—CH═CH—(CH₂)₄—O—SO₃H;
CF₃—CH₂—CH₂—S—Ar—O—C(═O)—CH₂—CH═CH—(CH₂)₄—SH; CF₃—CH₂—CH₂—S—Ar—O—C(═O)—CH₂—CH═CH—(CH₂)₄—OH;
CF₃—CH₂—CH₂—S—Ar—O—C(═O)—CH₂—CH═CH—(CH₂)₄—N⁺R¹R²R³Z⁻;
CF₃—CH₂—CH₂—S—Ar—O—C(═O)—CH₂—CH═CH—(CH₂)₄—P⁺R¹R²R³Z⁻;
CF₃—CH₂—CH₂—S—Ar—O—C(═O)—CH₂—CH═CH—(CH₂)₄—O-glucoside;
CF₃—CH₂—CH₂—S—Ar—O—C(═O)—CH₂—CH═CH—(CH₂)₄—O—PO₃H
CF₃—CH₂—CH₂—S—Ar—O—C(═O)—CH₂—CH═CH—(CH₂)₅—COOH;
CF₃—CH₂—CH₂—S—Ar—O—C(═O)—CH₂—CH═CH—(CH₂)₅—SO₃H;
CF₃—CH₂—CH₂—S—Ar—O—C(═O)—CH₂—CH═CH—(CH₂)₅—O—SO₃H;
CF₃—CH₂—CH₂—S—Ar—O—C(═O)—CH₂—CH═CH—(CH₂)₅—SH; CF₃—CH₂—CH₂—S—Ar—O—C(═O)—CH₂—CH═CH—(CH₂)₅—OH;
CF₃—CH₂—CH₂—S—Ar—O—C(═O)—CH₂—CH═CH—(CH₂)₅—N⁺R¹R²R³Z⁻;
CF₃—CH₂—CH₂—S—Ar—O—C(═O)—CH₂—CH═CH—(CH₂)₅—P⁺R¹R²R³Z⁻;
CF₃—CH₂—CH₂—S—Ar—O—C(═O)—CH₂—CH═CH—(CH₂)₅—O-glucoside;
CF₃—CH₂—CH₂—S—Ar—O—C(═O)—CH₂—CH═CH—(CH₂)₅—O—PO₃H
CF₃—CH₂—CH₂—S—Ar—O—C(═O)—CH₂—CH═CH—(CH₂)₆—COOH;
CF₃—CH₂—CH₂—S—Ar—O—C(═O)—CH₂—CH═CH—(CH₂)₆—SO₃H;
CF₃—CH₂—CH₂—S—Ar—O—C(═O)—CH₂—CH═CH—(CH₂)₆—O—SO₃H;
CF₃—CH₂—CH₂—S—Ar—O—C(═O)—CH₂—CH═CH—(CH₂)₆—SH; CF₃—CH₂—CH₂—S—Ar—O—C(═O)—CH₂—CH═CH—(CH₂)₆—OH;
CF₃—CH₂—CH₂—S—Ar—O—C(═O)—CH₂—CH═CH—(CH₂)₆—N⁺R¹R²R³Z⁻;
CF₃—CH₂—CH₂—S—Ar—O—C(═O)—CH₂—CH═CH—(CH₂)₆—P⁺R¹R²R³Z⁻;
CF₃—CH₂—CH₂—S—Ar—O—C(═O)—CH₂—CH═CH—(CH₂)₆—O-glucoside;
CF₃—CH₂—CH₂—S—Ar—O—C(═O)—CH₂—CH═CH—(CH₂)₆—O—PO₃H
CF₃—CH₂—CH₂—S—Ar—O—C(═O)—CH₂—CH═CH—(CH₂)₇—COOH;
CF₃—CH₂—CH₂—S—Ar—O—C(═O)—CH₂—CH═CH—(CH₂)₇—SO₃H;
CF₃—CH₂—CH₂—S—Ar—O—C(═O)—CH₂—CH═CH—(CH₂)₇—O—SO₃H;
CF₃—CH₂—CH₂—S—Ar—O—C(═O)—CH₂—CH═CH—(CH₂)₇—SH; CF₃—CH₂—CH₂—S—Ar—O—C(═O)—CH₂—CH═CH—(CH₂)₇—OH;
CF₃—CH₂—CH₂—S—Ar—O—C(═O)—CH₂—CH═CH—(CH₂)₇—N⁺R¹R²R³Z⁻;
CF₃—CH₂—CH₂—S—Ar—O—C(═O)—CH₂—CH═CH—(CH₂)₇—P⁺R¹R²R³Z⁻;
CF₃—CH₂—CH₂—S—Ar—O—C(═O)—CH₂—CH═CH—(CH₂)₇—O-glucoside;
CF₃—CH₂—CH₂—S—Ar—O—C(═O)—CH₂—CH═CH—(CH₂)₇—O—PO₃H
CF₃—CH₂—CH₂—S—Ar—O—C(═O)—CH₂—CH═CH—(CH₂)₈—COOH;
CF₃—CH₂—CH₂—S—Ar—O—C(═O)—CH₂—CH═CH—(CH₂)₈—SO₃H;
CF₃—CH₂—CH₂—S—Ar—O—C(═O)—CH₂—CH═CH—(CH₂)₈—O—SO₃H;
CF₃—CH₂—CH₂—S—Ar—O—C(═O)—CH₂—CH═CH—(CH₂)₈—SH; CF₃—CH₂—CH₂—S—Ar—O—C(═O)—CH₂—CH═CH—(CH₂)₈—OH;
CF₃—CH₂—CH₂—S—Ar—O—C(═O)—CH₂—CH═CH—(CH₂)₈—N⁺R¹R²R³Z⁻;
CF₃—CH₂—CH₂—S—Ar—O—C(═O)—CH₂—CH═CH—(CH₂)₈—P⁺R¹R²R³Z⁻;
CF₃—CH₂—CH₂—S—Ar—O—C(═O)—CH₂—CH═CH—(CH₂)₈—O-glucoside;
CF₃—CH₂—CH₂—S—Ar—O—C(═O)—CH₂—CH═CH—(CH₂)₈—O—PO₃H
CF₃—CH₂—CH₂—S—Ar—O—C(═O)—CH₂—CH═CH—(CH₂)₉—COOH;
CF₃—CH₂—CH₂—S—Ar—O—C(═O)—CH₂—CH═CH—(CH₂)₉—SO₃H;
CF₃—CH₂—CH₂—S—Ar—O—C(═O)—CH₂—CH═CH—(CH₂)₉—O—SO₃H;
CF₃—CH₂—CH₂—S—Ar—O—C(═O)—CH₂—CH═CH—(CH₂)₉—SH; CF₃—CH₂—CH₂—S—Ar—O—C(═O)—CH₂—CH═CH—(CH₂)₉—OH;
CF₃—CH₂—CH₂—S—Ar—O—C(═O)—CH₂—CH═CH—(CH₂)₉—N⁺R¹R²R³Z⁻;
CF₃—CH₂—CH₂—S—Ar—O—C(═O)—CH₂—CH═CH—(CH₂)₉—P⁺R¹R²R³Z⁻;
CF₃—CH₂—CH₂—S—Ar—O—C(═O)—CH₂—CH═CH—(CH₂)₉—O-glucoside;
CF₃—CH₂—CH₂—S—Ar—O—C(═O)—CH₂—CH═CH—(CH₂)₉—O—PO₃H
CF₃—CH₂—CH₂—S—Ar—O—C(═O)—CH₂—CH═CH—(CH₂)₁₀—COOH;
CF₃—CH₂—CH₂—S—Ar—O—C(═O)—CH₂—CH═CH—(CH₂)₁₀—SO₃H;
CF₃—CH₂—CH₂—S—Ar—O—C(═O)—CH₂—CH═CH—(CH₂)₁₀—O—SO₃H;
CF₃—CH₂—CH₂—S—Ar—O—C(═O)—CH₂—CH═CH—(CH₂)₁₀—SH; CF₃—CH₂—CH₂—S—Ar—O—C(═O)—CH₂—CH═CH—(CH₂)₁₀—OH;
CF₃—CH₂—CH₂—S—Ar—O—C(═O)—CH₂—CH═CH—(CH₂)₁₀—N⁺R¹R²R³Z⁻;
CF₃—CH₂—CH₂—S—Ar—O—C(═O)—CH₂—CH═CH—(CH₂)₁₀—P⁺R¹R²R³Z⁻;
CF₃—CH₂—CH₂—S—Ar—O—C(═O)—CH₂—CH═CH—(CH₂)₁₀—O-glucoside;
CF₃—CH₂—CH₂—S—Ar—O—C(═O)—CH₂—CH═CH—(CH₂)₁₀—O—PO₃H
CF₃—CH₂—CH₂—S—Ar—O—C(═O)—CH₂—CH═CH—(CH₂)₁₁—COOH;
CF₃—CH₂—CH₂—S—Ar—O—C(═O)—CH₂—CH═CH—(CH₂)₁₁—SO₃H;
CF₃—CH₂—CH₂—S—Ar—O—C(═O)—CH₂—CH═CH—(CH₂)₁₁—O—SO₃H;
CF₃—CH₂—CH₂—S—Ar—O—C(═O)—CH₂—CH═CH—(CH₂)₁₁—SH; CF₃—CH₂—CH₂—S—Ar—O—C(═O)—CH₂—CH═CH—(CH₂)₁₁—OH;
CF₃—CH₂—CH₂—S—Ar—O—C(═O)—CH₂—CH═CH—(CH₂)₁₁—N⁺R¹R²R³Z⁻;
CF₃—CH₂—CH₂—S—Ar—O—C(═O)—CH₂—CH═CH—(CH₂)₁₁—P⁺R¹R²R³Z⁻;
CF₃—CH₂—CH₂—S—Ar—O—C(═O)—CH₂—CH═CH—(CH₂)₁₁—O-glucoside;
CF₃—CH₂—CH₂—S—Ar—O—C(═O)—CH₂—CH═CH—(CH₂)₁₁—O—PO₃H
CF₃—CH₂—CH₂—S—Ar—O—C(═O)—CH₂—CH═CH—(CH₂)₁₂—COOH;
CF₃—CH₂—CH₂—S—Ar—O—C(═O)—CH₂—CH═CH—(CH₂)₁₂—SO₃H;
CF₃—CH₂—CH₂—S—Ar—O—C(═O)—CH₂—CH═CH—(CH₂)₁₂—O—SO₃H;
CF₃—CH₂—CH₂—S—Ar—O—C(═O)—CH₂—CH═CH—(CH₂)₁₂—SH; CF₃—CH₂—CH₂—S—Ar—O—C(═O)—CH₂—CH═CH—(CH₂)₁₂—OH;
CF₃—CH₂—CH₂—S—Ar—O—C(═O)—CH₂—CH═CH—(CH₂)₁₂—N⁺R¹R²R³Z⁻;
CF₃—CH₂—CH₂—S—Ar—O—C(═O)—CH₂—CH═CH—(CH₂)₁₂—P⁺R¹R²R³Z⁻;
CF₃—CH₂—CH₂—S—Ar—O—C(═O)—CH₂—CH═CH—(CH₂)₁₂—O-glucoside;
CF₃—CH₂—CH₂—S—Ar—O—C(═O)—CH₂—CH═CH—(CH₂)₁₂—O—PO₃H
CF₃—CH₂—CH₂—S—Ar—O—C(═O)—CH₂—CH═CH—(CH₂)₁₃—COOH;
CF₃—CH₂—CH₂—S—Ar—O—C(═O)—CH₂—CH═CH—(CH₂)₁₃—SO₃H;
CF₃—CH₂—CH₂—S—Ar—O—C(═O)—CH₂—CH═CH—(CH₂)₁₃—O—SO₃H;
CF₃—CH₂—CH₂—S—Ar—O—C(═O)—CH₂—CH═CH—(CH₂)₁₃—SH; CF₃—CH₂—CH₂—S—Ar—O—C(═O)—CH₂—CH═CH—(CH₂)₁₃—OH;
CF₃—CH₂—CH₂—S—Ar—O—C(═O)—CH₂—CH═CH—(CH₂)₁₃—N⁺R¹R²R³Z⁻;
CF₃—CH₂—CH₂—S—Ar—O—C(═O)—CH₂—CH═CH—(CH₂)₁₃—P⁺R¹R²R³Z⁻;
CF₃—CH₂—CH₂—S—Ar—O—C(═O)—CH₂—CH═CH—(CH₂)₁₃—O-glucoside;
CF₃—CH₂—CH₂—S—Ar—O—C(═O)—CH₂—CH═CH—(CH₂)₁₃—O—PO₃H
CF₃—CH₂—CH₂—S—Ar—O—C(═O)—CH₂—CH═CH—(CH₂)₁₄—COOH;
CF₃—CH₂—CH₂—S—Ar—O—C(═O)—CH₂—CH═CH—(CH₂)₁₄—SO₃H;
CF₃—CH₂—CH₂—S—Ar—O—C(═O)—CH₂—CH═CH—(CH₂)₁₄—O—SO₃H;
CF₃—CH₂—CH₂—S—Ar—O—C(═O)—CH₂—CH═CH—(CH₂)₁₄—SH; CF₃—CH₂—CH₂—S—Ar—O—C(═O)—CH₂—CH═CH—(CH₂)₁₄—OH;
CF₃—CH₂—CH₂—S—Ar—O—C(═O)—CH₂—CH═CH—(CH₂)₁₄—N⁺R¹R²R³Z⁻;
CF₃—CH₂—CH₂—S—Ar—O—C(═O)—CH₂—CH═CH—(CH₂)₁₄—P⁺R¹R²R³Z⁻;
CF₃—CH₂—CH₂—S—Ar—O—C(═O)—CH₂—CH═CH—(CH₂)₁₄—O-glucoside;
CF₃—CH₂—CH₂—S—Ar—O—C(═O)—CH₂—CH═CH—(CH₂)₁₄—O—PO₃H
CF₃—CH₂—CH₂—S—Ar—O—C(═O)—CH₂—CH═CH—(CH₂)₁₅—COOH;
CF₃—CH₂—CH₂—S—Ar—O—C(═O)—CH₂—CH═CH—(CH₂)₁₅—SO₃H;
CF₃—CH₂—CH₂—S—Ar—O—C(═O)—CH₂—CH═CH—(CH₂)₁₅—O—SO₃H;
CF₃—CH₂—CH₂—S—Ar—O—C(═O)—CH₂—CH═CH—(CH₂)₁₅—SH; CF₃—CH₂—CH₂—S—Ar—O—C(═O)—CH₂—CH═CH—(CH₂)₁₅—OH;
CF₃—CH₂—CH₂—S—Ar—O—C(═O)—CH₂—CH═CH—(CH₂)₁₅—N⁺R¹R²R³Z⁻;
CF₃—CH₂—CH₂—S—Ar—O—C(═O)—CH₂—CH═CH—(CH₂)₁₅—P⁺R¹R²R³Z⁻;
CF₃—CH₂—CH₂—S—Ar—O—C(═O)—CH₂—CH═CH—(CH₂)₁₅—O-glucoside;
CF₃—CH₂—CH₂—S—Ar—O—C(═O)—CH₂—CH═CH—(CH₂)₁₅—O—PO₃H
CF₃—CH₂—CH₂—S—Ar—O—C(═O)—CH₂—CH═CH—(CH₂)₁₆—COOH;
CF₃—CH₂—CH₂—S—Ar—O—C(═O)—CH₂—CH═CH—(CH₂)₁₆—SO₃H;
CF₃—CH₂—CH₂—S—Ar—O—C(═O)—CH₂—CH═CH—(CH₂)₁₆—O—SO₃H;
CF₃—CH₂—CH₂—S—Ar—O—C(═O)—CH₂—CH═CH—(CH₂)₁₆—SH; CF₃—CH₂—CH₂—S—Ar—O—C(═O)—CH₂—CH═CH—(CH₂)₁₆—OH;
CF₃—CH₂—CH₂—S—Ar—O—C(═O)—CH₂—CH═CH—(CH₂)₁₆—N⁺R¹R²R³Z⁻;
CF₃—CH₂—CH₂—S—Ar—O—C(═O)—CH₂—CH═CH—(CH₂)₁₆—P⁺R¹R²R³Z⁻;
CF₃—CH₂—CH₂—S—Ar—O—C(═O)—CH₂—CH═CH—(CH₂)₁₆—O-glucoside;
CF₃—CH₂—CH₂—S—Ar—O—C(═O)—CH₂—CH═CH—(CH₂)₁₆—O—PO₃H
(CF₃)₂N—Ar—O—(CH₂)₃—COOH; (CF₃)₂N—Ar—O—(CH₂)₃—SO₃H; (CF₃)₂N—Ar—O—(CH₂)₃—O—SO₃H;
(CF₃)₂N—Ar—O—(CH₂)₃—SH; (CF₃)₂N—Ar—O—(CH₂)₃—OH; (CF₃)₂N—Ar—O—(CH₂)₃—N⁺R¹R²R³Z⁻;
(CF₃)₂N—Ar—O—(CH₂)₃—P⁺R¹R²R³Z⁻; (CF₃)₂N—Ar—O—(CH₂)₃—O-glucoside;
(CF₃)₂N—Ar—O—(CH₂)₃—O—PO₃H
(CF₃)₂N—Ar—O—(CH₂)₄—COOH; (CF₃)₂N—Ar—O—(CH₂)₄—SO₃H; (CF₃)₂N—Ar—O—(CH₂)₄—O—SO₃H;
(CF₃)₂N—Ar—O—(CH₂)₄—SH; (CF₃)₂N—Ar—O—(CH₂)₄—OH; (CF₃)₂N—Ar—O—(CH₂)₄—N⁺R¹R²R³Z⁻;
(CF₃)₂N—Ar—O—(CH₂)₄—P⁺R¹R²R³Z⁻; (CF₃)₂N—Ar—O—(CH₂)₄—O-glucoside;
(CF₃)₂N—Ar—O—(CH₂)₄—O—PO₃H
(CF₃)₂N—Ar—O—(CH₂)₅—COOH; (CF₃)₂N—Ar—O—(CH₂)₅—SO₃H; (CF₃)₂N—Ar—O—(CH₂)₅—O—SO₃H;
(CF₃)₂N—Ar—O—(CH₂)₅—SH; (CF₃)₂N—Ar—O—(CH₂)₅—OH; (CF₃)₂N—Ar—O—(CH₂)₅—N⁺R¹R²R³Z⁻;
(CF₃)₂N—Ar—O—(CH₂)₅—P⁺R¹R²R³Z⁻; (CF₃)₂N—Ar—O—(CH₂)₅—O-glucoside;
(CF₃)₂N—Ar—O—(CH₂)₅—O—PO₃H
(CF₃)₂N—Ar—O—(CH₂)₆—COOH; (CF₃)₂N—Ar—O—(CH₂)₆—SO₃H; (CF₃)₂N—Ar—O—(CH₂)₆—O—SO₃H;
(CF₃)₂N—Ar—O—(CH₂)₆—SH; (CF₃)₂N—Ar—O—(CH₂)₆—OH; (CF₃)₂N—Ar—O—(CH₂)₆—N⁺R¹R²R³Z⁻;
(CF₃)₂N—Ar—O—(CH₂)₆—P⁺R¹R²R³Z⁻; (CF₃)₂N—Ar—O—(CH₂)₆—O-glucoside;
(CF₃)₂N—Ar—O—(CH₂)₆—O—PO₃H
(CF₃)₂N—Ar—O—(CH₂)₇—COOH; (CF₃)₂N—Ar—O—(CH₂)₇—SO₃H; (CF₃)₂N—Ar—O—(CH₂)₇—O—SO₃H;
(CF₃)₂N—Ar—O—(CH₂)₇—SH; (CF₃)₂N—Ar—O—(CH₂)₇—OH; (CF₃)₂N—Ar—O—(CH₂)₇—N⁺R¹R²R³Z⁻;
(CF₃)₂N—Ar—O—(CH₂)₇—P⁺R¹R²R³Z⁻; (CF₃)₂N—Ar—O—(CH₂)₇—O-glucoside;
(CF₃)₂N—Ar—O—(CH₂)₇—O—PO₃H
(CF₃)₂N—Ar—O—(CH₂)₈—COOH; (CF₃)₂N—Ar—O—(CH₂)₈—SO₃H; (CF₃)₂N—Ar—O—(CH₂)₈—O—SO₃H;
(CF₃)₂N—Ar—O—(CH₂)₈—SH; (CF₃)₂N—Ar—O—(CH₂)₈—OH; (CF₃)₂N—Ar—O—(CH₂)₈—N⁺R¹R²R³Z⁻;
(CF₃)₂N—Ar—O—(CH₂)₈—P⁺R¹R²R³Z⁻; (CF₃)₂N—Ar—O—(CH₂)₈—O-glucoside;
(CF₃)₂N—Ar—O—(CH₂)₈—O—PO₃H
(CF₃)₂N—Ar—O—(CH₂)₉—COOH; (CF₃)₂N—Ar—O—(CH₂)₉—SO₃H; (CF₃)₂N—Ar—O—(CH₂)₉—O—SO₃H;
(CF₃)₂N—Ar—O—(CH₂)₉—SH; (CF₃)₂N—Ar—O—(CH₂)₉—OH; (CF₃)₂N—Ar—O—(CH₂)₉—N⁺R¹R²R³Z⁻;
(CF₃)₂N—Ar—O—(CH₂)₉—P⁺R¹R²R³Z⁻; (CF₃)₂N—Ar—O—(CH₂)₉—O-glucoside;
(CF₃)₂N—Ar—O—(CH₂)₉—O—PO₃H
(CF₃)₂N—Ar—O—(CH₂)₁₀—COOH; (CF₃)₂N—Ar—O—(CH₂)₁₀—SO₃H; (CF₃)₂N—Ar—O—(CH₂)₁₀—O—SO₃H;
(CF₃)₂N—Ar—O—(CH₂)₁₀—SH; (CF₃)₂N—Ar—O—(CH₂)₁₀—OH; (CF₃)₂N—Ar—O—(CH₂)₁₀—N⁺R¹R²R³Z⁻;
(CF₃)₂N—Ar—O—(CH₂)₁₀—P⁺R¹R²R³Z⁻; (CF₃)₂N—Ar—O—(CH₂)₁₀—O-
glucoside; (CF₃)₂N—Ar—O—(CH₂)₁₀—O—PO₃H
(CF₃)₂N—Ar—O—(CH₂)₁₁—COOH; (CF₃)₂N—Ar—O—(CH₂)₁₁—SO₃H; (CF₃)₂N—Ar—O—(CH₂)₁₁—O—SO₃H;
(CF₃)₂N—Ar—O—(CH₂)₁₁—SH; (CF₃)₂N—Ar—O—(CH₂)₁₁—OH; (CF₃)₂N—Ar—O—(CH₂)₁₁—N⁺R¹R²R³Z⁻;
(CF₃)₂N—Ar—O—(CH₂)₁₁—P⁺R¹R²R³Z⁻; (CF₃)₂N—Ar—O—(CH₂)₁₁—O-
glucoside; (CF₃)₂N—Ar—O—(CH₂)₁₁—O—PO₃H
(CF₃)₂N—Ar—O—(CH₂)₁₂—COOH; (CF₃)₂N—Ar—O—(CH₂)₁₂—SO₃H; (CF₃)₂N—Ar—O—(CH₂)₁₂—O—SO₃H;
(CF₃)₂N—Ar—O—(CH₂)₁₂—SH; (CF₃)₂N—Ar—O—(CH₂)₁₂—OH; (CF₃)₂N—Ar—O—(CH₂)₁₂—N⁺R¹R²R³Z⁻;
(CF₃)₂N—Ar—O—(CH₂)₁₂—P⁺R¹R²R³Z⁻; (CF₃)₂N—Ar—O—(CH₂)₁₂—O-
glucoside; (CF₃)₂N—Ar—O—(CH₂)₁₂—O—PO₃H
(CF₃)₂N—Ar—O—(CH₂)₁₃—COOH; (CF₃)₂N—Ar—O—(CH₂)₁₃—SO₃H; (CF₃)₂N—Ar—O—(CH₂)₁₃—O—SO₃H;
(CF₃)₂N—Ar—O—(CH₂)₁₃—SH; (CF₃)₂N—Ar—O—(CH₂)₁₃—OH; (CF₃)₂N—Ar—O—(CH₂)₁₃—N⁺R¹R²R³Z⁻;
(CF₃)₂N—Ar—O—(CH₂)₁₃—P⁺R¹R²R³Z⁻; (CF₃)₂N—Ar—O—(CH₂)₁₃—O-
glucoside; (CF₃)₂N—Ar—O—(CH₂)₁₃—O—PO₃H
(CF₃)₂N—Ar—O—(CH₂)₁₄—COOH; (CF₃)₂N—Ar—O—(CH₂)₁₄—SO₃H; (CF₃)₂N—Ar—O—(CH₂)₁₄—O—SO₃H;
(CF₃)₂N—Ar—O—(CH₂)₁₄—SH; (CF₃)₂N—Ar—O—(CH₂)₁₄—OH; (CF₃)₂N—Ar—O—(CH₂)₁₄—N⁺R¹R²R³Z⁻;
(CF₃)₂N—Ar—O—(CH₂)₁₄—P⁺R¹R²R³Z⁻; (CF₃)₂N—Ar—O—(CH₂)₁₄—O-
glucoside; (CF₃)₂N—Ar—O—(CH₂)₁₄—O—PO₃H
(CF₃)₂N—Ar—O—(CH₂)₁₅—COOH; (CF₃)₂N—Ar—O—(CH₂)₁₅—SO₃H; (CF₃)₂N—Ar—O—(CH₂)₁₅—O—SO₃H;
(CF₃)₂N—Ar—O—(CH₂)₁₅—SH; (CF₃)₂N—Ar—O—(CH₂)₁₅—OH; (CF₃)₂N—Ar—O—(CH₂)₁₅—N⁺R¹R²R³Z⁻;
(CF₃)₂N—Ar—O—(CH₂)₁₅—P⁺R¹R²R³Z⁻; (CF₃)₂N—Ar—O—(CH₂)₁₅—O-
glucoside; (CF₃)₂N—Ar—O—(CH₂)₁₅—O—PO₃H
(CF₃)₂N—Ar—O—(CH₂)₁₆—COOH; (CF₃)₂N—Ar—O—(CH₂)₁₆—SO₃H; (CF₃)₂N—Ar—O—(CH₂)₁₆—O—SO₃H;
(CF₃)₂N—Ar—O—(CH₂)₁₆—SH; (CF₃)₂N—Ar—O—(CH₂)₁₆—OH; (CF₃)₂N—Ar—O—(CH₂)₁₆—N⁺R¹R²R³Z⁻;
(CF₃)₂N—Ar—O—(CH₂)₁₆—P⁺R¹R²R³Z⁻; (CF₃)₂N—Ar—O—(CH₂)₁₆—O-
glucoside; (CF₃)₂N—Ar—O—(CH₂)₁₆—O—PO₃H
(CF₃)₂N—Ar—O—(CH₂)ₙ—O—(CH₂)_{n′}—COOH; (CF₃)₂N—Ar—O—(CH₂)ₙ—O—(CH₂)_{n′}—SO₃H;
(CF₃)₂N—Ar—O—(CH₂)ₙ—O—(CH₂)_{n′}—O—SO₃H; (CF₃)₂N—Ar—O—(CH₂)ₙ—O—(CH₂)_{n′}—SH;
(CF₃)₂N—Ar—O—(CH₂)ₙ—O—(CH₂)_{n′}—OH; (CF₃)₂N—Ar—O—(CH₂)ₙ—O—(CH₂)_{n′}—N⁺R¹R²R³Z⁻;
(CF₃)₂N—Ar—O—(CH₂)ₙ—O—(CH₂)_{n′}—P⁺R¹R²R³Z⁻; (CF₃)₂N—Ar—O—(CH₂)ₙ—O—(CH₂)_{n′}—O-
glucoside; (CF₃)₂N—Ar—O—(CH₂)ₙ—O—(CH₂)_{n′}—O—PO₃H
(CF₃)₂N—Ar—O—CH₂—CH═CH—(CH₂)₃—COOH; (CF₃)₂N—Ar—O—CH₂—CH═CH—(CH₂)₃—SO₃H;
(CF₃)₂N—Ar—O—CH₂—CH═CH—(CH₂)₃—O—SO₃H; (CF₃)₂N—Ar—O—CH₂—CH═CH—(CH₂)₃—SH;
(CF₃)₂N—Ar—O—CH₂—CH═CH—(CH₂)₃—OH; (CF₃)₂N—Ar—O—CH₂—CH═CH—(CH₂)₃—N⁺R¹R²R³Z⁻;
(CF₃)₂N—Ar—O—CH₂—CH═CH—(CH₂)₃—P⁺R¹R²R³Z⁻; (CF₃)₂N—Ar—O—CH₂—CH═CH—(CH₂)₃—O-
glucoside; (CF₃)₂N—Ar—O—CH₂—CH═CH—(CH₂)₃—O—PO₃H
(CF₃)₂N—Ar—O—CH₂—CH═CH—(CH₂)₄—COOH; (CF₃)₂N—Ar—O—CH₂—CH═CH—(CH₂)₄—SO₃H;
(CF₃)₂N—Ar—O—CH₂—CH═CH—(CH₂)₄—O—SO₃H; (CF₃)₂N—Ar—O—CH₂—CH═CH—(CH₂)₄—SH;
(CF₃)₂N—Ar—O—CH₂—CH═CH—(CH₂)₄—OH; (CF₃)₂N—Ar—O—CH₂—CH═CH—(CH₂)₄—N⁺R¹R²R³Z⁻;
(CF₃)₂N—Ar—O—CH₂—CH═CH—(CH₂)₄—P⁺R¹R²R³Z⁻; (CF₃)₂N—Ar—O—CH₂—CH═CH—(CH₂)₄—O-
glucoside; (CF₃)₂N—Ar—O—CH₂—CH═CH—(CH₂)₄—O—PO₃H
(CF₃)₂N—Ar—O—CH₂—CH═CH—(CH₂)₅—COOH; (CF₃)₂N—Ar—O—CH₂—CH═CH—(CH₂)₅—SO₃H;
(CF₃)₂N—Ar—O—CH₂—CH═CH—(CH₂)₅—O—SO₃H; (CF₃)₂N—Ar—O—CH₂—CH═CH—(CH₂)₅—SH;
(CF₃)₂N—Ar—O—CH₂—CH═CH—(CH₂)₅—OH; (CF₃)₂N—Ar—O—CH₂—CH═CH—(CH₂)₅—N⁺R¹R²R³Z⁻;
(CF₃)₂N—Ar—O—CH₂—CH═CH—(CH₂)₅—P⁺R¹R²R³Z⁻; (CF₃)₂N—Ar—O—CH₂—CH═CH—(CH₂)₅—O-
glucoside; (CF₃)₂N—Ar—O—CH₂—CH═CH—(CH₂)₅—O—PO₃H
(CF₃)₂N—Ar—O—CH₂—CH═CH—(CH₂)₆—COOH; (CF₃)₂N—Ar—O—CH₂—CH═CH—(CH₂)₆—SO₃H;
(CF₃)₂N—Ar—O—CH₂—CH═CH—(CH₂)₆—O—SO₃H; (CF₃)₂N—Ar—O—CH₂—CH═CH—(CH₂)₆—SH;
(CF₃)₂N—Ar—O—CH₂—CH═CH—(CH₂)₆—OH; (CF₃)₂N—Ar—O—CH₂—CH═CH—(CH₂)₆—N⁺R¹R²R³Z⁻;
(CF₃)₂N—Ar—O—CH₂—CH═CH—(CH₂)₆—P⁺R¹R²R³Z⁻; (CF₃)₂N—Ar—O—CH₂—CH═CH—(CH₂)₆—O-
glucoside; (CF₃)₂N—Ar—O—CH₂—CH═CH—(CH₂)₆—O—PO₃H
(CF₃)₂N—Ar—O—CH₂—CH═CH—(CH₂)₇—COOH; (CF₃)₂N—Ar—O—CH₂—CH═CH—(CH₂)₇—SO₃H;
(CF₃)₂N—Ar—O—CH₂—CH═CH—(CH₂)₇—O—SO₃H; (CF₃)₂N—Ar—O—CH₂—CH═CH—(CH₂)₇—SH;
(CF₃)₂N—Ar—O—CH₂—CH═CH—(CH₂)₇—OH; (CF₃)₂N—Ar—O—CH₂—CH═CH—(CH₂)₇—N⁺R¹R²R³Z⁻;
(CF₃)₂N—Ar—O—CH₂—CH═CH—(CH₂)₇—P⁺R¹R²R³Z⁻; (CF₃)₂N—Ar—O—CH₂—CH═CH—(CH₂)₇—O-
glucoside; (CF₃)₂N—Ar—O—CH₂—CH═CH—(CH₂)₇—O—PO₃H
(CF₃)₂N—Ar—O—CH₂—CH═CH—(CH₂)₈—COOH; (CF₃)₂N—Ar—O—CH₂—CH═CH—(CH₂)₈—SO₃H;
(CF₃)₂N—Ar—O—CH₂—CH═CH—(CH₂)₈—O—SO₃H; (CF₃)₂N—Ar—O—CH₂—CH═CH—(CH₂)₈—SH;
(CF₃)₂N—Ar—O—CH₂—CH═CH—(CH₂)₈—OH; (CF₃)₂N—Ar—O—CH₂—CH═CH—(CH₂)₈—N⁺R¹R²R³Z⁻;
(CF₃)₂N—Ar—O—CH₂—CH═CH—(CH₂)₈—P⁺R¹R²R³Z⁻; (CF₃)₂N—Ar—O—CH₂—CH═CH—(CH₂)₈—O-
glucoside; (CF₃)₂N—Ar—O—CH₂—CH═CH—(CH₂)₈—O—PO₃H
(CF₃)₂N—Ar—O—CH₂—CH═CH—(CH₂)₉—COOH; (CF₃)₂N—Ar—O—CH₂—CH═CH—(CH₂)₉—SO₃H;
(CF₃)₂N—Ar—O—CH₂—CH═CH—(CH₂)₉—O—SO₃H; (CF₃)₂N—Ar—O—CH₂—CH═CH—(CH₂)₉—SH;
(CF₃)₂N—Ar—O—CH₂—CH═CH—(CH₂)₉—OH; (CF₃)₂N—Ar—O—CH₂—CH═CH—(CH₂)₉—N⁺R¹R²R³Z⁻;
(CF₃)₂N—Ar—O—CH₂—CH═CH—(CH₂)₉—P⁺R¹R²R³Z⁻; (CF₃)₂N—Ar—O—CH₂—CH═CH—(CH₂)₉—O-
glucoside; (CF₃)₂N—Ar—O—CH₂—CH═CH—(CH₂)₉—O—PO₃H
(CF₃)₂N—Ar—O—CH₂—CH═CH—(CH₂)₁₀—COOH; (CF₃)₂N—Ar—O—CH₂—CH═CH—(CH₂)₁₀—SO₃H;
(CF₃)₂N—Ar—O—CH₂—CH═CH—(CH₂)₁₀—O—SO₃H; (CF₃)₂N—Ar—O—CH₂—CH═CH—(CH₂)₁₀—SH;
(CF₃)₂N—Ar—O—CH₂—CH═CH—(CH₂)₁₀—OH; (CF₃)₂N—Ar—O—CH₂—CH═CH—(CH₂)₁₀—N⁺R¹R²R³Z⁻;
(CF₃)₂N—Ar—O—CH₂—CH═CH—(CH₂)₁₀—P⁺R¹R²R³Z⁻; (CF₃)₂N—Ar—O—CH₂—CH═CH—(CH₂)₁₀—O-
glucoside; (CF₃)₂N—Ar—O—CH₂—CH═CH—(CH₂)₁₀—O—PO₃H
(CF₃)₂N—Ar—O—CH₂—CH═CH—(CH₂)₁₁—COOH; (CF₃)₂N—Ar—O—CH₂—CH═CH—(CH₂)₁₁—SO₃H;
(CF₃)₂N—Ar—O—CH₂—CH═CH—(CH₂)₁₁—O—SO₃H; (CF₃)₂N—Ar—O—CH₂—CH═CH—(CH₂)₁₁—SH;
(CF₃)₂N—Ar—O—CH₂—CH═CH—(CH₂)₁₁—OH; (CF₃)₂N—Ar—O—CH₂—CH═CH—(CH₂)₁₁—N⁺R¹R²R³Z⁻;
(CF₃)₂N—Ar—O—CH₂—CH═CH—(CH₂)₁₁—P⁺R¹R²R³Z⁻; (CF₃)₂N—Ar—O—CH₂—CH═CH—(CH₂)₁₁—O-
glucoside; (CF₃)₂N—Ar—O—CH₂—CH═CH—(CH₂)₁₁—O—PO₃H
(CF₃)₂N—Ar—O—CH₂—CH═CH—(CH₂)₁₂—COOH; (CF₃)₂N—Ar—O—CH₂—CH═CH—(CH₂)₁₂—SO₃H;
(CF₃)₂N—Ar—O—CH₂—CH═CH—(CH₂)₁₂—O—SO₃H; (CF₃)₂N—Ar—O—CH₂—CH═CH—(CH₂)₁₂—SH;
(CF₃)₂N—Ar—O—CH₂—CH═CH—(CH₂)₁₂—OH; (CF₃)₂N—Ar—O—CH₂—CH═CH—(CH₂)₁₂—N⁺R¹R²R³Z⁻;
(CF₃)₂N—Ar—O—CH₂—CH═CH—(CH₂)₁₂—P⁺R¹R²R³Z⁻; (CF₃)₂N—Ar—O—CH₂—CH═CH—(CH₂)₁₂—O-
glucoside; (CF₃)₂N—Ar—O—CH₂—CH═CH—(CH₂)₁₂—O—PO₃H
(CF₃)₂N—Ar—O—CH₂—CH═CH—(CH₂)₁₃—COOH; (CF₃)₂N—Ar—O—CH₂—CH═CH—(CH₂)₁₃—SO₃H;
(CF₃)₂N—Ar—O—CH₂—CH═CH—(CH₂)₁₃—O—SO₃H; (CF₃)₂N—Ar—O—CH₂—CH═CH—(CH₂)₁₃—SH;
(CF₃)₂N—Ar—O—CH₂—CH═CH—(CH₂)₁₃—OH; (CF₃)₂N—Ar—O—CH₂—CH═CH—(CH₂)₁₃—N⁺R¹R²R³Z⁻;
(CF₃)₂N—Ar—O—CH₂—CH═CH—(CH₂)₁₃—P⁺R¹R²R³Z⁻; (CF₃)₂N—Ar—O—CH₂—CH═CH—(CH₂)₁₃—O-
glucoside; (CF₃)₂N—Ar—O—CH₂—CH═CH—(CH₂)₁₃—O—PO₃H
(CF₃)₂N—Ar—O—CH₂—CH═CH—(CH₂)₁₄—COOH; (CF₃)₂N—Ar—O—CH₂—CH═CH—(CH₂)₁₄—SO₃H;
(CF₃)₂N—Ar—O—CH₂—CH═CH—(CH₂)₁₄—O—SO₃H; (CF₃)₂N—Ar—O—CH₂—CH═CH—(CH₂)₁₄—SH;
(CF₃)₂N—Ar—O—CH₂—CH═CH—(CH₂)₁₄—OH; (CF₃)₂N—Ar—O—CH₂—CH═CH—(CH₂)₁₄—N⁺R¹R²R³Z⁻;
(CF₃)₂N—Ar—O—CH₂—CH═CH—(CH₂)₁₄—P⁺R¹R²R³Z⁻; (CF₃)₂N—Ar—O—CH₂—CH═CH—(CH₂)₁₄—O-
glucoside; (CF₃)₂N—Ar—O—CH₂—CH═CH—(CH₂)₁₄—O—PO₃H
(CF₃)₂N—Ar—O—CH₂—CH═CH—(CH₂)₁₅—COOH; (CF₃)₂N—Ar—O—CH₂—CH═CH—(CH₂)₁₅—SO₃H;
(CF₃)₂N—Ar—O—CH₂—CH═CH—(CH₂)₁₅—O—SO₃H; (CF₃)₂N—Ar—O—CH₂—CH═CH—(CH₂)₁₅—SH;
(CF₃)₂N—Ar—O—CH₂—CH═CH—(CH₂)₁₅—OH; (CF₃)₂N—Ar—O—CH₂—CH═CH—(CH₂)₁₅—N⁺R¹R²R³Z⁻;
(CF₃)₂N—Ar—O—CH₂—CH═CH—(CH₂)₁₅—P⁺R¹R²R³Z⁻; (CF₃)₂N—Ar—O—CH₂—CH═CH—(CH₂)₁₅—O-
glucoside; (CF₃)₂N—Ar—O—CH₂—CH═CH—(CH₂)₁₅—O—PO₃H
(CF₃)₂N—Ar—O—CH₂—CH═CH—(CH₂)₁₆—COOH; (CF₃)₂N—Ar—O—CH₂—CH═CH—(CH₂)₁₆—SO₃H;
(CF₃)₂N—Ar—O—CH₂—CH═CH—(CH₂)₁₆—O—SO₃H; (CF₃)₂N—Ar—O—CH₂—CH═CH—(CH₂)₁₆—SH;
(CF₃)₂N—Ar—O—CH₂—CH═CH—(CH₂)₁₆—OH; (CF₃)₂N—Ar—O—CH₂—CH═CH—(CH₂)₁₆—N⁺R¹R²R³Z⁻;
(CF₃)₂N—Ar—O—CH₂—CH═CH—(CH₂)₁₆—OH; (CF₃)₂N—Ar—O—CH₂—CH═CH—(CH₂)₁₆—N⁺R¹R²R³Z⁻;
(CF₃)₂N—Ar—O—CH₂—CH═CH—(CH₂)₁₆—P⁺R¹R²R³Z⁻; (CF₃)₂N—Ar—O—CH₂—CH═CH—(CH₂)₁₆—O-
glucoside; (CF₃)₂N—Ar—O—CH₂—CH═CH—(CH₂)₁₆—O—PO₃H
(CF₃)₂N—Ar—O—C(═O)—(CH₂)₄—COOH; (CF₃)₂N—Ar—O—C(═O)—(CH₂)₄—SO₃H; (CF₃)₂N—Ar—O—C(═O)—(CH₂)₄—O—SO₃H;
(CF₃)₂N—Ar—O—C(═O)—(CH₂)₄—SH; (CF₃)₂N—Ar—O—C(═O)—(CH₂)₄—OH;
(CF₃)₂N—Ar—O—C(═O)—(CH₂)₄—N⁺R¹R²R³Z⁻; (CF₃)₂N—Ar—O—C(═O)—(CH₂)₄—P⁺R¹R²R³Z⁻;
(CF₃)₂N—Ar—O—C(═O)—(CH₂)₄—O-glucoside; (CF₃)₂N—Ar—O—C(═O)—(CH₂)₄—O—PO₃H
(CF₃)₂N—Ar—O—C(═O)—(CH₂)₅—COOH; (CF₃)₂N—Ar—O—C(═O)—(CH₂)₅—SO₃H; (CF₃)₂N—Ar—O—C(═O)—(CH₂)₅—O—SO₃H;
(CF₃)₂N—Ar—O—C(═O)—(CH₂)₅—SH; (CF₃)₂N—Ar—O—C(═O)—(CH₂)₅—OH;
(CF₃)₂N—Ar—O—C(═O)—(CH₂)₅—N⁺R¹R²R³Z⁻; (CF₃)₂N—Ar—O—C(═O)—(CH₂)₅—P⁺R¹R²R³Z⁻;
(CF₃)₂N—Ar—O—C(═O)—(CH₂)₅—O-glucoside; (CF₃)₂N—Ar—O—C(═O)—(CH₂)₅—O—PO₃H
(CF₃)₂N—Ar—O—C(═O)—(CH₂)₆—COOH; (CF₃)₂N—Ar—O—C(═O)—(CH₂)₆—SO₃H; (CF₃)₂N—Ar—O—C(═O)—(CH₂)₆—O—SO₃H;
(CF₃)₂N—Ar—O—C(═O)—(CH₂)₆—SH; (CF₃)₂N—Ar—O—C(═O)—(CH₂)₆—OH;
(CF₃)₂N—Ar—O—C(═O)—(CH₂)₆—N⁺R¹R²R³Z⁻; (CF₃)₂N—Ar—O—C(═O)—(CH₂)₆—P⁺R¹R²R³Z⁻; (CF₃)₂N—Ar—O—C(═O)—(CH₂)₆—O-
glucoside; (CF₃)₂N—Ar—O—C(═O)—(CH₂)₆—O—PO₃H
(CF₃)₂N—Ar—O—C(═O)—(CH₂)₇—COOH; (CF₃)₂N—Ar—O—C(═O)—(CH₂)₇—SO₃H; (CF₃)₂N—Ar—O—C(═O)—(CH₂)₇—O—SO₃H;
(CF₃)₂N—Ar—O—C(═O)—(CH₂)₇—SH; (CF₃)₂N—Ar—O—C(═O)—(CH₂)₇—OH;
(CF₃)₂N—Ar—O—C(═O)—(CH₂)₇—N⁺R¹R²R³Z⁻; (CF₃)₂N—Ar—O—C(═O)—(CH₂)₇—P⁺R¹R²R³Z⁻;
(CF₃)₂N—Ar—O—C(═O)—(CH₂)₇—O-glucoside; (CF₃)₂N—Ar—O—C(═O)—(CH₂)₇—O—PO₃H
(CF₃)₂N—Ar—O—C(═O)—(CH₂)₈—COOH; (CF₃)₂N—Ar—O—C(═O)—(CH₂)₈—SO₃H; (CF₃)₂N—Ar—O—C(═O)—(CH₂)₈—O—SO₃H;
(CF₃)₂N—Ar—O—C(═O)—(CH₂)₈—SH; (CF₃)₂N—Ar—O—C(═O)—(CH₂)₈—OH;
(CF₃)₂N—Ar—O—C(═O)—(CH₂)₈—N⁺R¹R²R³Z⁻; (CF₃)₂N—Ar—O—C(═O)—(CH₂)₈—P⁺R¹R²R³Z⁻;
(CF₃)₂N—Ar—O—C(═O)—(CH₂)₈—O-glucoside; (CF₃)₂N—Ar—O—C(═O)—(CH₂)₈—O—PO₃H
(CF₃)₂N—Ar—O—C(═O)—(CH₂)₉—COOH; (CF₃)₂N—Ar—O—C(═O)—(CH₂)₉—SO₃H; (CF₃)₂N—Ar—O—C(═O)—(CH₂)₉—O—SO₃H;
(CF₃)₂N—Ar—O—C(═O)—(CH₂)₉—SH; (CF₃)₂N—Ar—O—C(═O)—(CH₂)₉—OH;
(CF₃)₂N—Ar—O—C(═O)—(CH₂)₉—N⁺R¹R²R³Z⁻; (CF₃)₂N—Ar—O—C(═O)—(CH₂)₉—P⁺R¹R²R³Z⁻;
(CF₃)₂N—Ar—O—C(═O)—(CH₂)₉—O-glucoside; (CF₃)₂N—Ar—O—C(═O)—(CH₂)₉—O—PO₃H
(CF₃)₂N—Ar—O—C(═O)—(CH₂)₁₀—COOH; (CF₃)₂N—Ar—O—C(═O)—(CH₂)₁₀—SO₃H; (CF₃)₂N—Ar—O—C(═O)—(CH₂)₁₀—O—SO₃H;
(CF₃)₂N—Ar—O—C(═O)—(CH₂)₁₀—SH; (CF₃)₂N—Ar—O—C(═O)—(CH₂)₁₀—OH;
(CF₃)₂N—Ar—O—C(═O)—(CH₂)₁₀—N⁺R¹R²R³Z⁻; (CF₃)₂N—Ar—O—C(═O)—(CH₂)₁₀—P⁺R¹R²R³Z⁻;
(CF₃)₂N—Ar—O—C(═O)—(CH₂)₁₀—O-glucoside; (CF₃)₂N—Ar—O—C(═O)—(CH₂)₁₀—O—PO₃H
(CF₃)₂N—Ar—O—C(═O)—(CH₂)₁₁—COOH; (CF₃)₂N—Ar—O—C(═O)—(CH₂)₁₁—SO₃H; (CF₃)₂N—Ar—O—C(═O)—(CH₂)₁₁—O—SO₃H;
(CF₃)₂N—Ar—O—C(═O)—(CH₂)₁₁—SH; (CF₃)₂N—Ar—O—C(═O)—(CH₂)₁₁—OH;
(CF₃)₂N—Ar—O—C(═O)—(CH₂)₁₁—N⁺R¹R²R³Z⁻; (CF₃)₂N—Ar—O—C(═O)—(CH₂)₁₁—P⁺R¹R²R³Z⁻;
(CF₃)₂N—Ar—O—C(═O)—(CH₂)₁₁—O-glucoside; (CF₃)₂N—Ar—O—C(═O)—(CH₂)₁₁—O—PO₃H
(CF₃)₂N—Ar—O—C(═O)—(CH₂)₁₂—COOH; (CF₃)₂N—Ar—O—C(═O)—(CH₂)₁₂—SO₃H; (CF₃)₂N—Ar—O—C(═O)—(CH₂)₁₂—O—SO₃H;
(CF₃)₂N—Ar—O—C(═O)—(CH₂)₁₂—SH; (CF₃)₂N—Ar—O—C(═O)—(CH₂)₁₂—OH;
(CF₃)₂N—Ar—O—C(═O)—(CH₂)₁₂—N⁺R¹R²R³Z⁻; (CF₃)₂N—Ar—O—C(═O)—(CH₂)₁₂—P⁺R¹R²R³Z⁻;
(CF₃)₂N—Ar—O—C(═O)—(CH₂)₁₂—O-glucoside; (CF₃)₂N—Ar—O—C(═O)—(CH₂)₁₂—O—PO₃H
(CF₃)₂N—Ar—O—C(═O)—(CH₂)₁₃—COOH; (CF₃)₂N—Ar—O—C(═O)—(CH₂)₁₃—SO₃H; (CF₃)₂N—Ar—O—C(═O)—(CH₂)₁₃—O—SO₃H;
(CF₃)₂N—Ar—O—C(═O)—(CH₂)₁₃—SH; (CF₃)₂N—Ar—O—C(═O)—(CH₂)₁₃—OH;
(CF₃)₂N—Ar—O—C(═O)—(CH₂)₁₃—N⁺R¹R²R³Z⁻; (CF₃)₂N—Ar—O—C(═O)—(CH₂)₁₃—P⁺R¹R²R³Z⁻;
(CF₃)₂N—Ar—O—C(═O)—(CH₂)₁₃—O-glucoside; (CF₃)₂N—Ar—O—C(═O)—(CH₂)₁₃—O—PO₃H
(CF₃)₂N—Ar—O—C(═O)—(CH₂)₁₄—COOH; (CF₃)₂N—Ar—O—C(═O)—(CH₂)₁₄—SO₃H; (CF₃)₂N—Ar—O—C(═O)—(CH₂)₁₄—O—SO₃H;
(CF₃)₂N—Ar—O—C(═O)—(CH₂)₁₄—SH; (CF₃)₂N—Ar—O—C(═O)—(CH₂)₁₄—OH;
(CF₃)₂N—Ar—O—C(═O)—(CH₂)₁₄—N⁺R¹R²R³Z⁻; (CF₃)₂N—Ar—O—C(═O)—(CH₂)₁₄—P⁺R¹R²R³Z⁻;
(CF₃)₂N—Ar—O—C(═O)—(CH₂)₁₄—O-glucoside; (CF₃)₂N—Ar—O—C(═O)—(CH₂)₁₄—O—PO₃H
(CF₃)₂N—Ar—O—C(═O)—(CH₂)₁₅—COOH; (CF₃)₂N—Ar—O—C(═O)—(CH₂)₁₅—SO₃H; (CF₃)₂N—Ar—O—C(═O)—(CH₂)₁₅—O—SO₃H;
(CF₃)₂N—Ar—O—C(═O)—(CH₂)₁₅—SH; (CF₃)₂N—Ar—O—C(═O)—(CH₂)₁₅—OH;
(CF₃)₂N—Ar—O—C(═O)—(CH₂)₁₅—N⁺R¹R²R³Z⁻; (CF₃)₂N—Ar—O—C(═O)—(CH₂)₁₅—P⁺R¹R²R³Z⁻;
(CF₃)₂N—Ar—O—C(═O)—(CH₂)₁₅—O-glucoside; (CF₃)₂N—Ar—O—C(═O)—(CH₂)₁₅—O—PO₃H
(CF₃)₂N—Ar—O—C(═O)—(CH₂)₁₆—COOH; (CF₃)₂N—Ar—O—C(═O)—(CH₂)₁₆—SO₃H; (CF₃)₂N—Ar—O—C(═O)—(CH₂)₁₆—O—SO₃H;
(CF₃)₂N—Ar—O—C(═O)—(CH₂)₁₆—SH; (CF₃)₂N—Ar—O—C(═O)—(CH₂)₁₆—OH;
(CF₃)₂N—Ar—O—C(═O)—(CH₂)₁₆—N⁺R¹R²R³Z⁻; (CF₃)₂N—Ar—O—C(═O)—(CH₂)₁₆—P⁺R¹R²R³Z⁻;
(CF₃)₂N—Ar—O—C(═O)—(CH₂)₁₆—O-glucoside; (CF₃)₂N—Ar—O—C(═O)—(CH₂)₁₆—O—PO₃H
(CF₃)₂N—Ar—O—C(═O)—(CH₂)ₙ—O—(CH₂)_{n′}—COOH; (CF₃)₂N—Ar—O—C(═O)—(CH₂)ₙ—O—(CH₂)_{n′}—SO₃H;
(CF₃)₂N—Ar—O—C(═O)—(CH₂)ₙ—O—(CH₂)_{n′}—O—SO₃H; (CF₃)₂N—Ar—O—C(═O)—(CH₂)ₙ—O—(CH₂)_{n′}—SH;
(CF₃)₂N—Ar—O—C(═O)—(CH₂)ₙ—O—(CH₂)_{n′}—OH; (CF₃)₂N—Ar—O—C(═O)—(CH₂)ₙ—O—(CH₂)_{n′}—N⁺R¹R²R³Z⁻;
(CF₃)₂N—Ar—O—C(═O)—(CH₂)ₙ—O—(CH₂)_{n′}—P⁺R¹R²R³Z⁻;
(CF₃)₂N—Ar—O—C(═O)—(CH₂)ₙ—O—(CH₂)_{n′}—O-glucoside; (CF₃)₂N—Ar—O—C(═O)—(CH₂)ₙ—O—(CH₂)_{n′}—O—PO₃H
(CF₃)₂N—Ar—O—C(═O)—CH₂—CH═CH—(CH₂)₃—COOH; (CF₃)₂N—Ar—O—C(═O)—CH₂—CH═CH—(CH₂)₃—SO₃H;
(CF₃)₂N—Ar—O—C(═O)—CH₂—CH═CH—(CH₂)₃—O—SO₃H; (CF₃)₂N—Ar—O—C(═O)—CH₂—CH═CH—(CH₂)₃—SH;
(CF₃)₂N—Ar—O—C(═O)—CH₂—CH═CH—(CH₂)₃—OH;
(CF₃)₂N—Ar—O—C(═O)—CH₂—CH═CH—(CH₂)₃—N⁺R¹R²R³Z⁻; (CF₃)₂N—Ar—O—C(═O)—CH₂—CH═CH—(CH₂)₃—P⁺R¹R²R³Z⁻;
(CF₃)₂N—Ar—O—C(═O)—CH₂—CH═CH—(CH₂)₃—O-
glucoside; (CF₃)₂N—Ar—O—C(═O)—CH₂—CH═CH—(CH₂)₃—O—PO₃H
(CF₃)₂N—Ar—O—C(═O)—CH₂—CH═CH—(CH₂)₄—COOH; (CF₃)₂N—Ar—O—C(═O)—CH₂—CH═CH—(CH₂)₄—SO₃H;
(CF₃)₂N—Ar—O—C(═O)—CH₂—CH═CH—(CH₂)₄—O—SO₃H; (CF₃)₂N—Ar—O—C(═O)—CH₂—CH═CH—(CH₂)₄—SH;
(CF₃)₂N—Ar—O—C(═O)—CH₂—CH═CH—(CH₂)₄—OH;
(CF₃)₂N—Ar—O—C(═O)—CH₂—CH═CH—(CH₂)₄—N⁺R¹R²R³Z⁻; (CF₃)₂N—Ar—O—C(═O)—CH₂—CH═CH—(CH₂)₄—P⁺R¹R²R³Z⁻;
(CF₃)₂N—Ar—O—C(═O)—CH₂—CH═CH—(CH₂)₄—O-
glucoside; (CF₃)₂N—Ar—O—C(═O)—CH₂—CH═CH—(CH₂)₄—O—PO₃H
(CF₃)₂N—Ar—O—C(═O)—CH₂—CH═CH—(CH₂)₅—COOH; (CF₃)₂N—Ar—O—C(═O)—CH₂—CH═CH—(CH₂)₅—SO₃H;
(CF₃)₂N—Ar—O—C(═O)—CH₂—CH═CH—(CH₂)₅—O—SO₃H; (CF₃)₂N—Ar—O—C(═O)—CH₂—CH═CH—(CH₂)₅—SH;
(CF₃)₂N—Ar—O—C(═O)—CH₂—CH═CH—(CH₂)₅—OH;
(CF₃)₂N—Ar—O—C(═O)—CH₂—CH═CH—(CH₂)₅—N⁺R¹R²R³Z⁻; (CF₃)₂N—Ar—O—C(═O)—CH₂—CH═CH—(CH₂)₅—P⁺R¹R²R³Z⁻;
(CF₃)₂N—Ar—O—C(═O)—CH₂—CH═CH—(CH₂)₅—O-
glucoside; (CF₃)₂N—Ar—O—C(═O)—CH₂—CH═CH—(CH₂)₅—O—PO₃H
(CF₃)₂N—Ar—O—C(═O)—CH₂—CH═CH—(CH₂)₆—COOH; (CF₃)₂N—Ar—O—C(═O)—CH₂—CH═CH—(CH₂)₆—SO₃H;
(CF₃)₂N—Ar—O—C(═O)—CH₂—CH═CH—(CH₂)₆—O—SO₃H; (CF₃)₂N—Ar—O—C(═O)—CH₂—CH═CH—(CH₂)₆—SH;
(CF₃)₂N—Ar—O—C(═O)—CH₂—CH═CH—(CH₂)₆—OH;
(CF₃)₂N—Ar—O—C(═O)—CH₂—CH═CH—(CH₂)₆—N⁺R¹R²R³Z⁻; (CF₃)₂N—Ar—O—C(═O)—CH₂—CH═CH—(CH₂)₆—P⁺R¹R²R³Z⁻;
(CF₃)₂N—Ar—O—C(═O)—CH₂—CH═CH—(CH₂)₆—O-
glucoside; (CF₃)₂N—Ar—O—C(═O)—CH₂—CH═CH—(CH₂)₆—O—PO₃H
(CF₃)₂N—Ar—O—C(═O)—CH₂—CH═CH—(CH₂)₇—COOH; (CF₃)₂N—Ar—O—C(═O)—CH₂—CH═CH—(CH₂)₇—SO₃H;
(CF₃)₂N—Ar—O—C(═O)—CH₂—CH═CH—(CH₂)₇—O—SO₃H; (CF₃)₂N—Ar—O—C(═O)—CH₂—CH═CH—(CH₂)₇—SH;
(CF₃)₂N—Ar—O—C(═O)—CH₂—CH═CH—(CH₂)₇—OH;
(CF₃)₂N—Ar—O—C(═O)—CH₂—CH═CH—(CH₂)₇—N⁺R¹R²R³Z⁻; (CF₃)₂N—Ar—O—C(═O)—CH₂—CH═CH—(CH₂)₇—P⁺R¹R²R³Z⁻;
(CF₃)₂N—Ar—O—C(═O)—CH₂—CH═CH—(CH₂)₇—O-
glucoside; (CF₃)₂N—Ar—O—C(═O)—CH₂—CH═CH—(CH₂)₇—O—PO₃H
(CF₃)₂N—Ar—O—C(═O)—CH₂—CH═CH—(CH₂)₈—COOH; (CF₃)₂N—Ar—O—C(═O)—CH₂—CH═CH—(CH₂)₈—SO₃H;
(CF₃)₂N—Ar—O—C(═O)—CH₂—CH═CH—(CH₂)₈—O—SO₃H; (CF₃)₂N—Ar—O—C(═O)—CH₂—CH═CH—(CH₂)₈—SH;
(CF₃)₂N—Ar—O—C(═O)—CH₂—CH═CH—(CH₂)₈—OH;
(CF₃)₂N—Ar—O—C(═O)—CH₂—CH═CH—(CH₂)₈—N⁺R¹R²R³Z⁻; (CF₃)₂N—Ar—O—C(═O)—CH₂—CH═CH—(CH₂)₈—P⁺R¹R²R³Z⁻;
(CF₃)₂N—Ar—O—C(═O)—CH₂—CH═CH—(CH₂)₈—O-
glucoside; (CF₃)₂N—Ar—O—C(═O)—CH₂—CH═CH—(CH₂)₈—O—PO₃H
(CF₃)₂N—Ar—O—C(═O)—CH₂—CH═CH—(CH₂)₉—COOH; (CF₃)₂N—Ar—O—C(═O)—CH₂—CH═CH—(CH₂)₉—SO₃H;
(CF₃)₂N—Ar—O—C(═O)—CH₂—CH═CH—(CH₂)₉—O—SO₃H; (CF₃)₂N—Ar—O—C(═O)—CH₂—CH═CH—(CH₂)₉—SH;
(CF₃)₂N—Ar—O—C(═O)—CH₂—CH═CH—(CH₂)₉—OH;
(CF₃)₂N—Ar—O—C(═O)—CH₂—CH═CH—(CH₂)₉—N⁺R¹R²R³Z⁻; (CF₃)₂N—Ar—O—C(═O)—CH₂—CH═CH—(CH₂)₉—P⁺R¹R²R³Z⁻;
(CF₃)₂N—Ar—O—C(═O)—CH₂—CH═CH—(CH₂)₉—O-
glucoside; (CF₃)₂N—Ar—O—C(═O)—CH₂—CH═CH—(CH₂)₉—O—PO₃H
(CF₃)₂N—Ar—O—C(═O)—CH₂—CH═CH—(CH₂)₁₀—COOH; (CF₃)₂N—Ar—O—C(═O)—CH₂—CH═CH—(CH₂)₁₀—SO₃H;
(CF₃)₂N—Ar—O—C(═O)—CH₂—CH═CH—(CH₂)₁₀—O—SO₃H;
(CF₃)₂N—Ar—O—C(═O)—CH₂—CH═CH—(CH₂)₁₀—SH; (CF₃)₂N—Ar—O—C(═O)—CH₂—CH═CH—(CH₂)₁₀—OH;
(CF₃)₂N—Ar—O—C(═O)—CH₂—CH═CH—(CH₂)₁₀—N⁺R¹R²R³Z⁻; (CF₃)₂N—Ar—O—C(═O)—CH₂—CH═CH—(CH₂)₁₀—P⁺R¹R²R³Z⁻;
(CF₃)₂N—Ar—O—C(═O)—CH₂—CH═CH—(CH₂)₁₀—O-
glucoside; (CF₃)₂N—Ar—O—C(═O)—CH₂—CH═CH—(CH₂)₁₀—O—PO₃H
(CF₃)₂N—Ar—O—C(═O)—CH₂—CH═CH—(CH₂)₁₁—COOH; (CF₃)₂N—Ar—O—C(═O)—CH₂—CH═CH—(CH₂)₁₁—SO₃H;
(CF₃)₂N—Ar—O—C(═O)—CH₂—CH═CH—(CH₂)₁₁—O—SO₃H;
(CF₃)₂N—Ar—O—C(═O)—CH₂—CH═CH—(CH₂)₁₁—SH; (CF₃)₂N—Ar—O—C(═O)—CH₂—CH═CH—(CH₂)₁₁—OH;
(CF₃)₂N—Ar—O—C(═O)—CH₂—CH═CH—(CH₂)₁₁—N⁺R¹R²R³Z⁻; (CF₃)₂N—Ar—O—C(═O)—CH₂—CH═CH—(CH₂)₁₁—P⁺R¹R²R³Z⁻;
(CF₃)₂N—Ar—O—C(═O)—CH₂—CH═CH—(CH₂)₁₁—O-
glucoside; (CF₃)₂N—Ar—O—C(═O)—CH₂—CH═CH—(CH₂)₁₁—O—PO₃H
(CF₃)₂N—Ar—O—C(═O)—CH₂—CH═CH—(CH₂)₁₂—COOH; (CF₃)₂N—Ar—O—C(═O)—CH₂—CH═CH—(CH₂)₁₂—SO₃H;
(CF₃)₂N—Ar—O—C(═O)—CH₂—CH═CH—(CH₂)₁₂—O—SO₃H;
(CF₃)₂N—Ar—O—C(═O)—CH₂—CH═CH—(CH₂)₁₂—SH; (CF₃)₂N—Ar—O—C(═O)—CH₂—CH═CH—(CH₂)₁₂—OH;
(CF₃)₂N—Ar—O—C(═O)—CH₂—CH═CH—(CH₂)₁₂—N⁺R¹R²R³Z⁻; (CF₃)₂N—Ar—O—C(═O)—CH₂—CH═CH—(CH₂)₁₂—P⁺R¹R²R³Z⁻;
(CF₃)₂N—Ar—O—C(═O)—CH₂—CH═CH—(CH₂)₁₂—O-
glucoside; (CF₃)₂N—Ar—O—C(═O)—CH₂—CH═CH—(CH₂)₁₂—O—PO₃H
(CF₃)₂N—Ar—O—C(═O)—CH₂—CH═CH—(CH₂)₁₃—COOH; (CF₃)₂N—Ar—O—C(═O)—CH₂—CH═CH—(CH₂)₁₃—SO₃H;
(CF₃)₂N—Ar—O—C(═O)—CH₂—CH═CH—(CH₂)₁₃—O—SO₃H;
(CF₃)₂N—Ar—O—C(═O)—CH₂—CH═CH—(CH₂)₁₃—SH; (CF₃)₂N—Ar—O—C(═O)—CH₂—CH═CH—(CH₂)₁₃—OH;
(CF₃)₂N—Ar—O—C(═O)—CH₂—CH═CH—(CH₂)₁₃—N⁺R¹R²R³Z⁻; (CF₃)₂N—Ar—O—C(═O)—CH₂—CH═CH—(CH₂)₁₃—P⁺R¹R²R³Z⁻;
(CF₃)₂N—Ar—O—C(═O)—CH₂—CH═CH—(CH₂)₁₃—O-
glucoside; (CF₃)₂N—Ar—O—C(═O)—CH₂—CH═CH—(CH₂)₁₃—O—PO₃H
(CF₃)₂N—Ar—O—C(═O)—CH₂—CH═CH—(CH₂)₁₄—COOH; (CF₃)₂N—Ar—O—C(═O)—CH₂—CH═CH—(CH₂)₁₄—SO₃H;
(CF₃)₂N—Ar—O—C(═O)—CH₂—CH═CH—(CH₂)₁₄—O—SO₃H;
(CF₃)₂N—Ar—O—C(═O)—CH₂—CH═CH—(CH₂)₁₄—SH; (CF₃)₂N—Ar—O—C(═O)—CH₂—CH═CH—(CH₂)₁₄—OH;
(CF₃)₂N—Ar—O—C(═O)—CH₂—CH═CH—(CH₂)₁₄—N⁺R¹R²R³Z⁻; (CF₃)₂N—Ar—O—C(═O)—CH₂—CH═CH—(CH₂)₁₄—P⁺R¹R²R³Z⁻;
(CF₃)₂N—Ar—O—C(═O)—CH₂—CH═CH—(CH₂)₁₄—O-
glucoside; (CF₃)₂N—Ar—O—C(═O)—CH₂—CH═CH—(CH₂)₁₄—O—PO₃H
(CF₃)₂N—Ar—O—C(═O)—CH₂—CH═CH—(CH₂)₁₅—COOH; (CF₃)₂N—Ar—O—C(═O)—CH₂—CH═CH—(CH₂)₁₅—SO₃H;
(CF₃)₂N—Ar—O—C(═O)—CH₂—CH═CH—(CH₂)₁₅—O—SO₃H;
(CF₃)₂N—Ar—O—C(═O)—CH₂—CH═CH—(CH₂)₁₅—SH; (CF₃)₂N—Ar—O—C(═O)—CH₂—CH═CH—(CH₂)₁₅—OH;
(CF₃)₂N—Ar—O—C(═O)—CH₂—CH═CH—(CH₂)₁₅—N⁺R¹R²R³Z⁻; (CF₃)₂N—Ar—O—C(═O)—CH₂—CH═CH—(CH₂)₁₅—P⁺R¹R²R³Z⁻;
(CF₃)₂N—Ar—O—C(═O)—CH₂—CH═CH—(CH₂)₁₅—O-
glucoside; (CF₃)₂N—Ar—O—C(═O)—CH₂—CH═CH—(CH₂)₁₅—O—PO₃H
(CF₃)₂N—Ar—O—C(═O)—CH₂—CH═CH—(CH₂)₁₆—COOH; (CF₃)₂N—Ar—O—C(═O)—CH₂—CH═CH—(CH₂)₁₆—SO₃H;
(CF₃)₂N—Ar—O—C(═O)—CH₂—CH═CH—(CH₂)₁₆—O—SO₃H;
(CF₃)₂N—Ar—O—C(═O)—CH₂—CH═CH—(CH₂)₁₆—SH; (CF₃)₂N—Ar—O—C(═O)—CH₂—CH═CH—(CH₂)₁₆—OH;
(CF₃)₂N—Ar—O—C(═O)—CH₂—CH═CH—(CH₂)₁₆—N⁺R¹R²R³Z⁻; (CF₃)₂N—Ar—O—C(═O)—CH₂—CH═CH—(CH₂)₁₆—P⁺R¹R²R³Z⁻;
(CF₃)₂N—Ar—O—C(═O)—CH₂—CH═CH—(CH₂)₁₆—O-
glucoside; (CF₃)₂N—Ar—O—C(═O)—CH₂—CH═CH—(CH₂)₁₆—O—PO₃H
(CF₃—CH₂—CH₂)₂N—Ar—O—(CH₂)₃—COOH;
(CF₃—CH₂—CH₂)₂N—Ar—O—(CH₂)₃—SO₃H; (CF₃—CH₂—CH₂)₂N—Ar—O—(CH₂)₃—O—SO₃H;
(CF₃—CH₂—CH₂)₂N—Ar—O—(CH₂)₃—SH; (CF₃—CH₂—CH₂)₂N—Ar—O—(CH₂)₃—OH;
(CF₃—CH₂—CH₂)₂N—Ar—O—(CH₂)₃—N⁺R¹R²R³Z⁻; (CF₃—CH₂—CH₂)₂N—Ar—O—(CH₂)₃—P⁺R¹R²R³Z⁻;
(CF₃—CH₂—CH₂)₂N—Ar—O—(CH₂)₃—O-glucoside;
(CF₃—CH₂—CH₂)₂N—Ar—O—(CH₂)₃—O—PO₃H
(CF₃—CH₂—CH₂)₂N—Ar—O—(CH₂)₄—COOH;
(CF₃—CH₂—CH₂)₂N—Ar—O—(CH₂)₄—SO₃H; (CF₃—CH₂—CH₂)₂N—Ar—O—(CH₂)₄—O—SO₃H;
(CF₃—CH₂—CH₂)₂N—Ar—O—(CH₂)₄—SH; (CF₃—CH₂—CH₂)₂N—Ar—O—(CH₂)₄—OH;
(CF₃—CH₂—CH₂)₂N—Ar—O—(CH₂)₄—N⁺R¹R²R³Z⁻; (CF₃—CH₂—CH₂)₂N—Ar—O—(CH₂)₄—P⁺R¹R²R³Z⁻;
(CF₃—CH₂—CH₂)₂N—Ar—O—(CH₂)₄—O-glucoside;
(CF₃—CH₂—CH₂)₂N—Ar—O—(CH₂)₄—O—PO₃H
(CF₃—CH₂—CH₂)₂N—Ar—O—(CH₂)₅—COOH;
(CF₃—CH₂—CH₂)₂N—Ar—O—(CH₂)₅—SO₃H; (CF₃—CH₂—CH₂)₂N—Ar—O—(CH₂)₅—O—SO₃H;
(CF₃—CH₂—CH₂)₂N—Ar—O—(CH₂)₅—SH; (CF₃—CH₂—CH₂)₂N—Ar—O—(CH₂)₅—OH;
(CF₃—CH₂—CH₂)₂N—Ar—O—(CH₂)₅—N⁺R¹R²R³Z⁻; (CF₃—CH₂—CH₂)₂N—Ar—O—(CH₂)₅—P⁺R¹R²R³Z⁻;
(CF₃—CH₂—CH₂)₂N—Ar—O—(CH₂)₅—O-glucoside;
(CF₃—CH₂—CH₂)₂N—Ar—O—(CH₂)₅—O—PO₃H
(CF₃—CH₂—CH₂)₂N—Ar—O—(CH₂)₆—COOH;
(CF₃—CH₂—CH₂)₂N—Ar—O—(CH₂)₆—SO₃H; (CF₃—CH₂—CH₂)₂N—Ar—O—(CH₂)₆—O—SO₃H;
(CF₃—CH₂—CH₂)₂N—Ar—O—(CH₂)₆—SH; (CF₃—CH₂—CH₂)₂N—Ar—O—(CH₂)₆—OH;
(CF₃—CH₂—CH₂)₂N—Ar—O—(CH₂)₆—N⁺R¹R²R³Z⁻; (CF₃—CH₂—CH₂)₂N—Ar—O—(CH₂)₆—P⁺R¹R²R³Z⁻;
(CF₃—CH₂—CH₂)₂N—Ar—O—(CH₂)₆—O-glucoside;
(CF₃—CH₂—CH₂)₂N—Ar—O—(CH₂)₆—O—PO₃H
(CF₃—CH₂—CH₂)₂N—Ar—O—(CH₂)₇—COOH; (CF₃—CH₂—CH₂)₂N—Ar—O—(CH₂)₇—SO₃H;
(CF₃—CH₂—CH₂)₂N—Ar—O—(CH₂)₇—O—SO₃H;
(CF₃—CH₂—CH₂)₂N—Ar—O—(CH₂)₇—SH; (CF₃—CH₂—CH₂)₂N—Ar—O—(CH₂)₇—OH;
(CF₃—CH₂—CH₂)₂N—Ar—O—(CH₂)₇—N⁺R¹R²R³Z⁻; (CF₃—CH₂—CH₂)₂N—Ar—O—(CH₂)₇—P⁺R¹R²R³Z⁻;
(CF₃—CH₂—CH₂)₂N—Ar—O—(CH₂)₇—O-glucoside;
(CF₃—CH₂—CH₂)₂N—Ar—O—(CH₂)₇—O—PO₃H
(CF₃—CH₂—CH₂)₂N—Ar—O—(CH₂)₈—COOH; (CF₃—CH₂—CH₂)₂N—Ar—O—(CH₂)₈—SO₃H;
(CF₃—CH₂—CH₂)₂N—Ar—O—(CH₂)₈—O—SO₃H;
(CF₃—CH₂—CH₂)₂N—Ar—O—(CH₂)₈—SH; (CF₃—CH₂—CH₂)₂N—Ar—O—(CH₂)₈—OH;
(CF₃—CH₂—CH₂)₂N—Ar—O—(CH₂)₈—N⁺R¹R²R³Z⁻; (CF₃—CH₂—CH₂)₂N—Ar—O—(CH₂)₈—P⁺R¹R²R³Z⁻;
(CF₃—CH₂—CH₂)₂N—Ar—O—(CH₂)₈—O-glucoside;
(CF₃—CH₂—CH₂)₂N—Ar—O—(CH₂)₈—O—PO₃H
(CF₃—CH₂—CH₂)₂N—Ar—O—(CH₂)₉—COOH; (CF₃—CH₂—CH₂)₂N—Ar—O—(CH₂)₉—SO₃H;
(CF₃—CH₂—CH₂)₂N—Ar—O—(CH₂)₉—O—SO₃H;
(CF₃—CH₂—CH₂)₂N—Ar—O—(CH₂)₉—SH; (CF₃—CH₂—CH₂)₂N—Ar—O—(CH₂)₉—OH;
(CF₃—CH₂—CH₂)₂N—Ar—O—(CH₂)₉—N⁺R¹R²R³Z⁻; (CF₃—CH₂—CH₂)₂N—Ar—O—(CH₂)₉—P⁺R¹R²R³Z⁻;
(CF₃—CH₂—CH₂)₂N—Ar—O—(CH₂)₉—O-glucoside;
(CF₃—CH₂—CH₂)₂N—Ar—O—(CH₂)₉—O—PO₃H
(CF₃—CH₂—CH₂)₂N—Ar—O—(CH₂)₁₀—COOH; (CF₃—CH₂—CH₂)₂N—Ar—O—(CH₂)₁₀—SO₃H;
(CF₃—CH₂—CH₂)₂N—Ar—O—(CH₂)₁₀—O—SO₃H; (CF₃—CH₂—CH₂)₂N—Ar—O—(CH₂)₁₀—SH; (CF₃—CH₂—CH₂)₂N—Ar—O—(CH₂)₁₀—OH;
(CF₃—CH₂—CH₂)₂N—Ar—O—(CH₂)₁₀—N⁺R¹R²R³Z⁻; (CF₃—CH₂—CH₂)₂N—Ar—O—(CH₂)₁₀—P⁺R¹R²R³Z⁻;
(CF₃—CH₂—CH₂)₂N—Ar—O—(CH₂)₁₀—O-
glucoside; (CF₃—CH₂—CH₂)₂N—Ar—O—(CH₂)₁₀—O—PO₃H
(CF₃—CH₂—CH₂)₂N—Ar—O—(CH₂)₁₁—COOH; (CF₃—CH₂—CH₂)₂N—Ar—O—(CH₂)₁₁—SO₃H;
(CF₃—CH₂—CH₂)₂N—Ar—O—(CH₂)₁₁—O—SO₃H; (CF₃—CH₂—CH₂)₂N—Ar—O—(CH₂)₁₁—SH; (CF₃—CH₂—CH₂)₂N—Ar—O—(CH₂)₁₁—OH;
(CF₃—CH₂—CH₂)₂N—Ar—O—(CH₂)₁₁—N⁺R¹R²R³Z⁻; (CF₃—CH₂—CH₂)₂N—Ar—O—(CH₂)₁₁—P⁺R¹R²R³Z⁻;
(CF₃—CH₂—CH₂)₂N—Ar—O—(CH₂)₁₁—O-
glucoside; (CF₃—CH₂—CH₂)₂N—Ar—O—(CH₂)₁₁—O—PO₃H
(CF₃—CH₂—CH₂)₂N—Ar—O—(CH₂)₁₂—COOH; (CF₃—CH₂—CH₂)₂N—Ar—O—(CH₂)₁₂—SO₃H;
(CF₃—CH₂—CH₂)₂N—Ar—O—(CH₂)₁₂—O—SO₃H; (CF₃—CH₂—CH₂)₂N—Ar—O—(CH₂)₁₂—SH; (CF₃—CH₂—CH₂)₂N—Ar—O—(CH₂)₁₂—OH;
(CF₃—CH₂—CH₂)₂N—Ar—O—(CH₂)₁₂—N⁺R¹R²R³Z⁻; (CF₃—CH₂—CH₂)₂N—Ar—O—(CH₂)₁₂—P⁺R¹R²R³Z⁻;
(CF₃—CH₂—CH₂)₂N—Ar—O—(CH₂)₁₂—O-
glucoside; (CF₃—CH₂—CH₂)₂N—Ar—O—(CH₂)₁₂—O—PO₃H
(CF₃—CH₂—CH₂)₂N—Ar—O—(CH₂)₁₃—COOH; (CF₃—CH₂—CH₂)₂N—Ar—O—(CH₂)₁₃—SO₃H;
(CF₃—CH₂—CH₂)₂N—Ar—O—(CH₂)₁₃—O—SO₃H; (CF₃—CH₂—CH₂)₂N—Ar—O—(CH₂)₁₃—SH; (CF₃—CH₂—CH₂)₂N—Ar—O—(CH₂)₁₃—OH;
(CF₃—CH₂—CH₂)₂N—Ar—O—(CH₂)₁₃—N⁺R¹R²R³Z⁻; (CF₃—CH₂—CH₂)₂N—Ar—O—(CH₂)₁₃—P⁺R¹R²R³Z⁻;
(CF₃—CH₂—CH₂)₂N—Ar—O—(CH₂)₁₃—O-
glucoside; (CF₃—CH₂—CH₂)₂N—Ar—O—(CH₂)₁₃—O—PO₃H
(CF₃—CH₂—CH₂)₂N—Ar—O—(CH₂)₁₄—COOH; (CF₃—CH₂—CH₂)₂N—Ar—O—(CH₂)₁₄—SO₃H;
(CF₃—CH₂—CH₂)₂N—Ar—O—(CH₂)₁₄—O—SO₃H; (CF₃—CH₂—CH₂)₂N—Ar—O—(CH₂)₁₄—SH; (CF₃—CH₂—CH₂)₂N—Ar—O—(CH₂)₁₄—OH;
(CF₃—CH₂—CH₂)₂N—Ar—O—(CH₂)₁₄—N⁺R¹R²R³Z⁻; (CF₃—CH₂—CH₂)₂N—Ar—O—(CH₂)₁₄—P⁺R¹R²R³Z⁻;
(CF₃—CH₂—CH₂)₂N—Ar—O—(CH₂)₁₄—O-
glucoside; (CF₃—CH₂—CH₂)₂N—Ar—O—(CH₂)₁₄—O—PO₃H
(CF₃—CH₂—CH₂)₂N—Ar—O—(CH₂)₁₅—COOH; (CF₃—CH₂—CH₂)₂N—Ar—O—(CH₂)₁₅—SO₃H;
(CF₃—CH₂—CH₂)₂N—Ar—O—(CH₂)₁₅—O—SO₃H; (CF₃—CH₂—CH₂)₂N—Ar—O—(CH₂)₁₅—SH; (CF₃—CH₂—CH₂)₂N—Ar—O—(CH₂)₁₅—OH;
(CF₃—CH₂—CH₂)₂N—Ar—O—(CH₂)₁₅—N⁺R¹R²R³Z⁻; (CF₃—CH₂—CH₂)₂N—Ar—O—(CH₂)₁₅—P⁺R¹R²R³Z⁻;
(CF₃—CH₂—CH₂)₂N—Ar—O—(CH₂)₁₅—O-
glucoside; (CF₃—CH₂—CH₂)₂N—Ar—O—(CH₂)₁₅—O—PO₃H
(CF₃—CH₂—CH₂)₂N—Ar—O—(CH₂)₁₆—COOH; (CF₃—CH₂—CH₂)₂N—Ar—O—(CH₂)₁₆—SO₃H;
(CF₃—CH₂—CH₂)₂N—Ar—O—(CH₂)₁₆—O—SO₃H; (CF₃—CH₂—CH₂)₂N—Ar—O—(CH₂)₁₆—SH; (CF₃—CH₂—CH₂)₂N—Ar—O—(CH₂)₁₆—OH;
(CF₃—CH₂—CH₂)₂N—Ar—O—(CH₂)₁₆—N⁺R¹R²R³Z⁻; (CF₃—CH₂—CH₂)₂N—Ar—O—(CH₂)₁₆—P⁺R¹R²R³Z⁻;
(CF₃—CH₂—CH₂)₂N—Ar—O—(CH₂)₁₆—O-
glucoside; (CF₃—CH₂—CH₂)₂N—Ar—O—(CH₂)₁₆—O—PO₃H
(CF₃—CH₂—CH₂)₂N—Ar—O—(CH₂)ₙ—O—(CH₂)_{n′}—COOH; (CF₃—CH₂—CH₂)₂N—Ar—O—(CH₂)ₙ—O—(CH₂)_{n′}—SO₃H;
(CF₃—CH₂—CH₂)₂N—Ar—O—(CH₂)ₙ—O—(CH₂)_{n′}—O—SO₃H; (CF₃—CH₂—CH₂)₂N—Ar—O—(CH₂)ₙ—O—(CH₂)_{n′}—SH;
(CF₃—CH₂—CH₂)₂N—Ar—O—(CH₂)ₙ—O—(CH₂)_{n′}—OH;
(CF₃—CH₂—CH₂)₂N—Ar—O—(CH₂)ₙ—O—(CH₂)_{n′}—N⁺R¹R²R³Z⁻; (CF₃—CH₂—CH₂)₂N—Ar—O—(CH₂)ₙ—O—(CH₂)_{n′}—P⁺R¹R²R³Z⁻;
(CF₃—CH₂—CH₂)₂N—Ar—O—(CH₂)ₙ—O—(CH₂)_{n′}—O-
glucoside; (CF₃—CH₂—CH₂)₂N—Ar—O—(CH₂)ₙ—O—(CH₂)_{n′}—O—PO₃H
(CF₃)₂N—Ar—O—C(═O)—CH₂—CH═CH—(CH₂)₃—COOH; (CF₃)₂N—Ar—O—C(═O)—CH₂—CH═CH—(CH₂)₃—SO₃H;
(CF₃)₂N—Ar—O—C(═O)—CH₂—CH═CH—(CH₂)₃—O—SO₃H; (CF₃)₂N—Ar—O—C(═O)—CH₂—CH═CH—(CH₂)₃—SH;
(CF₃)₂N—Ar—O—C(═O)—CH₂—CH═CH—(CH₂)₃—OH;
(CF₃)₂N—Ar—O—C(═O)—CH₂—CH═CH—(CH₂)₃—N⁺R¹R²R³Z⁻; (CF₃)₂N—Ar—O—C(═O)—CH₂—CH═CH—(CH₂)₃—P⁺R¹R²R³Z⁻;
(CF₃)₂N—Ar—O—C(═O)—CH₂—CH═CH—(CH₂)₃—O-
glucoside; (CF₃)₂N—Ar—O—C(═O)—CH₂—CH═CH—(CH₂)₃—O—PO₃H
(CF₃)₂N—Ar—O—C(═O)—CH₂—CH═CH—(CH₂)₄—COOH; (CF₃)₂N—Ar—O—C(═O)—CH₂—CH═CH—(CH₂)₄—SO₃H;
(CF₃)₂N—Ar—O—C(═O)—CH₂—CH═CH—(CH₂)₄—O—SO₃H; (CF₃)₂N—Ar—O—C(═O)—CH₂—CH═CH—(CH₂)₄—SH;
(CF₃)₂N—Ar—O—C(═O)—CH₂—CH═CH—(CH₂)₄—OH;
(CF₃)₂N—Ar—O—C(═O)—CH₂—CH═CH—(CH₂)₄—N⁺R¹R²R³Z⁻; (CF₃)₂N—Ar—O—C(═O)—CH₂—CH═CH—(CH₂)₄—P⁺R¹R²R³Z⁻;
(CF₃)₂N—Ar—O—C(═O)—CH₂—CH═CH—(CH₂)₄—O-
glucoside; (CF₃)₂N—Ar—O—C(═O)—CH₂—CH═CH—(CH₂)₄—O—PO₃H
(CF₃)₂N—Ar—O—C(═O)—CH₂—CH═CH—(CH₂)₅—COOH; (CF₃)₂N—Ar—O—C(═O)—CH₂—CH═CH—(CH₂)₅—SO₃H;
(CF₃)₂N—Ar—O—C(═O)—CH₂—CH═CH—(CH₂)₅—O—SO₃H; (CF₃)₂N—Ar—O—C(═O)—CH₂—CH═CH—(CH₂)₅—SH;
(CF₃)₂N—Ar—O—C(═O)—CH₂—CH═CH—(CH₂)₅—OH;
(CF₃)₂N—Ar—O—C(═O)—CH₂—CH═CH—(CH₂)₅—N⁺R¹R²R³Z⁻; (CF₃)₂N—Ar—O—C(═O)—CH₂—CH═CH—(CH₂)₅—P⁺R¹R²R³Z⁻;
(CF₃)₂N—Ar—O—C(═O)—CH₂—CH═CH—(CH₂)₅—O-
glucoside; (CF₃)₂N—Ar—O—C(═O)—CH₂—CH═CH—(CH₂)₅—O—PO₃H
(CF₃)₂N—Ar—O—C(═O)—CH₂—CH═CH—(CH₂)₆—COOH; (CF₃)₂N—Ar—O—C(═O)—CH₂—CH═CH—(CH₂)₆—SO₃H;
(CF₃)₂N—Ar—O—C(═O)—CH₂—CH═CH—(CH₂)₆—O—SO₃H; (CF₃)₂N—Ar—O—C(═O)—CH₂—CH═CH—(CH₂)₆—SH;
(CF₃)₂N—Ar—O—C(═O)—CH₂—CH═CH—(CH₂)₆—OH;
(CF₃)₂N—Ar—O—C(═O)—CH₂—CH═CH—(CH₂)₆—N⁺R¹R²R³Z⁻; (CF₃)₂N—Ar—O—C(═O)—CH₂—CH═CH—(CH₂)₆—P⁺R¹R²R³Z⁻;
(CF₃)₂N—Ar—O—C(═O)—CH₂—CH═CH—(CH₂)₆—O-
glucoside; (CF₃)₂N—Ar—O—C(═O)—CH₂—CH═CH—(CH₂)₆—O—PO₃H
(CF₃)₂N—Ar—O—C(═O)—CH₂—CH═CH—(CH₂)₇—COOH; (CF₃)₂N—Ar—O—C(═O)—CH₂—CH═CH—(CH₂)₇—SO₃H;
(CF₃)₂N—Ar—O—C(═O)—CH₂—CH═CH—(CH₂)₇—O—SO₃H; (CF₃)₂N—Ar—O—C(═O)—CH₂—CH═CH—(CH₂)₇—SH;
(CF₃)₂N—Ar—O—C(═O)—CH₂—CH═CH—(CH₂)₇—OH;
(CF₃)₂N—Ar—O—C(═O)—CH₂—CH═CH—(CH₂)₇—N⁺R¹R²R³Z⁻; (CF₃)₂N—Ar—O—C(═O)—CH₂—CH═CH—(CH₂)₇—P⁺R¹R²R³Z⁻;
(CF₃)₂N—Ar—O—C(═O)—CH₂—CH═CH—(CH₂)₇—O-
glucoside; (CF₃)₂N—Ar—O—C(═O)—CH₂—CH═CH—(CH₂)₇—O—PO₃H
(CF₃)₂N—Ar—O—C(═O)—CH₂—CH═CH—(CH₂)₈—COOH; (CF₃)₂N—Ar—O—C(═O)—CH₂—CH═CH—(CH₂)₈—SO₃H;
(CF₃)₂N—Ar—O—C(═O)—CH₂—CH═CH—(CH₂)₈—O—SO₃H; (CF₃)₂N—Ar—O—C(═O)—CH₂—CH═CH—(CH₂)₈—SH;
(CF₃)₂N—Ar—O—C(═O)—CH₂—CH═CH—(CH₂)₈—OH;
(CF₃)₂N—Ar—O—C(═O)—CH₂—CH═CH—(CH₂)₈—N⁺R¹R²R³Z⁻; (CF₃)₂N—Ar—O—C(═O)—CH₂—CH═CH—(CH₂)₈—P⁺R¹R²R³Z⁻;
(CF₃)₂N—Ar—O—C(═O)—CH₂—CH═CH—(CH₂)₈—O-
glucoside; (CF₃)₂N—Ar—O—C(═O)—CH₂—CH═CH—(CH₂)₈—O—PO₃H
(CF₃)₂N—Ar—O—C(═O)—CH₂—CH═CH—(CH₂)₉—COOH; (CF₃)₂N—Ar—O—C(═O)—CH₂—CH═CH—(CH₂)₉—SO₃H;
(CF₃)₂N—Ar—O—C(═O)—CH₂—CH═CH—(CH₂)₉—O—SO₃H; (CF₃)₂N—Ar—O—C(═O)—CH₂—CH═CH—(CH₂)₉—SH;
(CF₃)₂N—Ar—O—C(═O)—CH₂—CH═CH—(CH₂)₉—OH;
(CF₃)₂N—Ar—O—C(═O)—CH₂—CH═CH—(CH₂)₉—N⁺R¹R²R³Z⁻; (CF₃)₂N—Ar—O—C(═O)—CH₂—CH═CH—(CH₂)₉—P⁺R¹R²R³Z⁻;
(CF₃)₂N—Ar—O—C(═O)—CH₂—CH═CH—(CH₂)₉—O-
glucoside; (CF₃)₂N—Ar—O—C(═O)—CH₂—CH═CH—(CH₂)₉—O—PO₃H
(CF₃)₂N—Ar—O—C(═O)—CH₂—CH═CH—(CH₂)₁₀—COOH; (CF₃)₂N—Ar—O—C(═O)—CH₂—CH═CH—(CH₂)₁₀—SO₃H;
(CF₃)₂N—Ar—O—C(═O)—CH₂—CH═CH—(CH₂)₁₀—O—SO₃H;
(CF₃)₂N—Ar—O—C(═O)—CH₂—CH═CH—(CH₂)₁₀—SH; (CF₃)₂N—Ar—O—C(═O)—CH₂—CH═CH—(CH₂)₁₀—OH;
(CF₃)₂N—Ar—O—C(═O)—CH₂—CH═CH—(CH₂)₁₀—N⁺R¹R²R³Z⁻; (CF₃)₂N—Ar—O—C(═O)—CH₂—CH═CH—(CH₂)₁₀—P⁺R¹R²R³Z⁻;
(CF₃)₂N—Ar—O—C(═C)—CH₂—CH═CH—(CH₂)₁₀—O-
glucoside; (CF₃)₂N—Ar—O—C(═O)—CH₂—CH═CH—(CH₂)₁₀ —O—PO₃H
(CF₃)₂N—Ar—O—C(═O)—CH₂—CH═CH—(CH₂)₁₁—COOH; (CF₃)₂N—Ar—O—C(═O)—CH₂—CH═CH—(CH₂)₁₁—SO₃H;
(CF₃)₂N—Ar—O—C(═O)—CH₂—CH═CH—(CH₂)₁₁—O—SO₃H;
(CF₃)₂N—Ar—O—C(═O)—CH₂—CH═CH—(CH₂)₁₁—SH; (CF₃)₂N—Ar—O—C(═O)—CH₂—CH═CH—(CH₂)₁₁—OH;
(CF₃)₂N—Ar—O—C(═O)—CH₂—CH═CH—(CH₂)₁₁—N⁺R¹R²R³Z⁻; (CF₃)₂N—Ar—O—C(═O)—CH₂—CH═CH—(CH₂)₁₁—P⁺R¹R²R³Z⁻;
(CF₃)₂N—Ar—O—C(═O)—CH₂—CH═CH—(CH₂)₁₁—O-
glucoside; (CF₃)₂N—Ar—O—C(═O)—CH₂—CH═CH—(CH₂)₁₁—O—PO₃H
(CF₃)₂N—Ar—O—C(═O)—CH₂—CH═CH—(CH₂)₁₂—COOH; (CF₃)₂N—Ar—O—C(═O)—CH₂—CH═CH—(CH₂)₁₂—SO₃H;
(CF₃)₂N—Ar—O—C(═O)—CH₂—CH═CH—(CH₂)₁₂—O—SO₃H;
(CF₃)₂N—Ar—O—C(═O)—CH₂—CH═CH—(CH₂)₁₂—SH; (CF₃)₂N—Ar—O—C(═O)—CH₂—CH═CH—(CH₂)₁₂—OH;
(CF₃)₂N—Ar—O—C(═O)—CH₂—CH═CH—(CH₂)₁₂—N⁺R¹R²R³Z⁻; (CF₃)₂N—Ar—O—C(═O)—CH₂—CH═CH—(CH₂)₁₂—P⁺R¹R²R³Z⁻;
(CF₃)₂N—Ar—O—C(═O)—CH₂—CH═CH—(CH₂)₁₂—O-
glucoside; (CF₃)₂N—Ar—O—C(═O)—CH₂—CH═CH—(CH₂)₁₂—O—PO₃H
(CF₃)₂N—Ar—O—C(═O)—CH₂—CH═CH—(CH₂)₁₃—COOH; (CF₃)₂N—Ar—O—C(═O)—CH₂—CH═CH—(CH₂)₁₃—SO₃H;
(CF₃)₂N—Ar—O—C(═O)—CH₂—CH═CH—(CH₂)₁₃—O—SO₃H;
(CF₃)₂N—Ar—O—C(═O)—CH₂—CH═CH—(CH₂)₁₃—SH; (CF₃)₂N—Ar—O—C(═O)—CH₂—CH═CH—(CH₂)₁₃—OH;
(CF₃)₂N—Ar—O—C(═O)—CH₂—CH═CH—(CH₂)₁₃—N⁺R¹R²R³Z⁻; (CF₃)₂N—Ar—O—C(═O)—CH₂—CH═CH—(CH₂)₁₃—P⁺R¹R²R³Z⁻;
(CF₃)₂N—Ar—O—C(═O)—CH₂—CH═CH—(CH₂)₁₃—O-
glucoside; (CF₃)₂N—Ar—O—C(═O)—CH₂—CH═CH—(CH₂)₁₃—O—PO₃H
(CF₃)₂N—Ar—O—C(═O)—CH₂—CH═CH—(CH₂)₁₄—COOH; (CF₃)₂N—Ar—O—C(═O)—CH₂—CH═CH—(CH₂)₁₄—SO₃H;
(CF₃)₂N—Ar—O—C(═O)—CH₂—CH═CH—(CH₂)₁₄—O—SO₃H;
(CF₃)₂N—Ar—O—C(═O)—CH₂—CH═CH—(CH₂)₁₄—SH; (CF₃)₂N—Ar—O—C(═O)—CH₂—CH═CH—(CH₂)₁₄—OH;
(CF₃)₂N—Ar—O—C(═O)—CH₂—CH═CH—(CH₂)₁₄—N⁺R¹R²R³Z⁻; (CF₃)₂N—Ar—O—C(═O)—CH₂—CH═CH—(CH₂)₁₄—P⁺R¹R²R³Z⁻;
(CF₃)₂N—Ar—O—C(═O)—CH₂—CH═CH—(CH₂)₁₄—O-
glucoside; (CF₃)₂N—Ar—O—C(═O)—CH₂—CH═CH—(CH₂)₁₄—O—PO₃H
(CF₃)₂N—Ar—O—C(═O)—CH₂—CH═CH—(CH₂)₁₅—COOH; (CF₃)₂N—Ar—O—C(═O)—CH₂—CH═CH—(CH₂)₁₅—SO₃H;
(CF₃)₂N—Ar—O—C(═O)—CH₂—CH═CH—(CH₂)₁₅—O—SO₃H;
(CF₃)₂N—Ar—O—C(═O)—CH₂—CH═CH—(CH₂)₁₅—SH; (CF₃)₂N—Ar—O—C(═O)—CH₂—CH═CH—(CH₂)₁₅—OH;
(CF₃)₂N—Ar—O—C(═O)—CH₂—CH═CH—(CH₂)₁₅—N⁺R¹R²R³Z⁻; (CF₃)₂N—Ar—O—C(═O)—CH₂—CH═CH—(CH₂)₁₅—P⁺R¹R²R³Z⁻;
(CF₃)₂N—Ar—O—C(═C)—CH₂—CH═CH—(CH₂)₁₅—O-
glucoside; (CF₃)₂N—Ar—O—C(═O)—CH₂—CH═CH—(CH₂)₁₅—O—PO₃H
(CF₃)₂N—Ar—O—C(═O)—CH₂—CH═CH—(CH₂)₁₆—COOH; (CF₃)₂N—Ar—O—C(═O)—CH₂—CH═CH—(CH₂)₁₆—SO₃H;
(CF₃)₂N—Ar—O—C(═O)—CH₂—CH═CH—(CH₂)₁₆—O—SO₃H;
(CF₃)₂N—Ar—O—C(═O)—CH₂—CH═CH—(CH₂)₁₆—SH; (CH₃)₂N—Ar—O—C(═O)—CH₂—CH═CH—(CH₂)₁₆—OH;
(CF₃)₂N—Ar—O—C(═O)—CH₂—CH═CH—(CH₂)₁₆—N⁺R¹R²R³Z⁻; (CF₃)₂N—Ar—O—C(═O)—CH₂—CH═CH—(CH₂)₁₆—P⁺R¹R²R³Z⁻;
(CF₃)₂N—Ar—O—C(═C)—CH₂—CH═CH—(CH₂)₁₆—O-
glucoside; (CF₃)₂N—Ar—O—C(═O)—CH₂—CH═CH—(CH₂)₁₆—O—PO₃H
(CF₃—CH₂—CH₂)₂N—Ar—O—C(═C)—(CH₂)₃—COOH; (CF₃—CH₂—CH₂)₂N—Ar—O—C(═O)—(CH₂)₃—SO₃H;
(CF₃—CH₂—CH₂)₂N—Ar—O—C(═O)—(CH₂)₃—O—SO₃H; (CF₃—CH₂—CH₂)₂N—Ar—O—C(═O)—(CH₂)₃—SH;
(CF₃—CH₂—CH₂)₂N—Ar—O—C(═O)—(CH₂)₃—OH; (CF₃—CH₂—CH₂)₂N—Ar—O—C(50 O)—(CH₂)₃—N⁺R¹R²R³Z⁻;
(CF₃—CH₂—CH₂)₂N—AR—O—C(═O)—(CH₂)₃—P⁺R¹R²R³Z⁻;
(CF₃—CH₂—CH₂)₂N—Ar—O—C(═O)—(CH₂)₃—O-glucoside; (CF₃—CH₂—CH₂)₂N—Ar—O—C(═O)—(CH₂)₃—O—PO₃H
(CF₃—CH₂—CH₂)₂N—Ar—O—C(═C)—(CH₂)₄—COOH; (CF₃—CH₂—CH₂)₂N—Ar—O—C(═O)—(CH₂)₄—SO₃H;
(CF₃—CH₂—CH₂)₂N—Ar—O—C(═O)—(CH₂)₄—O—SO₃H; (CF₃—CH₂—CH₂)₂N—Ar—O—C(═O)—(CH₂)₄—SH;
(CF₃—CH₂—CH₂)₂N—Ar—O—C(═O)—(CH₂)₄—OH; (CF₃—CH₂—CH₂)₂N—Ar—O—C(50 O)—(CH₂)₄—N⁺R¹R²R³Z⁻;
(CF₃—CH₂—CH₂)₂N—AR—O—C(═O)—(CH₂)₄—P⁺R¹R²R³Z⁻;
(CF₃—CH₂—CH₂)₂N—Ar—O—C(═O)—(CH₂)₄—O-glucoside; (CF₃—CH₂—CH₂)₂N—Ar—O—C(═O)—(CH₂)₄—O—PO₃H
(CF₃—CH₂—CH₂)₂N—Ar—O—C(═C)—(CH₂)₅—COOH; (CF₃—CH₂—CH₂)₂N—Ar—O—C(═O)—(CH₂)₅—SO₃H;
(CF₃—CH₂—CH₂)₂N—Ar—O—C(═O)—(CH₂)₅—O—SO₃H; (CF₃—CH₂—CH₂)₂N—Ar—O—C(═O)—(CH₂)₅—SH;
(CF₃—CH₂—CH₂)₂N—Ar—O—C(═O)—(CH₂)₅—OH; (CF₃—CH₂—CH₂)₂N—Ar—O—C(50 O)—(CH₂)₅—N⁺R¹R²R³Z⁻;
(CF₃—CH₂—CH₂)₂N—AR—O—C(═O)—(CH₂)₅—P⁺R¹R²R³Z⁻;
(CF₃—CH₂—CH₂)₂N—Ar—O—C(═O)—(CH₂)₅—O-glucoside; (CF₃—CH₂—CH₂)₂N—Ar—O—C(═O)—(CH₂)₅—O—PO₃H
(CF₃—CH₂—CH₂)₂N—Ar—O—C(═C)—(CH₂)₆—COOH; (CF₃—CH₂—CH₂)₂N—Ar—O—C(═O)—(CH₂)₆—SO₃H;
(CF₃—CH₂—CH₂)₂N—Ar—O—C(═O)—(CH₂)₆—O—SO₃H; (CF₃—CH₂—CH₂)₂N—Ar—O—C(═O)—(CH₂)₆—SH;
(CF₃—CH₂—CH₂)₂N—Ar—O—C(═O)—(CH₂)₆—OH; (CF₃—CH₂—CH₂)₂N—Ar—O—C(50 O)—(CH₂)₆—N⁺R¹R²R³Z⁻;
(CF₃—CH₂—CH₂)₂N—AR—O—C(═O)—(CH₂)₆—P⁺R¹R²R³Z⁻;
(CF₃—CH₂—CH₂)₂N—Ar—O—C(═O)—(CH₂)₆—O-glucoside; (CF₃—CH₂—CH₂)₂N—Ar—O—C(═O)—(CH₂)₆—O—PO₃H
(CF₃—CH₂—CH₂)₂N—Ar—O—C(═C)—(CH₂)₇—COOH; (CF₃—CH₂—CH₂)₂N—Ar—O—C(═O)—(CH₂)₇—SO₃H;
(CF₃—CH₂—CH₂)₂N—Ar—O—C(═O)—(CH₂)₇—O—SO₃H; (CF₃—CH₂—CH₂)₂N—Ar—O—C(═O)—(CH₂)₇—SH;
(CF₃—CH₂—CH₂)₂N—Ar—O—C(═O)—(CH₂)₇—OH; (CF₃—CH₂—CH₂)₂N—Ar—O—C(50 O)—(CH₂)₇—N⁺R¹R²R³Z⁻;
(CF₃—CH₂—CH₂)₂N—AR—O—C(═O)—(CH₂)₇—P⁺R¹R²R³Z⁻;
(CF₃—CH₂—CH₂)₂N—Ar—O—C(═O)—(CH₂)₇—O-glucoside; (CF₃—CH₂—CH₂)₂N—Ar—O—C(═O)—(CH₂)₇—O—PO₃H
(CF₃—CH₂—CH₂)₂N—Ar—O—C(═C)—(CH₂)₈—COOH; (CF₃—CH₂—CH₂)₂N—Ar—O—C(═O)—(CH₂)₈—SO₃H;
(CF₃—CH₂—CH₂)₂N—Ar—O—C(═O)—(CH₂)₈—O—SO₃H; (CF₃—CH₂—CH₂)₂N—Ar—O—C(═O)—(CH₂)₈—SH;
(CF₃—CH₂—CH₂)₂N—Ar—O—C(═O)—(CH₂)₈—OH; (CF₃—CH₂—CH₂)₂N—Ar—O—C(50 O)—(CH₂)₈—N⁺R¹R²R³Z⁻;
(CF₃—CH₂—CH₂)₂N—AR—O—C(═O)—(CH₂)₈—P⁺R¹R²R³Z⁻;
(CF₃—CH₂—CH₂)₂N—Ar—O—C(═O)—(CH₂)₈—O-glucoside; (CF₃—CH₂—CH₂)₂N—Ar—O—C(═O)—(CH₂)₈—O—PO₃H
(CF₃—CH₂—CH₂)₂N—Ar—O—C(═C)—CH₂)₉—COOH; (CF₃—CH₂—CH₂)₂N—Ar—O—C(═O)—(CH₂)₉—SO₃H;
(CF₃—CH₂—CH₂)₂N—Ar—O—C(═O)—(CH₂)₉—O—SO₃H; (CF₃—CH₂—CH₂)₂N—Ar—O—C(═O)—(CH₂)₉—SH;
(CF₃—CH₂—CH₂)₂N—Ar—O—C(═O)—(CH₂)₉—OH; (CF₃—CH₂—CH₂)₂N—Ar—O—C(50 O)—(CH₂)₉—N⁺R¹R²R³Z⁻;
(CF₃—CH₂—CH₂)₂N—AR—O—C(═O)—CH₂)₉—P⁺R¹R²R³Z⁻;
(CF₃—CH₂—CH₂)₂N—Ar—O—C(═O)—(CH₂)₉—O-glucoside; (CF₃—CH₂—CH₂)₂N—Ar—O—C(═O)—(CH₂)₉—O—PO₃H
(CF₃—CH₂—CH₂)₂N—Ar—O—C(═C)—(CH₂)₁₀—COOH; (CF₃—CH₂—CH₂)₂N—Ar—O—C(═O)—(CH₂)₁₀—SO₃H;
(CF₃—CH₂—CH₂)₂N—Ar—O—C(═O)—(CH₂)₁₀—O—SO₃H; (CF₃—CH₂—CH₂)₂N—Ar—O—C(═O)—(CH₂)₁₀—SH;
(CF₃—CH₂—CH₂)₂N—Ar—O—C(═O)—(CH₂)₁₀—OH; (CF₃—CH₂—CH₂)₂N—Ar—O—C(50 O)—(CH₂)₁₀—N⁺R¹R²R³Z⁻;
(CF₃—CH₂—CH₂)₂N—AR—O—C(═O)—(CH₂)₁₀—P⁺R¹R²R³Z⁻;
(CF₃—CH₂—CH₂)₂N—Ar—O—C(═O)—(CH₂)₁₀—O-glucoside; (CF₃—CH₂—CH₂)₂N—Ar—O—C(═O)—(CH₂)₁₀—O—PO₃H
(CF₃—CH₂—CH₂)₂N—Ar—O—C(═O)—(CH₂)₁₁—COOH; (CF₃—CH₂—CH₂)₂N—Ar—O—C(═O)—(CH₂)₁₁—SO₃H;
(CF₃—CH₂—CH₂)₂N—Ar—O—C(═O)—(CH₂)₁₁—O—SO₃H; (CF₃—CH₂—CH₂)₂N—Ar—O—C(═O)—(CH₂)₁₁—SH;
(CF₃—CH₂—CH₂)₂N—Ar—O—C(═O)—(CH₂)₁₁—OH; (CF₃—CH₂—CH₂)₂N—Ar—O—C(═O)—(CH₂)₁₁—N⁺R¹R²R³Z⁻;
(CF₃—CH₂—CH₂)₂N—Ar—O—C(═O)—(CH₂)₁₁—P⁺R¹R²R³Z⁻;
(CF₃—CH₂—CH₂)₂N—Ar—O—C(═O)—(CH₂)₁₁—O-glucoside; (CF₃—CH₂—CH₂)₂N—Ar—O—C(═O)—(CH₂)₁₁—O—PO₃H
(CF₃—CH₂—CH₂)₂N—Ar—O—C(═O)—(CH₂)₁₂—COOH; (CF₃—CH₂—CH₂)₂N—Ar—O—C(═O)—(CH₂)₁₂—SO₃H;
(CF₃—CH₂—CH₂)₂N—Ar—O—C(═O)—(CH₂)₁₂—O—SO₃H; (CF₃—CH₂—CH₂)₂N—Ar—O—C(═O)—(CH₂)₁₂—SH;
(CF₃—CH₂—CH₂)₂N—Ar—O—C(═O)—(CH₂)₁₂—OH; (CF₃—CH₂—CH₂)₂N—Ar—O—C(═O)—(CH₂)₁₂—N⁺R¹R²R³Z⁻;
(CF₃—CH₂—CH₂)₂N—Ar—O—C(═O)—(CH₂)₁₂—P⁺R¹R²R³Z⁻;
(CF₃—CH₂—CH₂)₂N—Ar—O—C(═O)—(CH₂)₁₂—O-glucoside; (CF₃—CH₂—CH₂)₂N—Ar—O—C(═O)—(CH₂)₁₂—O—PO₃H
(CF₃—CH₂—CH₂)₂N—Ar—O—C(═O)—(CH₂)₁₃—COOH; (CF₃—CH₂—CH₂)₂N—Ar—O—C(═O)—(CH₂)₁₃—SO₃H;
(CF₃—CH₂—CH₂)₂N—Ar—O—C(═O)—(CH₂)₁₃—O—SO₃H; (CF₃—CH₂—CH₂)₂N—Ar—O—C(═O)—(CH₂)₁₃—SH;
(CF₃—CH₂—CH₂)₂N—Ar—O—C(═O)—(CH₂)₁₃—OH; (CF₃—CH₂—CH₂)₂N—Ar—O—C(═O)—(CH₂)₁₃—N⁺R¹R²R³Z⁻;
(CF₃—CH₂—CH₂)₂N—Ar—O—C(═O)—(CH₂)₁₃—P⁺R¹R²R³Z⁻;
(CF₃—CH₂—CH₂)₂N—Ar—O—C(═O)—(CH₂)₁₃—O-glucoside; (CF₃—CH₂—CH₂)₂N—Ar—O—C(═O)—(CH₂)₁₃—O—PO₃H
(CF₃—CH₂—CH₂)₂N—Ar—O—C(═O)—(CH₂)₁₄—COOH; (CF₃—CH₂—CH₂)₂N—Ar—O—C(═O)—(CH₂)₁₄—SO₃H;
(CF₃—CH₂—CH₂)₂N—Ar—O—C(═O)—(CH₂)₁₄—O—SO₃H; (CF₃—CH₂—CH₂)₂N—Ar—O—C(═O)—(CH₂)₁₄—SH;
(CF₃—CH₂—CH₂)₂N—Ar—O—C(═O)—(CH₂)₁₄—OH; (CF₃—CH₂—CH₂)₂N—Ar—O—C(═O)—(CH₂)₁₄—N⁺R¹R²R³Z⁻;
(CF₃—CH₂—CH₂)₂N—Ar—O—C(═O)—(CH₂)₁₄—P⁺R¹R²R³Z⁻;
(CF₃—CH₂—CH₂)₂N—Ar—O—C(═O)—(CH₂)₁₄—O-glucoside; (CF₃—CH₂—CH₂)₂N—Ar—O—C(═O)—(CH₂)₁₄—O—PO₃H
(CF₃—CH₂—CH₂)₂N—Ar—O—C(═O)—(CH₂)₁₅—COOH; (CF₃—CH₂—CH₂)₂N—Ar—O—C(═O)—(CH₂)₁₅—SO₃H;
(CF₃—CH₂—CH₂)₂N—Ar—O—C(═O)—(CH₂)₁₅—O—SO₃H; (CF₃—CH₂—CH₂)₂N—Ar—O—C(═O)—(CH₂)₁₅—SH;
(CF₃—CH₂—CH₂)₂N—Ar—O—C(═O)—(CH₂)₁₅—OH; (CF₃—CH₂—CH₂)₂N—Ar—O—C(═O)—(CH₂)₁₅—N⁺R¹R²R³Z⁻;
(CF₃—CH₂—CH₂)₂N—Ar—O—C(═O)—(CH₂)₁₅—P⁺R¹R²R³Z⁻;
(CF₃—CH₂—CH₂)₂N—Ar—O—C(═O)—(CH₂)₁₅—O-glucoside; (CF₃—CH₂—CH₂)₂N—Ar—O—C(═O)—(CH₂)₁₅—O—PO₃H
(CF₃—CH₂—CH₂)₂N—Ar—O—C(═O)—(CH₂)₁₆—COOH; (CF₃—CH₂—CH₂)₂N—Ar—O—C(═O)—(CH₂)₁₆—SO₃H;
(CF₃—CH₂—CH₂)₂N—Ar—O—C(═O)—(CH₂)₁₆—O—SO₃H; (CF₃—CH₂—CH₂)₂N—Ar—O—C(═O)—(CH₂)₁₆—SH;
(CF₃—CH₂—CH₂)₂N—Ar—O—C(═O)—(CH₂)₁₆—OH; (CF₃—CH₂—CH₂)₂N—Ar—O—C(═O)—(CH₂)₁₆—N⁺R¹R²R³Z⁻;
(CF₃—CH₂—CH₂)₂N—Ar—O—C(═O)—(CH₂)₁₆—P⁺R¹R²R³Z⁻;
(CF₃—CH₂—CH₂)₂N—Ar—O—C(═O)—(CH₂)₁₆—O-glucoside; (CF₃—CH₂—CH₂)₂N—Ar—O—C(═O)—(CH₂)₁₆—O—PO₃H
(CF₃—CH₂—CH₂)₂N—Ar—O—C(═O)—(CH₂)ₙ—O—(CH₂)_{n′}—COOH; (CF₃—CH₂—CH₂)₂N—Ar—O—C(═O)—(CH₂)ₙ—O—(CH₂)_{n′}—SO₃H;
(CF₃—CH₂—CH₂)₂N—Ar—O—C(═O)—(CH₂)ₙ—O—(CH₂)_{n′}—O—SO₃H;
(CF₃—CH₂—CH₂)₂N—Ar—O—C(═O)—(CH₂)ₙ—O—(CH₂)_{n′}—SH; (CF₃—CH₂—CH₂)₂N—Ar—O—C(═O)—(CH₂)ₙ—O—(CH₂)_{n′}—OH;
(CF₃—CH₂—CH₂)₂N—Ar—O—C(═O)—(CH₂)ₙ—O—(CH₂)_{n′}—N⁺R¹R²R³Z⁻;
(CF₃—CH₂—CH₂)₂N—Ar—O—C(═O)—(CH₂)ₙ—O—(CH₂)_{n′}—P⁺R¹R²R³Z⁻; (CF₃—CH₂—CH₂)₂N—Ar—O—C(═O)—(CH₂)ₙ—O—(CH₂)_{n′}—O-
glucoside; (CF₃—CH₂—CH₂)₂N—Ar—O—C(═O)—(CH₂)ₙ—O—(CH₂)_{n′}—O—PO₃H
(CF₃—CH₂—CH₂)₂N—Ar—O—C(═O)—CH₂—CH═CH—(CH₂)₃—COOH;
(CF₃—CH₂—CH₂)₂N—Ar—O—C(═O)—CH₂—CH═CH—(CH₂)₃—SO₃H;
(CF₃—CH₂—CH₂)₂N—Ar—O—C(═O)—CH₂—CH═CH—(CH₂)₃—O—SO₃H;
(CF₃—CH₂—CH₂)₂N—Ar—O—C(═O)—CH₂—CH═CH—(CH₂)₃—SH; (CF₃—CH₂—CH₂)₂N—Ar—O—C(═O)—CH₂—CH═CH—(CH₂)₃—OH;
(CF₃—CH₂—CH₂)₂N—Ar—O—C(═O)—CH₂—CH═CH—(CH₂)₃—N⁺R¹R²R³Z⁻;
(CF₃—CH₂—CH₂)₂N—Ar—O—C(═O)—CH₂—CH═CH—(CH₂)₃—P⁺R¹R²R³Z⁻;
(CF₃—CH₂—CH₂)₂N—Ar—O—C(═O)—CH₂—CH═CH—(CH₂)₃—O-glucoside;
(CF₃—CH₂—CH₂)₂N—Ar—O—C(═O)—CH₂—CH═CH—(CH₂)₃—O—PO₃H
(CF₃—CH₂—CH₂)₂N—Ar—O—C(═O)—CH₂—CH═CH—(CH₂)₄—COOH;
(CF₃—CH₂—CH₂)₂N—Ar—O—C(═O)—CH₂—CH═CH—(CH₂)₄—SO₃H;
(CF₃—CH₂—CH₂)₂N—Ar—O—C(═O)—CH₂—CH═CH—(CH₂)₄—O—SO₃H;
(CF₃—CH₂—CH₂)₂N—Ar—O—C(═O)—CH₂—CH═CH—(CH₂)₄—SH; (CF₃—CH₂—CH₂)₂N—Ar—O—C(═O)—CH₂—CH═CH—(CH₂)₄—OH;
(CF₃—CH₂—CH₂)₂N—Ar—O—C(═O)—CH₂—CH═CH—(CH₂)₄—N⁺R¹R²R³Z⁻;
(CF₃—CH₂—CH₂)₂N—Ar—O—C(═O)—CH₂—CH═CH—(CH₂)₄—P⁺R¹R²R³Z⁻;
(CF₃—CH₂—CH₂)₂N—Ar—O—C(═O)—CH₂—CH═CH—(CH₂)₄—O-glucoside;
(CF₃—CH₂—CH₂)₂N—Ar—O—C(═O)—CH₂—CH═CH—(CH₂)₄—O—PO₃H
(CF₃—CH₂—CH₂)₂N—Ar—O—C(═O)—CH₂—CH═CH—(CH₂)₅—COOH;
(CF₃—CH₂—CH₂)₂N—Ar—O—C(═O)—CH₂—CH═CH—(CH₂)₅—SO₃H;
(CF₃—CH₂—CH₂)₂N—Ar—O—C(═O)—CH₂—CH═CH—(CH₂)₅—O—SO₃H;
(CF₃—CH₂—CH₂)₂N—Ar—O—C(═O)—CH₂—CH═CH—(CH₂)₅—SH; (CF₃—CH₂—CH₂)₂N—Ar—O—C(═O)—CH₂—CH═CH—(CH₂)₅—OH;
(CF₃—CH₂—CH₂)₂N—Ar—O—C(═O)—CH₂—CH═CH—(CH₂)₅—N⁺R¹R²R³Z⁻;
(CF₃—CH₂—CH₂)₂N—Ar—O—C(═O)—CH₂—CH═CH—(CH₂)₅—P⁺R¹R²R³Z⁻;
(CF₃—CH₂—CH₂)₂N—Ar—O—C(═O)—CH₂—CH═CH—(CH₂)₅—O-glucoside;
(CF₃—CH₂—CH₂)₂N—Ar—O—C(═O)—CH₂—CH═CH—(CH₂)₅—O—PO₃H
(CF₃—CH₂—CH₂)₂N—Ar—O—C(═O)—CH₂—CH═CH—(CH₂)₆—COOH;
(CF₃—CH₂—CH₂)₂N—Ar—O—C(═O)—CH₂—CH═CH—(CH₂)₆—SO₃H;
(CF₃—CH₂—CH₂)₂N—Ar—O—C(═O)—CH₂—CH═CH—(CH₂)₆—O—SO₃H;
(CF₃—CH₂—CH₂)₂N—Ar—O—C(═O)—CH₂—CH═CH—(CH₂)₆—SH; (CF₃—CH₂—CH₂)₂N—Ar—O—C(═O)—CH₂—CH═CH—(CH₂)₆—OH;
(CF₃—CH₂—CH₂)₂N—Ar—O—C(═O)—CH₂—CH═CH—(CH₂)₆—N⁺R¹R²R³Z⁻;
(CF₃—CH₂—CH₂)₂N—Ar—O—C(═O)—CH₂—CH═CH—(CH₂)₆—P⁺R¹R²R³Z⁻;
(CF₃—CH₂—CH₂)₂N—Ar—O—C(═O)—CH₂—CH═CH—(CH₂)₆—O-glucoside;
(CF₃—CH₂—CH₂)₂N—Ar—O—C(═O)—CH₂—CH═CH—(CH₂)₆—O—PO₃H
(CF₃—CH₂—CH₂)₂N—Ar—O—C(═O)—CH₂—CH═CH—(CH₂)₇—COOH;
(CF₃—CH₂—CH₂)₂N—Ar—O—C(═O)—CH₂—CH═CH—(CH₂)₇—SO₃H;
(CF₃—CH₂—CH₂)₂N—Ar—O—C(═O)—CH₂—CH═CH—(CH₂)₇—O—SO₃H;
(CF₃—CH₂—CH₂)₂N—Ar—O—C(═O)—CH₂—CH═CH—(CH₂)₇—SH; (CF₃—CH₂—CH₂)₂N—Ar—O—C(═O)—CH₂—CH═CH—(CH₂)₇—OH;
(CF₃—CH₂—CH₂)₂N—Ar—O—C(═O)—CH₂—CH═CH—(CH₂)₇—N⁺R¹R²R³Z⁻;
(CF₃—CH₂—CH₂)₂N—Ar—O—C(═O)—CH₂—CH═CH—(CH₂)₇—P⁺R¹R²R³Z⁻;
(CF₃—CH₂—CH₂)₂N—Ar—O—C(═O)—CH₂—CH═CH—(CH₂)₇—O-glucoside;
(CF₃—CH₂—CH₂)₂N—Ar—O—C(═O)—CH₂—CH═CH—(CH₂)₇—O—PO₃H
(CF₃—CH₂—CH₂)₂N—Ar—O—C(═O)—CH₂—CH═CH—(CH₂)₈—COOH;
(CF₃—CH₂—CH₂)₂N—Ar—O—C(═O)—CH₂—CH═CH—(CH₂)₈—SO₃H;
(CF₃—CH₂—CH₂)₂N—Ar—O—C(═O)—CH₂—CH═CH—(CH₂)₈—O—SO₃H;
(CF₃—CH₂—CH₂)₂N—Ar—O—C(═O)—CH₂—CH═CH—(CH₂)₈—SH; (CF₃—CH₂—CH₂)₂N—Ar—O—C(═O)—CH₂—CH═CH—(CH₂)₈—OH;
(CF₃—CH₂—CH₂)₂N—Ar—O—C(═O)—CH₂—CH═CH—(CH₂)₈—N⁺R¹R²R³Z⁻;
(CF₃—CH₂—CH₂)₂N—Ar—O—C(═O)—CH₂—CH═CH—(CH₂)₈—P⁺R¹R²R³Z⁻;
(CF₃—CH₂—CH₂)₂N—Ar—O—C(═O)—CH₂—CH═CH—(CH₂)₈—O-glucoside;
(CF₃—CH₂—CH₂)₂N—Ar—O—C(═O)—CH₂—CH═CH—(CH₂)₈—O—PO₃H
(CF₃—CH₂—CH₂)₂N—Ar—O—C(═O)—CH₂—CH═CH—(CH₂)₉—COOH;
(CF₃—CH₂—CH₂)₂N—Ar—O—C(═O)—CH₂—CH═CH—(CH₂)₉—SO₃H;
(CF₃—CH₂—CH₂)₂N—Ar—O—C(═O)—CH₂—CH═CH—(CH₂)₉—O—SO₃H;
(CF₃—CH₂—CH₂)₂N—Ar—O—C(═O)—CH₂—CH═CH—(CH₂)₉—SH; (CF₃—CH₂—CH₂)₂N—Ar—O—C(═O)—CH₂—CH═CH—(CH₂)₉—OH;
(CF₃—CH₂—CH₂)₂N—Ar—O—C(═O)—CH₂—CH═CH—(CH₂)₉—N⁺R¹R²R³Z⁻;
(CF₃—CH₂—CH₂)₂N—Ar—O—C(═O)—CH₂—CH═CH—(CH₂)₉—P⁺R¹R²R³Z⁻;
(CF₃—CH₂—CH₂)₂N—Ar—O—C(═O)—CH₂—CH═CH—(CH₂)₉—O-glucoside;
(CF₃—CH₂—CH₂)₂N—Ar—O—C(═O)—CH₂—CH═CH—(CH₂)₉—O—PO₃H
(CF₃—CH₂—CH₂)₂N—Ar—O—C(═O)—CH₂—CH═CH—(CH₂)₁₀—COOH;
(CF₃—CH₂—CH₂)₂N—Ar—O—C(═O)—CH₂—CH═CH—(CH₂)₁₀—SO₃H;
(CF₃—CH₂—CH₂)₂N—Ar—O—C(═O)—CH₂—CH═CH—(CH₂)₁₀—O—SO₃H;
(CF₃—CH₂—CH₂)₂N—Ar—O—C(═O)—CH₂—CH═CH—(CH₂)₁₀—SH;
(CF₃—CH₂—CH₂)₂N—Ar—O—C(═O)—CH₂—CH═CH—(CH₂)₁₀—OH;
(CF₃—CH₂—CH₂)₂N—Ar—O—C(═O)—CH₂—CH═CH—(CH₂)₁₀—N⁺R¹R²R³Z⁻;
(CF₃—CH₂—CH₂)₂N—Ar—O—C(═O)—CH₂—CH═CH—(CH₂)₁₀—P⁺R¹R²R³Z⁻;
(CF₃—CH₂—CH₂)₂N—Ar—O—C(═O)—CH₂—CH═CH—(CH₂)₁₀—O-
glucoside; (CF₃—CH₂—CH₂)₂N—Ar—O—C(═O)—CH₂—CH═CH—(CH₂)₁₀—O—PO₃H
(CF₃—CH₂—CH₂)₂N—Ar—O—C(═O)—CH₂—CH═CH—(CH₂)₁₁—COOH;
(CF₃—CH₂—CH₂)₂N—Ar—O—C(═O)—CH₂—CH═CH—(CH₂)₁₁—SO₃H;
(CF₃—CH₂—CH₂)₂N—Ar—O—C(═O)—CH₂—CH═CH—(CH₂)₁₁—O—SO₃H;
(CF₃—CH₂—CH₂)₂N—Ar—O—C(═O)—CH₂—CH═CH—(CH₂)₁₁—SH;
(CF₃—CH₂—CH₂)₂N—Ar—O—C(═O)—CH₂—CH═CH—(CH₂)₁₁—OH;
(CF₃—CH₂—CH₂)₂N—Ar—O—C(═O)—CH₂—CH═CH—(CH₂)₁₁—N⁺R¹R²R³Z⁻;
(CF₃—CH₂—CH₂)₂N—Ar—O—C(═O)—CH₂—CH═CH—(CH₂)₁₁—P⁺R¹R²R³Z⁻;
(CF₃—CH₂—CH₂)₂N—Ar—O—C(═O)—CH₂—CH═CH—(CH₂)₁₁—O-
glucoside; (CF₃—CH₂—CH₂)₂N—Ar—O—C(═O)—CH₂—CH═CH—(CH₂)₁₁—O—PO₃H
(CF₃—CH₂—CH₂)₂N—Ar—O—C(═O)—CH₂—CH═CH—(CH₂)₁₂—COOH;
(CF₃—CH₂—CH₂)₂N—Ar—O—C(═O)—CH₂—CH═CH—(CH₂)₁₂—SO₃H;
(CF₃—CH₂—CH₂)₂N—Ar—O—C(═O)—CH₂—CH═CH—(CH₂)₁₂—O—SO₃H;
(CF₃—CH₂—CH₂)₂N—Ar—O—C(═O)—CH₂—CH═CH—(CH₂)₁₂—SH;
(CF₃—CH₂—CH₂)₂N—Ar—O—C(═O)—CH₂—CH═CH—(CH₂)₁₂—OH;
(CF₃—CH₂—CH₂)₂N—Ar—O—C(═O)—CH₂—CH═CH—(CH₂)₁₂—N⁺R¹R²R³Z⁻;
(CF₃—CH₂—CH₂)₂N—Ar—O—C(═O)—CH₂—CH═CH—(CH₂)₁₂—P⁺R¹R²R³Z⁻;
(CF₃—CH₂—CH₂)₂N—Ar—O—C(═O)—CH₂—CH═CH—(CH₂)₁₂—O-
glucoside; (CF₃—CH₂—CH₂)₂N—Ar—O—C(═O)—CH₂—CH═CH—(CH₂)₁₂—O—PO₃H
(CF₃—CH₂—CH₂)₂N—Ar—O—C(═O)—CH₂—CH═CH—(CH₂)₁₃—COOH;
(CF₃—CH₂—CH₂)₂N—Ar—O—C(═O)—CH₂—CH═CH—(CH₂)₁₃—SO₃H;
(CF₃—CH₂—CH₂)₂N—Ar—O—C(═O)—CH₂—CH═CH—(CH₂)₁₃—O—SO₃H;
(CF₃—CH₂—CH₂)₂N—Ar—O—C(═O)—CH₂—CH═CH—(CH₂)₁₃—SH;
(CF₃—CH₂—CH₂)₂N—Ar—O—C(═O)—CH₂—CH═CH—(CH₂)₁₃—OH;
(CF₃—CH₂—CH₂)₂N—Ar—O—C(═O)—CH₂—CH═CH—(CH₂)₁₃—N⁺R¹R²R³Z⁻;
(CF₃—CH₂—CH₂)₂N—Ar—O—C(═O)—CH₂—CH═CH—(CH₂)₁₃—P⁺R¹R²R³Z⁻;
(CF₃—CH₂—CH₂)₂N—Ar—O—C(═O)—CH₂—CH═CH—(CH₂)₁₃—O-
glucoside; (CF₃—CH₂—CH₂)₂N—Ar—O—C(═O)—CH₂—CH═CH—(CH₂)₁₃—O—PO₃H
(CF₃—CH₂—CH₂)₂N—Ar—O—C(═O)—CH₂—CH═CH—(CH₂)₁₄—COOH;
(CF₃—CH₂—CH₂)₂N—Ar—O—C(═O)—CH₂—CH═CH—(CH₂)₁₄—SO₃H;
(CF₃—CH₂—CH₂)₂N—Ar—O—C(═O)—CH₂—CH═CH—(CH₂)₁₄—O—SO₃H;
(CF₃—CH₂—CH₂)₂N—Ar—O—C(═O)—CH₂—CH═CH—(CH₂)₁₄—SH;
(CF₃—CH₂—CH₂)₂N—Ar—O—C(═O)—CH₂—CH═CH—(CH₂)₁₄—OH;
(CF₃—CH₂—CH₂)₂N—Ar—O—C(═O)—CH₂—CH═CH—(CH₂)₁₄—N⁺R¹R²R³Z⁻;
(CF₃—CH₂—CH₂)₂N—Ar—O—C(═O)—CH₂—CH═CH—(CH₂)₁₄—P⁺R¹R²R³Z⁻;
(CF₃—CH₂—CH₂)₂N—Ar—O—C(═O)—CH₂—CH═CH—(CH₂)₁₄—O-
glucoside; (CF₃—CH₂—CH₂)₂N—Ar—O—C(═O)—CH₂—CH═CH—(CH₂)₁₄—O—PO₃H
(CF₃—CH₂—CH₂)₂N—Ar—O—C(═O)—CH₂—CH═CH—(CH₂)₁₅—COOH;
(CF₃—CH₂—CH₂)₂N—Ar—O—C(═O)—CH₂—CH═CH—(CH₂)₁₅—SO₃H;
(CF₃—CH₂—CH₂)₂N—Ar—O—C(═O)—CH₂—CH═CH—(CH₂)₁₅—O—SO₃H;
(CF₃—CH₂—CH₂)₂N—Ar—O—C(═O)—CH₂—CH═CH—(CH₂)₁₅—SH;
(CF₃—CH₂—CH₂)₂N—Ar—O—C(═O)—CH₂—CH═CH—(CH₂)₁₅—OH;
(CF₃—CH₂—CH₂)₂N—Ar—O—C(═O)—CH₂—CH═CH—(CH₂)₁₅—N⁺R¹R²R³Z⁻;
(CF₃—CH₂—CH₂)₂N—Ar—O—C(═O)—CH₂—CH═CH—(CH₂)₁₅—P⁺R¹R²R³Z⁻;
(CF₃—CH₂—CH₂)₂N—Ar—O—C(═O)—CH₂—CH═CH—(CH₂)₁₅—O-
glucoside; (CF₃—CH₂—CH₂)₂N—Ar—O—C(═O)—CH₂—CH═CH—(CH₂)₁₅—O—PO₃H
(CF₃—CH₂—CH₂)₂N—Ar—O—C(═O)—CH₂—CH═CH—(CH₂)₁₆—COOH;
(CF₃—CH₂—CH₂)₂N—Ar—O—C(═O)—CH₂—CH═CH—(CH₂)₁₆—SO₃H;
(CF₃—CH₂—CH₂)₂N—Ar—O—C(═O)—CH₂—CH═CH—(CH₂)₁₆—O—SO₃H;
(CF₃—CH₂—CH₂)₂N—Ar—O—C(═O)—CH₂—CH═CH—(CH₂)₁₆—SH;
(CF₃—CH₂—CH₂)₂N—Ar—O—C(═O)—CH₂—CH═CH—(CH₂)₁₆—OH;
(CF₃—CH₂—CH₂)₂N—Ar—O—C(═O)—CH₂—CH═CH—(CH₂)₁₆—N⁺R¹R²R³Z⁻;
(CF₃—CH₂—CH₂)₂N—Ar—O—C(═O)—CH₂—CH═CH—(CH₂)₁₆—P⁺R¹R²R³Z⁻;
(CF₃—CH₂—CH₂)₂N—Ar—O—C(═O)—CH₂—CH═CH—(CH₂)₁₆—O-
glucoside; (CF₃—CH₂—CH₂)₂N—Ar—O—C(═O)—CH₂—CH═CH—(CH₂)₁₆—O—PO₃H

The compounds which can be used in accordance with the invention as surfactants are particularly suitable for use as hydrophobicising agents or oliophobicising agents.

Areas of use are, for example, the surface modification of textiles, paper, glass, porous building materials or adsorbents. In paints, coatings, inks, photographic coatings (for photographic plates, films and papers), special coatings for semiconductor photolithography (photoresists, top antireflective coatings, bottom antireflective coatings) or other preparations for surface coating, the compounds according to the invention and the compounds to be employed in accordance with the invention can advantageously be employed with one or more of the following functions: antifogging agent, dispersant, emulsion stabiliser, antifoam, deaerating agent, antistatic, flame retardant, gloss enhancer, lubricant, pigment- or filler-compatibility enhancer, scratch-resistance enhancer, substrate adhesion enhancer, surface-adhesion reducer, skin preventer, hydrophobicising agent, oliophobicising agent, UV stabiliser, wetting agent, flow-control agent, viscosity reducer, migration inhibitor, drying accelerator. In printing inks, the compounds according to the invention and the compounds to be employed in accordance with the invention can likewise advantageously be employed and have one or more of the following functions: antifoam, deaerating agent, friction-control agent, wetting agent, flow-control agent, pigment-compatibility enhancer, print-resolution enhancer, drying accelerator.

The present invention therefore furthermore relates to the use of the compounds according to the invention or the compounds to be employed in accordance with the invention as additives in preparations for surface coating, such as printing inks, paints, coatings, photographic coatings, special coatings for semiconductor photolithography, such as photoresists, top antireflective coatings, bottom antireflective coatings, or in additive preparations for addition to corresponding preparations.

A further use according to the invention of compounds according to the invention or compounds to be employed in accordance with the invention is the use as interface promoter or emulsifier. These properties can advantageously be utilised, in particular, for the preparation of fluoropolymers by means of emulsion polymerisation.

Compounds according to the invention and compounds to be employed in accordance with the invention can be employed as foam stabiliser, in particular in preparations which are known as “fire-extinguishing foams”. The invention therefore furthermore relates to the use of compounds according to the invention or compounds to be employed in accordance with the invention as foam stabiliser and/or for supporting film formation, in particular in aqueous film-forming fire-extinguishing foams, both synthetic and also protein-based, and also for alcohol-resistant formulations (AFFF and AFFF-AR, FP, FFFP and FFFP-AR fire-extinguishing foams).

Compounds according to the invention and compounds to be employed in accordance with the invention can also be used as antistatics. The antistatic action is of particular importance in the treatment of textiles, in particular clothing, carpets and carpeting, upholstery in furniture and automobiles, non-woven textile materials, leather goods, papers and cardboard articles, wood and wood-based materials, mineral substrates, such as stone, cement, concrete, plaster, ceramics (glazed and unglazed tiles, earthenware, porcelain) and glasses, and for plastics and metallic substrates. The present application relates to the corresponding use.

For metallic substrates, the present invention additionally also relates to the use of compounds according to the invention in anticorrosion agents.

The present invention furthermore also relates to the use thereof as mould-release agents in plastics processing.

In general, compounds according to the invention and compounds to be employed in accordance with the invention are suitable as protection agents against spots and soiling, stain releases, antifogging agents, lubricants, and as abrasion-resistance and mechanical wear-resistance enhancers.

Compounds according to the invention and compounds to be employed in accordance with the invention can advantageously be employed as additives in cleaning compositions and spot removers for textiles (in particular clothing, carpets and carpeting, upholstery in furniture and automobiles) and hard surfaces (in particular kitchen surfaces, sanitary installations, tiles, glass) and in polishes and waxes (in particular for furniture, flooring and automobiles) with one or more of the following functions: wetting agent, flow-control agent, hydrophobicising agent, oliophobicising agent, protection agent against spots and soiling, lubricant, antifoam, deaerating agent, drying accelerator. In the case of cleaning compositions and spot removers, the use as detergent or dirt emulsifier and dispersant is additionally also an advantageous embodiment of the present invention. The invention therefore furthermore relates to the use of compounds according to the invention or compounds to be employed in accordance with the invention in cleaning compositions and spot removers or as wetting agent, flow-control agent, hydrophobicising agent, oliophobicising agent, protection agent against spots and soiling, lubricant, antifoam, deaerating agent or drying accelerator.

The compounds according to the invention and compounds to be employed in accordance with the invention can also advantageously be used as additives in polymeric materials (plastics) with one or more of the following functions: lubricant, internal-friction reducer, UV stabiliser, hydrophobicising agent, oliophobicising agent, protection agent against spots and soiling, coupling agent for fillers, flame retardant, migration inhibitor (in particular against migration of plasticisers), antifogging agent.

On use as additives in liquid media for cleaning, etching, reactive modification and/or substance deposition on metal surfaces (in particular also electroplating and anodisation) or semiconductor surfaces (in particular for semiconductor photolithography), compounds according to the invention and compounds to be employed in accordance with the invention act as developer, stripper, edge bead remover, etching and cleaning composition, as wetting agent and/or deposited film quality enhancer. In the case of electroplating processes (in particular chrome plating), the present invention additionally also relates to the function as fume inhibitor with or without foam action.

In addition, the compounds which can be used in accordance with the invention as surfactants are suitable for washing and cleaning applications, in particular of textiles. Cleaning and polishing of hard surfaces is also a possible area of application for the compounds which can be used in accordance with the invention as surfactants. Furthermore, the compounds which can be used in accordance with the invention as surfactants can advantageously be employed in cosmetic products, such as, for example, foam baths and hair shampoos, or as emulsifiers in creams and lotions. The compounds according to the invention and the compounds to be employed in accordance with the invention can likewise advantageously be employed as additives in hair- and bodycare products (for example hair rinses and hair conditioners), with one or more of the following functions: wetting agent, foaming agent, lubricant, antistatic, skin-grease resistance enhancer.

Compounds according to the invention and compounds to be employed in accordance with the invention act as additives in herbicides, pesticides and fungicides with one or more of the following functions: substrate wetting agent, adjuvant, foam inhibitor, dispersant, emulsion stabiliser.

Compounds according to the invention and compounds to be employed in accordance with the invention can likewise beneficially be employed as additives in adhesives, with one or more of the following functions: wetting agent, penetration agent, substrate adhesion enhancer, antifoam. Compounds according to the invention and compounds to be employed in accordance with the invention can also serve as additives in lubricants and hydraulic fluids, with one or more of the following functions: wetting agent, corrosion inhibitor. In the case of lubricants, the use as dispersant (in particular for fluoropolymer particles) is additionally also an essential aspect.

On use as additives in putty and filling compositions, compounds according to the invention and compounds to be employed in accordance with the invention can act with one or more of the following functions: hydrophobicising agent, oliophobicising agent, protection agent against soiling, weathering-resistance enhancer, UV stabiliser, silicone bleeding inhibitor.

A further area of application for the compounds which can be used in accordance with the invention as surfactants is flotation, i.e. the recovery and separation of ores and minerals from dead rock. To this end, they are employed as additives in preparations for ore processing, in particular flotation and leaching solutions, with one or more of the following functions: wetting agent, foaming agent, foam inhibitor. A related use is also as additives in agents for the stimulation of oil wells, with one or more of the following functions: wetting agent, foaming agent, emulsifier.

In addition, they can be employed as additives in de-icing agents or icing inhibitors.

In addition, preferred compounds which can be used in accordance with the invention as surfactants can also be employed as emulsifiers or dispersion assistants in foods. Further fields of application are in metal treatment, as leather auxiliaries, construction chemistry and in crop protection.

Surfactants according to the invention are furthermore also suitable as antimicrobial active compound, in particular as reagents for antimicrobial surface modification. Of particular advantage for this application is the use of compounds of the formula I or II or III where X stands for a cationic polar group or a polymerisable group.

The compounds to be used in accordance with the invention can be prepared by methods known per se to the person skilled in the art from the literature.

The group Rf, in which Rf stands for CF₃—(CH₂)ᵣ—, CF₃—(CH₂)ᵣ—O—, CF₃—(CH₂)ᵣ—S—, CF₃CF₂—S—, SF₅—(CH₂)ᵣ— or [CF₃—(CH₂)ᵣ]₂N— or [CF₃—(CH₂)ᵣ]NH— or (CF₃)₂N—(CH₂)ᵣ—, with indices as described above, can be introduced by means of substitution reactions on aromatic compounds. If Rf is used in the following schemes, the definition given here applies, unless stated otherwise.

Thus, the CF₃ groups can be obtained by reaction of aromatic carboxylic acids with HF and SF₄ under superatmospheric pressure and at elevated temperature, as indicated in the following scheme.

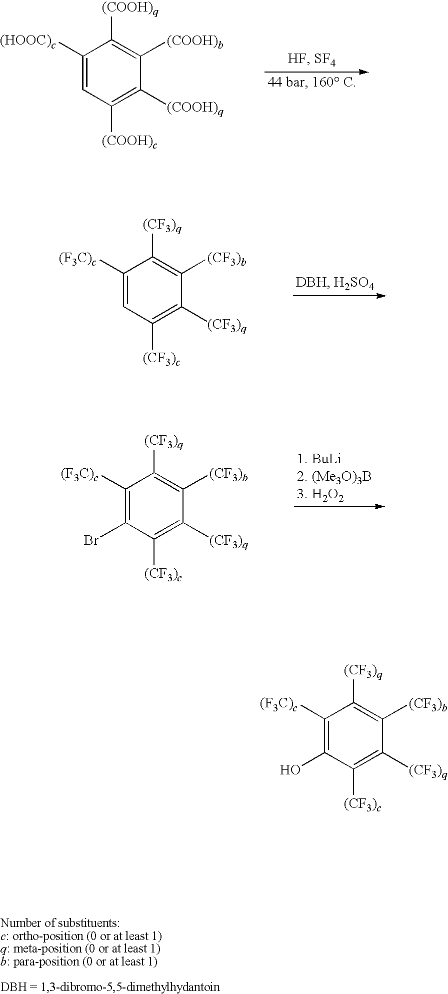

The modification of commercial p-nitropentafluorosulfuranyl compounds can be carried out as described in P. Kirsch et al., Angewandte Chemie 1999, 111, 2174-2178. Corresponding reactions are described in greater detail in the example part. The m,m-bispentafluorosulfuranyl compounds are accessible as described in W. A. Sheppard, J. Am. Chem. Soc. 1962, 84, 3064-3072 or U.S. Pat. No. 3,073,861 or U.S. Pat. No. 3,135,736:

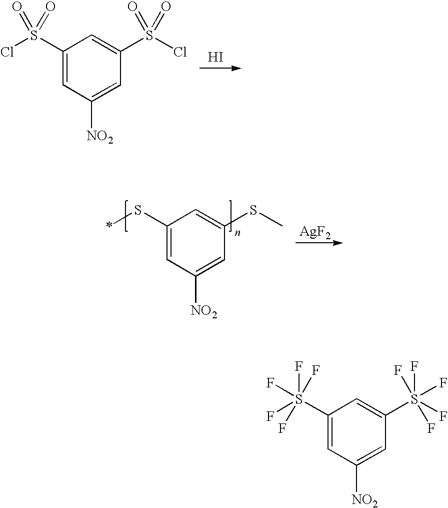

The corresponding disclosure of the said methods in the cited references thus expressly also belongs to the disclosure content of the present application.

Aromatic trifluoromethyl thioethers and pentafluoroethyl thioethers are accessible by substitution of iodinated aromatic compounds or etherification of thiophenols, as indicated in the following schemes:

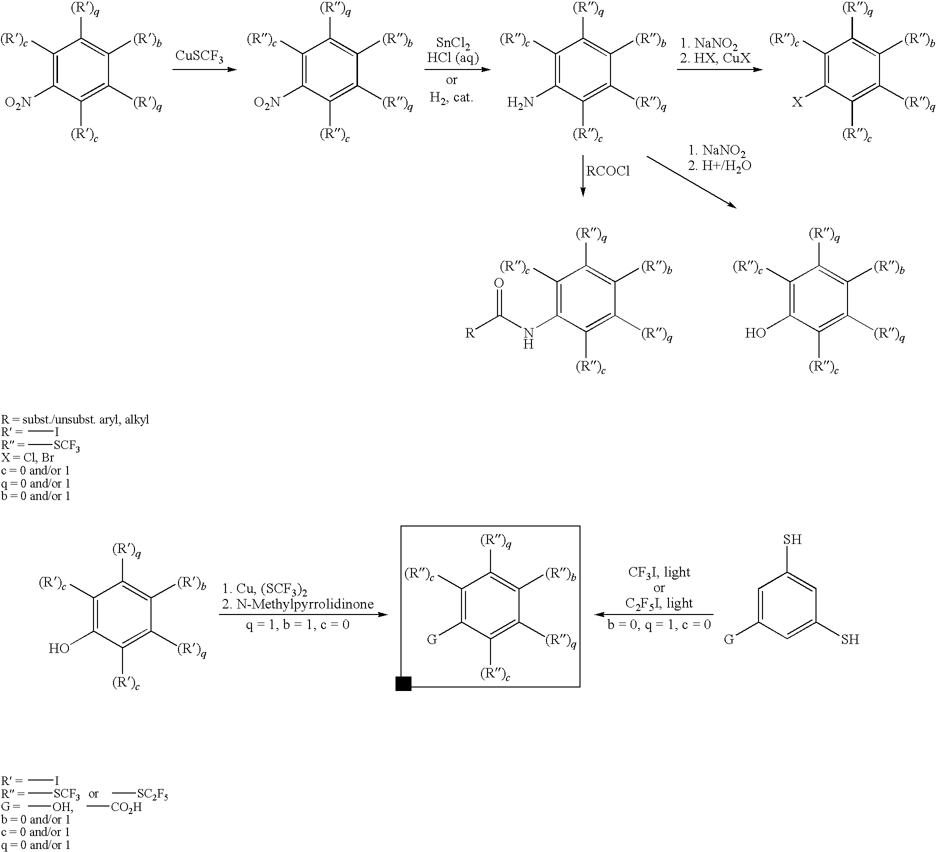

Trifluoromethoxyaromatic compounds can be obtained by reaction of phenols with carbon tetrachloride and hydrogen fluoride.

The amine building block [CF₃—(CH₂)ᵣ]₂N—, where r stands for an integer selected from the range from 0 to 5, can be introduced with the aid of the Gabriel synthesis (Organikum: Organisch-Chemisches Grundpraktikum [Basic Practical Organic Chemistry], 16th Edn., VEB Deutscher Verlag der Wissenschaften, Berlin, 1986), followed by liberation of the primary amine by reaction with hydrazine. Subsequent alkylation of this amine using CF₃(CH₂)ᵣHal and debenzylation gives the tertiary amino alcohol as key building block.

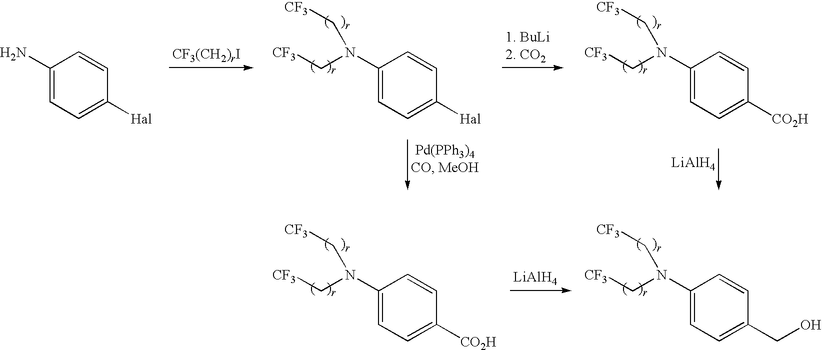

(CF₃)₂N substituents can be obtained in accordance with F. S. Fawcett; J. Am. Chem. Soc. 84 (No. 22) (1962) 4275-4285 starting from isocyanates by reaction with fluorophosgene and subsequent fluorination using SF₄/HF or starting from isothiocyanates by reaction with mercury difluoride and subsequent reaction with fluorophosgene, and subsequent fluorination using SF₄/HF:

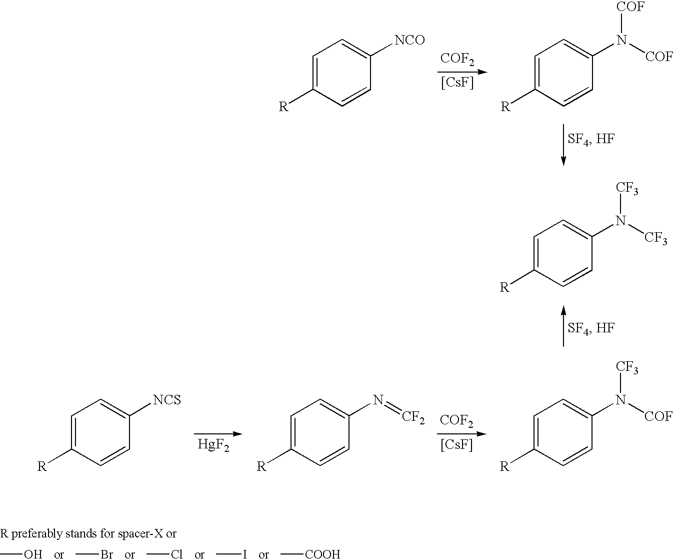

An alternative route for the preparation of bistrifluoromethylanilines starts from aromatic aldehydes and is described in detail in R. E. Banks, J. Chem. Soc. Perkin Trans. 1 (1973) 80-82:

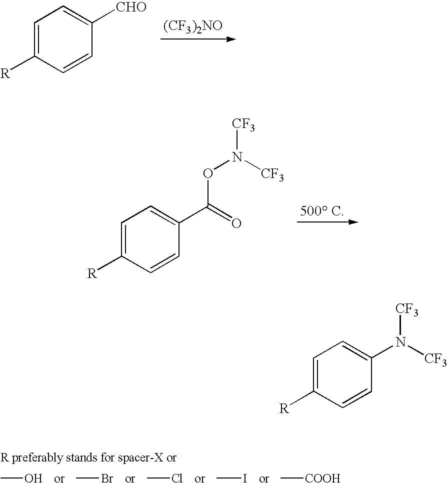

The end group CF₃NH— in compounds CF₃NH—R can be introduced by methods known from the literature by reaction of corresponding compounds Cl₂C═N—R with an excess of HF (corresponding syntheses are described, for example, in Petrow et al., Zh. Obshch. Khim. 29 (1959) 2169-2173 or E. Kuhle, Angew. Chem. 89 (No. 11) (1977), 797-804). Alternatively, trifluoromethyl isocyanate can be reacted with an alcohol to give a compound CF₃—NHC(═O)—O—R (in accordance with Knunyants et al. Mendeleev Chem. J. 22 (1977) 15-105 or Motornyi et al., Zh. Obshch. Khim. 29 (1959) 2157-2122). The corresponding starting materials are each obtainable by methods known from the literature, or compounds of the Cl₂C═N—R type can be obtained by reaction of compounds R—NH—CHO with chlorine and SOCl₂, and the radicals R in the products can be chemically modified by established methods.

Trifluoromethoxyaromatic compounds can be obtained by reaction of phenols with carbon tetrachloride and hydrogen fluoride.

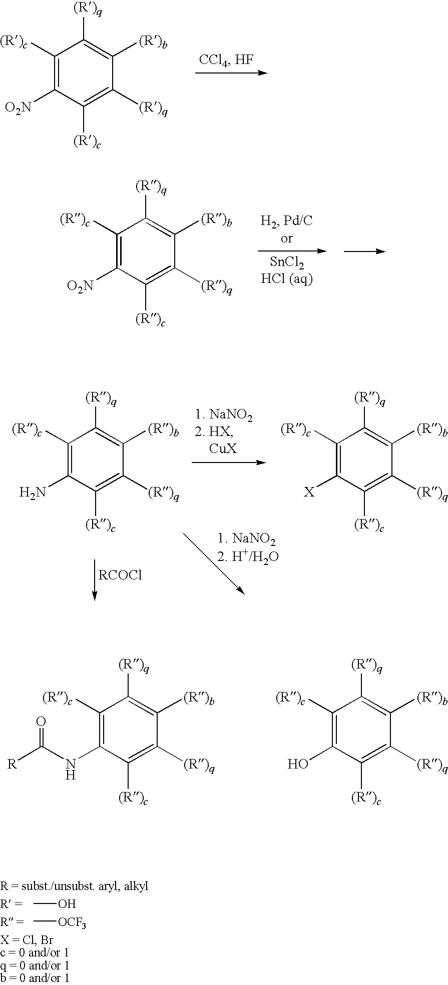

The following scheme shows a specific example:

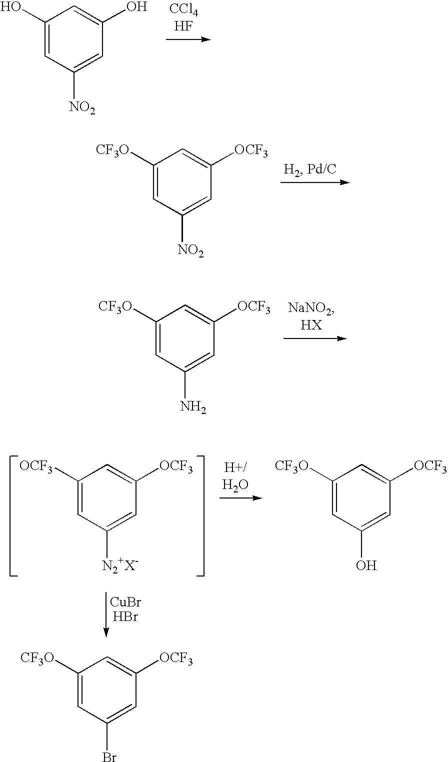

The starting material nitroresorcinol can be prepared in accordance with the following literature:
Funke; Krucker; BSCFAS; Bull. Soc. Chim. Fr.; 1953; 744, 746 or
Grosheintz; Fischer; JACSAT; J. Am. Chem. Soc.; 70; 1948; 1476, 1478.

The corresponding disclosure of the said methods in the references cited here thus expressly also belongs to the disclosure content of the present application.

The introduction of the hydrophilic, anionic, cationic, reactive or polymerisable component is possible via the corresponding co-fluorinated compounds, such as, for example, alcohols, aldehydes, carboxylic acids or alkenes, by methods known to the person skilled in the art. Examples are revealed by the following schemes and the example part:

The same reaction steps can also be carried out analogously with the respective other fluorinated end groups according to the invention. The choice of suitable solvents and reaction conditions for all reactions described here presents the person skilled in the art with absolutely no difficulties (Organikum: Organisch-Chemisches Grundpraktikum [Basic Practical Organic Chemistry], 16th Edn., VEB Deutscher Verlag der Wissenschaften, Berlin, 1986).

The following schemes show possible derivatisations of aryl-Rf building blocks from commercial or literature-known compounds.

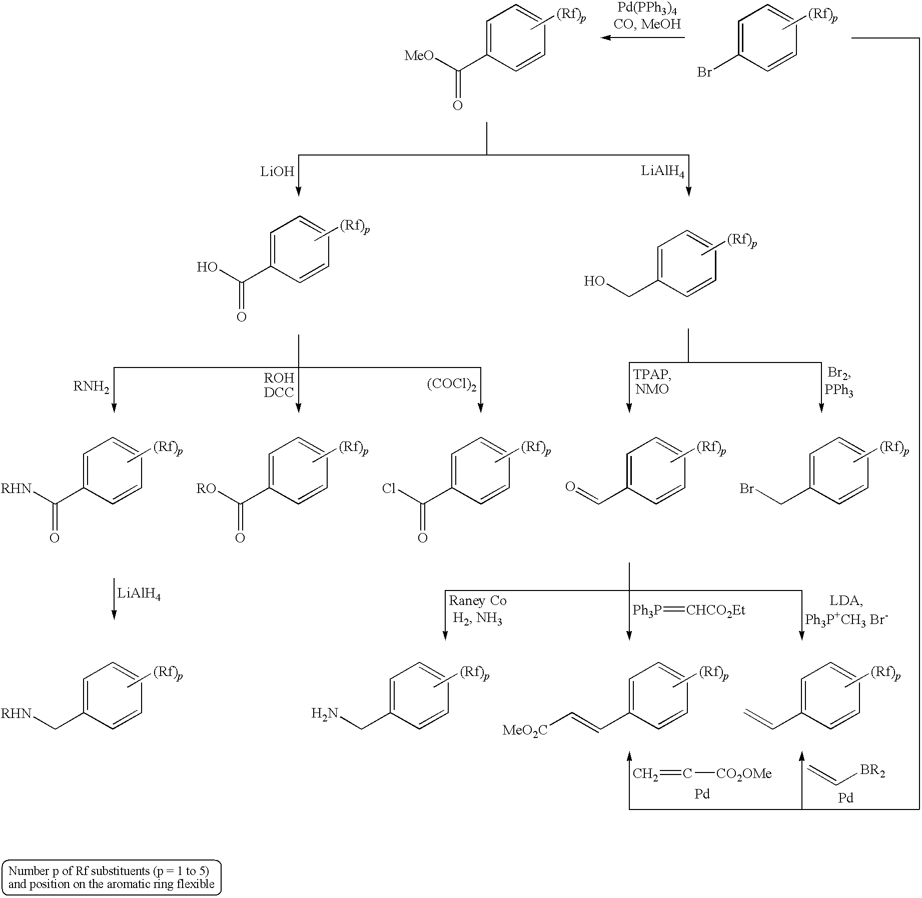

The bonding of a spacer to aryl-Rf or further links via various functionalities are shown in Schemes I to VIII:

### I. Etherification by Mitsunobu Reaction:

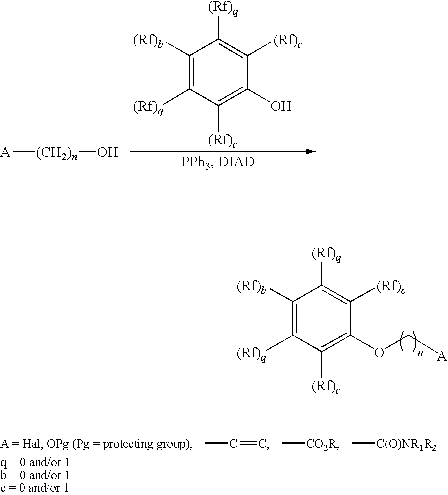

### II. Linking Via Thioether or Sulfone Unit

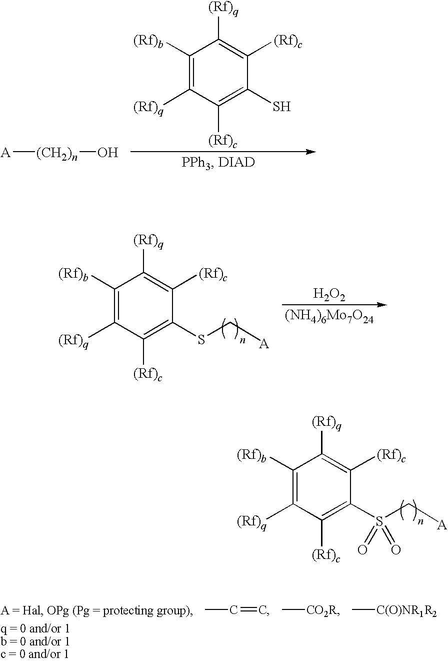

### III. Amine Formation

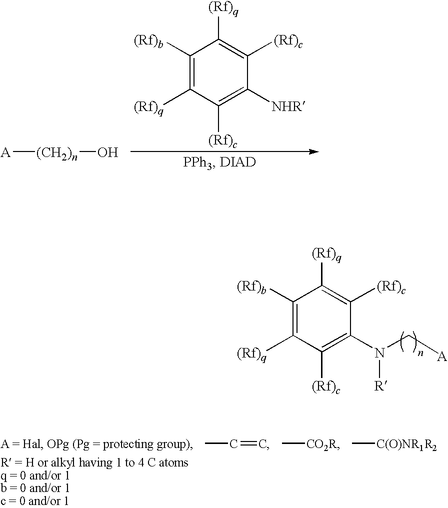

### IV. Esterification or Amide Formation

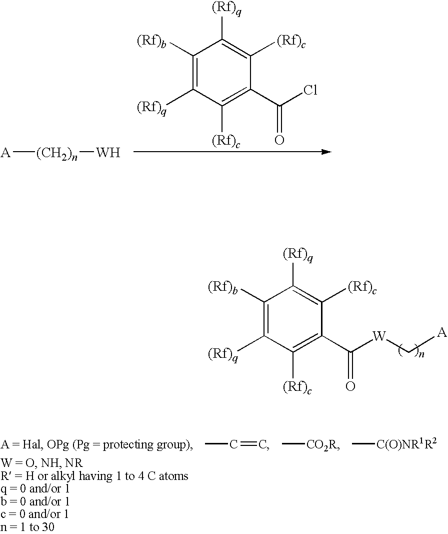

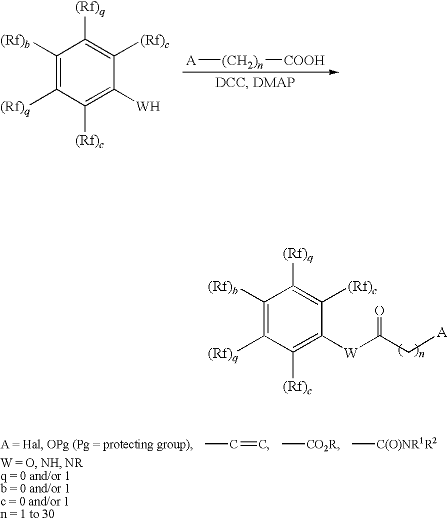

### V. Linking Via Sulfonic Acid Esters and Amides

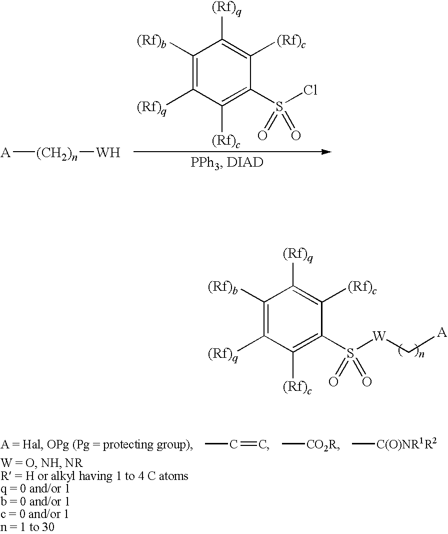

The arylsulfonyl chloride is obtained from the corresponding aromatic compound by reaction with ClSO₃H.

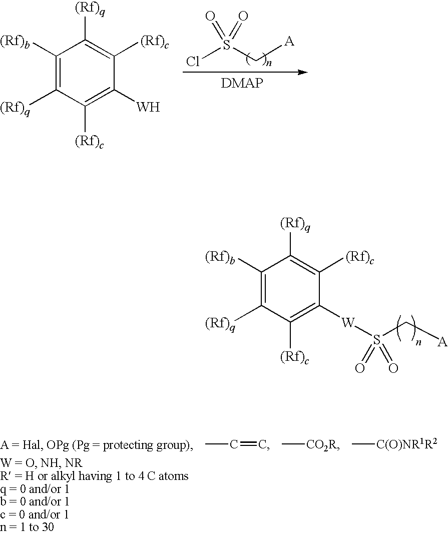

### VI. Linking Via Keto Function

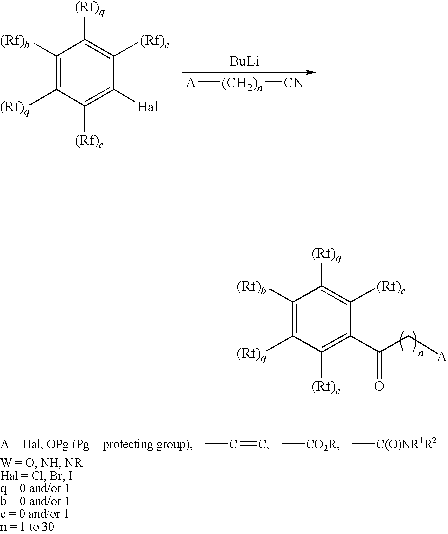

### VII. Linking Via Isocyanates or Isothiocyanates

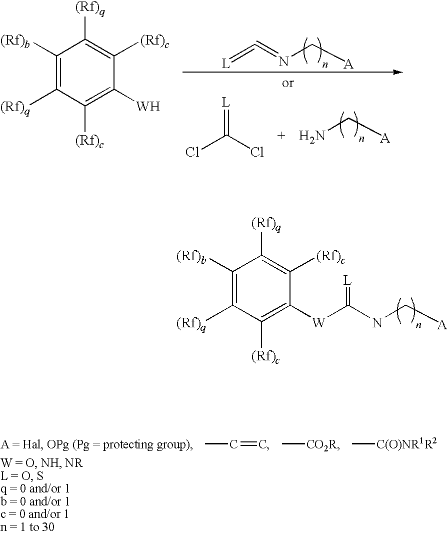

### VIII. Linking Via Heck Reaction

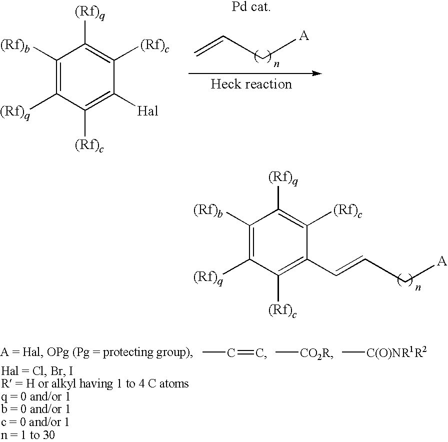

The following schemes show chain extensions, which can be carried out independently of Rf:

### Chain Extension by Cross-Metathesis:

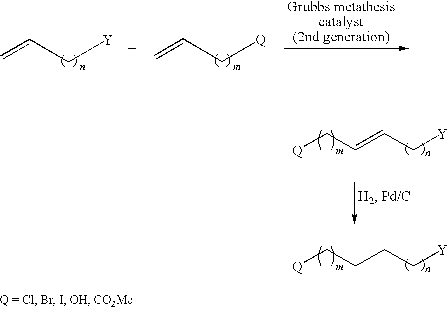

### Chain Extension by Free-Radical Thiol Addition:

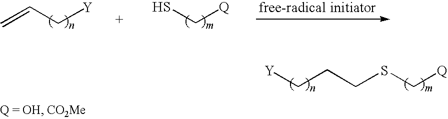

### Chain Extension by Wittig Reaction:

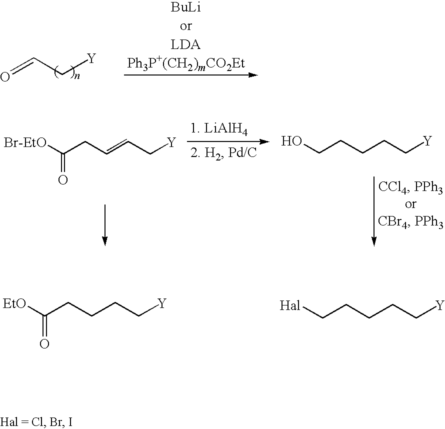

### Chain Extension by Williamson Ether Synthesis:

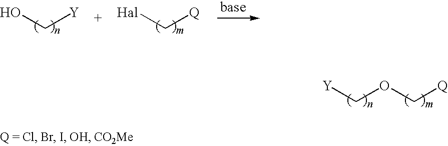

### Chain Extension by Thioether Synthesis:

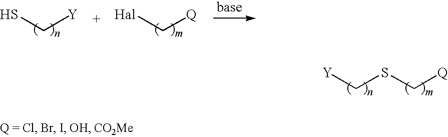

In addition, chain extensions via ester or amide formation are possible/can be carried out.

Derivatisation for Pg e.g. OBn:

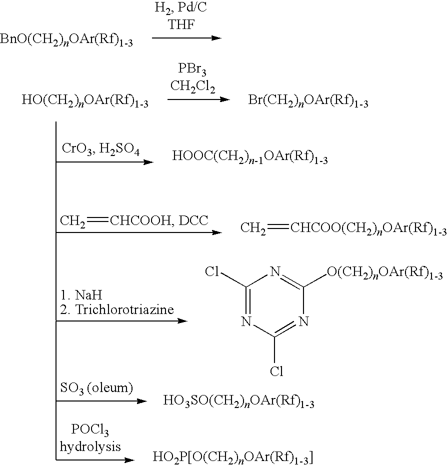

### Derivatisation for Hal=Br:

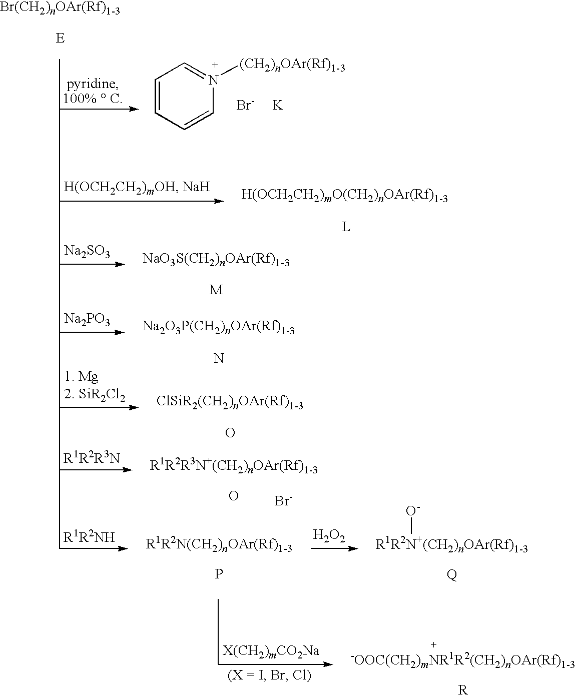

The present invention therefore furthermore relates to a process for the preparation of a compound of the formula I, as defined above, characterised in that the synthesis is carried out via the intermediate of a compound of the formula II

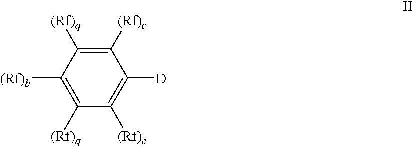

where
Rf stands for CF₃—(CH₂)ᵣ—, CF₃—(CH₂)ᵣ—O—, CF₃—(CH₂)ᵣ—S—, CF₃CF₂—S—, SF₅—(CH₂)ᵣ—, [CF₃—(CH₂)ᵣ]₂N—, [CF₃—(CH₂)ᵣ]NH— or (CF₃)₂N—(CH₂)ᵣ—,
D stands for OH, NH₂ or Br,
b stands for 0 or 1 and c stands for 0 or 1,
q stands for 0 or 1, where at least one radical from b and q stands for 1, and
r stands for 0, 1, 2, 3, 4 or 5.

Apart from the preferred compounds mentioned in the description, the use thereof, compositions and processes, further preferred combinations of the subject-matters according to the invention are disclosed in the claims.

The disclosures in the cited references thus expressly also belong to the disclosure content of the present application.

The following examples explain the present invention in greater detail without restricting the scope of protection. In particular, the features, properties and advantages described in the Examples of the compounds on which the particular examples are based can also be applied to other substances and compounds which are not mentioned in detail, but fall within the scope of protection, so long as nothing to the contrary is stated elsewhere. In addition, the invention can be carried out throughout the claimed range and is not restricted to the examples mentioned here.

### EXAMPLES

List of abbreviations used:

Bn: benzyl
DBH: 1,3-dibromo-5,5-dimethylhydantoin
DCM: dichloromethane
DMAP: 4-(dimethylamino)pyridine
Me: methyl
MTB: methyl tert-butyl ether
RT room temperature (20° C.)
THF: tetrahydrofuran
PE: petroleum ether
DCC N,N′-dicyclohexylcarbodiimide
TPAP tetra-n-propylammonium perruthenate
TLC thin-layer chromatography

### Example 1

### Example 1a

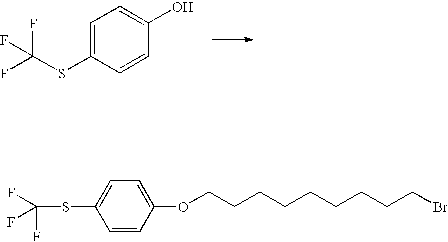

10.0 g (51.5 mmol) of 4-trifluoromethylthiophenol, 12.64 g (56.6 mmol) of 9-bromo-1-nonanol and 14.9 g (56.6 mmol) of triphenylphosphine are initially introduced in 200 ml of THF. 11.0 ml of diisopropyl azodicarboxylate are added dropwise at max. 15° C. with cooling, and the mixture is stirred at room temperature for 24 hrs. The solvent is stripped off in a rotary evaporator, and the crude product is purified by column chromatography (eluent: heptane/DCM 1/1) over silica gel, giving a yellow oil.

### Example 1b

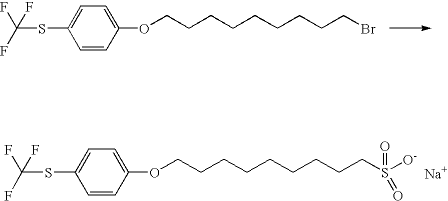

19.3 g (45.4 mmol) of 1-(9-bromononyloxy)-4-(trifluoromethylthio)benzene and 7.4 g (59.1 mmol) of sodium sulfite are dissolved in a mixture of 140 ml of ethanol and 140 ml of deionised water, and the mixture is heated at 100° C. for 20 hrs. The mixture is subsequently extracted with a little MTB ether/heptane (1:1). The aqueous phase is acidified using conc. sulfuric acid (pH=0) and extracted five times with MTB ether. The combined organic phases are dried over sodium sulfate and filtered.

The solvent is distilled off in a rotary evaporator. The intermediate sulfonic acid crude product (yellow oil) is employed directly in the next step.

### Example 1c

A mixture of 1.9 g (47.3 mmol) of NaOH pellets in 73 ml of EtOH is added at RT to 16.8 g (39.4 mmol) of the sulfonic acid crude product.

The reaction mixture is heated under reflux for one hour (oil bath 95° C.). The mixture is subsequently cooled to RT, and the colourless solid formed is filtered off with suction and dried at 80° C. in vacuo for 7 days.

¹H-NMR (400 MHz, D₂O) δ=7.39 (d, 2H); 6.73 (d, 2H); 3.75 (t, 2H); 2.78 (m, 2H); 1.73-1.55 (m, 4H); 1.37-1.10 (m, 10H) ppm.

¹⁹F-NMR (400 MHz, D₂O) δ=−43.85 (s, 3F, CF₃) ppm.

### Example 2

### Example 2a

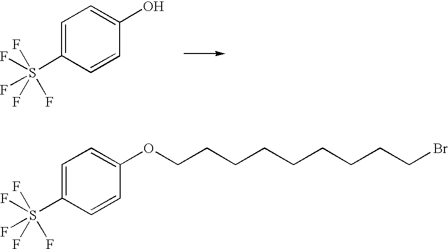

10.0 g (45.4 mmol) of 4-pentafluorothiophenol, 11.7 g (50 mmol) of 9-bromo-1-nonanol and 13.1 g (50 mmol) of triphenylphosphine are initially introduced in 180 ml of THF. 9.7 ml of diisopropyl azodicarboxylate are added dropwise at max. 15° C. with cooling, and the mixture is stirred at room temperature for 24 hrs. The solvent is stripped off in a rotary evaporator, and the crude product is purified by column chromatography (eluent: heptane/DCM 1/1) over silica gel, giving a colourless oil.

### Example 2b

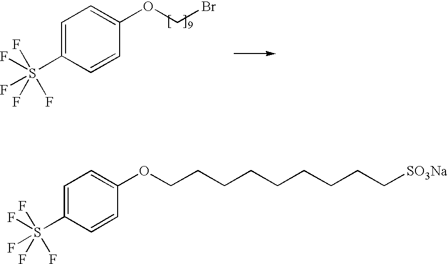

16.5 g (37.6 mmol) of 1-(9-bromononyloxy)-4-(trifluoromethylthio)benzene and 6.2 g (48.9 mmol) of sodium sulfite are dissolved in a mixture of 150 ml of ethanol and 150 ml of deionised water, and the mixture is heated at 100° C. for 20 hrs. The mixture is subsequently extracted with a little MTB ether/heptane (1:1). The aqueous phase is acidified using conc. sulfuric acid (pH=0) and extracted five times with MTB ether. The combined organic phases are dried over sodium sulfate and filtered.

The solvent is distilled off in a rotary evaporator. The intermediate sulfonic acid crude product (colourless oil) is employed directly in the next step.

### Example 2c

A mixture of 1.3 g (32.9 mmol) of NaOH pellets in 76 ml of EtOH is added at RT to 14.05 g (32.9 mmol) of the sulfonic acid crude product.

The reaction mixture is heated under reflux for one hour (oil bath 95° C.). The mixture is subsequently cooled to RT, and the colourless solid formed is filtered off with suction and dried at 80° C. in vacuo for 7 days.

¹H-NMR (400 MHz, D₂O) δ=7.49 (d, 2H); 6.72 (d, 2H); 3.80 (t, 2H); 2.78 (m, 2H); 1.71-1.55 (m, 4H); 1.35-1.12 (m, 10H) ppm.

¹⁹F-NMR (400 MHz, D₂O) δ=86.75 (q, 1F); 64.32 (d, 4F) SF₅ ppm.

### Example 3

### Example 3a

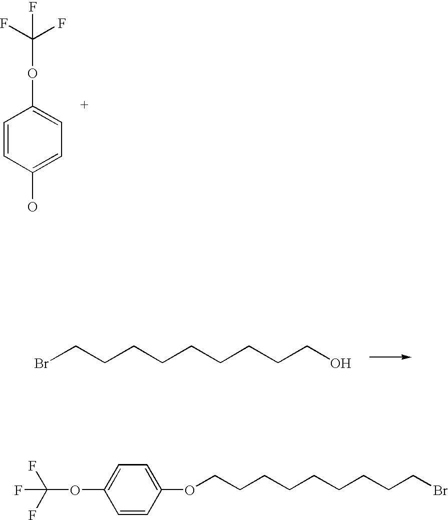

10.0 g (56.1 mmol) of 4-trifluoromethoxyphenol, 14.5 g (62 mmol) of 9-bromo-1-nonanol and 16.2 g (62 mmol) of triphenylphosphine are initially introduced in 230 ml of THF. 12.5 ml of diisopropyl azodicarboxylate are added dropwise at max. 15° C. with cooling, and the mixture is stirred at room temperature for 24 hrs. The solvent is stripped off in a rotary evaporator, and the crude product is purified by column chromatography (eluent: heptane/DCM 1/1) over silica gel, giving a colourless oil.

### Example 3b

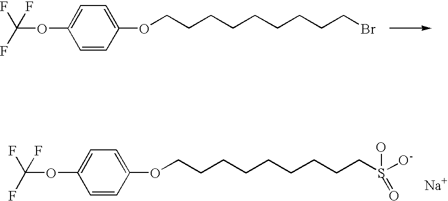

22.9 g (58.1 mmol) of 1-(9-bromononyloxy)-4-(trifluoromethylthio)benzene and 9.5 g (75.5 mmol) of sodium sulfite are dissolved in a mixture of 140 ml of ethanol and 140 ml of deionised water, and the mixture is heated at 100° C. for 20 hrs. The mixture is subsequently extracted with a little MTB ether/heptane (1:1). The aqueous phase is acidified using conc. sulfuric acid (pH=0) and extracted five times with MTB ether. The combined organic phases are dried over sodium sulfate and filtered.

The solvent is distilled off in a rotary evaporator. The intermediate sulfonic acid crude product (yellow oil) is employed directly in the next step.

### Example 3c

A mixture of 2.13 g (53.2 mmol) of NaOH pellets in 82 ml of EtOH is added at RT to 17.95 g (44.3 mmol) of the sulfonic acid crude product. The reaction mixture is heated under reflux for one hour (oil bath 95° C.). The mixture is subsequently cooled to RT, and the colourless solid formed is filtered off with suction and dried at 80° C. in vacuo for 7 days.

¹H-NMR (400 MHz, D₂O) δ=7.29 (d, 2H); 7.04 (d, 2H); 4.08 (t, 2H); 2.88 (m, 2H); 1.80-1.66 (m, 4H); 1.48-1.29 (m, 10H) ppm.

¹⁹F-NMR (400 MHz, D₂O) δ=−58.21 (s, 3F, CF₃) ppm.

### Trifluoromethyl-Substituted Aromatic Compounds

### Example 4

### p-Trifluoromethylphenoxy Substitution

### Example 4a

### Preparation of 1-(9-bromononyloxy)-4-trifluoromethylbenzene

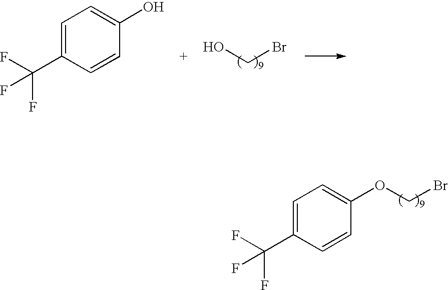

10.0 g (61.68 mmol) of 4-hydroxybenzotrifluoride, 15.14 g (67.86 mmol) of 9-bromo-1-nonanol and 17.8 g (67.86 mmol) of triphenylphosphine are initially introduced in 250 ml of THF. 13.19 ml of diisopropyl azodicarboxylate are added dropwise at max. 15° C. with cooling, and the mixture is stirred at room temperature for 24 hrs. The solvent is stripped off in a rotary evaporator, and the crude product is purified by column chromatography (eluent: heptane/DCM 1/1) over silica gel, giving a yellow oil.

¹H-NMR (300 MHz, CDCl₃) δ=7.52 (d, 2H); 6.93 (d, 2H); 3.97 (t, 2H); 3.40 (t, 2H); 1.95-1.72 (m, 4H); 1.53-1.24 (m, 10H) ppm.

¹⁹F-NMR (250 MHz, CDCl₃) δ=−63.34 (s, 3F, CF₃) ppm.

### Example 4b

### Preparation of 9-(4-trifluoromethylphenoxy)nonane-1-sulfonic Acid Sodium Salt

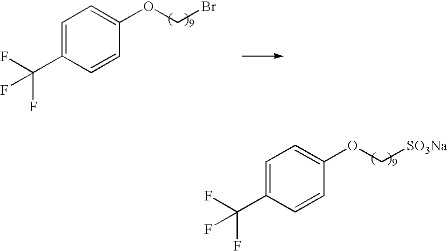

22.2 g (59.18 mmol) of 1-(9-bromononyloxy)-4-trifluoromethylthiobenzene and 9.7 g (76.94 mmol) of sodium sulfite are dissolved in a mixture of 140 ml of ethanol and 140 ml of deionised water, and the mixture is heated at 100° C. for 20 hrs. The mixture is subsequently extracted with a little MTB ether/heptane (1:1). The aqueous phase is acidified using conc. sulfuric acid (pH=0) and extracted five times with MTB ether. The combined organic phases are dried over sodium sulfate and filtered.

The solvent is distilled off in a rotary evaporator. The intermediate sulfonic acid crude product (yellow oil) is investigated by spectroscopy.

¹H-NMR (300 MHz, DMSO-d₆) δ=7.62 (d, 2H); 7.11 (d, 2H); 4.04 (t, 2H); 2.60-2.51 (m, 2H); 1.73 (quin, 2H); 1.65-1.51 (m, 2H); 1.49-1.18 (m, 10H) ppm.

¹⁹F-NMR (250 MHz, DMSO-d₆) δ=−57.87 (s, 3F, CF₃) ppm.

### Example 4c

A mixture of 2.13 g (53.18 mmol) of NaOH pellets in 82 ml of EtOH is added at RT to 17.95 g (44.31 mmol) of the sulfonic acid crude product. The reaction mixture is heated under reflux for one hour (oil bath 95° C.). The mixture is subsequently cooled to RT, and the colourless solid formed is filtered off with suction and dried at 80° C. in vacuo for 7 days.

¹H-NMR (400 MHz, DMSO-d₆) δ=7.63 (d, 2H); 7.11 (d, 2H); 4.04 (t, 2H); 2.39 (m, 2H); 1.73 (quin, 2H); 1.61-1.51 (m, 2H); 1.46-1.19 (m, 10H) ppm.

¹³C-NMR (100 MHz, DMSO-d₆) δ=161.5; 126.9; 126.8; 121.0-120.7; 114.9; 67.9; 51.5; 28.8-25.1 ppm.

¹⁹F-NMR (280 MHz, D₂O) δ=−61.14 (s, 3F, CF₃) ppm.

### Example 4d

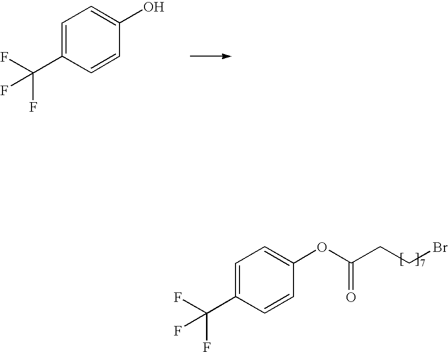

13.8 g of 8-bromooctanoic acid (62 mmol), 0.76 g of 4-(N,N-dimethylamino)pyridine (6.2 mmol; 0.1 eq), 13 g of DCC (62 mmol) and 8 ml of triethylamine (62 mmol) are initially introduced in 150 ml of dichloromethane in a 250 ml round-bottomed flask, the mixture is stirred at RT for 2 hours, and 10 g of p-(trifluoromethyl)phenol (62 mmol) in 50 ml of DCM are then slowly added dropwise at 0° C. The reaction mixture is stirred at RT (TLC check). 200 ml of H₂O are added to the reaction mixture, which is acidified slightly (pH=5-6). The phases are separated, and the aqueous phase is extracted three times with 100 ml of DCM each time. The combined organic phases are washed with saturated NaCl solution and dried over sodium sulfate. After the drying agent has been filtered off, the solvent is removed in vacuo. Purification of the crude product by column chromatography (PE/MTBE 20/1 to 1/1) gives a yellowish oil.

### Example 4e

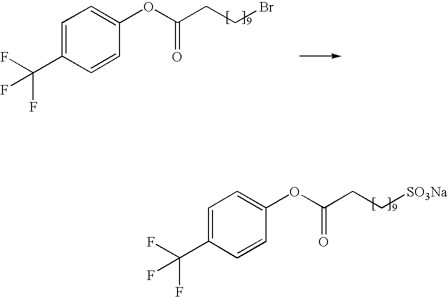

10 g of 8-bromooctyl 4-trifluoromethylbenzoate (26.2 mmol, 1 eq) and 4 g of sodium sulfite (31.5 mmol, 1.2 eq) are initially introduced in 100 ml of ethanol and 100 ml of water in a 500 ml one-necked flask and heated to 100° C. The mixture is stirred under reflux overnight (20 hrs), and the conversion is monitored by TLC. When the reaction is complete, the mixture is washed by shaking with a little MTBE/heptane 1:1 in order to remove the starting material and impurities. The aqueous phase is then concentrated in a freeze drier and freed from inorganic salts by column chromatography with MTBE/MeOH 1/1. The product is a colourless solid, which is dried at 30° C. and 0.1 bar in a vacuum drying cabinet.

¹H-NMR (300 MHz, DMSO-d₆) δ=7.62 (d, 2H); 7.32 (d, 2H); 3.40 (t, 2H); 2.34 (m, 2H); 1.83-1.35 (m, 4H); 1.45-1.20 (m, 12H) ppm.

### Example 5

### p-Trifluoromethylbenzoyl Substitution

### Example 5a

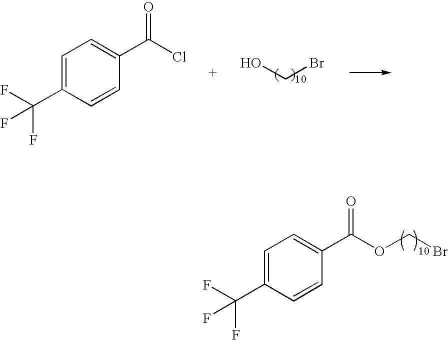

8 g of 10-bromodecan-1-ol (33.56 mmol, 1 eq) are initially introduced in 300 ml of dry dichloromethane at RT, and 3.74 g of triethylamine (36.9 mmol, 1.1 eq) are added. The reaction mixture is subsequently cooled to 0° C., and 7 g of 4-trifluoromethylbenzoyl chloride (33.56 mmol, 1 eq) in 30 ml of dichloromethane are added via a dropping funnel at such a rate that the internal temperature does not exceed 10° C. When the addition is complete, the reaction mixture is slowly warmed to RT. When the reaction is complete (TLC check), the reaction mixture is quenched using saturated NaHCO₃ solution. The organic phase is dried over Na₂SO₄ and evaporated in a rotary evaporator. A brownish oil forms, which is adsorbed onto silica gel and purified by column chromatography (PE). The product is a yellowish oil.

¹H-NMR (300 MHz, CDCl₃) δ=8.12 (d, 2H); 7.90 (d, 2H); 4.49 (t, 2H); 3.38 (t, 2H); 1.86-1.65 (m, 4H); 1.49-1.20 (m, 12H) ppm.

### Example 5b

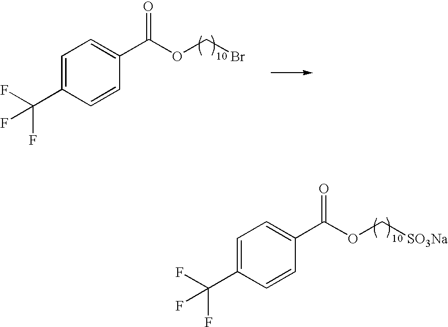

15 g of 10-bromodecyl 4-trifluoromethylbenzoate (36.65 mmol, 1 eq) and 5.6 g of sodium sulfite (44 mmol, 1.2 eq) are initially introduced in 140 ml of ethanol and 140 ml of water in a 500 ml one-necked flask and heated to 100° C. The mixture is stirred under reflux overnight (20 hrs), and the conversion is monitored by TLC. When the reaction is complete, the mixture is washed by shaking with a little MTBE/heptane 1:1 in order to remove the starting material and impurities. The aqueous phase is then concentrated in a freeze drier and freed from inorganic salts by column chromatography with MTBE/MeOH 1/1. The product is a colourless solid, which is dried at 30° C. and 0.1 bar in a vacuum drying cabinet.

¹H-NMR (400 MHz, DMSO-d₆) δ=8.10 (d, 2H); 7.90 (d, 2H); 4.39 (t, 2H); 2.39 (t, 2H); 1.73 (quin, 2H); 1.61-1.51 (m, 2H); 1.46-1.19 (m, 12H) ppm.

### Example 5c

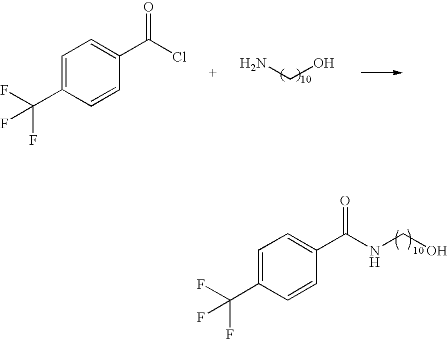

6 g of 10-aminodecan-1-ol (34.6 mmol, 1 eq) are initially introduced in 250 ml of dry toluene at RT, and 4.2 g of triethylamine (41.5 mmol, 1.2 eq) are added. The reaction mixture is subsequently cooled to 0° C., and 7.2 g of 4-trifluoromethylbenzoyl chloride (34.6 mmol, 1 eq) in 30 ml of dichloromethane are slowly added via a dropping funnel. When the addition is complete, the reaction mixture is slowly warmed to RT. When the reaction is complete (TLC check), the reaction mixture is quenched using saturated NaHCO₃ solution. The organic phase is dried over Na₂SO₄ and evaporated in a rotary evaporator. A brownish oil forms, which is adsorbed onto silica gel and purified by column chromatography (PE). The product is a yellowish oil.

¹H-NMR (300 MHz, CDCl₃) δ=8.08 (d, 2H); 7.96 (d, 2H); 4.48 (s, 1H), 3.59 (t, 2H); 3.45 (t, 2H); 1.54 (quin., 2H); 1.49-1.25 (m, 14H) ppm.

The amide can alternatively be prepared from the commercial ethyl ester in THF in the presence of catalytic amounts of N-heterocyclic carbenes: M. Movassaghi et al. Org. Lett. 2005, 12, 2453-2456.

### Example 5d

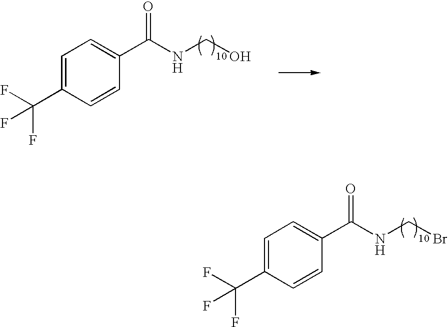

4 g of N-(10-hydroxydecyl)-4-trifluoromethylbenzamide (11.6 mmol, 1 eq) are initially introduced in 100 ml of dry dichloromethane at RT, and 4.6 g of triphenylphosphine (17.4 mmol, 1.5 eq) are added. The reaction mixture is subsequently cooled to 0° C., and 6.54 g of tetrabromomethane (19.2 mmol, 1.7 eq), dissolved in 15 ml of dichloromethane, are added via a syringe at a controlled rate such that the internal temperature does not exceed 10° C. When the addition is complete, the reaction mixture is slowly warmed to RT. When the reaction is complete (TLC check), the reaction mixture is quenched using saturated NaHCO₃ solution. The organic phase is dried over Na₂SO₄ and evaporated in a rotary evaporator. A brown residue forms, which is adsorbed onto silica gel and purified by column chromatography (PE/MTBE 20/1). The product is a pale-yellowish oil.

¹H-NMR (300 MHz, CDCl₃) δ=8.08 (d, 2H); 7.96 (d, 2H); 6.30 (s, 1H); 3.44 (m, 4H); 1.84 (m, 2H); 1.59-1.18 (m, 14H) ppm.

The alcohol can be converted into the corresponding bromide using Ph₃P—Br₂ analogously to M. A. Casadei et al. Tetrahedron 1988, 44, 2601-2606; using PBr₃ in ethyl acetate in accordance with A. Pain et al. Acta Pol. Pharm. 2003, 60, 285-292 or in accordance with Y. Liu et al. Bioorg. Med. Chem. 2006, 14, 2935-2941 (Br₂/PCl₃).

### Example 5e

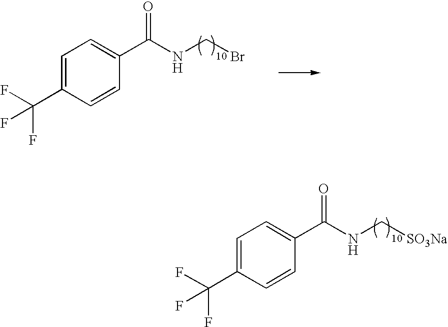

10 g of N-(10-bromodecyl)-4-trifluoromethylbenzamide (24.5 mmol, 1 eq) and 3.74 g of sodium sulfite (29.4 mmol, 1.2 eq) are initially introduced in 90 ml of ethanol and 90 ml of water in a 250 ml one-necked flask and heated to 100° C. The mixture is stirred under reflux overnight (20 hrs), and the conversion is monitored by TLC. When the reaction is complete, the mixture is washed by shaking with a little MTBE/heptane 1:1 in order to remove the starting material and impurities. The aqueous phase is then concentrated in a freeze drier and freed from inorganic salts by column chromatography with MTBE/MeOH 1/1. The product is a colourless solid, which is dried at 30° C. and 0.1 bar in a vacuum drying cabinet.

¹H-NMR (300 MHz, DMSO-d₆) δ=8.05 (d, 2H); 7.90 (d, 2H); 3.45 (t, 2H); 2.39 (t, 2H); 1.73 (quin, 2H); 1.60-1.50 (m, 2H); 1.45-1.18 (m, 12H) ppm.

### Example 6

### p-Trifluoromethylphenylamine Substitution

### Example 6a

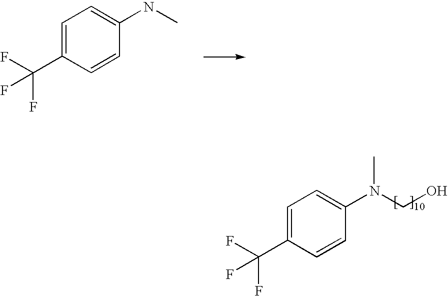

16.5 g of sodium iodide (110 mmol) and 19.3 g of 10-chlorodecan-1-ol (100 mmol) are initially introduced in 500 ml of acetone in a 1000 ml round-bottomed flask and stirred under reflux for 24 h. The residue is subsequently filtered off, and the filtrate is evaporated in a rotary evaporator. The residue is taken up using 300 ml of butanol, 13.8 g of potassium carbonate (100 mmol) and 16.6 g of N,N-(p-trifluoromethyl)phenylmethylamine (95 mmol, procedure: see: E. B. Siogren et al. J. Med. Chem. 1991, 34, 11, 3295-3301) are added, and the mixture is heated to reflux. The reaction mixture is stirred at 60° C. with TLC monitoring. Some of the solvent is then removed in vacuo, the residue is taken up in water, and the solution is extracted with 4×100 ml of MTBE. The combined organic phases are washed with 1×200 ml of H₂O and 1×200 ml of saturated NaCl solution and dried over Na₂SO₄, and finally the solvent is removed in vacuo.

¹H-NMR (300 MHz, CDCl₃) δ=7.41 (d, 2H); 6.73 (d, 2H); 3.58 (t, 2H); 3.35 (m, 2H); 3.09 (s, 3H), 2.60 (s, 1H); 1.84 (m, 2H); 1.57 (m, 2H); 1.49-1.19 (m, 12H) ppm.

### Example 6b

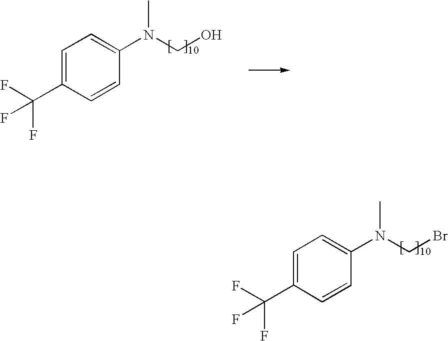

6 g of (10-hydroxydecyl)methyl-(4-trifluoromethylphenyl)amine (18.1 mmol, 1 eq) are initially introduced in 180 ml of dry dichloromethane at RT, and 7.1 g of triphenylphosphine (27.2 mmol, 1.5 eq) are added. The reaction mixture is subsequently cooled to 0° C., and 10.2 g of tetrabromomethane (30.8 mmol, 1.7 eq), dissolved in 40 ml of dichloromethane, are added via a dropping funnel at a controlled rate such that the internal temperature does not exceed 10° C. When the addition is complete, the reaction mixture is slowly warmed to RT. When the reaction is complete (TLC check), the reaction mixture is quenched using saturated NaHCO₃ solution. The organic phase is dried over Na₂SO₄ and evaporated in a rotary evaporator. A brown residue forms, which is adsorbed onto silica gel and purified by column chromatography (PE/MTBE 3/1). The product is a pale-yellowish oil.

¹H-NMR (300 MHz, CDCl₃) δ=7.41 (d, 2H); 6.73 (d, 2H); 3.42 (t, 2H); 3.35 (m, 2H); 3.09 (s, 3H), 1.84 (m, 2H); 1.54 (m, 2H); 1.41-1.15 (m, 12H) ppm.

### Example 6c

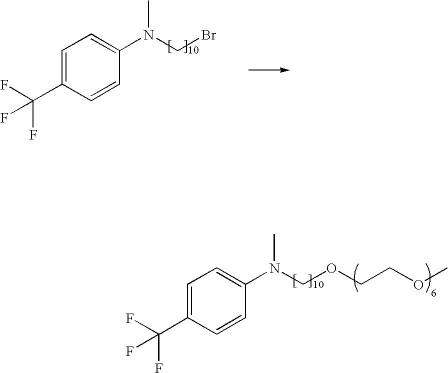

A solution of 4.1 g (13.95 mmol) of hexaethylene glycol monomethyl ether in 40 ml of THF is added dropwise at a controlled rate at RT to a suspension of 0.6 g of NaH (60%, 13.95 mmol) in 60 ml of THF. The mixture is stirred at RT for 2 hrs, and 5 g (12.7 mmol) of (10-bromodecyl)-methyl-(4-trifluoromethylphenyl)amine are subsequently added at RT. The reaction mixture is stirred for 10 hrs and then quenched using sat. NH₄Cl solution. The phases are separated, and the organic phase is evaporated to dryness.

¹H-NMR (400 MHz, CDCl₃) δ=7.44 (d, 2H); 6.66 (d, 2H); 3.75-3.69 (m, 4H); 3.69-3.53 (m, 16H); 3.51 (s, 3H); 3.44-3.37 (m, 6H); 3.08 (m, 5H), 2.84 (s, 1H); 1.74 (m, 2H); 1.59-1.19 (m, 12H) ppm.

### Example 6d

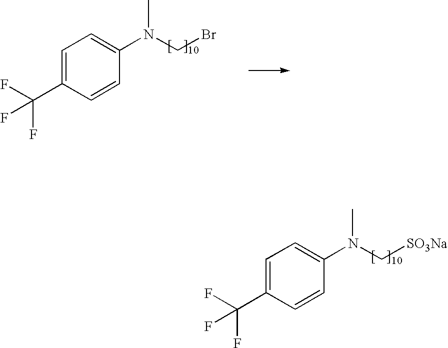

3 g of (10-bromodecyl)methyl-(4-trifluoromethylphenyl)amine (7.6 mmol, 1 eq) and 1.15 g of sodium sulfite (9.1 mmol, 1.2 eq) are initially introduced in 20 ml of ethanol and 20 ml of water in a 100 ml one-necked flask and heated to 100° C. The mixture is stirred under reflux overnight (20 hrs), and the conversion is monitored by TLC. When the reaction is complete, the mixture is washed by shaking with a little MTBE/heptane 1:1 in order to remove the starting material and impurities. The aqueous phase is then concentrated in a freeze drier and freed from inorganic salts by column chromatography with MTBE/MeOH 1/1. The product is a colourless solid, which is dried at 30° C. and 0.1 bar in a vacuum drying cabinet.

¹H-NMR (400 MHz, CDCl₃) δ=7.42 (d, 2H); 6.74 (d, 2H); 3.42 (t, 2H); 3.09 (s, 3H), 2.39 (m, 2H); 1.84 (m, 2H); 1.54 (m, 2H); 1.41-1.15 (m, 12H) ppm.

### Example 6e

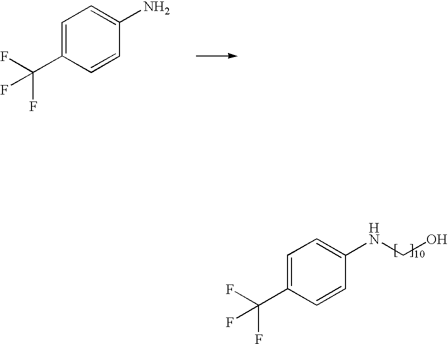

Chlorodecan-1-ol is synthesised in accordance with G. van Koten et al. J. Org. Chem. 1998, 63, 4282-4290 from the corresponding aniline and 1,10-decanediol with ruthenium catalysis or in accordance with F. Jourdain et al. Tetrahedron Lett. 1994, 35, 1545-1548 from 10-chlorodecan-1-ol by nucleophilic substitution.

16.5 g of sodium iodide (110 mmol) and 19.3 g of 10-chlorodecan-1-ol (100 mmol) are initially introduced in 500 ml of acetone in a 1000 ml round-bottomed flask and stirred under reflux for 24 h. The residue is subsequently filtered off, and the filtrate is evaporated in a rotary evaporator. The residue is taken up in toluene, and 32.2 g of p-(trifluoromethyl)aniline (200 mmol) are added, and the mixture is heated to reflux. When conversion is complete (TLC check), the reaction mixture is cooled, and the solvent is stripped off. The residue is adsorbed onto silica gel, and the crude product is purified by column chromatography.

### Example 6f

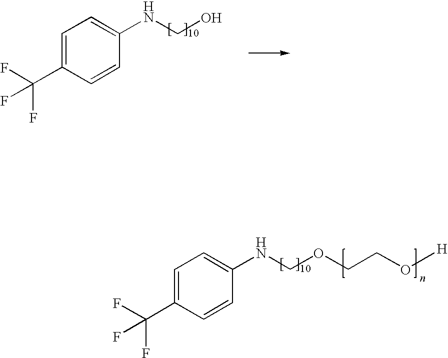

10 g of the amino alcohol are converted into the corresponding oligoethylene glycol in an autoclave in the presence of a catalytic amount of hydrotalcite (0.5 g) under an ethylene oxide atmosphere, giving a yellow oil, which has an average ethylene glycol:amino alcohol ratio of about 6 (n about 6).

¹H-NMR (400 MHz, CDCl₃) δ=7.44 (d, 2H); 6.66 (d, 2H); 3.75-3.69 (m, 4H); 3.69-3.53 (m, ˜16H); 3.44-3.37 (m, ˜6H); 3.09 (m, 2H), 2.82 (s, 2H); 1.74 (m, 2H); 1.59-1.19 (m, 12H) ppm.

### Example 6g

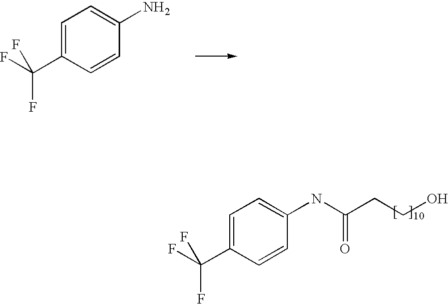

A solution of 5 g (18.6 mmol) of 10-bromodecanoyl chloride (preparation in accordance with Y. Ohtsuka Chem. Pharm. Bull. 1983, 31, 454) is added dropwise at 0° C. to 3 g of 4-trifluoromethylaniline (18.6 mmol) in 50 ml of THF and 3.1 ml of triethylamine (22.3 mmol). The mixture is subsequently warmed to RT and stirred for 2 hrs. 20 ml of 1 N NaOH solution are subsequently added at RT, and the mixture is stirred vigorously. When the reaction is complete, the mixture is diluted with MTBE and quenched using saturated NH₄Cl solution, and the organic phase is dried over Na₂SO₄ and evaporated in a rotary evaporator. The crude product is purified by column chromatography.

¹H-NMR (400 MHz, CDCl₃) δ=7.72 (d, 2H); 7.60 (d, 2H); 6.25 (s, 2H); 3.58 (t, 2H); 2.35 (m, 2H); 1.73 (m, 2H); 1.59-1.20 (m, 14H) ppm.

### Example 6h

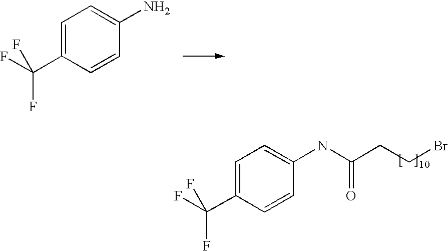

A solution of 4.6 g (18.6 mmol) of 10-acetoxydecanoyl chloride (preparation in accordance with G. Goto Chem. Pharm. Bull. 1985, 33, 4422) is added dropwise at 0° C. to 3 g of 4-trifluoromethylaniline (18.6 mmol) in 50 ml of THF and 3.1 ml of triethylamine (22.3 mmol). The mixture is subsequently warmed to RT and stirred for 2 hrs. 20 ml of 1 N NaOH solution are subsequently added at RT, and the mixture is stirred vigorously. When the reaction is complete, the mixture is diluted with MTBE and quenched using saturated NH₄Cl solution, and the organic phase is dried over Na₂SO₄ and evaporated in a rotary evaporator. The crude product is purified by column chromatography.

¹H-NMR (400 MHz, CDCl₃) δ=7.72 (d, 2H); 7.59 (d, 2H); 3.35 (t, 2H); 2.34 (m, 2H); 1.83-1.35 (m, 4H); 1.45-1.20 (m, 12H) ppm.

### Example 6i

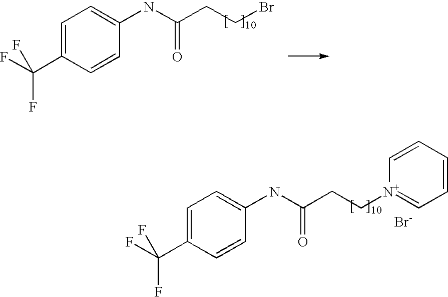

N-(4-Trifluoromethylphenyl)-12-bromododecanamide (4 g; 9.5 mmol) is heated under reflux for 2 days in 40 ml of pyridine. When the reaction is complete, the excess pyridine is stripped off. The surfactant product (1-[11-(4-trifluoromethylphenylcarbamoyl)undecyl]pyridinium bromide) obtained in this way can, if necessary, be purified by recrystallisation.

¹H-NMR (400 MHz, CDCl₃) δ=9.79 (s, 1H); 8.79 (m, 1H); 8.44 (m, 1H); 8.01-7.55 (m, 7H); 4.61 (t, 2H); 2.34 (t, 2H); 2.08 (m, 2H); 1.72 (m, 2H), 1.45-1.20 (m, 12H) ppm.

2,4-Bistrifluoromethylbenzylamine can also be converted into the corresponding surfactant in accordance with Example 4e.

### Example 7

### p-Trifluoromethylbenzenethiol Substitution

### Example 7a

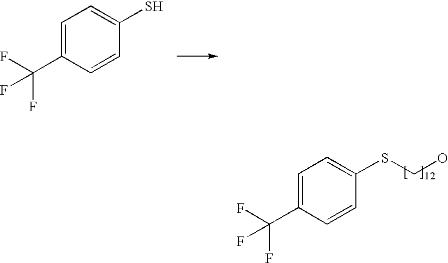

10.1 g of sodium iodide (67.3 mmol, 1.2 eq) and 13.6 g of 12-chlorododecan-1-ol (61.7 mmol, 1.1 eq) are initially introduced in 500 ml of acetone in a 1000 ml round-bottomed flask and stirred under reflux for 24 h. The residue is subsequently filtered off, and 8.53 g of potassium carbonate (61.7 mmol) and 10 g of p-(trifluoromethyl)thiophenol (56 mmol) are added to the filtrate, and the mixture is heated to reflux. The mixture is stirred under reflux until conversion is complete. Some of the acetone is then removed in vacuo, the residue is taken up in water, and the solution is extracted with 4×100 ml of MTBE. The combined org. phases are washed with 1×100 ml of H₂O and 1×100 ml of sat. NaCl solution and dried over Na₂SO₄, and the solvent is removed in a rotary evaporator. The crude product is purified by column chromatography on silica gel (MTBE/heptane ⅕ to ⅓).

¹H-NMR (400 MHz, CDCl₃) δ=7.52-7.45 (m, 4H); 3.58 (t, 2H); 2.93 (t, 2H); 2.26 (s, 1H); 1.81-1.77 (m, 2H); 1.59-1.47 (m, 4H); 1.38-1.19 (m, 10H) ppm.

### Example 7b

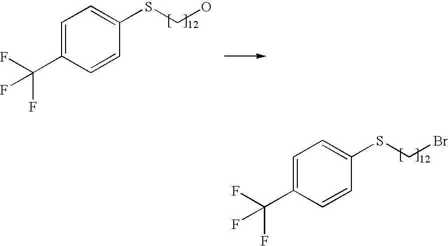

15 g of 12-(4-trifluoromethylphenylsulfanyl)dodecan-1-ol (41.4 mmol, 1 eq) are initially introduced in 300 ml of dry dichloromethane at RT, and 16.3 g of triphenylphosphine (62 mmol, 1.5 eq) are added. The reaction mixture is subsequently cooled to 0° C., and 23.3 g of tetrabromomethane (70.4 mmol, 1.7 eq), dissolved in 70 ml of dichloromethane, are added via a dropping funnel at a controlled rate such that the internal temperature does not exceed 10° C. When the addition is complete, the reaction mixture is slowly warmed to RT. When the reaction is complete (TLC check), the reaction mixture is quenched using saturated NaHCO₃ solution. The organic phase is dried over Na₂SO₄ and evaporated in a rotary evaporator. A brown residue forms, which is adsorbed onto silica gel and purified by column chromatography (PE). The product is a pale-yellowish oil.

¹H-NMR (300 MHz, CDCl₃) δ=7.52-7.45 (m, 4H); 3.50 (t, 2H); 2.92 (t, 2H); 1.87-1.75 (m, 4H); 1.55-1.48 (m, 2H); 1.37-1.16 (m, 10H) ppm.

### Example 7c

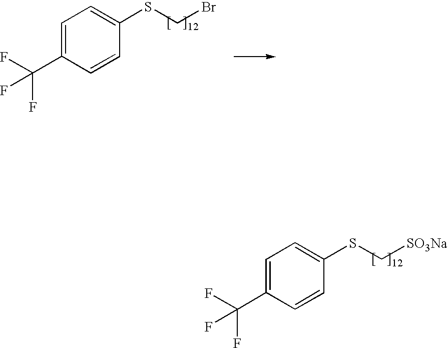

11 g of 1-(12-bromododecylsulfanyl)-4-trifluoromethylbenzene (25.9 mmol, 1 eq) and 3.9 g of sodium sulfite (31 mmol, 1.2 eq) are initially introduced in 40 ml of ethanol and 40 ml of water in a 500 ml one-necked flask and heated to 100° C. The mixture is stirred under reflux overnight (20 hrs), and the conversion is monitored by TLC. When the reaction is complete, the mixture is washed by shaking with a little MTBE/heptane 1:1 in order to remove the starting material and impurities. The aqueous phase is then acidified to pH=0 using a little sulfuric acid and extracted with 15×100 ml of MTBE. The combined organic phases are dried over Na₂SO₄ and filtered, and the solvent is subsequently removed in vacuo, giving a still-moist sulfonic acid, which is taken up in 120 ml of methanol, 1.24 g of solid NaOH are added, and the mixture is boiled under reflux for 1 hour. When the reaction mixture has cooled, the resultant suspension is evaporated, redissolved in MeOH/MTBE 1:1 and filtered through silica gel. The solvent is removed in vacuo. The product is a colourless solid.

¹H-NMR (300 MHz, CDCl₃) δ=7.52-7.45 (m, 4H); 2.92 (t, 2H); 2.39 (t, 2H); 1.87-1.75 (m, 4H); 1.55-1.19 (m, 12H) ppm.

### Example 7d

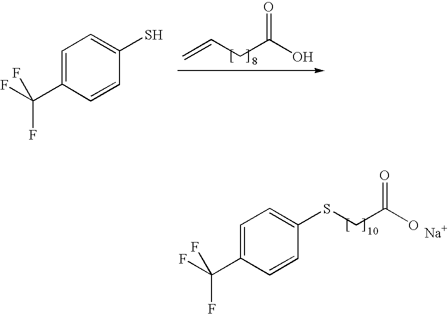

8 g of 4-trifluoromethylbenzenethiol (44.9 mmol) are transferred into the reaction vessel. 60 ml of ethanol in which 8.3 g of undecenoic acid are dissolved are added to the frozen thiol, followed by 80 mg of AIBN. The vessel is sealed using a ground-glass cap and safeguarded cap clip. Dissolved oxygen is removed by means of three freeze-pump-thaw cycles. The reaction mixture is left in an 80° C. oil bath for 2 h. The success of the reaction is determined by means of ¹H- and ¹³C-NMR. To this end, all volatile constituents (=excess thiol, ethanol) in the inert-gas equipment are removed in vacuo at a water-bath temperature of about 40° C. The white residue remaining, which is crystalline at room temperature, is dissolved in methanol at RT, and water is added to the filtered solution until cloudiness remains. The mixture is cooled using acetone/dry ice, giving white crystal-line agglomerates of the product, which are filtered off with suction and dried. The intermediate carboxylic acid is dissolved in 100 ml of methanol, and 1.8 g of NaOH pellets are added, and the mixture is subsequently heated at 65° C. for one hour and then cooled. The product, sodium 11-(4-trifluoromethylphenylsulfanyl)undecanoate, is obtained after the solvent has been removed in a rotary evaporator.

### Example 7e

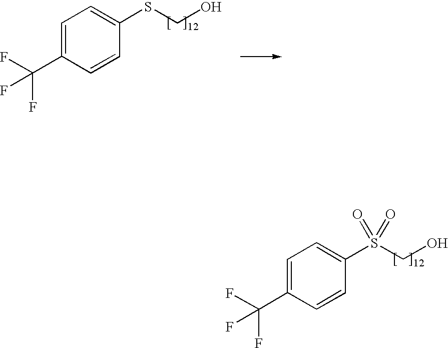

A freshly prepared ice-cold solution of 1.6 g of (NH₄)₆Mo₇O₂₄*4H₂O (1.38 mmol, 0.1 eq) and 80 g of 30% hydrogen peroxide solution (69 mmol, 5 eq) is added dropwise at room temperature to a solution of 5 g of 12-(4-trifluoromethylphenylsulfanyl)dodecan-1-ol (13.8 mmol) in 120 ml of ethanol. The reaction is stirred at room temperature. In order to achieve complete conversion of starting material, the same amount of ammonium molybdate solution is again added. When conversion is complete (TLC check: the sulfoxide forming as intermediate has converted completely to the sulfone), the reaction mixture is diluted with MTBE and washed with dist. water. The aqueous phase is extracted three times with MTBE, and the combined organic phases are washed with saturated NaCl solution, dried over sodium sulfate and evaporated. The crude product is purified by column chromatography.

### Example 8

### Example 8a

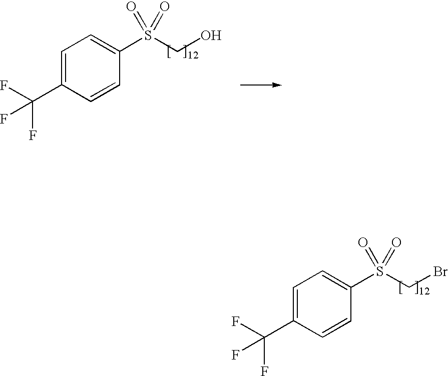

4.4 g of 12-(4-trifluoromethylbenzenesulfonyl)dodecan-1-ol (11.2 mmol, 1 eq) are initially introduced in 100 ml of dry dichloromethane at RT, and 4.4 g of triphenylphosphine (16.8 mmol, 1.5 eq) are added. The reaction mixture is subsequently cooled to 0° C., and 6.3 g of tetrabromomethane (19 mmol, 1.7 eq), dissolved in 10 ml of dichloromethane, are added via a dropping funnel at a controlled rate such that the internal temperature does not exceed 10° C. When the addition is complete, the reaction mixture is slowly warmed to RT. When the reaction is complete (TLC check), the reaction mixture is quenched using saturated NaHCO₃ solution. The organic phase is dried over Na₂SO₄ and evaporated in a rotary evaporator. A brown residue forms, which is adsorbed onto silica gel and purified by column chromatography (PE). The product is a pale-yellowish oil.

### Example 8b

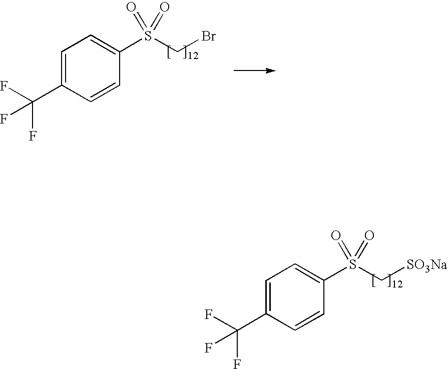

4.5 g of 1-(12-bromododecane-1-sulfonyl)-4-trifluoromethylbenzene (9.9 mmol, 1 eq) and 1.5 g of sodium sulfite (11.9 mmol, 1.2 eq) are initially introduced in 30 ml of ethanol and 30 ml of water in a 100 ml one-necked flask and heated to 100° C. The mixture is stirred under reflux overnight (20 hrs), and the conversion is monitored by TLC. When the reaction is complete, the mixture is washed by shaking with a little MTBE/heptane 1:1 in order to remove the starting material and impurities. The aqueous phase is then concentrated in a freeze drier and freed from inorganic salts by column chromatography with MTBE/MeOH 1/1. The product is a colourless solid, which is dried at 30° C. and 0.1 bar in a vacuum drying cabinet.

### Example 9

### p-Trifluoromethylphenyl keto Substitution

### Example 9a

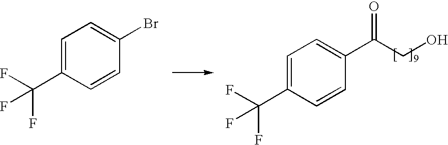

A 15% solution of n-butyllithium in hexane (4 ml, 44 mmol) is slowly added dropwise at −78° C. to a solution of 1-bromo-4-trifluoromethylbenzene (9 g, 40 mmol) in 300 ml of THF. The reaction mixture is warmed to −55° C. over the course of 30 min, then re-cooled to −78° C., and 10.9 g of 10-acetoxydecanoyl chloride (preparation see: G. Goto Chem. Pharm. Bull. 1985, 33, 4422) in 50 ml of THF are subsequently added dropwise. The reaction mixture is warmed to 0° C. over the course of 2 hrs and, when conversion is complete (TLC check), quenched using 1 N HCl solution. The phases are separated, and the organic phase is washed with saturated NaHCO₃ solution and dried over Na₂SO₄. The crude product is taken up in 100 ml of THF, and 1.2 g (50 mmol, 1.25 eq) of LiOH are added, and the mixture is stirred at RT for one hr. When the saponification of the acetoxy group is complete, the mixture is diluted with MTBE and washed with saturated NaCl solution, the phases are separated, and the organic phase is dried over Na₂SO₄. The crude product is purified by column chromatography (PE/MTBE 10/1).

### Example 9b

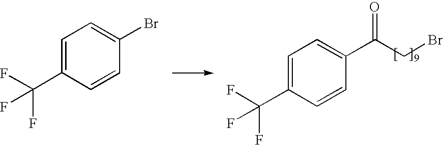

A 15% solution of n-butyllithium in hexane (4 ml, 44 mmol) is slowly added dropwise at −78° C. to a solution of 1-bromo-4-trifluoromethylbenzene (9 g, 40 mmol) in 300 ml of THF. The reaction mixture is warmed to −55° C. over the course of 30 min, then re-cooled to −78° C., and 11.9 g of 10-bromodecanoyl chloride in 50 ml of THF (preparation see: B. L. Feringa J. Am. Chem. Soc. 2004, 126, 13884) are subsequently added dropwise. The reaction mixture is warmed to 0° C. over the course of 2 hrs and, when conversion is complete (TLC check), quenched using 1 N HCl solution. The phases are separated, and the organic phase is washed with saturated NaHCO₃ solution, dried over Na₂SO₄ and evaporated. The crude product is purified by column chromatography (PE/MTBE: 20/1).

### Example 9c

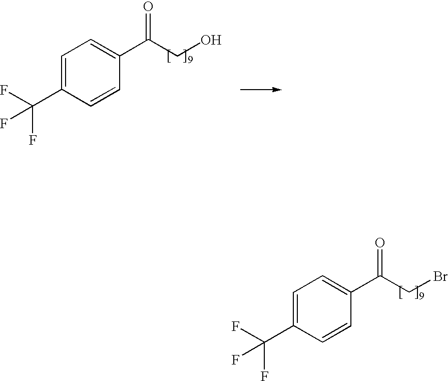

10 g of 10-hydroxy-1-(4-trifluoromethylphenyl)decan-1-one (31.6 mmol; 1 eq) are initially introduced in 200 ml of dry DCM, and 12.4 g of triphenylphosphine (47.4 mmol; 1.5 eq) and subsequently, in portions, 17.8 g of tetrabromomethane (53.7 mmol; 1.7 eq) are added. The reaction is stirred at RT for 24 hours and then quenched using saturated NaHCO₃ soln. The phases are separated, and the organic phase is dried over sodium sulfate and evaporated in a rotary evaporator. The crude product formed is purified by column chromatography with petroleum ether.

### Example 10

### (p-Trifluoromethylphenyl)methanol Substitution

### Example 10a

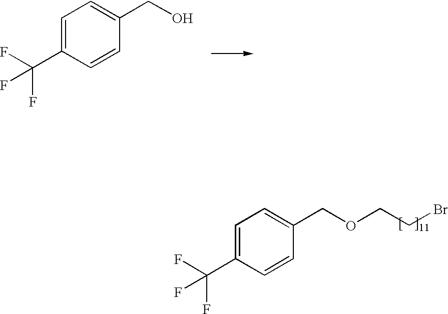

10.0 g of 4-trifluoromethylbenzyl alcohol (56.8 mmol), 16.6 g (62.5 mmol) of 12-bromododecan-1-ol and 16.4 g (62.5 mmol) of triphenylphosphine are initially introduced in 400 ml of THF. 12.2 ml of diisopropyl azodicarboxylate are added dropwise at max. 15° C. with cooling, and the mixture is stirred at room temperature for 24 hrs. The solvent is stripped off in a rotary evaporator, and the crude product is purified by column chromatography (eluent: heptane) over silica gel, giving a yellow oil.

### Example 10b

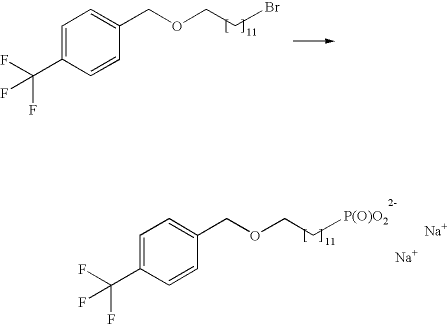

A solution of 6 g of 1-(12-bromododecyloxymethyl)-4-trifluoromethylbenzene (14.2 mmol) in 142 ml of (MeO)₃P is heated under reflux. The reflux condenser is flushed with water at 50° C., and the MeBr forming is expelled by a stream of argon. After the mixture has cooled, the reaction solution is evaporated in vacuo. The crude product is taken up in methanol, aqueous NaOH is added, and the mixture is stirred at RT for 2 hrs. The reaction mixture is re-evaporated and purified by column chromatography (MTBE/MeOH), giving a colourless solid.

¹H-NMR (400 MHz, CDCl₃) δ=7.58-7.48 (m, 4H); 4.48 (s, 2H); 3.45 (t, 2H); 1.70-1.56 (m, 4H); 1.48-1.22 (m, 14H) ppm.

(2,4-Bistrifluoromethylphenyl)methanol can also be converted into the corresponding sulfonate surfactant in accordance with Example 10.

### Example 11

### 8-Sulfooctyl 4-trifluoromethylbenzenesulfonate Sodium Salt

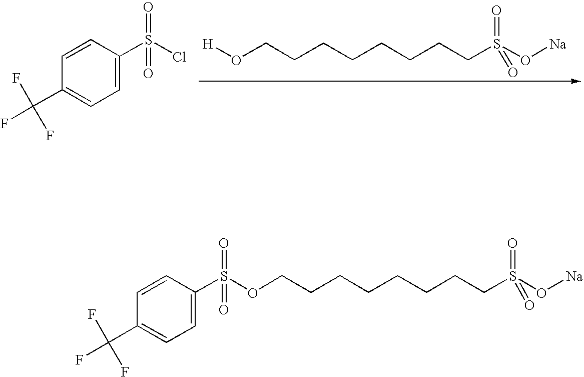

2.50 g of 8-hydroxyoctane-1-sulfonic acid sodium salt (accessible analogously to Varveri, F. S., Nikokavouras, J., Mantaka-Marketou, A. E., Micha-Screttas, M.; Monatsh. Chem.; 120; 1989; 967-972; then sodium salt formation using NaOH) are metered into a solution of 2.50 g of 4-trifluoromethylbenzenesulfonyl chloride in 12 ml of THF under nitrogen with stirring at 0° C., and the mixture is stirred at this temperature for 6 hrs. In order to isolate the product, the reaction mixture is filtered, and the solvent is removed from the eluate forming at 20-30° C. in vacuo, giving a pale-beige, amorphous solid.

¹H-NMR (DMSO; 300 MHz): 1.27-1.29 (4H, m), 1.34 (2H, m), 1.61-1.67 (4H, m), 3.96-3.99 (2H, t), 4.11 (2H, t), 7.91 (2H, m), 8.24 (2H, m)

MS: 399/400 (M⁺-Na⁺).

### Example 12

### Mono(4-trifluoromethylphenyl) octane-1,8-disulfonate Sodium Salt

### A) 4-Trifluoromethylphenyl 8-chlorosulfonyloctane-1-sulfonate

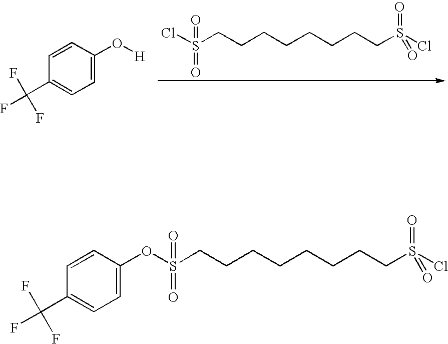

3.4 g of octane-1,8-disulfonyl dichloride (accessible by chlorination of the sodium salts of the disulfonic acid (CAS 168648-99-5; 168648-95-1; 155885-19-1) or of the potassium salt of the disulfonic acid (CAS 118218-21-6), for example using thionyl chloride) and 1.05 g of triethylamine are metered into a solution of 1.62 g of 4-trifluoromethylphenol in 15 ml of dichloromethane under nitrogen at 0° C. with stirring, and the mixture is stirred at this temperature for 3 hrs. In order to isolate the product, the reaction mixture is filtered, and the eluate is freed from solvent in vacuo at 10-20° C. The oil formed is employed as crude product in the next step.

### B) Mono(4-trifluoromethylphenyl) octane-1,8-disulfonate Sodium Salt

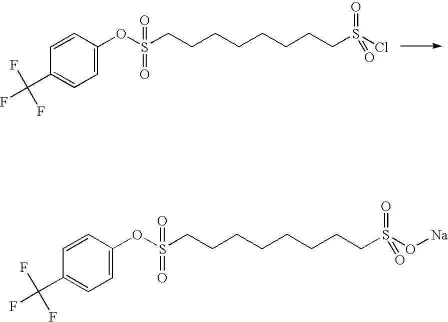

0.4 g of powdered NaOH is metered in small portions over the course of 10 min into a solution of 4.3 g of 4-trifluoromethylphenyl 8-chlorosulfonyloctane-1-sulfonate in 15 ml of THF under nitrogen at 0° C. with stirring, and the mixture is stirred at this temperature for 2 hrs. In order to isolate the product, the solvent is removed at 20° C. in vacuo, giving a pale-yellow, amorphous solid.

¹H-NMR (DMSO; 300 MHz): 1.21-1.24 (2H, m), 1.40 (2H, m), 1.62 (2H, m), 1.88-1.92 (2H, m), 3.15 (2H, t), 4.11 (2H, t), 7.04 (2H, m), 7.36 (2H, m)

MS: 418 (M⁺-Na⁺).

### Example 13

### Mono[10-(4-trifluoromethylphenyl)decyl]sulfate Sodium Salt

### A) 1-((E)-10-benzyloxydec-1-enyl)-4-trifluoromethylbenzene

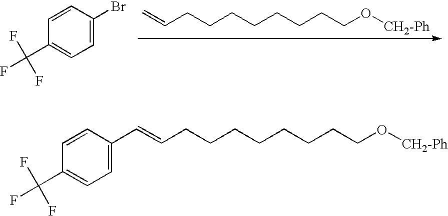

3.1 g of dec-9-enyloxymethylbenzene, 15 mg of Pd(AcAc)₂, 11 mg (0.05 mmol) of DAB-Cy (DAB-Cy=1,4-dicyclohexyldiazabutadiene; CAS 3673-06-1), 4.9 g of Cs₂CO₃ and 16 mg of n-Bu₄NBr are metered into a solution of 2.3 g of 1-bromo-4-trifluoromethylbenzene in 15 ml of N,N-dimethylacetamide under nitrogen and with stirring at 20° C., and the mixture is then warmed to 100° C., stirred at this temperature for 8 hrs and then re-cooled to 20° C.

In order to isolate the product, the reaction mixture is filtered, the solvent is removed in vacuo, and the oil formed is employed as crude product in the next step.

The benzyl ether intermediate can also be obtained from 1-bromo-4-trifluoromethylbenzene using other Heck catalysts, and the benzyl ether intermediate can also be obtained from 1-chloro-4-trifluoromethylbenzene if other catalysts are selected.

### B) 10-(4-Trifluoromethylphenyl)decan-1-ol

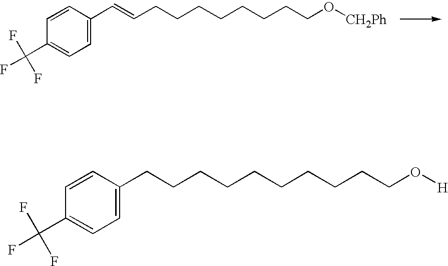

0.4 g of Pd/C (5%) is added to a solution of 3.9 g of 1-((E)-10-benzyloxydec-1-enyl)-4-trifluoromethylbenzene in 20 ml of ethanol under nitrogen at 20° C. with stirring, 1.5 bar of hydrogen are injected, and the mixture is then stirred at 40° C. for 3 hrs. In order to isolate the product, the reaction mixture is filtered, the solvent is removed in vacuo, and the oil formed is employed as crude product in the next step.

### C) Mono[10-(4-trifluoromethylphenyl)decyl]sulfate Sodium Salt

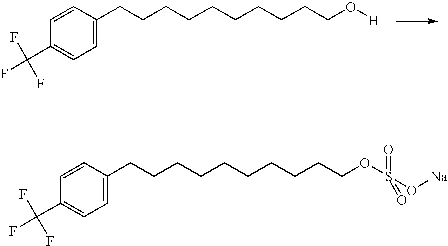

2.0 g of chlorosulfonic acid are carefully metered into a solution of 3.0 g of 10-(4-trifluoromethylphenyl)decan-1-ol in 15 ml of 1,2-dichloroethane under nitrogen at 0° C. with stirring, and the mixture is stirred at this temperature for 0.5 hr, then at 20-25° C. for 2 hrs. In order to isolate the sulfuric acid monoester, the solvent is removed from the reaction mixture in vacuo at 25-40° C. In order to form the sodium salt of the sulfuric acid monoester, 0.4 g of NaOH—dissolved in 10 ml of ethanol—is added to the isolated residue, the mixture is stirred for 30 min, and the solvent is then removed in vacuo at 20-30° C., giving a colourless, amorphous solid.

¹H-NMR (DMSO; 300 MHz): 1.26 (2H, m), 1.60 (2H, m), 1.64 (2H, m), 1.68-1.70 (2H, m), 2.60 (2H, t), 4.11 (2H, t), 7.34 (2H, m), 7.53 (2H, m).

### Example 14

### Example 14a

### m,p,m-Tris(trifluoromethyl) Substitution

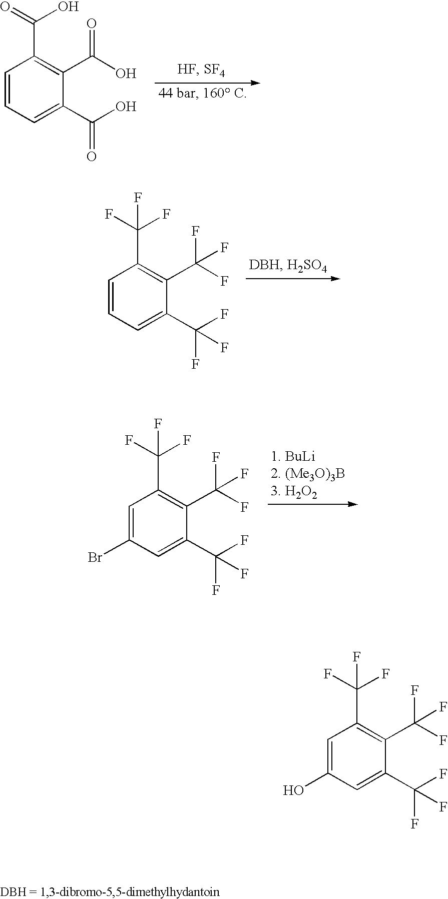

### Example 14b

### Alternative Preparation of the m,p,m-tris(trifluoromethyl) Substitution

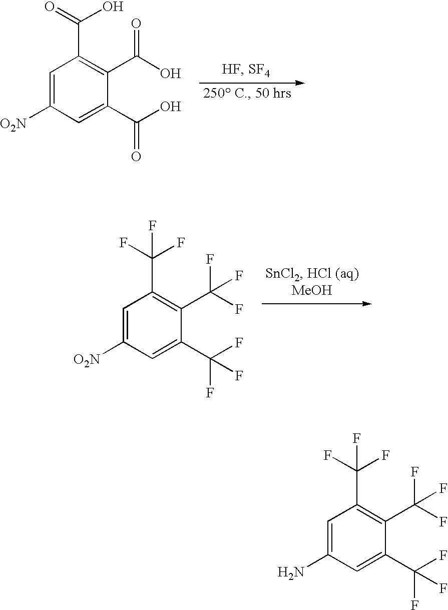

### Example 14c

### m,m-Bis(trifluoromethyl) Substitution

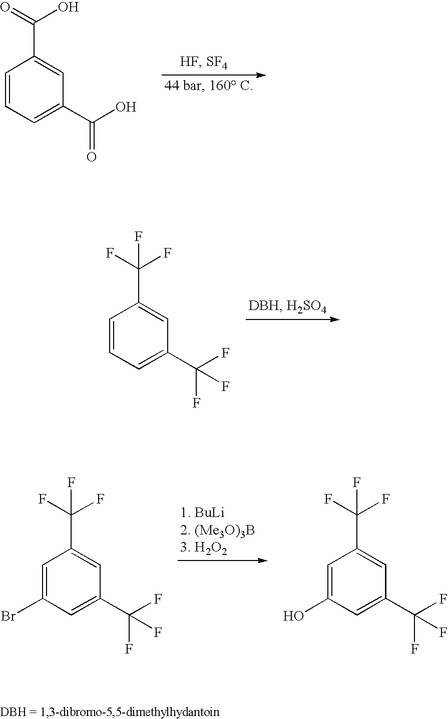

### Example 14d

### Derivatisation of the m,p,m-tris(trifluoromethyl)-Substituted Compounds

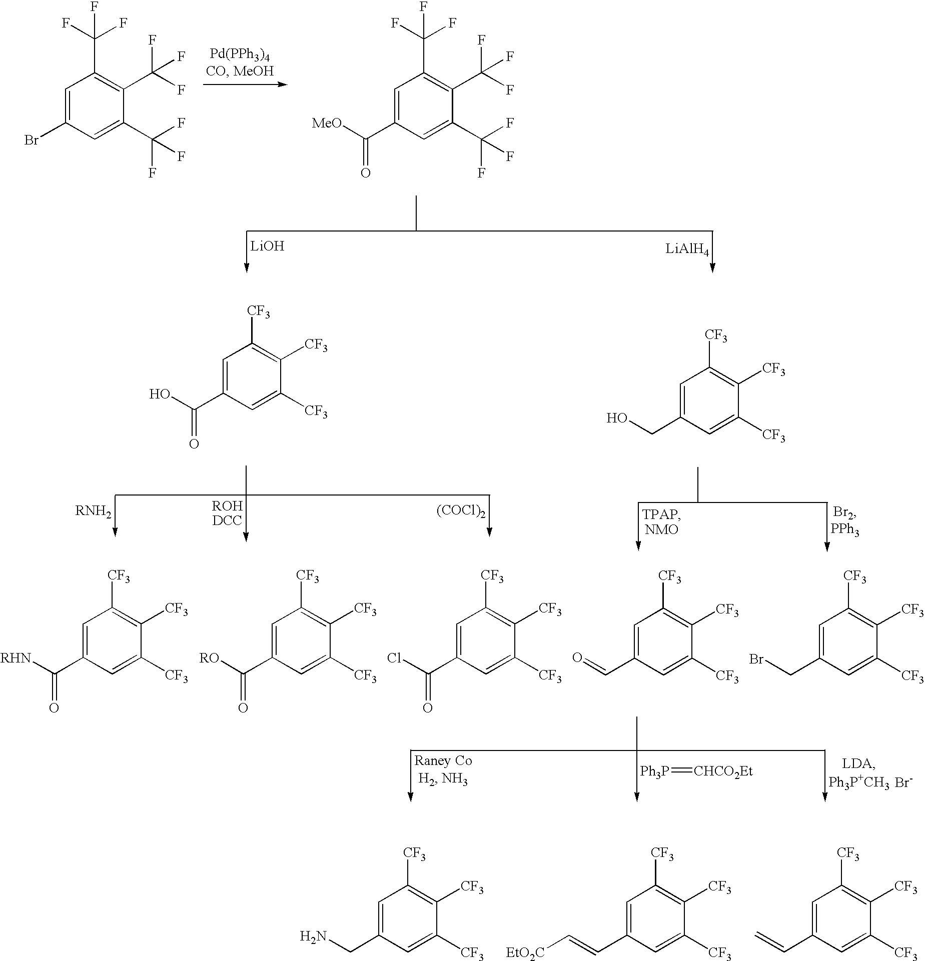

### Example 15

### Pentafluorosulfuranyl Compounds

### Example 15a

### p-Pentafluorosulfuranyl Compounds and Derivatisation Thereof

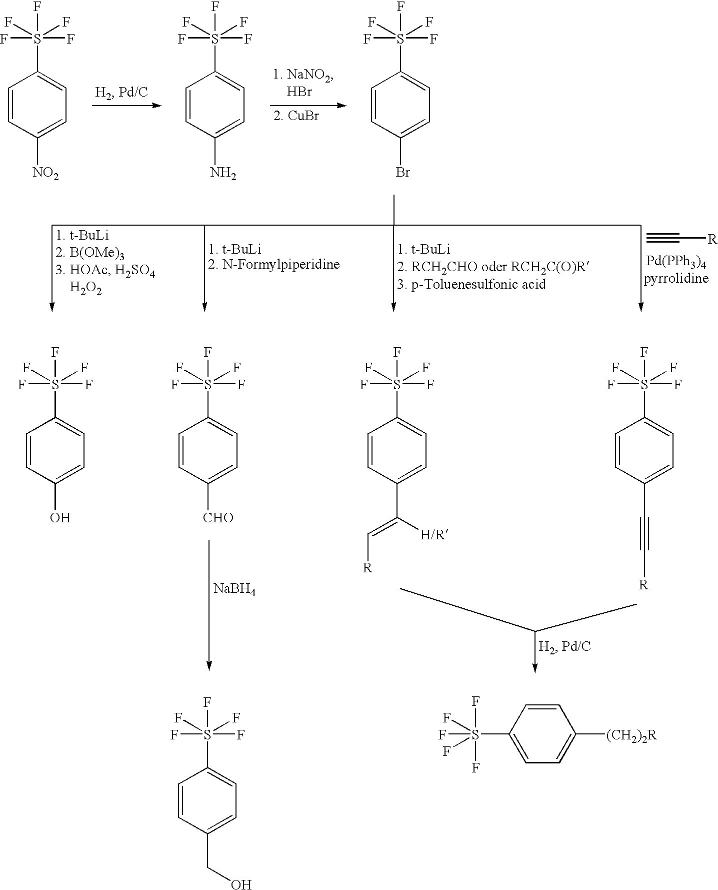

### Example 15b

### m,m-Bispentafluorosulfuranyl Compounds and Derivatisation Thereof

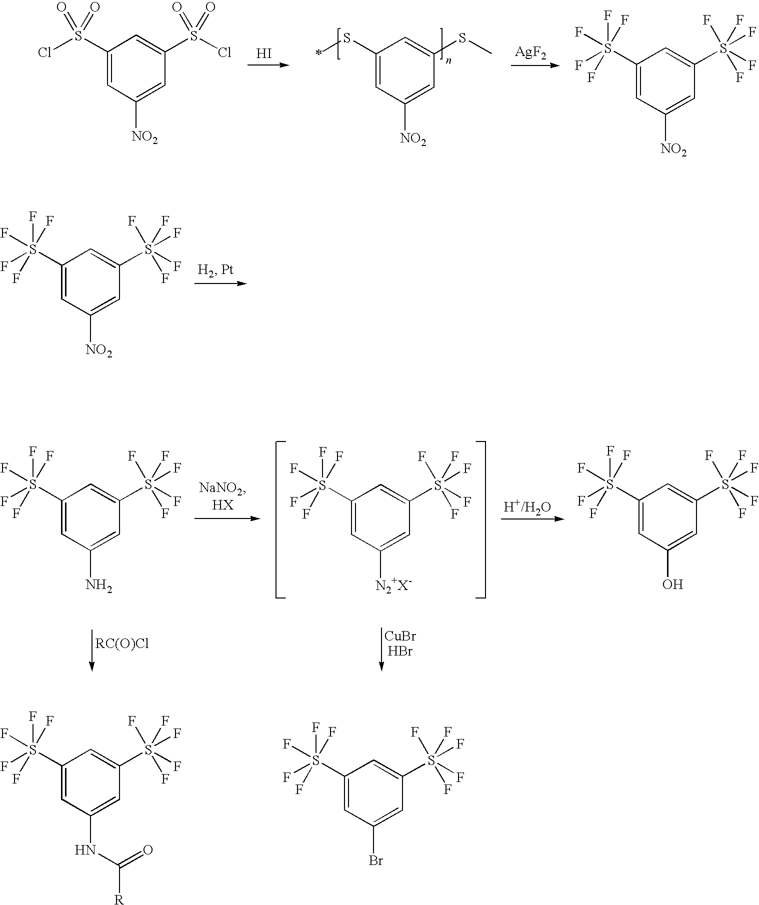

### Example 16

### Trifluoromethyl Thioethers

### Example 16a

### Preparation of the Phenol or Benzoic Acid Derivative as Parent Structure

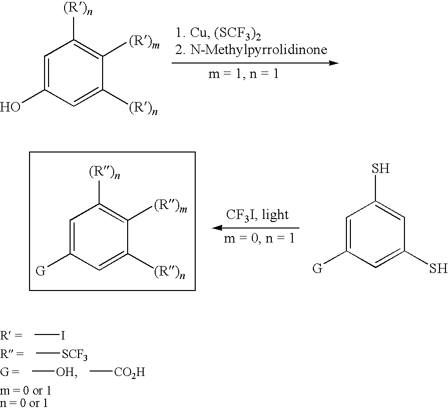

### Example 16b

### Preparation of the Trifluoromethyl Thioethers Via Iodinated Aromatic Compounds and Derivatisation of the Compounds

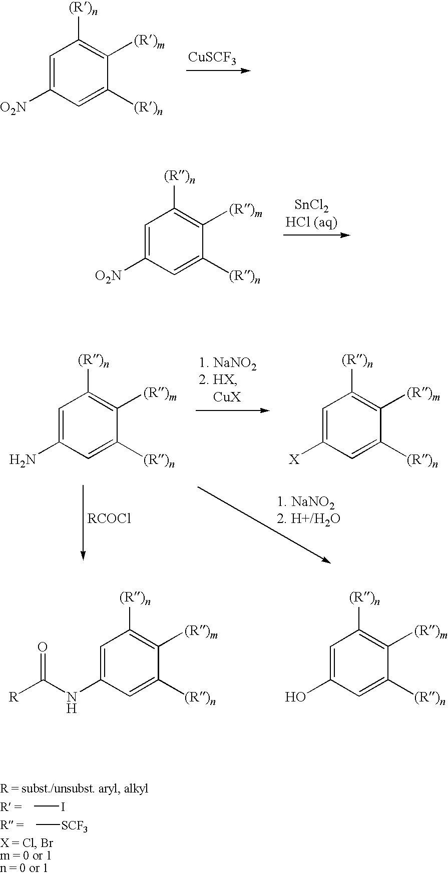

### Example 16c

### Preparation of Tetrakis(Trifluoromethyl Thioethers) from Pentaiodobenzene and Derivatisation of the Compounds

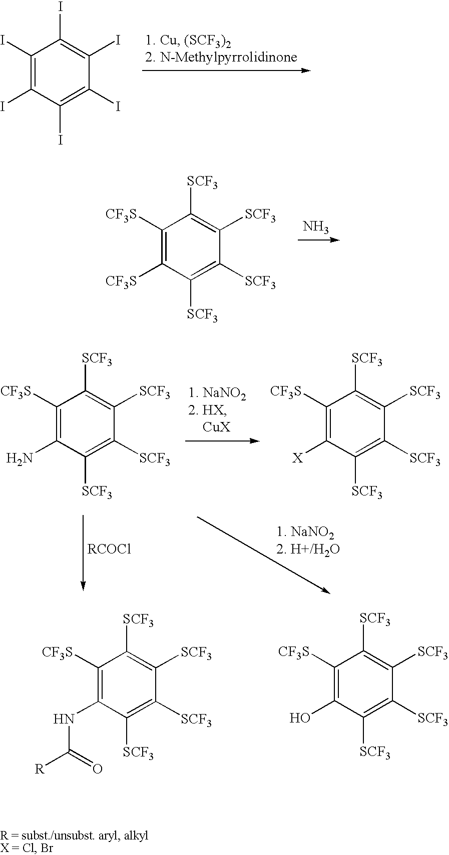

### Example 17

### Preparation of the Perfluoroethyl Thioethers

Instead of the compounds containing CF₃S groups, corresponding compounds containing C₂F₅S groups can also be prepared as described above analogously to Examples 10a-10c if the corresponding reagents for introduction of the trifluoromethyl group are replaced by corresponding pentafluoroethyl reagents in the syntheses. For example, the syntheses succeed if C₂F₅I is employed instead of CF₃I.

### Example 18

### Trifluoromethoxy Compounds

The trifluoromethoxy unit can be obtained by reaction of phenolic OH groups with carbon tetrachloride and hydrogen fluoride and reacted further in accordance with the following scheme:

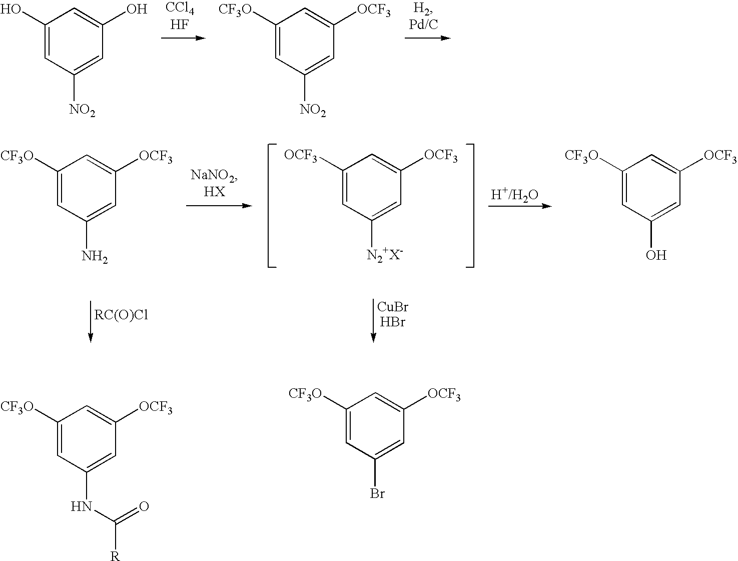

### Example 19

### Determination of the Biochemical Degradability

The biochemical degradability of the compounds is determined by the Zahn-Wellens test corresponding to the European Commission publication: Classification, Packaging and Labelling of Dangerous Substances in the European Union, Part II—Testing Methods, Annex V—Methods for the Determination of Physico-Chemical Properties, Toxicity and Ecotoxicity, Part B, Biochemical Degradability—Zahn-Wellens Test (C.9.), January 1997, pages 353-357.

| | |
|---|---|
| Batch volume: | 1.5 l |
| Activated sludge concentration: | 1 g of solids/l |
| Origin of the sludge: | treatment plant of Merck KGaA; |
| | Darmstadt (not adapted) |
| Amount of test substances used: | about 100 to 200 mg/l as DOC |
| Aeration: | with purified air |
| Work-up of the samples: | filtration (medium-hard filter) |
| Determination of the DOC: | by the difference method using a |
| | Dimatec instrument |

Further details on the method are given in the above publication and also the OECD Guideline for the testing of chemicals, section 3, degradation and accumulation, method 302 B, page 1-8, adopted: 17.07.92, the contents of which in this respect expressly belong to the disclosure content of the present application.

In addition, besides the degradation of the compound per se in the test, the degradation of the fluorine-containing groups is also observed via a fluoride determination:

| | |
|---|---|
| Method: | ion chromatography |
| Instrument: | Dionex 120 |
| Detector type: | conductivity detector |
| Column: | AS9HC |
| Eluent: | sodium carbonate solution, 9 mmol/l |
| Flow rate: | 1 ml/min |
| Literature: | EN ISO 10304-2 |

### Example 20

### Determination of the Surface Tension

| | |
|---|---|
| Instrument: | Krüss tensiometer (model K12) |
| Temperature of the | 20° C. |
| measurement solutions: | |
| Measurement module employed: | ring |
| Concentration of the | about 0.5 to 3.0 g/l in deionised |
| measurement solutions: | water |

Further details on the method are given by the European Commission publication: Classification, Packaging and Labelling of Dangerous Substances in the European Union, Part II—Testing Methods, Annex V—Methods for the Determination of Physico-Chemical Properties, Toxicity and Ecotoxicity, Part A, Surface Tension (A.5), January 1997, pages 51-57, and also the OECD Guideline for the testing of chemicals, section 1, physical-chemical properties, method 115, page 1-7, adopted: 27.07.95, the contents of which in this respect expressly belong to the disclosure content of the present application.

1. A method of reducing the surface energy of a surface comprising applying a compound having an end group Y, where Y stands for 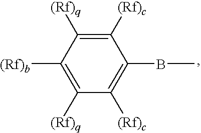
where
Rf stands for CF₃—(CH₂)ᵣ—, CF₃—(CH₂)ᵣ—O—, CF₃—(CH₂)ᵣ—S—, CF₃CF₂—S—, SF₅—(CH₂)ᵣ—, [CF₃—(CH₂)ᵣ]₂N—, [CF₃—(CH₂)ᵣ]NH— or (CF₃)₂N—(CH₂)ᵣ—,
B stands for a single bond, O, NH, NR, CH₂, C(O)—O, C(O), S, CH₂—O, O—C(O), N—C(O), C(O)—N, O—C(O)—N, N—C(O)—N, O—SO₂ or SO₂—O,
R stands for alkyl having 1 to 4 C atoms,
b stands for 0 or 1 and c stands for 0 or 1,
q stands for 0 or 1, where at least one radical from b and q stands for 1, and
r stands for 0, 1, 2, 3, 4 or 5,
as end group in surface-active compounds.

2. A method according to claim 1, characterised in that the end group Y in the surface-active compounds is bonded to a saturated or unsaturated, optionally aromatic, optionally heteroatom-containing, branched or unbranched, optionally substituted hydrocarbon unit.

3. A method according to claim 1, characterised in that the end group Y occurs a number of times in the surface-active compound, and the surface-active compound is preferably an oligomer or polymer.

4. A method according to claim 1, characterised in that the end group Y occurs only once, twice or three times in the surface-active compound, and the surface-active compound is preferably a low-molecular-weight compound of the formula I
Y-spacer-X I
where
Y stands for 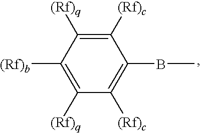
where
Rf stands for CF₃—(CH₂)ᵣ—, CF₃—(CH₂)ᵣ—O—, CF₃—(CH₂)ᵣ—S—, CF₃CF₂—S—, SF₅—(CH₂)ᵣ—, [CF₃—(CH₂)ᵣ]₂N—, [CF₃—(CH₂)ᵣ]NH— or (CF₃)₂N—(CH₂)ᵣ—,
B stands for a single bond, O, NH, NR, CH₂, C(O)—O, C(O), S, CH₂—O, O—C(O), N—C(O), C(O)—N, O—C(O)—N, N—C(O)—N, O—SO₂ or SO₂—O,
R stands for alkyl having 1 to 4 C atoms,
b stands for 0 or 1 and c stands for 0 or 1,
q stands for 0 or 1, where at least one radical from b and q stands for 1,
r stands for 0, 1, 2, 3, 4 or 5,
spacer stands for a saturated or unsaturated, optionally aromatic, optionally heteroatom-containing, branched or unbranched, optionally substituted hydrocarbon unit, and
X stands for a cationic, nonionic, amphoteric or anionic polar group or a polymerisable group or a functional group.

5. A method according to claim 1, characterised in that the end group Y is present in compounds of the formulae Ia to Ig
Y—(CH₂)ₙ—X Ia
Y—CH₂—CH(Hal)—(CH₂)₍ₙ₋₁₎—X Ib
Y—(CH₂)ₙ₋₁CH═CH—(CH₂)_{(n′-1)}—X Ic
Y—(CH₂)ₙ₋₁—Ar—(CH₂)_{(n′-1)}—X Id
Y—(CH₂)ₙ₋₁—C≡C—(CH₂)ₙ—X Ie
Y—(CH₂)ₙ-Q-(CH₂)_{n′}—X If
Y—[(CH₂)_{z}—O]—[(CH₂)ₓ—O]_{y}—(CH₂)ₜ—X Ig
in which Y stands for 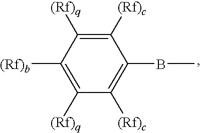
where
Rf stands for CF₃—(CH₂)ᵣ—, CF₃—(CH₂)ᵣ—O—, CF₃—(CH₂)ᵣ—S—, CF₃CF₂—S—, SF₅—(CH₂)ᵣ—, [CF₃—(CH₂)ᵣ]₂N—, [CF₃—(CH₂)ᵣ]NH— or (CF₃)₂N—(CH₂)ᵣ—,
B stands for a single bond, O, NH, NR, CH₂, C(O)—O, C(O), S, CH₂—O, O—C(O), N—C(O), C(O)—N, O—C(O)—N, N—C(O)—N, O—SO₂ or SO₂—O,
R stands for alkyl having 1 to 4 C atoms,
b stands for 0 or 1 and c stands for 0 or 1,
q stands for 0 or 1, where at least one radical from b and q stands for 1,
r stands for 0, 1, 2, 3, 4 or 5,
x and t, independently of one another, stand for 1-5,
y stands for 0-5,
z stands for 1-12,
n and n′, independently of one another, stand for an integer from the range 1 to 30,
X stands for a cationic, nonionic, amphoteric or anionic polar group or a polymerisable group,
(Hal) stands for F, Cl, Br or I,
Q stands for O, S or N, and
Ar stands for aryl,
and corresponding salts of the compounds of one of the formulae Ia to Ig.

6. A method according to claim 1, characterised in that q stands for 0 and all c and b each stand for 1.

7. A method according to claim 1, characterised in that all b and q each stand for 0 and c in each case stands for 1.

8. A method according to claim 1, characterised in that all c and q each stand for 0 and b stands for 1.

9. A method according to claim 1, characterised in that r stands for 0, 1 or 2, preferably for 0.

10. A method according to claim 1, characterised in that the surface-active compound is employed as hydrophobicising agent or oliophobicising agent, in particular in the surface modification of textiles, paper, glass, porous building materials or adsorbents.

11. A method according to claim 1, characterised in that the surface-active compound is used as additive in preparations for surface coating, such as printing inks, paints, coatings, photographic coatings, special coatings for semiconductor photolithography, such as photoresists, top antireflective coatings, bottom antireflective coatings, or in additive preparations for addition to corresponding preparations.

12. A method according to claim 1, characterised in that the surface-active compound is used as antistatic, in particular in the treatment of textiles, such as clothing, carpets and carpeting, upholstery in furniture and automobiles, non-woven textile materials, leather goods, papers and cardboard articles, wood and wood-based materials, mineral substrates, such as stone, cement, concrete, plaster, ceramics, such as glazed and unglazed tiles, earthenware, porcelain, and glasses, and for plastics and metallic substrates.

13. A method according to claim 1, characterised in that the surface-active compound is used as foam stabiliser and/or for supporting film formation, in particular in fire-extinguishing foams.

14. A method according to claim 1, characterised in that the surface-active compound is used as interface promoter or emulsifier, in particular for the preparation of fluoropolymers.

15. A method according to claim 1, characterised in that the surface-active compound is used as antimicrobial active compound, in particular as reagent for antimicrobial surface modification.

16. A method according to claim 1, characterised in that X stands for an anionic polar group selected from —COOM, —SO₃M, —OSO₃M, —PO₃M₂, —OPO₃M₂, —(OCH₂CHR)ₘ—O—(CH₂)ₒ—COOM, —(OCH₂CHR)ₘ—O—(CH₂)ₒ—SO₃M, —(OCH₂CHR)ₘ—O—(CH₂)ₒ—OSO₃M, —(OCH₂CHR)ₘ—O—(CH₂)ₒ—PO₃M₂, —(OCH₂CHR)ₘ—O—(CH₂)ₒ—OPO₃M₂,
where M stands for H or an alkali metal ion, preferably Li⁺, Na⁺ or K⁺, or NH₄⁺ or tetra-C₁₋₆-alkylammonium or tetra-C₁₋₆-alkylphosphonium,
R stands for H or C₁₋₄-alkyl,
m stands for an integer from the range from 1 to 1000, and
o stands for an integer selected from 1, 2, 3 or 4.

17. A method according to claim 1, characterised in that X stands for a cationic polar group selected from —NR¹R²R³⁺Z⁻, —PR¹R²R³⁺Z⁻, 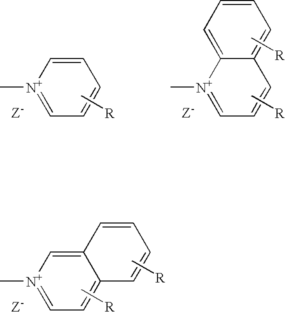
where R stands for H or C₁₋₄-alkyl in any desired position,
Z⁻ stands for Cl⁻, Br⁻, I⁻, CH₃SO₃⁻, CF₃SO₃⁻, CH₃PhSO₃⁻, PhSO₃⁻,
R¹, R² and R³ each, independently of one another, stand for H, C₁₋₃₀-alkyl, Ar or —CH₂Ar, and
Ar stands for an unsubstituted or mono- or polysubstituted aromatic ring or condensed ring system having 6 to 18 C atoms, in which, in addition, one or two CH groups may be replaced by N.

18. A method according to claim 1, characterised in that X stands for a nonionic polar group selected from —Cl, —Br, —I, —(OCH₂CHR)ₘ—OH, —(OCH₂CHR)ₘ—SH, —O-(glycoside)ₒ, —(OCH₂CHR)ₘ—OCH₂—CHOH—CH₂—OH, —(OCH₂CHR)ₘ—OCH₂Ar(—NCO)ₚ, —(OCH₂CHR)ₘ—OAr(—NCO)ₚ, —SiR¹R²Z, —SiR¹Z₂, —SiZ₃, —R²—COZ, —(OCH₂CHR)ₘ—SO₂CH═CH₂, —SO₂Z, 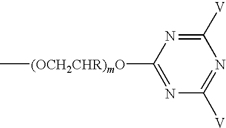
m stands for an integer from the range from 0 to 1000,
n stands for 0 or 1,
o stands for an integer from the range from 1 to 10,
p stands for 1 or 2,
R stands for H or C₁₋₄-alkyl,
R¹ and R² each, independently of one another, stand for C₁₋₃₀-alkyl, Ar or —CH₂Ar,
Ar stands for an unsubstituted or mono- or polysubstituted aromatic ring or condensed ring system having 6 to 18 C atoms, in which, in addition, one or two CH groups may be replaced by N,
glycoside stands for an etherified carbohydrate, preferably for a mono-, di-, tri- or oligoglucoside,
all Z each, independently of one another, stand for —H, —Cl, —F, —NR¹R², —OR¹ or —N-imidazolyl, and
V stands for Cl or F.

19. A method according to claim 1, characterised in that X stands for a polymerisable group selected from —(OCH₂CHR)ₘOCOCR═CH₂, —(OCH₂CHR)ₘ—OCR═CH₂, 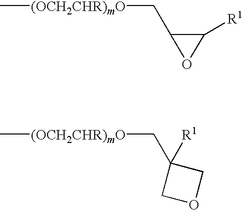
where m stands for an integer from the range from 0 to 1000, and R and R¹ stand for H or C₁₋₄-alkyl or Y-spacer-(OCH₂CHR)ₘ—OCH₂—.

20. A method according to claim 1, characterised in that X stands for a functional group selected from —CR²═CR³R⁴, —C≡CR², —CHO, —C(═O)CH₃, —COOH, —OH, —SH, —Cl, —Br, —I, where R², R³ and R⁴ each, independently of one another, stand for H or Y-spacer- or C₁₋₄-alkyl.

21. A method according to claim 1, characterised in that X stands for an amphoteric group selected from the functional groups of acetyldiamines, N-alkylamino acids, betaines, amine oxides or corresponding derivatives thereof.

22. Compound of one of the formulae Ia to Ig
Y—(CH₂)ₙ—X Ia
Y—CH₂—CH(Hal)—(CH₂)₍ₙ₋₁₎—X Ib
Y—(CH₂)ₙ₋₁CH═CH—(CH₂)_{(n′-1)}—X Ic
Y—(CH₂)ₙ₋₁—Ar—(CH₂)_{(n′-1)}⁻X Id
Y—(CH₂)ₙ₋₁—C≡C—(CH₂)ₙ—X Ie
Y—(CH₂)ₙ-Q-(CH₂)_{n′}—X If
Y—[(CH₂)_{z}—O]—[(CH₂)ₓ—O]_{y}—(CH₂)ₜ—X Ig
in which Y stands for 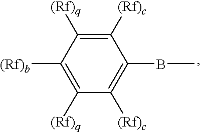
where
Rf stands for CF₃—(CH₂)ᵣ—, CF₃—(CH₂)ᵣ—O—, CF₃—(CH₂)ᵣ—S—, CF₃CF₂—S—, SF₅—(CH₂)ᵣ—, [CF₃—(CH₂)ᵣ]₂N—, [CF₃—(CH₂)ᵣ]NH— or (CF₃)₂N—(CH₂)ᵣ—,
B stands for a single bond, O, NH, NR, CH₂, C(O)—O, C(O), S, CH₂—O, O—C(O), N—C(O), C(O)—N, O—C(O)—N, N—C(O)—N, O—SO₂ or SO₂—O,
R stands for alkyl having 1 to 4 C atoms,
b stands for 0 or 1 and c stands for 0 or 1,
q stands for 0 or 1, where at least one radical from b and q stands for 1,
r stands for 0, 1, 2, 3, 4 or 5,
x and t, independently of one another, stand for 1-5,
y stands for 0-5,
z stands for 1-12,
n and n′, independently of one another, stand for an integer from the range 1 to 30,
X stands for a cationic, nonionic, amphoteric or anionic polar group or a polymerisable group,
(Hal) stands for F, Cl, Br or I,
Q stands for O, S or N, and
Ar stands for aryl,
and corresponding salts of the compounds of one of the formulae Ia to Ig.

23. Compound according to claim 22, characterised in that c stands for 0 and all q and b each stand for 1.

24. Compound according to claim 22, characterised in that all c and b each stand for 0 and q in each case stands for 1.

25. Compound according to claim 22, characterised in that all c and q each stand for 0 and b stands for 1.

26. Compound according to claim 22, characterised in that r stands for 0, 1 or 2, preferably for 0.

27. Compound according to claim 22, characterised in that X stands for an anionic polar group selected from —COOM, —SO₃M, —OSO₃M, —PO₃M₂, —OPO₃M₂, —(OCH₂CHR)ₘ—O—(CH₂)ₒ—COOM, —(OCH₂CHR)ₘ—O—(CH₂)ₒ—SO₃M, (OCH₂CHR)ₘ—O—(CH₂)ₒ—OSO₃M, (OCH₂CHR)ₘ—O—(CH₂)ₒ—PO₃M₂, —(OCH₂CHR)ₘ—O—(CH₂)ₒ—OPO₃M₂,
where M stands for H or an alkali metal ion, preferably Li⁺, Na⁺ or K⁺, or NH₄⁺ or tetra-C₁₋₆-alkylammonium or tetra-C₁₋₆-alkylphosphonium,
R stands for H or C₁₋₄-alkyl,
m stands for an integer from the range from 1 to 1000, and
o stands for an integer selected from 1, 2, 3 or 4.

28. Compound according to claim 22, characterised in that X stands for a cationic polar group selected from —NR¹R²R³⁺Z⁻, —PR¹R²R³⁺Z⁻, 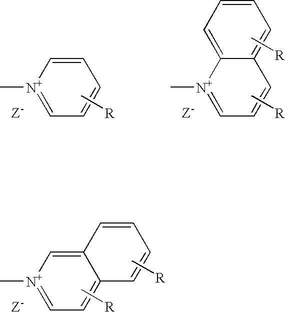
where R stands for H or C₁₋₄-alkyl in any desired position,
Z⁻ stands for Cl⁻, Br⁻, I⁻, CH₃SO₃⁻, CF₃SO₃⁻, CH₃PhSO₃⁻, PhSO₃⁻,
R¹, R² and R³ each, independently of one another, stand for H, C₁₋₃₀-alkyl, Ar or —CH₂Ar, and
Ar stands for an unsubstituted or mono- or polysubstituted aromatic ring or condensed ring system having 6 to 18 C atoms, in which, in addition, one or two CH groups may be replaced by N.

29. Compound according to claim 22, characterised in that X stands for a nonionic polar group selected from —Cl, —Br, —I, —(OCH₂CHR)ₘ—OH, —(OCH₂CHR)ₘ—SH, —O-(glycoside)ₒ, —(OCH₂CHR)ₘ—OCH₂—CHOH—CH₂—OH, —(OCH₂CHR)ₘ—OCH₂Ar(—NCO)ₚ, —(OCH₂CHR)ₘ—OAr(—NCO)ₚ, —SiR¹R²Z, —SiR¹Z₂, —SiZ₃, —COZ, (OCH₂CHR)ₘ—SO₂CH═CH₂, —SO₂Z, 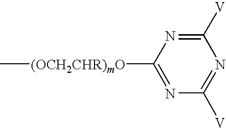
m stands for an integer from the range from 0 to 1000,
n stands for 0 or 1,
o stands for an integer from the range from 1 to 10,
p stands for 1 or 2,
R stands for H or C₁₋₄-alkyl,
R¹ and R² each, independently of one another, stand for C₁₋₃₀-alkyl, Ar or —CH₂Ar,
Ar stands for an unsubstituted or mono- or polysubstituted aromatic ring or condensed ring system having 6 to 18 C atoms, in which, in addition, one or two CH groups may be replaced by N,
glycoside stands for an etherified carbohydrate, preferably for a mono-, di-, tri- or oligoglucoside,
all Z each, independently of one another, stand for —H, —Cl, —F, —NR¹R², —OR¹, —N-imidazolyl, and
V stands for Cl or F.

30. Compound according to claim 22, characterised in that X stands for a polymerisable group selected from —(OCH₂CHR)ₘOCOCR═CH₂, —(OCH₂CHR)ₘ—OCR═CH₂, 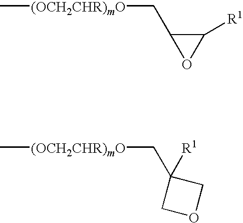
where m stands for an integer from the range from 0 to 1000, and R and R¹ stand for H or C₁₋₄-alkyl or Y-spacer-(OCH₂CHR)ₘ—OCH₂—.

31. Compound according to claim 22, characterised in that X stands for a functional group selected from —CR²═CR³R⁴, —C≡CR², —CHO, —C(═O)CH₃, —COOH, —OH, —SH, —Cl, —Br, —I, where R², R³ and R⁴ each, independently of one another, stand for H or Y-spacer- or C₁₋₄-alkyl.

32. Compound according to claim 22, characterised in that X stands for an amphoteric group selected from the functional groups of acetyldiamines, N-alkylamino acids, betaines, amine oxides or corresponding derivatives thereof.

33. Compound according to claim 22, characterised in that n stands for an integer from the range 4 to 28, preferably from the range 4 to 16.

34. Process for the preparation of a compound of the formula I according to claim 4, characterised in that the synthesis is carried out via the intermediate of a compound of the formula II 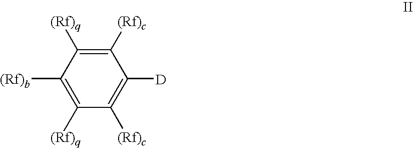
where
Rf stands for CF₃—(CH₂)ᵣ—, CF₃—(CH₂)ᵣ—O—, CF₃—(CH₂)ᵣ—S—, CF₃CF₂—S—, SF₅—(CH₂)ᵣ—, [CF₃—(CH₂)ᵣ]₂N—, [CF₃—(CH₂)ᵣ]NH— or (CF₃)₂N—(CH₂)ᵣ—,
D stands for OH, NH₂ or Br,
b stands for 0 or 1 and c stands for 0 or 1,
q stands for 0 or 1, where at least one radical from b and q stands for 1, and
r stands for 0, 1, 2, 3, 4 or 5.

35. Composition comprising at least one surface-active compound containing at least one end group Y, where Y stands for 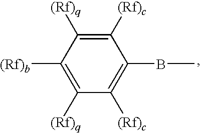
where
Rf stands for CF₃—(CH₂)ᵣ—, CF₃—(CH₂)ᵣ—O—, CF₃—(CH₂)ᵣ—S—, CF₃CF₂—S—, SF₅—(CH₂)ᵣ—, [CF₃—(CH₂)ᵣ]₂N—, [CF₃—(CH₂)ᵣ]NH— or (CF₃)₂N—(CH₂)ᵣ—,
B stands for a single bond, O, NH, NR, CH₂, C(O)—O, C(O), S, CH₂—O, O—C(O), N—C(O), C(O)—N, O—C(O)—N, N—C(O)—N, O—SO₂ or SO₂—O,
R stands for alkyl having 1 to 4 C atoms,
b stands for 0 or 1 and c stands for 0 or 1,
q stands for 0 or 1, where at least one radical from b and q stands for 1, and
r stands for 0, 1, 2, 3, 4 or 5, and a vehicle which is suitable for the particular application and optionally further specific active compounds.

36. Composition according to claim 35, characterised in that the composition is a paint or coating composition, fire-extinguishing composition, lubricant, washing or cleaning composition, de-icer or hydrophobicising agent for textile finishing or glass treatment, printing ink, paint, photographic coating, special coating for semiconductor photolithography, such as a photoresist, top antireflective coating, bottom antireflective coating, or an additive preparation for addition to corresponding preparations.

37. Process for the preparation of a composition according to claim 35, characterised in that the surface-active compound is mixed with the vehicle which is suitable for the particular application and optionally further specific active compounds.
